(19) United States
(12) Patent Application Publication
(10) Pub. No.: US 2026/0070899 A1
(43) Pub. Date: Mar. 12, 2026

(54) N-(2-(4-CYANOTHIAZOLIDIN-3-YL)-2-OXOETHYL)-QUINOLINE-4-CARBOXAMIDES

(71) Applicant: AstraZeneca AB, Södertälje (SE)

(72) Inventors: Jonas BRÅNALT, Gothenburg (SE); Jakob DANIELSSON, Gothenburg (SE); Marlene FREDENWALL, Gothenburg (SE); Björn HOLM, Gothenburg (SE); Maria JOHANSSON, Gothenburg (SE); Anneli NORDQVIST, Gothenburg (SE); Marianne SWANSON, Gothenburg (SE); Anna TOMBERG, Gothenburg (SE); Susanne WINIWARTER, Gothenburg (SE)

(21) Appl. No.: 18/871,228

(22) PCT Filed: Jun. 20, 2023
(86) PCT No.: PCT/EP2023/066561

§ 371 Date: Dec. 3, 2024

Related U.S. Application Data
(60) Provisional application No. 63/366,699, filed on Jun. 21, 2022.

(51) Int. Cl. C07D 417/12; A61K 31/4709; A61K 31/5377; C07D 417/14

(52) U.S. Cl. CPC C07D 417/12; A61K 31/4709; A61K 31/5377; C07D 417/14

(57) ABSTRACT

Compounds having the structure of Formula (I) and pharmaceutically acceptable salts thereof, wherein X¹, R¹, R², R³, R⁴, R⁵, and R⁶ are as defined in the specification; pharmaceutical compositions comprising such compounds and salts; use of such compounds and salts to treat or prevent Prolyl endopeptidase fibroblast activation protein (FAP)-mediated conditions; kits comprising such compounds and salts; and methods for manufacturing such compounds and salts.

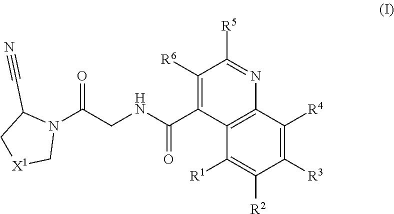

### CROSS-REFERENCE TO RELATED PATENT APPLICATION

This specification claims the benefit of priority to U.S. Provisional Patent Application No. 63/366,699 (filed 21 Jun. 2022). The entire text of the above-referenced patent application is incorporated by reference into this specification.

### FIELD

The present disclosure relates generally to N-(2-(4-cyanothiazolidin-3-yl)-2-oxoethyl)-quinoline-4-carboxamides and pharmaceutically acceptable salts thereof. The specification further relates to pharmaceutical compositions comprising such compounds and salts; use of such compounds and salts to treat or prevent Prolyl endopeptidase fibroblast activation protein (FAP)-mediated conditions; kits comprising such compounds and salts; and methods for manufacturing such compounds and salts.

### BACKGROUND

FAP, a type II transmembrane serine protease, is expressed by fibroblast like cells involved in tissue remodeling and healing. In the context of non-alcoholic steatohepatitis (NASH), FAP is upregulated on the cell surface of activated hepatic stellate cells involved in the fibrosis formation (Hepatology 1999, 29, 1768), a major aspect of NASH that predicts disease outcome (Gastroenterology 2020, 158, 1611). FAP also can be present as a shedded plasma protease. Increased levels of circulating FAP are associated with NASH disease severity (Diabetes Res Clin Pract 2015, 108, 466).

FAP has a consensus cleavage motif after Gly-Pro and exhibits both endopeptidase and exopeptidase activity. Known enzymatic activities include cleavage of collagens (Hepatology 1999, 29, 1768), α2-antiplasmin (α2AP) (Blood 2004 103, 3783), and fibroblast growth factor 21 (FGF21) (Biochem J 2016, 473, 605). FAP activity at the cell surface of activated fibroblasts (including cleavage of collagens) generates a pro-fibrotic environment. FAP cleavage of α2AP gives a more efficient cross-linking of α2AP to fibrin and results in reduced fibrin clearance. FAP cleavage of FGF21 inactivates FGF21 metabolic effects (Biochem J 2016, 473, 605). All these activities are associated with a worsening of NASH disease and inhibiting FAP has the potential to treat NASH and other conditions by affecting multiple mechanisms.

Inhibition of FAP activity is a presently unexploited therapeutic approach for treating NASH and other diseases associated with such activity. No approved pharmacological agents that inhibit FAP activity generally, or that inhibit FAP activity specifically, are currently available. Accordingly, there is a need for FAP inhibitors, particularly FAP inhibitors that have pharmacologically appropriate selectivity and bioavailability and therefore are suitable for administration to a subject in need of such treatment. The present disclosure addresses this large unmet need by providing such compounds together with corresponding pharmaceutical compositions and methods for the treatment or prevention of NASH and related conditions.

### SUMMARY

In one aspect, the present disclosure provides compounds having the structure of Formula (I):

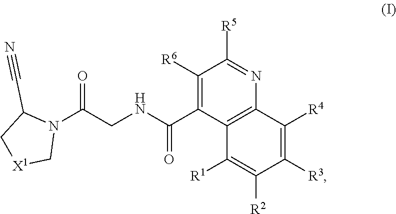

or a pharmaceutically acceptable salt thereof, wherein:
X¹ is selected from the group consisting of —S—, —S(O)—, and —S(O)₂—;
R¹ is selected from the group consisting of hydrogen, halogen, hydroxy, C₁₋₃-alkyl, and C₁₋₆-alkoxy;
R² is selected from the group consisting of
(a) C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, cyano, C₁₋₆-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(c) phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy; and
(d) a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(e) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
   (ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
   (iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy;
R³ is selected from the group consisting of hydrogen, halogen, and C₁₋₃-alkyl;
R⁴ is selected from the group consisting of hydrogen, halogen, and C₁₋₃-alkyl;
R⁵ is selected from the group consisting of hydrogen, halogen, and C₁₋₃-alkyl; and
R⁶ is selected from the group consisting of hydrogen, halogen, and C₁₋₃-alkyl.

In another aspect, the present disclosure provides compounds having the structure of Formula (II), (III-A), (III-B), (III-C), (III-D), (III-E), (IV), or (IV-A) as further defined herein, and pharmaceutically acceptable salts thereof.

In another aspect, the present disclosure provides pharmaceutical compositions comprising a therapeutically-effective amount of a compound having the structure of Formula (I), (II), (III-A), (III-B), (III-C), (III-D), (III-E), (IV), or (IV-A) as further defined herein, or a pharmaceutically acceptable salt thereof, and a pharmaceutically acceptable carrier.

In another aspect, the present disclosure provides pharmaceutical compositions comprising therapeutically-effective amounts of a compound having the structure of Formula (I), (II), (III-A), (III-B), (III-C), (III-D), (III-E), (IV), or (IV-A) as further defined herein, or a pharmaceutically acceptable salt thereof; a second pharmacological agent; and a pharmaceutically acceptable carrier.

In another aspect, the present disclosure provides methods for treating or preventing an FAP-mediated condition by administering a therapeutically effective amount of a compound having the structure of Formula (I), (II), (III-A), (III-B), (III-C), (III-D), (III-E), (IV), or (IV-A) as further defined herein, or pharmaceutically acceptable salt thereof, to a subject in need thereof. In one aspect, the FAP-mediated condition is selected from the group consisting of liver disease, type 2 diabetes mellitus, cardiovascular conditions, obesity, obesity-related conditions, fibrosis, keloid disorder, inflammation, and cancer. In another aspect, the FAP-mediated condition is liver disease, particularly nonalcoholic steatohepatitis (NASH).

In another aspect, the present disclosure provides compounds having the structure of Formula (I), (II), (III-A), (III-B), (III-C), (III-D), (III-E), (IV), or (IV-A) as further defined herein, or pharmaceutically acceptable salts thereof, for use as medicaments for treating or preventing an FAP-mediated condition.

In another aspect, the present disclosure provides use of compounds having the structure of Formula (I), (II), (III-A), (III-B), (III-C), (III-D), (III-E), (IV), or (IV-A) as further defined herein, or pharmaceutically acceptable salts thereof, to prepare medicaments for treating or preventing an FAP-mediated condition.

In another aspect, the present disclosure provides kits comprising a compound having the structure of Formula (I), (II), (III-A), (III-B), (III-C), (III-D), (III-E), (IV), or (IV-A) as further defined herein, or pharmaceutically acceptable salt thereof.

In another aspect, the present disclosure provides methods for preparing compounds having the structure of Formula (I), (II), (III-A), (III-B), (III-C), (III-D), (III-E), (IV), or (IV-A) as further defined herein, or pharmaceutically acceptable salts thereof.

### DETAILED DESCRIPTION

Many embodiments are detailed throughout the specification and will be apparent to a reader skilled in the art. The specification is not to be interpreted as being limited to any particular embodiment(s) described herein.

### I. Definitions

With respect to the embodiments disclosed in this specification, the following terms have the meanings set forth below:

Reference to “a” or “an” means “one or more.” Throughout, the plural and singular should be treated as interchangeable, other than the indication of number.

Unless the context requires otherwise, the words “comprise” or “comprises” or “comprising” are used on the basis and clear understanding that they are to be interpreted inclusively, rather than exclusively, and that Applicants intend each of those words to be so interpreted in construing this patent, including the claims below.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I).

The term “hydroxy” (alone or in combination with another term(s)) means —OH.

The term “cyano” (alone or in combination with another term(s)) means —CN.

The term “alkyl” (alone or in combination with another term(s)) means a straight- or branched-chain saturated hydrocarbyl substituent (i.e., a substituent containing only carbon and hydrogen). Alkyl typically contains from 1 to about 20 carbon atoms, more typically from 1 to about 12 carbon atoms, even more typically from 1 to about 8 carbon atoms, and still even more typically from 1 to about 6 carbon atoms. Examples of such substituents include methyl, ethyl, propyl (including n-propyl and isopropyl), butyl (including n-butyl, isobutyl, sec-butyl, and tert-butyl), pentyl (including n-pentyl, iso-amyl, and 2,2-dimethylpropyl), and hexyl.

The term “cycloalkyl” (alone or in combination with another term(s)) means a saturated carbocyclyl substituent containing from 3 to about 14 carbon ring atoms, more typically from 3 to about 12 carbon ring atoms, and even more typically from 3 to about 8 carbon ring atoms. A cycloalkyl includes a single carbon ring, which typically contains from 3 to 6 carbon ring atoms. Examples of single-ring cycloalkyls include cyclopropyl, cyclobutyl, cyclopentyl, and cyclohexyl.

The term “alkoxy” (alone or in combination with another term(s)) means an alkylether substituent, i.e., alkyl-O—. Examples of alkoxy include methoxy (CH₃—O—), ethoxy, n-propoxy, iso-propoxy, n-butoxy, iso-butoxy, sec-butoxy, and tert-butoxy. Thus, for example:
(i) the term “alkoxyalkyl” (alone or in combination with another term(s)) means alkyl substituted with alkoxy such as “methoxymethyl” which may be depicted as:

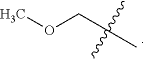 [SMILES: *COC]

(ii) the term “cycloalkylalkoxy” (alone or in combination with another term(s)) means alkoxy substituted with cycloalkyl such as “cyclopropylmethoxy” which may be depicted as:

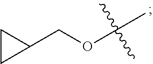 [SMILES: *OCC1CC1]

The term “cycloalkoxy” (alone or in combination with another term(s)) means a cycloalkylether substituent, i.e., cycloalkyl-O—. Examples of cycloalkoxy include cyclopropoxy, cyclobutoxy, cyclopentoxy, and cyclohexoxy. Thus, for example, the term “alkylcycloalkoxy” (alone or in combination with another term(s)) means cycloalkoxy substituted with alkyl such as “methylcyclopropoxy” which may be depicted as:

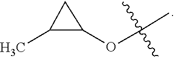 [SMILES: *OC1CC1C]

In some instances, the number of carbon atoms in a substituent (e.g., alkyl, cycloalkyl, etc.) is indicated by the prefix “Cx-y-”, wherein x is the minimum and y is the maximum number of carbon atoms in the substituent. Thus, for example, “C1-6-alkyl” refers to an alkyl substituent containing from 1 to 6 carbon atoms. Illustrating further, C3-6-cycloalkyl refers to a cycloalkyl substituent containing from 3 to 6 carbon ring atoms.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Where more than one hydrogen is replaced with a halogen, the halogens may be the identical or different. Examples of haloalkyls include fluoromethyl, difluoromethyl, trifluoromethyl, difluoroethyl, 1,1,1-trifluoroethyl, pentafluoroethyl, difluoropropyl, heptafluoropropyl chloromethyl, dichloromethyl, trichloromethyl, difluorochloromethyl, dichlorofluoromethyl, and dichloropropyl. Similarly, “haloalkoxy” means an alkoxy substituent wherein at least one hydrogen radical is replaced by a halogen radical. Where more than one hydrogen is replaced with a halogen, the halogens may be the identical or different. Examples of haloalkoxy substituents include fluoromethoxy, difluoromethoxy, trifluoromethoxy (also known as “perfluoromethyloxy”), 1,1,1-trifluoroethoxy, and chloromethoxy.

The term “heterocyclyl” (alone or in combination with another term(s)) means a saturated, partially saturated, or completely unsaturated (i.e., heteroaryl”) ring structure containing a total of 3 to 14 ring atoms. At least one of the ring atoms is a heteroatom (i.e., oxygen, nitrogen, or sulfur) with the remaining ring atoms being independently selected from the group consisting of carbon, oxygen, nitrogen, and sulfur. Heterocyclyl includes monocyclic saturated, partially unsaturated, and completely unsaturated ring structures having, for example, 4 to 7 members, such as 4 to 6 members, 5 to 7 members, or 5 or 6 members, where at least one member and up to 4 members, particularly 1, 2, or 3 members of the ring are heteroatoms selected from N, O and S, and the remaining ring atoms are carbon atoms, in stable combinations known to those of skill in the art. Heterocyclyl further includes bicyclic ring structures fused together (i.e., fused bicyclic) where at least one such ring contains a heteroatom as a ring atom (i.e., nitrogen, oxygen, or sulfur).

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted”, a non-hydrogen radical is in the place of a hydrogen radical on a carbon or nitrogen of the substituent. Thus, for example, a substituted alkyl substituent is an alkyl substituent wherein at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitutions on a substituent, each non-hydrogen radical may be identical or different (unless otherwise stated).

If a substituent is described as being “optionally substituted”, the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent.

If substituents are described as being “independently selected” from a group, each substituent is selected independent of the other. Each substituent therefore may be identical to or different from the other substituent(s).

The term “pharmaceutically acceptable” is used adjectivally in this specification to mean that the modified noun is appropriate for use as a pharmaceutical product or as a part of a pharmaceutical product. For example, “pharmaceutically acceptable salts” are salts that are suitable for use in mammals, particularly humans, and include salts with an inorganic base, organic base, inorganic acid, organic acid, or basic or acidic amino acid that are suitable for use in mammals, particularly humans.

A “therapeutically effective amount” of a pharmacological agent is an amount that is sufficient to effect beneficial or desired results, including clinical results, and, as such, will depend upon the situation in which it is being administered. Where the pharmacological agent is being administered to treat liver disease, for example, a therapeutically effective amount of the agent is an amount of the agent that is sufficient, either alone or in combination with additional therapies, to provide an anti-liver disease effect in a subject as compared to the response obtained without administration of the agent.

The term “preventing” is readily understood by an ordinarily skilled physician and, with respect to treatment of a particular condition, can include is intended to have its normal meaning and includes primary prophylaxis to prevent the development of the condition and secondary prophylaxis whereby the condition has already developed and the patient is temporarily or permanently protected against exacerbation or worsening of the disease or the development of new symptoms associated with the condition.

The terms “treating” is readily understood by an ordinarily skilled physician and, with respect to treatment of a particular condition, can include (1) diminishing the extent or cause of the condition being treated, and/or (2) alleviating or ameliorating one or more symptoms associated with that condition. Treatment of liver disease, for example, can include stabilizing (i.e., not worsening), delaying, or slowing the spread or progression of the liver disease; prolonging survival as compared to expected survival if not receiving treatment; and/or otherwise ameliorating or palliating the cancer or the severity of the liver disease, in whole or in part.

### II. Compounds

### A. Compounds of Formula (I)

In one embodiment, the present disclosure provides compounds having the structure of Formula (I):

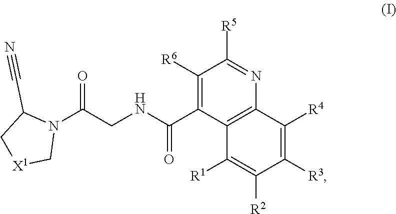

or a pharmaceutically acceptable salt thereof, wherein:
X¹ is selected from the group consisting of —S—, —S(O)—, and —S(O)₂—;
R¹ is selected from the group consisting of hydrogen, halogen, hydroxy, C₁₋₃-alkyl, and C₁₋₆-alkoxy;
R² is selected from the group consisting of
(a) C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, cyano, C₁₋₆-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(c) phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy; and
(d) a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(e) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
   (ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
   (iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy;
R³ is selected from the group consisting of hydrogen, halogen, and C₁₋₃-alkyl;
R⁴ is selected from the group consisting of hydrogen, halogen, and C₁₋₃-alkyl;
R⁵ is selected from the group consisting of hydrogen, halogen, and C₁₋₃-alkyl; and
R⁶ is selected from the group consisting of hydrogen, halogen, and C₁₋₃-alkyl.

In some embodiments, the present disclosure provides compounds having the structure of Formula (II):

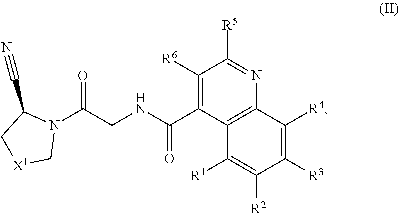

and pharmaceutically acceptable salts thereof, wherein X¹, R¹, R², R³, R⁴, R⁵, and R⁶ are as defined above for the compounds of Formula (I). In one aspect, X¹ is —S—. In another aspect, X¹ is —S(O)—. In another aspect, X¹ is —S(O)₂—.

In some embodiments, R¹ is selected from the group consisting of hydrogen, halogen, and C₁₋₃-alkyl. In one aspect, R¹ is selected from the group consisting of hydrogen, chloro, fluoro, and methyl. In another aspect, R¹ is hydrogen. In another aspect, R¹ is chloro. In another aspect, R¹ is fluoro. In another aspect, R¹ is methyl.

In some embodiments, R³ is selected from the group consisting of hydrogen, chloro, fluoro, and methyl. In one aspect, R³ is hydrogen. In another aspect, R³ is chloro. In another aspect, R³ is fluoro. In another aspect, R³ is methyl.

In some embodiments, R⁴ is selected from the group consisting of hydrogen, halogen, and methyl. In one aspect, R⁴ is hydrogen. In another aspect, R⁴ is chloro. In another aspect, R⁴ is fluoro. In another aspect, R⁴ is methyl.

In some embodiments, R⁵ is selected from the group consisting of hydrogen, chloro, fluoro, and methyl. In one aspect, R⁵ is hydrogen. In another aspect, R⁵ is fluoro. In another aspect, R⁵ is chloro. In another aspect, R⁵ is methyl.

In some embodiments, R⁶ is selected from the group consisting of hydrogen, chloro, fluoro, and methyl. In one aspect, R⁶ is hydrogen. In another aspect, R⁶ is chloro. In another aspect, R⁶ is fluoro. In another aspect, R⁶ is methyl.

In some embodiments, one of the R¹, R³, R⁴, R⁵, and R⁶ substituents is selected from the group consisting of halogen and C₁₋₃-alkyl, and the remaining R¹, R³, R⁴, R⁵, and R⁶ substituents are all hydrogen. In one aspect, one of the R¹, R³, R⁴, R⁵, and R⁶ substituents is selected from the group consisting of chloro, fluoro, and methyl, and the remaining R¹, R³, R⁴, R⁵, and R⁶ substituents are all hydrogen. In another aspect, one of the R¹, R³, R⁴, R⁵, and R⁶ substituents is selected from the group consisting of chloro and fluoro, and the remaining R¹, R³, R⁴, R⁵, and R⁶ substituents are all hydrogen. In another aspect, one of the R¹, R³, R⁴, R⁵, and R⁶ substituents is methyl, and the remaining R¹, R³, R⁴, R⁵, and R⁶ substituents are all hydrogen.

In some embodiments, at least two of the R¹, R³, R⁴, R⁵, and R⁶ substituents are independently selected from the group consisting of halogen and C₁₋₃-alkyl, and the remaining R¹, R³, R⁴, R⁵, and R⁶ substituents are all hydrogen.

In some embodiments, two of the R¹, R³, R⁴, R⁵, and R⁶ substituents are independently selected from the group consisting of chloro, fluoro, and methyl, and the remaining R¹, R³, R⁴, R⁵, and R⁶ substituents are all hydrogen.

In some embodiments, at least one of the R¹, R³, R⁴, R⁵, and R⁶ substituents is chloro.

In some embodiments, at least one of the R¹, R³, R⁴, R⁵, and R⁶ substituents is fluoro.

In some embodiments, at least one of the R¹, R³, R⁴, R⁵, and R⁶ substituents is methyl.

In some embodiments, the R¹, R³, R⁴, R⁵, and R⁶ substituents are hydrogen, and X¹ is selected from the group consisting of —S—, —S(O)—, and —S(O)₂—.

In some embodiments, the present disclosure provides compounds having the structure of Formula (III-A):

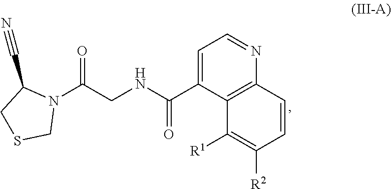

and pharmaceutically acceptable salts thereof, wherein R¹ and R² are as defined in the various embodiments described above.

In some embodiments, the present disclosure provides compounds having the structure of Formula (III-B):

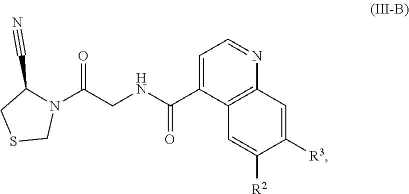

and pharmaceutically acceptable salts thereof, wherein R² and R³ are as defined in the various embodiments described above.

In some embodiments, the present disclosure provides compounds having the structure of Formula (III-C):

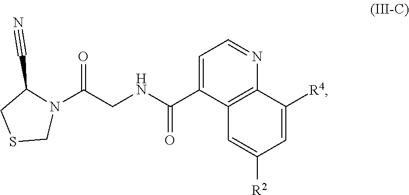

and pharmaceutically acceptable salts thereof, wherein R² and R⁴ are as defined in the various embodiments described above.

In some embodiments, the present disclosure provides compounds having the structure of Formula (III-D):

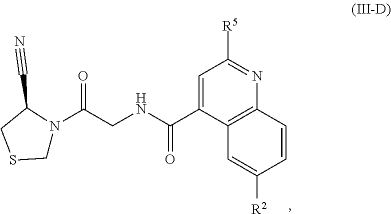

and pharmaceutically acceptable salts thereof, wherein R² and R⁵ are as defined in the various embodiments described above.

In some embodiments, the present disclosure provides compounds having the structure of Formula (III-E):

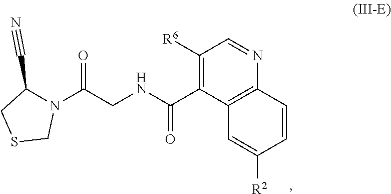

and pharmaceutically acceptable salts thereof, wherein R² and R⁶ are as defined in the various embodiments described above.

In some embodiments, the present disclosure provides compounds having the structure of Formula (IV):

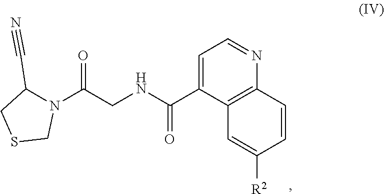

and pharmaceutically acceptable salts thereof, wherein R² is as defined in the various embodiments described above.

In some embodiments, the present disclosure provides compounds having the structure of Formula (IV-A):

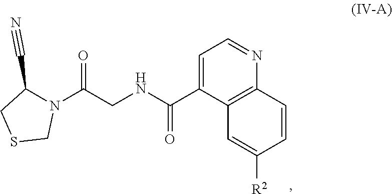

and pharmaceutically acceptable salts thereof, wherein R² is as defined in the various embodiments described above.

### B. R² is C₁₋₆-Alkyl

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl. In one aspect, R² is C₁₋₃-alkyl, wherein the C₁₋₃-alkyl is optionally substituted with one or more substituents independently selected from the group consisting of halogen, hydroxy, cyano, C₃₋₆-cycloalkyl, halo-C₃₋₆-cycloalkyl, C₁₋₃-alkoxy, halo-C₁₋₃-alkoxy, C₃₋₆-cycloalkoxy, halo-C₃₋₆-cycloalkoxy, C₁₋₃-alkoxy-C₁₋₃-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₃-alkyl. In another aspect, R² is C₁₋₆-alkyl, wherein the C₁₋₆-alkyl is optionally substituted with one or more substituents independently selected from the group consisting of halogen, hydroxy, C₁₋₆-alkoxy, halo-C₁₋₆-alkoxy, C₃₋₆-cycloalkoxy, C₁₋₆-alkoxy-C₁₋₆-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl. In another aspect, R² is C₁₋₃-alkyl, wherein the C₁₋₃-alkyl is optionally substituted with one or more substituents independently selected from the group consisting of halogen, hydroxy, C₁₋₃-alkoxy, halo-C₁₋₃-alkoxy, C₃₋₆-cycloalkoxy, C₁₋₃-alkoxy-C₁₋₃-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₆-alkyl.

In some embodiments, R² is C₁₋₆-alkyl. In one aspect, R² is C₁₋₃-alkyl. In one aspect, R² is methyl.

In some embodiments, R² is C₁₋₆-alkyl, wherein the C₁₋₆-alkyl is optionally substituted with one or more halogen. In one aspect, R² is C₁₋₆-alkyl, wherein the C₁₋₆-alkyl is optionally substituted with one or more fluoro.

In some embodiments, R² is C₁₋₆-alkyl, wherein the C₁₋₆-alkyl is optionally substituted with one or more hydroxy.

In some embodiments, R² is C₁₋₆-alkyl, wherein the C₁₋₆-alkyl is optionally substituted with one or more C₁₋₆-alkoxy.

In some embodiments, R² is C₁₋₆-alkyl, wherein the C₁₋₆-alkyl is optionally substituted with one or more halo-C₁₋₆-alkoxy.

In some embodiments, R² is C₁₋₆-alkyl, wherein the C₁₋₆-alkyl is optionally substituted with one or more C₃₋₆-cycloalkoxy.

In some embodiments, R² is C₁₋₆-alkyl, wherein the C₁₋₆-alkyl is optionally substituted with one or more C₁₋₆-alkoxy-C₁₋₆-alkoxy.

In some embodiments, R² is C₁₋₆-alkyl, wherein the C₁₋₆-alkyl is optionally substituted with one or more tetrahydrofuranyl, wherein the tetrahydrofuranyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more tetrahydrofuranyloxy, wherein the tetrahydrofuranyloxy is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more tetrahydropyranyl, wherein the tetrahydropyranyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more morpholinyl, wherein the morpholinyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more pyrazolyl, wherein the pyrazolyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more isoxazolyl, wherein the isoxazolyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more triazolyl, wherein the triazolyl is optionally substituted with one or more substituents independently selected from the group consisting of halogen, C₁₋₆-alkyl, and halo-C₁₋₆-alkyl.

In some embodiments, R² is C₁₋₆-alkyl, wherein the C₁₋₆-alkyl is optionally substituted with one or more pyridinyl, wherein the pyridinyl is optionally substituted with one or more substituents independently selected from the group consisting of halogen, C₁₋₆-alkyl, and halo-C₁₋₆-alkyl.

In some embodiments, R² is selected from the group consisting of methyl, fluoromethyl, difluoromethyl, trifluoromethyl, hydroxyethyl, hydroxypropyl, ethoxymethyl, difluoroethoxymethyl, methoxyethyl, dimethoxyethyl, difluoropropyl, trifluoropropyl, methoxypropyl, methoxyethoxymethyl, cyclopropoxymethyl, morpholinylmethyl, tetrahydrofuranylmethyl, tetrahydrofuranyloxymethyl, tetrahydropyranylmethyl, pyrazolylmethyl, dimethylpyrazolylmethyl, methylisoxazolylmethyl, methylisoxazolylethyl, methyltriazolylmethyl, dimethyltriazolylmethyl, dimethylpyridinylmethyl, methylpyridinylethyl, fluoropyridinylethyl, chloropyridinylethyl, and trifluoromethylpyridinylethyl.

In some embodiments, the compounds and pharmaceutically acceptable salts are selected from the group consisting of:
N-(2-((R)-4-Cyanothiazolidin-3-yl)-2-oxoethyl)-6-((RS)-1-(3-fluoropyridin-2-yl)-ethyl)quinoline-4-carboxamide (Example 12);
(R)—N-(2-(4-Cyanothiazolidin-3-yl)-2-oxoethyl)-6-(2-hydroxypropan-2-yl)quinoline-4-carboxamide (Example 16);
(R)—N-(2-(4-Cyanothiazolidin-3-yl)-2-oxoethyl)-6-(trifluoromethyl)-quinoline-4-carboxamide (Example 17);
(R)—N-(2-(4-Cyanothiazolidin-3-yl)-2-oxoethyl)-6-(2-methoxypropan-2-yl)quinoline-4-carboxamide (Example 18);
(R)—N-(2-(4-Cyanothiazolidin-3-yl)-2-oxoethyl)-6-((2,5-dimethyl-2H-1,2,3-triazol-4-yl)-methyl)quinoline-4-carboxamide (Example 19);
(R)—N-(2-(4-Cyanothiazolidin-3-yl)-2-oxoethyl)-6-(morpholino-methyl)quinoline-4-carboxamide (Example 24);
N-(2-((R)-4-Cyanothiazolidin-3-yl)-2-oxoethyl)-6-((RS)-1-hydroxyethyl)quinoline-4-carboxamide (Example 25);
N-(2-((R)-4-Cyanothiazolidin-3-yl)-2-oxoethyl)-6-((RS)-1-methoxyethyl)quinoline-4-carboxamide (Example 26);
(R)—N-(2-(4-cyanothiazolidin-3-yl)-2-oxoethyl)-6-(ethoxymethyl)-quinoline-4-carboxamide (Example 27);
(R)—N-(2-(4-cyanothiazolidin-3-yl)-2-oxoethyl)-6-methylquinoline-4-carboxamide (Example 28);
(R)—N-(2-(4-cyanothiazolidin-3-yl)-2-oxoethyl)-6-(fluoromethyl)-quinoline-4-carboxamide (Example 29);
N-(2-((R)-4-Cyanothiazolidin-3-yl)-2-oxoethyl)-6-((RS)-1,2-dimethoxyethyl)quinoline-4-carboxamide (Example 30);
(R)—N-(2-(4-Cyanothiazolidin-3-yl)-2-oxoethyl)-6-((2-methoxyethoxy)-methyl)quinoline-4-carboxamide (Example 31);
N-(2-((R)-4-Cyanothiazolidin-3-yl)-2-oxoethyl)-6-((((RS)-tetrahydrofuran-3-yl)oxy)-methyl)quinoline-4-carboxamide (Example 35);
(R)—N-(2-(4-Cyanothiazolidin-3-yl)-2-oxoethyl)-6-((2,2-difluoroethoxy)methyl)quinoline-4-carboxamide (Example 36);
(R)—N-(2-(4-Cyanothiazolidin-3-yl)-2-oxoethyl)-6-(cyclopropoxy-methyl)quinoline-4-carboxamide (Example 37);
(R)—N-(2-(4-Cyanothiazolidin-3-yl)-2-oxoethyl)-6-((4-methylisoxazol-3-yl)methyl)-quinoline-4-carboxamide (Example 38);
(R)—N-(2-(4-Cyanothiazolidin-3-yl)-2-oxoethyl)-6-((4,6-dimethylpyridin-3-yl)methyl)-quinoline-4-carboxamide (Example 39);
(R)—N-(2-(4-Cyanothiazolidin-3-yl)-2-oxoethyl)-6-((1,3-dimethyl-1H-pyrazol-5-yl)-methyl)quinoline-4-carboxamide (Example 40);
(R)—N-(2-(4-Cyanothiazolidin-3-yl)-2-oxoethyl)-6-((2-methyl-2H-1,2,3-triazol-4-yl)-methyl)quinoline-4-carboxamide (Example 41);
(R)—N-(2-(4-Cyanothiazolidin-3-yl)-2-oxoethyl)-6-((5-methylisoxazol-3-yl)-methyl)quinoline-4-carboxamide (Example 42);
N-(2-((R)-4-Cyanothiazolidin-3-yl)-2-oxoethyl)-6-((RS)-1-(5-methylisoxazol-3-yl)-ethyl)quinoline-4-carboxamide (Example 46);
6-((RS)-1-(5-Chloropyridin-2-yl)ethyl)-N-(2-((R)-4-cyanothiazolidin-3-yl)-2-oxoethyl)quinoline-4-carboxamide (Example 48);
N-(2-((R)-4-Cyanothiazolidin-3-yl)-2-oxoethyl)-6-((RS)-1-(3-methylisoxazol-5-yl)-ethyl)quinoline-4-carboxamide (Example 49);
N-(2-((R)-4-Cyanothiazolidin-3-yl)-2-oxoethyl)-6-((RS)-1-(6-methylpyridin-3-yl)-ethyl)quinoline-4-carboxamide (Example 51);
N-(2-((R)-4-Cyanothiazolidin-3-yl)-2-oxoethyl)-6-((RS)-1-(2-(trifluoromethyl)pyridin-4-yl)-ethyl)quinoline-4-carboxamide (Example 52);
(R)—N-(2-(4-Cyanothiazolidin-3-yl)-2-oxoethyl)-6-(2,2-difluoropropyl)quinoline-4-carboxamide (Example 53);
(R)—N-(2-(4-Cyanothiazolidin-3-yl)-2-oxoethyl)-6-((tetrahydro-2H-pyran-4-yl)-methyl)quinoline-4-carboxamide (Example 56);
(R)—N-(2-(4-Cyanothiazolidin-3-yl)-2-oxoethyl)-6-(3,3,3-trifluoropropyl)quinoline-4-carboxamide (Example 58);
N-(2-((R)-4-Cyanothiazolidin-3-yl)-2-oxoethyl)-6-(((RS)-tetrahydrofuran-3-yl)-methyl)quinoline-4-carboxamide (Example 65);
(R)—N-(2-(4-Cyanothiazolidin-3-yl)-2-oxoethyl)-6-(difluoromethyl)-quinoline-4-carboxamide (Example 71);
(R)-6-((1H-Pyrazol-1-yl)methyl)-N-(2-(4-cyanothiazolidin-3-yl)-2-oxoethyl)quinoline-4-carboxamide (Example 80);
(R)—N-(2-(4-cyanothiazolidin-3-yl)-2-oxoethyl)-6-(3-methoxypropyl)quinoline-4-carboxamide (Example 81);
(R)—N-(2-(4-cyanothiazolidin-3-yl)-2-oxoethyl)-6-(2-methoxyethyl)quinoline-4-carboxamide (Example 82);

and pharmaceutically acceptable salts thereof.

### C. R² is C₃₋₆-Cycloalkyl

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl. In one aspect, R² is C₃₋₆-cycloalkyl, wherein the C₃₋₆-cycloalkyl is optionally substituted with one or more substituents independently selected from the group consisting of halogen, hydroxy, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl. In another aspect, R² is C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl. In another aspect, R² is C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl.

In some embodiments, R² is optionally substituted cyclopropyl.

In some embodiments, R² is optionally substituted cyclobutyl.

In some embodiments, R² is optionally substituted cyclopentyl.

In some embodiments, R² is optionally substituted cyclohexyl.

In some embodiments, R² is unsubstituted C₃₋₆-cycloalkyl.

In some embodiments, R² is C₃₋₆-cycloalkyl, wherein the C₃₋₆-cycloalkyl is optionally substituted with one or more halogen.

In some embodiments, R² is C₃₋₆-cycloalkyl, wherein the C₃₋₆-cycloalkyl is optionally substituted with one or more cyano.

In some embodiments, R² is C₃₋₆-cycloalkyl, wherein the C₃₋₆-cycloalkyl is optionally substituted with one or more C₁₋₆-alkoxy.

In some embodiments, R² is C₃₋₆-cycloalkyl, wherein the C₃₋₆-cycloalkyl is optionally substituted with one or more isoxazolyl, wherein the isoxazolyl is optionally substituted with one or more substituents independently selected from the group consisting of halogen, C₁₋₆-alkyl, and halo-C₁₋₆-alkyl.

In some embodiments, R² is C₃₋₆-cycloalkyl, wherein the C₃₋₆-cycloalkyl is optionally substituted with one or more pyridinyl, wherein the pyridinyl is optionally substituted with one or more substituents independently selected from the group consisting of halogen, C₁₋₆-alkyl, and halo-C₁₋₆-alkyl.

In some embodiments, R² is selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl.

In some embodiments, the compounds and pharmaceutically acceptable salts are selected from the group consisting of:
(R)—N-(2-(4-Cyanothiazolidin-3-yl)-2-oxoethyl)-6-(1-(2-(trifluoromethyl)pyridin-4-yl)-cyclopropyl)quinoline-4-carboxamide (Example 13);
(R)—N-(2-(4-Cyanothiazolidin-3-yl)-2-oxoethyl)-6-(1-(6-methylpyridin-3-yl)-cyclopropyl)quinoline-4-carboxamide (Example 43);
(R)—N-(2-(4-Cyanothiazolidin-3-yl)-2-oxoethyl)-6-(1-(3-fluoropyridin-2-yl)-cyclopropyl)quinoline-4-carboxamide (Example 44);
(R)—N-(2-(4-Cyanothiazolidin-3-yl)-2-oxoethyl)-6-(1-(5-methylisoxazol-3-yl)-cyclopropyl)quinoline-4-carboxamide (Example 45);
(R)-6-(1-(5-Chloropyridin-2-yl)cyclopropyl)-N-(2-(4-cyanothiazolidin-3-yl)-2-oxoethyl)quinoline-4-carboxamide (Example 47);
(R)—N-(2-(4-Cyanothiazolidin-3-yl)-2-oxoethyl)-6-(1-(3-methylisoxazol-5-yl)-cyclopropyl)quinoline-4-carboxamide (Example 50);
6-((1r,4R*)-4-Cyanocyclohexyl)-N-(2-((R)-4-cyanothiazolidin-3-yl)-2-oxoethyl)-quinoline-4-carboxamide Isomer 1 (Example 54);
6-((1r,4R*)-4-Cyanocyclohexyl)-N-(2-((R)-4-cyanothiazolidin-3-yl)-2-oxoethyl)-quinoline-4-carboxamide Isomer 2 (Example 55);
(R)-6-(1-Cyanocyclohexyl)-N-(2-(4-cyanothiazolidin-3-yl)-2-oxoethyl)quinoline-4-carboxamide (Example 60);
N-(2-((R)-4-Cyanothiazolidin-3-yl)-2-oxoethyl)-6-((1r,3R*)-1-fluoro-3-methoxy-cyclobutyl)quinoline-4-carboxamide Isomer 1 (Example 62);
N-(2-((R)-4-Cyanothiazolidin-3-yl)-2-oxoethyl)-6-((1r,3R*)-1-fluoro-3-methoxy-cyclobutyl)quinoline-4-carboxamide Isomer 2 (Example 63);
(R)-6-(1-Cyanocyclopropyl)-N-(2-(4-cyanothiazolidin-3-yl)-2-oxoethyl)quinoline-4-carboxamide (Example 70);
(R)—N-(2-(4-Cyanothiazolidin-3-yl)-2-oxoethyl)-6-(1-fluoro-cyclobutyl)quinoline-4-carboxamide (Example 75);
(R)—N-(2-(4-Cyanothiazolidin-3-yl)-2-oxoethyl)-6-(1-ethoxycyclopropyl)quinoline-4-carboxamide (Example 76);
(R)—N-(2-(4-Cyanothiazolidin-3-yl)-2-oxoethyl)-6-cyclopropylquinoline-4-carboxamide (Example 77);
N-(2-((R)-4-Cyanothiazolidin-3-yl)-2-oxoethyl)-6-((1r,4R*)-4-methoxycyclohexyl)-quinoline-4-carboxamide Isomer 1 (Example 78);
N-(2-((R)-4-Cyanothiazolidin-3-yl)-2-oxoethyl)-6-((1r,4R*)-4-methoxycyclohexyl)-quinoline-4-carboxamide Isomer 2 (Example 79);

and pharmaceutically acceptable salts thereof.

### D. R² is Phenyl

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy. In one aspect, R² is phenyl, wherein the phenyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, and C₁₋₃-alkoxy-C₁₋₃-alkoxy.

In some embodiments, R² is phenyl, wherein the phenyl is optionally substituted with one or more halogen.

In some embodiments, R² is phenyl, wherein the phenyl is optionally substituted with one or more C₁₋₆-alkyl.

In some embodiments, R² is phenyl, wherein the phenyl is optionally substituted with one or more halo-C₁₋₆-alkyl.

In some embodiments, R² is phenyl, wherein the phenyl is optionally substituted with one or more C₁₋₆-alkoxy.

In some embodiments, R² is phenyl, wherein the phenyl is optionally substituted with one or more halo-C₁₋₆-alkoxy.

In some embodiments, R² is phenyl, wherein the phenyl is optionally substituted with one or more C₁₋₆-alkoxy-C₁₋₆-alkoxy.

In some embodiments, the compound is (R)—N-(2-(4-cyanothiazolidin-3-yl)-2-oxoethyl)-6-(4-(3-methoxy-propoxy)phenyl)quinoline-4-carboxamide, or a pharmaceutically acceptable salt thereof (Example 72).

### E. R² is 5-, 6-, or 7-Membered Ring Heterocyclyl

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is a 5- or 6-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

In some embodiments, R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is a 5-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is a 5-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is a 6-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is a 6-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is a 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is a 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is selected from the group consisting of tetrahydrofuranyl, furanyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy. In one aspect, R² is selected from the group consisting of tetrahydrofuranyl, furanyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

In some embodiments, R² is tetrahydrofuranyl, wherein the tetrahydrofuranyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

In some embodiments, R² is tetrahydropyranyl, wherein the tetrahydropyranyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

In some embodiments, R² is dihydropyranyl, wherein the dihydropyranyl, is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

In some embodiments, R² is 1,4-dioxanyl, wherein the 1,4-dioxanyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

In some embodiments, R² is 3-oxabicyclo[4.1.0]heptane, wherein the 3-oxabicyclo[4.1.0]heptane is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

In some embodiments, R² is selected from the group consisting of tetrahydrofuranyl, furanyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane.

In some embodiments, the compounds and pharmaceutically acceptable salts are selected from the group consisting of:
(R)—N-(2-(4-Cyanothiazolidin-3-yl)-2-oxoethyl)-6-(furan-3-yl)quinoline-4-carboxamide (Example 14);
(R)—N-(2-(4-Cyanothiazolidin-3-yl)-2-oxoethyl)-6-(4-hydroxy-tetrahydro-2H-pyran-4-yl)-quinoline-4-carboxamide (Example 15);
(R)—N-(2-(4-Cyanothiazolidin-3-yl)-2-oxoethyl)-6-(tetrahydro-2H-pyran-4-yl)quinoline-4-carboxamide (Example 20);
(R)—N-(2-(4-Cyanothiazolidin-3-yl)-2-oxoethyl)-6-(4-methyl-tetrahydro-2H-pyran-4-yl)-quinoline-4-carboxamide (Example 21);
(R)—N-(2-(4-Cyanothiazolidin-3-yl)-2-oxoethyl)-6-(3,6-dihydro-2H-pyran-4-yl)quinoline-4-carboxamide (Example 22);
(R)—N-(2-(4-Cyanothiazolidin-3-yl)-2-oxoethyl)-6-(4-ethoxytetrahydro-2H-pyran-4-yl)-quinoline-4-carboxamide (Example 23);
N-(2-((R)-4-Cyanothiazolidin-3-yl)-2-oxoethyl)-6-((RS)-tetrahydro-2H-pyran-2-yl)-quinoline-4-carboxamide (Example 32);
N-(2-((R)-4-Cyanothiazolidin-3-yl)-2-oxoethyl)-6-((RS)-1,4-dioxan-2-yl)quinoline-4-carboxamide (Example 33);
N-(2-((R)-4-Cyanothiazolidin-3-yl)-2-oxoethyl)-6-((RS)-tetrahydrofuran-2-yl)quinoline-4-carboxamide (Example 34);
(R)—N-(2-(4-Cyanothiazolidin-3-yl)-2-oxoethyl)-6-(4-ethynyl-tetrahydro-2H-pyran-4-yl)-quinoline-4-carboxamide (Example 57);
(R)-6-(4-Cyanotetrahydro-2H-pyran-4-yl)-N-(2-(4-cyanothiazolidin-3-yl)-2-oxoethyl)-quinoline-4-carboxamide (Example 59);
N-(2-((R)-4-Cyanothiazolidin-3-yl)-2-oxoethyl)-6-(5,5-dimethyltetrahydrofuran-3-yl)-quinoline-4-carboxamide (Example 61);
(R)—N-(2-(4-Cyanothiazolidin-3-yl)-2-oxoethyl)-6-(4-fluorotetrahydro-2H-pyran-4-yl)-quinoline-4-carboxamide (Example 64);
(R)—N-(2-(4-Cyanothiazolidin-3-yl)-2-oxoethyl)-6-(4-methoxy-tetrahydro-2H-pyran-4-yl)quinoline-4-carboxamide (Example 66);
N-(2-((R)-4-Cyanothiazolidin-3-yl)-2-oxoethyl)-6-((RS)-tetrahydrofuran-3-yl)quinoline-4-carboxamide (Example 67);
N-(2-((R)-4-Cyanothiazolidin-3-yl)-2-oxoethyl)-6-((RS)-2,2-dimethyltetrahydro-2H-pyran-4-yl)quinoline-4-carboxamide (Example 68);
6-((1RS,6SR)-3-Oxabicyclo[4.1.0]heptan-6-yl)-N-(2-((R)-4-cyanothiazolidin-3-yl)-2-oxoethyl)quinoline-4-carboxamide (Example 69);

and pharmaceutically acceptable salts thereof.

### F. R² is —OR⁷

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
(ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
(iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

In some embodiments, R² is —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
(ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
(iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

In some embodiments, R² is —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
(ii) the tetrahydropyranyl is optionally substituted with one or C₁₋₆-alkyl.

In some embodiments, R² is —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
(ii) the tetrahydropyranyl is optionally substituted with one or C₁₋₃-alkyl.

In some embodiments, R² is —OR⁷, wherein R⁷ is C₁₋₆-alkyl, wherein the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy.

In some embodiments, R² is —OR⁷, wherein R⁷ is C₁₋₄-alkyl, wherein the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy.

In some embodiments, R² is —OR⁷, wherein R⁷ is C₁₋₄-alkyl, wherein the C₁₋₄-alkyl is optionally substituted with one or more substituents independently selected from the group consisting of fluoro.

In some embodiments, R² is —OR⁷, wherein R⁷ is phenyl, wherein the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

In some embodiments, R² is —OR⁷, wherein R⁷ phenyl.

In some embodiments, R² is —OR⁷, wherein R⁷ is tetrahydropyranyl; wherein the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

In some embodiments, R² is —OR⁷, wherein R⁷ tetrahydropyranyl; wherein the tetrahydropyranyl is optionally substituted with one or more C₁₋₃-alkyl.

In some embodiments, R² is —OR⁷, wherein R⁷ tetrahydropyranyl.

In some embodiments, R² is —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

In some embodiments, the compounds and pharmaceutically acceptable salts are selected from the group consisting of:
(R)—N-(2-(4-Cyanothiazolidin-3-yl)-2-oxoethyl)-6-phenoxyquinoline-4-carboxamide (Example 1);
(R)—N-(2-(4-Cyanothiazolidin-3-yl)-2-oxoethyl)-6-(2,2-difluoroethoxy)quinoline-4-carboxamide (Example 2);
(R)—N-(2-(4-Cyanothiazolidin-3-yl)-2-oxoethyl)-6-(2-fluoro-2-methylpropoxy)quinoline-4-carboxamide (Example 3);
(R)—N-(2-(4-Cyanothiazolidin-3-yl)-2-oxoethyl)-6-(2-methoxyethoxy)quinoline-4-carboxamide (Example 4);
(R)—N-(2-(4-Cyanothiazolidin-3-yl)-2-oxoethyl)-6-(cyclopropyl-methoxy)quinoline-4-carboxamide (Example 5);
(R)—N-(2-(4-Cyanothiazolidin-3-yl)-2-oxoethyl)-6-((tetrahydro-2H-pyran-4-yl)oxy)-quinoline-4-carboxamide (Example 6);
(R)—N-(2-(4-Cyanothiazolidin-3-yl)-2-oxoethyl)-6-((1,3-difluoropropan-2-yl)oxy)-quinoline-4-carboxamide (Example 7);
(R)—N-(2-(4-Cyanothiazolidin-3-yl)-2-oxoethyl)-6-((3,3-difluorocyclobutyl)-methoxy)quinoline-4-carboxamide (Example 8);
N-(2-((R)-4-Cyanothiazolidin-3-yl)-2-oxoethyl)-6-(((RS)-2,2-dimethyltetrahydro-2H-pyran-4-yl)oxy)quinoline-4-carboxamide (Example 9);
(R)-6-(Cyanomethoxy)-N-(2-(4-cyanothiazolidin-3-yl)-2-oxoethyl)quinoline-4-carboxamide (Example 10);
(R)—N-(2-(4-Cyanothiazolidin-3-yl)-2-oxoethyl)-6-(2,2-difluoropropoxy)quinoline-4-carboxamide (Example 11);
(R)—N-(2-(4-Cyanothiazolidin-3-yl)-2-oxoethyl)-6-propoxyquinoline-4-carboxamide (Example 73);
(R)—N-(2-(4-Cyanothiazolidin-3-yl)-2-oxoethyl)-6-(2-fluoroethoxy)-quinoline-4-carboxamide (Example 74);

and pharmaceutically acceptable salts thereof.

### G. Additional Embodiments

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₁₋₃-alkyl, wherein the C₁₋₃-alkyl is optionally substituted with one or more substituents independently selected from the group consisting of halogen, hydroxy, cyano, C₃₋₆-cycloalkyl, halo-C₃₋₆-cycloalkyl, C₁₋₃-alkoxy, halo-C₁₋₃-alkoxy, C₃₋₆-cycloalkoxy, halo-C₃₋₆-cycloalkoxy, C₁₋₃-alkoxy-C₁₋₃-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₃-alkyl;
(b) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(e) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
   (ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
   (iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl; and
(b) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(c) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
   (ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
   (iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₁₋₆-alkyl, wherein the C₁₋₆-alkyl is optionally substituted with one or more substituents independently selected from the group consisting of halogen, hydroxy, C₁₋₆-alkoxy, halo-C₁₋₆-alkoxy, C₃₋₆-cycloalkoxy, C₁₋₆-alkoxy-C₁₋₆-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy; and
(e) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
   (ii) the tetrahydropyranyl is optionally substituted with one or C₁₋₆-alkyl.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(b) a 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy; and
(c) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
   (ii) the tetrahydropyranyl is optionally substituted with one or C₁₋₆-alkyl.
      In one aspect, R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy. In another aspect, R² is a 5-membered ring heterocyclyl, wherein the heterocyclyl ring: (i) is a saturated or partially saturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, and C₂₋₃-alkynyl, C₁₋₃-alkoxy. In another aspect, R² is a 6-membered ring heterocyclyl, wherein the heterocyclyl ring: (i) is a saturated or partially saturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy. In another aspect, R² is a 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₁₋₃-alkyl, wherein the C₁₋₃-alkyl is optionally substituted with one or more substituents independently selected from the group consisting of halogen, hydroxy, C₁₋₃-alkoxy, halo-C₁₋₃-alkoxy, C₃₋₆-cycloalkoxy, C₁₋₃-alkoxy-C₁₋₃-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 5-, 6-, or 7-membered ring heterocyclyl selected from the group consisting of tetrahydrofuranyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(e) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
   (ii) the tetrahydropyranyl is optionally substituted with one or C₁₋₃-alkyl.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(b) a 5-, 6-, or 7-membered ring heterocyclyl selected from the group consisting of tetrahydrofuranyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(c) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
   (ii) the tetrahydropyranyl is optionally substituted with one or C₁₋₃-alkyl.
      In one aspect, R² is optionally substituted cyclopropyl. In another aspect, R² is optionally substituted cyclobutyl. In another aspect, R² is optionally substituted cyclopentyl. In another aspect, R² is optionally substituted cyclohexyl.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) optionally substituted C₁₋₃-alkyl selected from the group consisting of methyl, fluoromethyl, difluoromethyl, trifluoromethyl, hydroxyethyl, hydroxypropyl, ethoxymethyl, difluoroethoxymethyl, methoxyethyl, dimethoxyethyl, difluoropropyl, trifluoropropyl, methoxypropyl, methoxyethoxymethyl, cyclopropoxymethyl, morpholinylmethyl, tetrahydrofuranylmethyl, tetrahydrofuranyloxymethyl, tetrahydropyranylmethyl, pyrazolylmethyl, dimethylpyrazolylmethyl, methylisoxazolylmethyl, methylisoxazolylethyl, methyltriazolylmethyl, dimethyltriazolylmethyl, dimethylpyridinylmethyl, methylpyridinylethyl, fluoropyridinylethyl, chloropyridinylethyl, and trifluoromethylpyridinylethyl.
(b) optionally substituted C₃₋₆-cycloalkyl selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl;
(c) optionally substituted 5-, 6-, or 7-membered ring heterocyclyl selected from the group consisting of tetrahydrofuranyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane; and
(d) —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of:
(a) optionally substituted C₃₋₆-cycloalkyl selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl;
(b) optionally substituted 5-, 6-, or 7-membered ring heterocyclyl selected from the group consisting of tetrahydrofuranyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane; and
(c) —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

In some embodiments, the present disclosure provides compounds having the structure of Formula (I), (II), (III-A), (III-B), (III-C), (III-D), (III-E), (IV), or (IV-A), or pharmaceutically acceptable salts thereof, wherein R² is selected from the group consisting of methoxycyclohexyl, tetrahydropyranyl, fluorobutoxy, and tetrahydropyranyloxy.

### H. Combination of Embodiments

Any embodiment of the compounds described in the present disclosure can be combined with any other suitable embodiment described herein to provide additional embodiments. For example, where one embodiment individually or collectively describes possible groups for R¹, R², R³, R⁴, R⁵, and/or R⁶ and a separate embodiment describes possible groups for R², it is understood that these embodiments can be combined to provide an additional embodiment describing the possible groups described for R¹, R², R³, R⁴, R⁵, and/or R⁶ together with the possible groups described for R². In other words, for any of the embodiments of the compounds described in the present disclosure, the R² substituent can be as defined in any of the embodiments of R² described in this specification.

### I. Further Embodiments

The compounds of the present disclosure have a pharmaceutically acceptable FAP inhibitory activity measured as described for the hFAP inhibition assay (tight binders) reported in the Examples below. In one aspect, the compounds have an FAP inhibitory activity at IC₅₀ concentrations below about 100 nM. In another aspect, the compounds have an FAP inhibitory activity at IC₅₀ concentrations below about 50 nM. In another aspect, the compounds have an FAP inhibitory activity at IC₅₀ concentrations below about 10 nM. In another aspect, the compounds have an FAP inhibitory activity at IC₅₀ concentrations below about 1 nM.

In some embodiments, the compounds of the present disclosure possess a pharmaceutically acceptable surface plasmon resonance (SPR) pK_{d} value measured as described for the SPR assay reported in the Examples below. In one aspect, the compounds have a surface plasmon resonance (SPR) pK_{d} value greater than about 7. In another aspect, the compounds have a surface plasmon resonance (SPR) pK_{d} value greater than about 8. In another aspect, the compounds have an SPR pK_{d} value greater than about 9. In another aspect, the compounds have an SPR pK_{d} value greater than about 10.

In some embodiments, the compounds of the present disclosure have a pharmaceutically acceptable selectivity for FAP relative to PREP measured as described for the hFAP inhibition assay (tight binders) and the hPREP inhibition assay reported in the Examples below. In one aspect, the compounds are at least about 50 times more selective for FAP relative to PREP. In another aspect, the compounds are at least about 100 times more selective for FAP relative to PREP. In another aspect, the compounds are at least about 1,000 times more selective for FAP relative to PREP. In another aspect, the compounds are at least about 10,000 times more selective for FAP relative to PREP. In another aspect, the compounds have a PREP IC₅₀ value greater than about 0.1 μM. In another aspect, the compounds have a PREP IC₅₀ value greater than about 1.0 μM. In another aspect, the compounds have a PREP IC₅₀ value greater than about 10.0 μM.

In some embodiments, the compounds of the present disclosure have a pharmaceutically acceptable selectivity for FAP relative to DPP7 measured as described for the hFAP inhibition assay (tight binders) and the DPP7 selectivity assay reported in the Examples below. In one aspect, the compounds are at least about 50 times more selective for FAP relative to DPP7. In another aspect, the compounds are at least about 100 times more selective for FAP relative to DPP7. In another aspect, the compounds are at least about 1,000 times more selective for FAP relative to DPP7. In another aspect, the compounds are at least about 10,000 times more selective for FAP relative to DPP7. In another aspect, the compounds have an IC₅₀ value for DPP7 that is greater than about 0.1 μM. In another aspect, the compounds have an IC₅₀ value for DPP7 that is greater than about 1 μM. In another aspect, the compounds have an IC₅₀ value for DPP7 that is greater than about 10 μM.

In some embodiments, the compounds of the present disclosure have a pharmaceutically acceptable selectivity for FAP relative to DPP8 and/or DPP9 measured as described for the hFAP inhibition assay (tight binders), DPP8 selectivity assay, and DPP9 selectivity assay reported in the Examples below. In one aspect, the compounds are selective for FAP relative to DPP8. In another aspect, the compounds are selective for FAP relative to DPP9. In another aspect, the compounds are selective for FAP relative to both DPP8 and DPP9. In one aspect, the compounds are at least about 50 times more selective for FAP relative to DPP8 and/or DPP9. In another aspect, the compounds are at least about 100 times more selective for FAP relative to DPP8 and/or DPP9. In another aspect, the compounds are at least about 500 times more selective for FAP relative to DPP8 and/or DPP9. In another aspect, the compounds are at least about 1,000 times more selective for FAP relative to DPP8 and/or DPP9. In another aspect, the compounds have an IC₅₀ value for DPP8 and/or DPP9 that is greater than about 0.01 μM. In another aspect, the compounds have an IC₅₀ value for DPP8 and/or DPP9 that is greater than about 0.1 μM. In another aspect, the compounds have an IC₅₀ value for DPP8 and/or DPP9 that is greater than about 0.4 μM.

In some embodiments, the compounds of the present disclosure have a pharmaceutically acceptable metabolic stability measured as described for the human liver microsomes (HLM) assay reported in the Examples below. In one aspect, the compounds have an HLM CLᵢₙₜ value less than about 300 μL/min/mg. In another aspect, the compounds have an HLM CLᵢₙₜ value less than about 100 μL/min/mg. In another aspect, the compounds have an HLM CLᵢₙₜ value less than about 50 μL/min/mg.

In some embodiments, the compounds of the present disclosure have a pharmaceutically acceptable metabolic stability measured as described for the rat hepatocytes (rHep) assay reported in the Examples below. In one aspect, the compounds have an rHep CLᵢₙₜ value less than about 300 μL/min/10⁶ cells. In another aspect, the compounds have an rHep CLᵢₙₜ value less than about 100 μL/min/10⁶ cells. In another aspect, the compounds have an rHep CLᵢₙₜ value less than about 50 μL/min/10⁶ cells.

In some embodiments, the compounds of the present disclosure have a pharmaceutically acceptable Caco-2 AB intrinsic permeability measured as described for the Caco-2 AB intrinsic permeability assay reported in the Examples below. In one aspect, the compounds have a Caco-2 intrinsic apparent permeability of at least about 0.1×10⁶ cm/s. In another aspect, the compounds have a Caco-2 intrinsic apparent permeability of at least about 0.5×10⁶ cm/s. In another aspect, the compounds have a Caco-2 intrinsic apparent permeability of at least about 1×10⁶ cm/s.

In some embodiments, the compounds of the present disclosure have a pharmaceutically acceptable Caco-2 bidirectional (ABBA) A to B apparent permeability measured as described for the Caco-2 bidirectional (ABBA) A to B apparent permeability assay reported in the Examples below. In one aspect, the compounds have a Caco-2 bidirectional (ABBA) A to B apparent permeability of at least about 0.1×10⁶ cm/s. In another aspect, the compounds have a Caco-2 bidirectional (ABBA) A to B apparent permeability of at least about 0.25×10⁶ cm/s. In another aspect, the compounds have a Caco-2 bidirectional (ABBA) A to B apparent permeability of at least about 0.5×10⁶ cm/s.

In some embodiments, the compounds of the present disclosure have a pharmaceutically acceptable kinetic solubility measured as described for the kinetic solubility assay reported in the Examples below. In one aspect, the compounds have a kinetic solubility of at least about 1 μM. In another aspect, the compounds have a kinetic solubility of at least about 10 μM. In another aspect, the compounds have a kinetic solubility of at least about 25 μM. In another aspect, the compounds have a kinetic solubility of at least about 50 μM.

### J. Salts

The compounds of the present disclosure may exist in salt form or in non-salt form (i.e., as a free base), and the present disclosure covers both salt forms and non-salt forms. The compounds may form acid addition salts or base addition salts. In general, an acid addition salt can be prepared using various inorganic or organic acids. Such salts can typically be formed by, for example, mixing the compound with an acid (e.g., a stoichiometric amount of an acid) using various methods known in the art. This mixing may occur in water, an organic solvent (e.g., ether, ethyl acetate, ethanol, methanol, isopropanol, or acetonitrile), or an aqueous/organic mixture. In another aspect, the acid addition salts are, for example, trifluoroacetate, formate, acetate or hydrochloric. In general, a base addition salt can be prepared using various inorganic or organic bases, for example an alkali or alkaline earth metal salt such as a sodium, calcium or magnesium salt, or other metal salts, such as potassium or zinc, or an ammonium salt, or a salt with an organic base such as methylamine, dimethylamine, trimethylamine, piperidine or morpholine. The skilled person will be aware of the general principles and techniques of preparing pharmaceutical salts, such as those described in, for example, J. Pharm. Sci. 1977 66, 1. Examples of pharmaceutically acceptable salts are also described in “Handbook of Pharmaceutical Salts: Properties, Selection, and Use” by Stahl and Wermuth (Wiley-VCH, Weinheim, Germany, 2002).

### K. Isomers

The compounds and salts of the present disclosure may exist in one or more geometrical, optical, enantiomeric, and diastereomeric forms, including, but not limited to, cis- and trans-forms, E- and Z-forms, and R-, S- and meso-forms. Unless otherwise stated a reference to a particular compound includes all such isomeric forms, including racemic and other mixtures thereof. Where appropriate such isomers can be separated from their mixtures by the application or adaptation of known methods (e.g., chromatographic techniques and recrystallisation techniques). Where appropriate such isomers can be prepared by the application or adaptation of known methods. In some embodiments, a single stereoisomer is obtained by isolating it from a mixture of isomers (e.g., a racemate) using, for example, chiral chromatographic separation. In other embodiments, a single stereoisomer is obtained through direct synthesis from, for example, a chiral starting material.

A particular enantiomer of a compound described herein may be more active than other enantiomers of the same compound. In one embodiment, the compound, or a pharmaceutically acceptable salt thereof, is a single enantiomer being in an enantiomeric excess (% ee) of ≥90, ≥95%, ≥96%, ≥97, ≥98% or ≥99%. In one aspect, the single enantiomer is present in an enantiomeric excess (% ee) of ≥99%.

In another embodiment, the present disclosure relates to a pharmaceutical composition comprising a compound, or a pharmaceutically acceptable salt thereof, which is a single enantiomer being in an enantiomeric excess (% ee) of ≥90, ≥95%, ≥96%, ≥97, ≥98% or ≥99%, or a pharmaceutically acceptable salt thereof, in association with one or more pharmaceutically acceptable excipients. In one aspect, the single enantiomer is present in an enantiomeric excess (% ee) of ≥99%.

### L. Additional Forms

The compounds and salts of the present disclosure may exist in various tautomeric forms and the specification encompasses all such tautomeric forms. “Tautomers” are structural isomers that exist in equilibrium resulting from the migration of a hydrogen atom.

The compounds of the present disclosure, and pharmaceutically acceptable salts thereof, may exist as solvates (such as a hydrates) as well as unsolvated forms, and the present specification covers all such solvates.

The compounds of the present disclosure, and pharmaceutically acceptable salts thereof, may exist in crystalline or amorphous form, and the present specification covers all such forms.

Compounds and salts of the present disclosure may be isotopically-labeled (or “radio-labeled”). In that instance, one or more atoms are replaced by an atom having an atomic mass or mass number different from the atomic mass or mass number typically found in nature. The specification encompasses isotopically-labelled forms of compounds disclosed herein. Examples of isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O and ³⁶Cl. The isotope that is used will depend on the specific application of that radio-labeled derivative. For example, for in vitro receptor labeling and competition assays, ³H or ⁴C are often useful. For radio-imaging applications, ¹¹C is often useful. In some embodiments, the radionuclide is ³H. In some embodiments, the radionuclide is ¹⁴C. In some embodiments, the radionuclide is ¹¹C.

### M. Intermediates

In some embodiments, the present disclosure provides additional compounds that are useful as intermediates for preparing the compounds of the present disclosure, and pharmaceutically acceptable salts thereof.

### III. Methods of Use

The disclosed compounds of the present disclosure, and pharmaceutically acceptable salts thereof, are inhibitors of Prolyl endopeptidase fibroblast activation protein (FAP) activity. FAP is an endopeptidase that enzymatically cleaves substrates involved in glucose and lipid metabolism, fibrinolysis, and collagen production.

FAP is believed to cleave and inactivate human Fibroblast Growth Factor 21 (FGF-21) (Biochem. J. 2016, 473, 605), a protein involved in the regulation of glucose and lipid metabolism. It is hypothesized that inhibition of FAP increases endogenous FGF-21 levels and signaling, and results, for example, in decreased steatosis, improved insulin sensitivity, improved glucose tolerance, reduced body weight, and/or reduced cardiovascular disease mortality.

FAP is also believed to cleave human α2-Antiplasmin (α2AP) (Blood 2004 103, 3783), a protein involved in the regulation of fibrosis and fibrinolysis. Tissue repair involves coagulation which results in fibrin deposition. The fibrin of a clot is usually lysed, primarily by plasmin when converted from its inactive form (plasminogen) by plasminogen activators. Fibrinolysis is inhibited by Plasminogen Activator Inhibitor-1 (PAI-1), Plasminogen Activator Inhibitor-2 (PAI-2), and α2AP, (Experimental & Molecular Medicine 2020, 52, 367) all of which are induced by tissue trauma. FAP converts α2AP into a form more effectively bound to fibrin, which reduces plasmin degradation of fibrin at the site of an injury. It is hypothesized that inhibition of FAP increases fibrinolysis and improves tissue regeneration at the site of injury (J. Thromb. Haemost. 2013, 11, 2029; Proteomics Clin. Appl. 2014, 8, 454).

FAP is further believed to promote collagen production and deposition and to play a role in increased fibrosis through altered extracellular matrix (ECM) turnover (J Biol Chem 2016, 8, 291). It is hypothesized that inhibition of FAP results in a decrease in collagen deposition and a reduction in inflammation (Inflamm. Bowel Dis. 2018, 18, 332).

In view of the above, it is hypothesized that inhibition of FAP collectively reduces fibrosis and inflammation by decreasing hepatic stellate cell activity and increasing fibrinolysis, and further provides positive metabolic effects through increased FGF21 signaling and improved glucose tolerance.

In some embodiments, therefore, the present disclosure provides a method for treating or preventing an FAP-mediated condition in a subject in need thereof by administering to the subject a therapeutically effective amount of a compound of Formula I, or a pharmaceutically acceptable salt thereof.

In some embodiments, the present disclosure provides a method for treating or preventing a condition characterized by overexpression of FAP in a subject in need thereof by administering to the subject a therapeutically effective amount of a compound of the present disclosure, or a pharmaceutically acceptable salt thereof.

In some embodiments, the present disclosure provides a method for treating or preventing liver disease in a subject in need thereof by administering to the subject a therapeutically effective amount of a compound of the present disclosure, or a pharmaceutically acceptable salt thereof. In one aspect, the liver disease is a fatty liver disease. In another aspect, the liver disease is Nonalcoholic Fatty Liver Disease (NAFLD). In another aspect, the NAFLD is selected from the group consisting of isolated steatosis, Nonalcoholic Steatohepatitis (NASH), liver fibrosis, and cirrhosis. In another aspect, the liver disease is end stage liver disease. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating liver disease in a subject in need thereof by administering to the subject a therapeutically effective amount of a compound of the present disclosure, or a pharmaceutically acceptable salt thereof, wherein the subject has a body mass index (BMI) of 27 kg/m² to 40 kg/m². In one aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a therapeutically effective amount of a compound of the present disclosure, or a pharmaceutically acceptable salt thereof, wherein the subject is also suffering from or susceptible to dyslipidemia. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a therapeutically effective amount of a compound of the present disclosure, or a pharmaceutically acceptable salt thereof, wherein the subject is also suffering from or susceptible to insulin resistance. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a therapeutically effective amount of a compound of the present disclosure, or a pharmaceutically acceptable salt thereof, wherein the subject is also suffering from or susceptible to at least one of Type 2 diabetes and renal insufficiency. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a therapeutically effective amount of a compound of the present disclosure, or a pharmaceutically acceptable salt thereof, wherein the subject is also suffering from or susceptible to Type 2 diabetes. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a therapeutically effective amount of a compound of the present disclosure, or a pharmaceutically acceptable salt thereof, wherein the subject is also suffering from or susceptible to renal insufficiency. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for reducing liver fat in a subject in need thereof by administering to the subject a therapeutically effective amount of a compound of the present disclosure, or a pharmaceutically acceptable salt thereof. In one aspect, the subject is suffering from or susceptible to NAFLD. In another aspect, the subject is suffering from or susceptible to NASH. In another aspect, the subject is suffering from or susceptible to liver fibrosis. In another aspect, the subject is suffering from or susceptible to cirrhosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing Nonalcoholic Fatty Liver Disease (NAFLD) in a subject in need thereof by administering to the subject a therapeutically effective amount of a compound of the present disclosure, or a pharmaceutically acceptable salt thereof. In one aspect, the NAFLD is Stage 1 NAFLD. In another aspect, the NAFLD is Stage 2 NAFLD. In another aspect, the NAFLD is Stage 3 NAFLD. In another aspect, the NAFLD is Stage 4 NAFLD. See, e.g., “The Diagnosis and Management of Nonalcoholic Fatty Liver Disease: Practice Guidance From the American Association for the Study of Liver Diseases,” Hepatology, 2018, Vol. 67, No. 1. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing Nonalcoholic Steatohepatitis (NASH) in a subject in need thereof by administering to the subject a therapeutically effective amount of a compound of the present disclosure, or a pharmaceutically acceptable salt thereof. In one aspect, the NASH is Stage 1 NASH. In another aspect, the NASH is Stage 2 NASH. In another aspect, the NASH is Stage 3 NASH. In another aspect, the NASH is Stage 4 NASH. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing liver fibrosis in a subject in need thereof by administering to the subject a therapeutically effective amount of a compound of the present disclosure, or a pharmaceutically acceptable salt thereof. In one aspect, the subject is suffering from Stage 3 liver fibrosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing cirrhosis in a subject in need thereof by administering to the subject a therapeutically effective amount of a compound of the present disclosure, or a pharmaceutically acceptable salt thereof. In one aspect, the subject is suffering from stage F4 cirrhosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing type 2 diabetes mellitus in a subject in need thereof by administering to the subject a therapeutically effective amount of a compound of the present disclosure, or a pharmaceutically acceptable salt thereof. In one aspect, the subject is a subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of improving glycemic control in a subject in need thereof by administering to the subject a therapeutically effective amount of a compound of the present disclosure, or a pharmaceutically acceptable salt thereof. In one aspect, the subject is a subject is suffering from type 2 diabetes. In another aspect, the subject is a subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject as a BMI of at least 40 kg/m². In another aspect, the subject is overweight. In another aspect, the subject is obese.

In some embodiments, the present disclosure provides a method of improving glycemic control in a subject with type 2 diabetes and diabetic kidney disease by administering to the subject a therapeutically effective amount of a compound of the present disclosure, or a pharmaceutically acceptable salt thereof. In one aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of improving glycemic control in a subject with type 2 diabetes and renal insufficiency by administering to the subject a therapeutically effective amount of a compound of the present disclosure, or a pharmaceutically acceptable salt thereof. In one aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of treating or preventing insulin resistance in a subject thereof by administering to the subject a therapeutically effective amount of a compound of the present disclosure, or a pharmaceutically acceptable salt thereof. In another aspect, the subject is a subject is suffering from type 2 diabetes. In another aspect, the subject is a subject is suffering from diabetic kidney disease. In another aspect, the subject is suffering from renal insufficiency. Insulin resistance can be measured, for example, using the Homeostatic Model Assessment of Insulin Resistance (HOMA-IR) and/or the MATSUDA index. The HOMA-IR is explained, for example, in Diabetologia 1985, 28, 412, which is herein incorporated by reference in its entirety. The MATSUDA index is explained, for example, in Diabetes Care 1999, 22, 1462, which is herein incorporated by reference in its entirety.

In some embodiments, the present disclosure provides a method of treating or preventing glucose intolerance in a subject in need thereof by administering to the subject a therapeutically effective amount of a compound of the present disclosure, or a pharmaceutically acceptable salt thereof. In one aspect, the subject is a subject is suffering from type 2 diabetes. In another aspect, the subject is a subject is suffering from diabetic kidney disease. In another aspect, the subject is suffering from renal insufficiency.

In some embodiments, the present disclosure provides a method of treating a cardiovascular condition in a subject in need of treatment by administering to the subject a therapeutically effective amount of a compound of the present disclosure, or a pharmaceutically acceptable salt thereof. In one aspect, the cardiovascular condition is selected from the group consisting of heart failure, cardiomyopathy, atherosclerosis, venous thromboembolism, and atrial fibrillation. In one aspect, the cardiovascular condition is heart failure. In another aspect, the cardiovascular condition is heart failure with preserved ejection fraction (HFpEF). In another aspect, the cardiovascular condition is cardiomyopathy. In another aspect, the cardiomyopathy is selected from the group consisting of hypertrophic cardiomyopathy, dilated cardiomyopathy, restrictive cardiomyopathy, hypertrophic cardiomyopathy, ischemic cardiomyopathy, ischemic cardiomyopathy, dilated cardiomyopathy, and idiopathic cardiomyopathy. In another aspect, the cardiovascular condition is atherosclerosis. In another aspect, the cardiovascular condition is venous thromboembolism. In another aspect, the cardiovascular condition is atrial fibrillation.

In some embodiments, the present disclosure provides a method of treating obesity or an obesity-related condition in a subject in need thereof by administering to the subject a therapeutically effective amount of a compound of the present disclosure, or a pharmaceutically acceptable salt thereof. In one aspect, the obesity-related condition is an obesity-related metabolic condition. In another aspect, the obesity-related condition is selected from the group consisting of insulin resistance, pre-diabetes, type 2 diabetes, glucose intolerance, increased fasting glucose, and glucagonomas. In another aspect, the obesity-related condition is dyslipidemia. In another aspect, the obesity-related condition is a cardiovascular condition is selected from the group consisting of heart failure, cardiomyopathy, atherosclerosis, venous thromboembolism, and atrial fibrillation. In another aspect, the obesity-related condition is renal disease.

In some embodiments, the present disclosure provides a method of reducing body weight in a subject in need thereof by administering to the subject a therapeutically effective amount of a compound of the present disclosure, or a pharmaceutically acceptable salt thereof. In one aspect, the subject is a subject is suffering from type 2 diabetes. In another aspect, the subject is a subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the subject's weight is reduced, for example, by at least about 5%, 10%, 15%, 20%, 25%, 30%, 35%, or 40%.

In some embodiments, the present disclosure provides a method of reducing body fat in a subject in need of treatment by administering to the subject a therapeutically effective amount of a compound of the present disclosure, or a pharmaceutically acceptable salt thereof. In another aspect, the subject is a subject is suffering from type 2 diabetes. In another aspect, the subject is a subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the fat is liver fat.

In some embodiments, the present disclosure provides a method for treating or preventing fibrosis in a subject in need thereof by administering to the subject a therapeutically effective amount of a compound of the present disclosure, or a pharmaceutically acceptable salt thereof. In one aspect, the fibrosis is interstitial lung disease. In another aspect, the fibrosis is interstitial lung disease with progressive fibrosis. In another aspect, the interstitial lung disease is pulmonary fibrosis. In another aspect, the interstitial lung disease is idiopathic pulmonary fibrosis (IPF).

In some embodiments, the present disclosure provides a method for promoting tissue remodeling in a subject in need thereof by administering to the subject a therapeutically effective amount of a compound of the present disclosure, or a pharmaceutically acceptable salt thereof. In one aspect, the subject has suffered cardiac tissue damage due to a myocardial infarction.

In some embodiments, the present disclosure provides a method of promoting wound healing and/or reducing adhesions in a subject in need thereof by administering to the subject a therapeutically effective amount of a compound of the present disclosure, or a pharmaceutically acceptable salt thereof. In one aspect, the administration of the compound promotes wound healing and/or reduces adhesions through increased fibrinolysis.

In some embodiments, the present disclosure provides a method for treating or preventing a keloid disorder in a subject in need thereof by administering to the subject a therapeutically effective amount of a compound of the present disclosure, or a pharmaceutically acceptable salt thereof. In one aspect, the keloid disorder is selected from the group consisting of scar formation, keloid tumors, and keloid scar.

In some embodiments, the present disclosure provides a method for treating or preventing inflammation in a subject in need thereof by administering to the subject a therapeutically effective amount of a compound of the present disclosure, or a pharmaceutically acceptable salt thereof. In one aspect, the inflammation is chronic inflammation. In one aspect, the chronic inflammation is selected from the group consisting of rheumatoid arthritis, osteoarthritis, and Crohn's disease. In another aspect, the chronic inflammation is rheumatoid arthritis.

In some embodiments, the present disclosure provides a method of treating cancer in a subject in need of treatment by administering to the subject a therapeutically effective amount of a compound of the present disclosure, or a pharmaceutically acceptable salt thereof. In one aspect, the cancer is selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer. In another aspect, the cancer is hepatocellular carcinoma.

The subject treated typically will be a human or non-human mammal, particularly a human. Suitable subjects can also include domestic or wild animals; companion animals (including dogs, cats, and the like); livestock (including horses, cows and other ruminants, pigs, poultry, rabbits, and the like); primates (including monkeys such as rhesus monkeys, cynomolgus (also known as crab-eating or long-tailed) monkeys, marmosets, tamarins, chimpanzees, macaques, and the like); and rodents (including rats, mice, gerbils, guinea pigs, and the like).

In some embodiments, the present disclosure provides the compounds of the present disclosure, or pharmaceutically acceptable salts thereof, for use as medicaments.

In some embodiments, the present disclosure provides for the use of the compounds of the Formula I, or pharmaceutically acceptable salts thereof, for treating or preventing an FAP-mediated condition as discussed above.

In some embodiments, the present disclosure provides for the use of the compounds of the Formula I, or pharmaceutically acceptable salts thereof, for the manufacture of medicaments for treating or preventing an FAP-mediated condition as discussed above.

### IV. Combination Therapies and Fixed-Dose Combinations

The compounds of the present disclosure may be used in the methods described above as either as single pharmacological agents or in combination with other pharmacological agents or techniques. Such combination therapies may be achieved by way of the simultaneous, sequential or separate dosing of the individual components of the treatment. These combination therapies (and corresponding combination products) employ the compounds of the present disclosure within the dosage ranges described in this application and the other pharmacological agent(s), typically within its approved dosage range(s).

In some embodiments, the present disclosure provides a combination suitable for use in the treatment of a condition selected from the previously discussed conditions, wherein the combination comprises a compound of the present disclosure, or a pharmaceutically acceptable salt thereof, and a sodium-glucose transport protein 2 (SGLT2) inhibitor. In one aspect, the SGLT2 inhibitor is selected from the group consisting of canagliflozin, dapagliflozin, empagliflozin, ertugliflozin, ipragliflozin, luseogliflozin, and remogliflozin. In another aspect, the SGLT2 inhibitor is dapagliflozin.

In some embodiments, the present disclosure provides a combination suitable for use in the treatment of a condition selected from the previously discussed conditions, wherein the combination comprises a compound of the present disclosure, or a pharmaceutically acceptable salt thereof, and metformin.

In some embodiments, the present disclosure provides a combination suitable for use in the treatment of a condition selected from the previously discussed conditions, wherein the combination comprises a compound of the present disclosure, or a pharmaceutically acceptable salt thereof, and a glucagon-like peptide-1 receptor (GLP1) agonist. In one aspect, the GLP1 agonist is selected from the group consisting of exenatide, liraglutide, lixisenatide, albiglutide, dulaglutide, and semaglutide.

In some embodiments, the present disclosure provides a combination suitable for use in the treatment of a condition selected from the previously discussed conditions, wherein the combination comprises a compound of the present disclosure, or a pharmaceutically acceptable salt thereof, and a dipeptidyl peptidase 4 (DPP4) inhibitor. In one aspect, the DPP4 inhibitor is selected from the group consisting of sitagliptin, vildagliptin, saxagliptin, linagliptin, gemigliptin, anagliptin, teneligliptin, alogliptin, trelagliptin, omarigliptin, evogliptin, gosogliptin, and dutogliptin.

In some embodiments, the present disclosure provides a combination suitable for use in the treatment of a condition selected from the previously discussed conditions, wherein the combination comprises a compound of the present disclosure, or a pharmaceutically acceptable salt thereof, and a peroxisome proliferator-activated receptor (PPAR) agonist. In one aspect, the PPAR agonist is a PPARα agonist. In another aspect, the PPAR agonist is a PPARγ agonist. In another aspect, the PPAR agonist is a PPARα/γ agonist. In another aspect, the PPAR agonist is selected from the group consisting of clofibrate, gemfibrozil, ciprofibrate, bezafibrate, and fenofibrate. In another aspect, the PPAR agonist is a thiazolidinedione. In another aspect, the thiazolidinedione is selected from the group consisting of pioglitazone, rosiglitazone, lobeglitazone, and rivoglitazone. In another aspect, the PPAR agonist stimulates liver expression of FGF21.

In some embodiments, the present disclosure provides a pharmaceutical composition comprising a compound of the present disclosure, or a pharmaceutically acceptable salt thereof; one or more pharmacological agents selected from SGLT2 inhibitors, metformin, GLP1 agonists, DPP4 inhibitors, and PPAR agonists; and a pharmaceutically acceptable diluent or carrier. Such a combination can be used for the manufacture of a medicament for use in the treatment of a condition selected from the previously discussed conditions. In one aspect, the pharmaceutical composition comprises an SGLT2 inhibitor. In another aspect, the pharmaceutical composition comprises metformin. In another aspect, the pharmaceutical composition comprises a GLP1 agonist. In another aspect, the pharmaceutical composition comprises a DPP4 inhibitor. In another aspect, the pharmaceutical composition comprises a PPAR agonist.

In some embodiments, the present disclosure provides a combination suitable for use in the treatment of cancer, wherein the combination comprises a compound of the present disclosure, or a pharmaceutically acceptable salt thereof, and an immune checkpoint inhibitor. In one aspect, the immune checkpoint inhibitor is selected from the group consisting of anti-PD-1 antibodies, anti-PD-Ll antibodies, anti-CTLA4 antibodies, TLR7 agonists, CD40 agonists, Lag-3 antagonists, and OX40 agonists. In another aspect, the immune checkpoint inhibitor is an anti-PD-1 antibody (e.g., pembrolizumab (Keytruda), nivolumab (Opdivo), cemiplimab (Libtayo), etc.). In another aspect, the immune checkpoint inhibitor is an anti-PD-Ll antibody (e.g., atezolizumab (Tecentriq), avelumab (Bavencio), durvalumab (Imfinzi), etc.). In another aspect, the immune checkpoint inhibitor is an anti-CTLA4 antibody (e.g., ipilimumab (Yervoy), tremelimumab, etc.). In another aspect, the cancer is selected from the group consisting of pancreatic cancer, colon cancer, and rectal cancer.

### V. Pharmaceutical Compositions

The compounds of the present disclosure, and pharmaceutically acceptable salts thereof, may be administered as pharmaceutical compositions, comprising one or more pharmaceutically acceptable excipients. Therefore, in some embodiments the present disclosure provides pharmaceutical compositions comprising a compound of the present disclosure, or a pharmaceutically acceptable salt thereof, and at least one pharmaceutically acceptable excipient.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth Edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous or intramuscular dosing), or as a suppository for rectal dosing. The compositions may be obtained by conventional procedures using conventional pharmaceutical excipients, well known in the art. Thus, compositions intended for oral use may contain, for example, one or more coloring, sweetening, flavoring and/or preservative agents.

The total daily dose will necessarily be varied depending upon the subject treated, the particular route of administration, any therapies being co-administered, and the severity of the illness being treated, and may include single or multiple doses. Specific dosages can be adjusted, for example, depending upon the condition being treated; the age, body weight, general health condition, sex, and diet of the subject; administration routes; dose intervals; excretion rate; and other drugs being co-administered to the subject. An ordinarily skilled physician provided with the disclosure of the present application will be able to determine appropriate dosages and regimens for administration of the therapeutic agent to the subject, and to adjust such dosages and regimens as necessary during the course of treatment, in accordance with methods well-known in the therapeutic arts. The compound of the present disclosure, or a pharmaceutically acceptable salt thereof, typically will be administered to a warm-blooded animal at a unit dose within the range 2.5 to 5000 mg/m² body area of the animal, or approximately 0.05 to 100 mg/kg, and this normally provides a therapeutically effective dose.

In some embodiments, the present disclosure provides pharmaceutical compositions for use in therapy, comprising a compound of the present disclosure, or a pharmaceutically acceptable salt thereof, and at least one pharmaceutically acceptable excipient.

In some embodiments, the present disclosure provides pharmaceutical compositions for use in the treatment of an FAP-mediated condition, comprising a compound of the present disclosure, or a pharmaceutically acceptable salt thereof, and at least one pharmaceutically acceptable excipient. In one aspect, the FAP-mediated condition is selected from the group consisting of liver disease, type 2 diabetes mellitus, cardiovascular conditions, obesity, obesity-related conditions, fibrosis, keloid disorder, inflammation, and cancer.

### VI. Kits

The present disclosure further provides kits comprising a unit dosage form comprising a compound of the present disclosure, or a pharmaceutically acceptable salt thereof, contained within a packaging material and a label or package insert which indicates that the unit dosage form can be used for treating one or more of the previously described conditions.

In some embodiments, the kit comprises a unit dosage form comprising a compound of the present disclosure, or a pharmaceutically acceptable salt thereof, contained within a packaging material and a label or package insert which indicates that the pharmaceutical composition can be used for treating an FAP-mediated condition. In another aspect, the FAP-mediated condition is liver disease. In another aspect, the liver disease is selected from the group consisting of fatty liver disease, end stage liver disease, and cirrhosis. In another aspect, the liver disease is selected from the group consisting of Nonalcoholic Steatohepatitis (NASH) and Nonalcoholic Fatty Liver Disease (NAFLD).

In some embodiments, kit comprises: (a) a first unit dosage form comprising a compound of the present disclosure, or a pharmaceutically acceptable salt thereof; (b) a second unit dosage form comprising a pharmacological agent selected from the group consisting of SGLT2 inhibitors, metformin, GLP1 agonists, DPP4 inhibitors, and PPAR agonists; (c) a container means for containing said first and second dosage forms; and (d) a label or package insert which indicates that the first unit dosage form and second unit dosage form can be used for treating an FAP-mediated condition.

### VII. Methods of Preparation

The present disclosure further provides processes for the preparation of the compounds of Formulae (I), (II), (III-A), (III-B), (III-C), (III-D), (III-E), (IV), and (IV-A), and pharmaceutically acceptable salts thereof. Reaction schemes 1 to 10 illustrate synthetic routes to these compounds wherein, unless otherwise stated, R¹, R², R³, R⁵, and R⁶ are as defined in Formula (I); R⁷ is an alkyl group (e.g., methyl, ethyl, tert-butyl, etc.); R⁸ and R⁹ are alkyl groups, aryl groups, or hydrogen; X¹ is S, S(O) or S(O)₂; X² and X³ are leaving groups (e.g., Cl, Br, I, OMs, OTs, OTf, etc.); and PG¹ is a protective group. One of skill in the art will appreciate that these methods are representative and are not inclusive of all possible methods for preparing the compounds of the present disclosure.

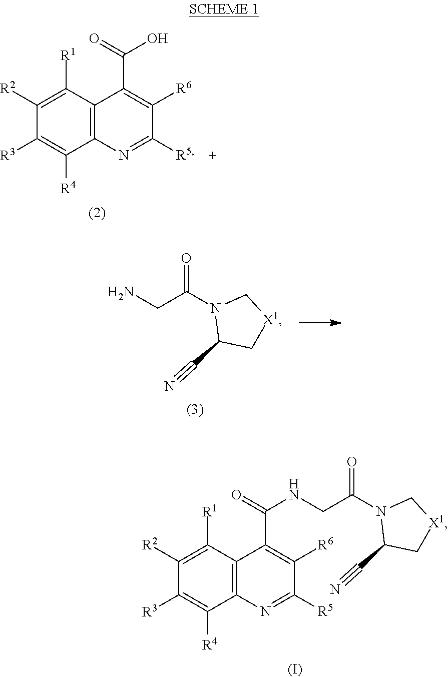

Scheme 1 illustrates a synthetic route to certain compounds of formula (I). A compound of formula (2) may be reacted with a compound of formula (3) to give a compound of formula (I). The reaction may be performed using suitable coupling reagents (e.g., HATU, HOBt/EDC, T3P, etc.) in the presence of a base, typically an organic base (such as DIPEA or TEA), using a solvent (such as DCM, DMF, EtOAc, MeCN, or mixtures thereof) at temperatures ranging from typically 0° C. to 60° C.

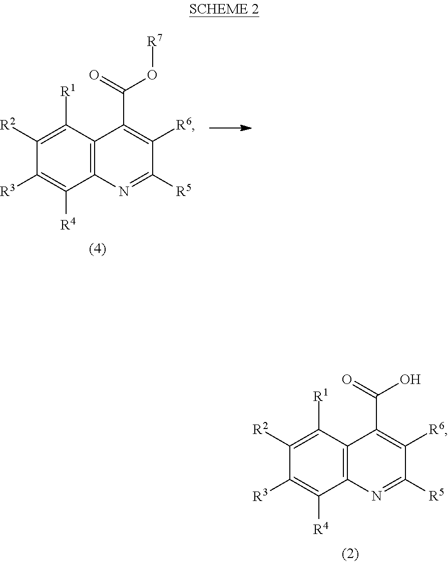

Scheme 2 illustrates a synthetic route to certain compounds of formula (2). A compound of formula (2) may be formed by reacting a compound of formula (4) with a base (e.g., NaOH, LiOH, etc.) in an organic solvent (e.g., 1,4-dioxane, THF, MeOH, or mixtures thereof), and optionally in the presence of water. The reaction may be performed in a temperature interval from 0° C. to reflux. Alternatively, for compounds of formula (4) where R⁷=tert-butyl, the reaction may be performed with a suitable acid (e.g., HCl, etc.) in a solvent (such as 1,4-dioxane, EtOAc, MeOH, water, or mixtures thereof). Alternatively, the reaction may be performed using carboxylic acids (such as TFA, etc.), neat, or in a solvent (such as DCM, etc.) at temperatures ranging from typically 0° C. to 60° C.

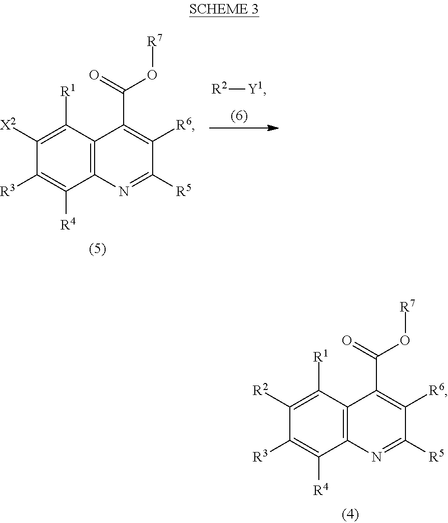

Scheme 3 illustrates synthetic routes to certain compounds of formula (4). A compound of formula (5) may be transformed into a compound of formula (4) by reaction with an alkyl halide, typically an alkyl bromide, of formula (6) wherein R² is as defined in formula (I) and Y¹ is as defined above. The reaction may be catalyzed with a Ni-catalyst (such as NiBr₂ DME, etc.) and a suitable ligand (such as dtbbpy, etc.), as well as a suitable photoredox catalyst (such as Ir[dF(CF₃)ppy]₂(dtbbpy)PF₆, etc.) in the presence of HSi(SiMe₃)₃ and a base (such as Na₂CO₃, etc.) in a suitable solvent (such as DME, etc.) typically at 20° C. while irradiated with blue light LED.

Alternatively, a compound of formula (4) may be formed by reacting a compound of formula (5) with an alkyl halide of formula (6) using methodology and conditions described in the following publications: MacMillan D. W. C. et. al. J. Am. Chem. Soc. 2020, 142, 11691-11697; Weix D. J. et. al. J. Am. Chem. Soc. 2010, 132, 920-921; Weix D. J. et. al. Nat. Chem. 2016, 8, 1126-1130; Gong H. et. al. J. Am. Chem. Soc. 2015, 137, 11562-11565 and Gong H. et. al. J. Am. Chem. Soc. 2018, 140, 14490-14497.

Alternatively, a compound of formula (4) may be formed by reacting a compound of formula (5) with an alkyl ether of formula (6) wherein R² is as defined in formula (I) and Y¹ is a hydrogen. The reaction may be catalyzed with a Ni-catalyst (such as NiBr₂ dtbbpy, etc.) as well as a suitable photoredox catalyst (such as TBADT, etc.) in the presence of a base (such as K₃PO₄, etc.) in a suitable solvent (such as MeCN, etc.), typically at 20° C. while irradiated with blue light LED.

Alternatively, a compound of formula (4) may be formed by reacting a compound of formula (5) with an aryl or alkyl boron reagent of formula (6), in which R² is as defined in formula (I) and Y¹ is a boron derivative (such as a boronic acid, trifluoroborate, etc.). The reaction may be catalyzed by a Pd-catalyst (such as PdCl₂(dppf)DCM, etc.) in the presence of a base (such as K₂CO₃, Na₂CO₃, etc.) in a suitable solvent (such as 1,4-dioxane, THF, etc.) at a temperature ranging from typically 20° C. to reflux.

Alternatively, a compound of formula (4) may be formed by reacting a compound of formula (5) with an alkyl boron reagent of formula (6), in which R² is as defined in formula (I) and Y¹ is a boron derivative (such as a boronic acid, trifluoroborate, etc.) using methodology and conditions described in the following publication: Harris M. R. et. al. Org. Lett. 2018, 20, 2867-2871.

Alternatively, a compound of formula (4) may be formed from a compound of formula (5) by reaction with an alcohol or a phenol of formula (6), wherein R² is as defined in formula (I) and Y¹ is a hydrogen. The reaction may be catalyzed with a suitable Pd-reagent (e.g., Pd(OAc)₂, etc.) with a suitable phosphine ligand (e.g., XPhos, t-BuXPhos, etc.) in the presence of a base (such as K₃PO₄, etc.) in a suitable solvent (such as toluene, 1,4-dioxane, etc.) and performed at elevated temperatures.

Alternatively, a compound of formula (4) may be formed from a compound of formula (5) by reaction with an alcohol or a phenol of formula (6), wherein R² is as defined in formula (I) and Y¹ is a hydrogen. The reaction may be promoted with a suitable Cu-reagent (e.g., CuBr, etc.) in the presence of a base (such as K₂CO₃, Cs₂CO₃, etc.) and a radical initiator (such as AIBN, etc.) in a suitable solvent (such as DMF, etc.) at elevated temperatures.

Alternatively, a compound of formula (4) may be formed by reacting a compound of formula (5) with a compound of formula (6) wherein R² is as defined in formula (I) and Y¹ is a hydrogen. The reaction may be catalyzed with a suitable Pd-reagent (e.g., Pd(OAc)₂, etc.) with a suitable phosphine ligand (e.g., BINAP, SPhos, etc.) in the presence of a base (such as NaHMDS, Cs₂CO₃, etc.) in a suitable solvent (such as 1,4-dioxane, etc.) and performed at elevated temperatures.

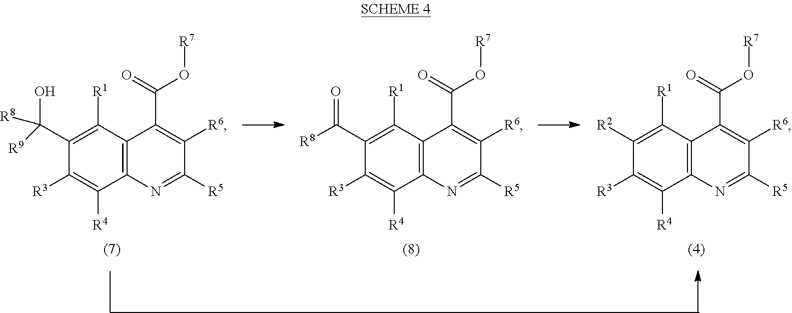

Scheme 4 illustrates synthetic routes to certain compounds of formula (4). A compound of formula (8) may be formed from a compound of formula (7) in which R⁸ is a hydrogen or an alkyl group, and R⁹ is a hydrogen, by reaction with an oxidation reagent (such as Dess-Martin periodinane, etc.). The reaction may be performed in a suitable solvent (such as DCM, etc.), typically at temperatures ranging from 0° C. to 40° C.

A compound of formula (4), in which R² is as defined in formula (I), may be formed by reacting a compound of formula (8) with a fluorinating reagent (such as DAST, etc.) in a suitable solvent (such as DCM, THF, etc.) at temperatures ranging from typically 0° C. to 60° C.

Alternatively, a compound of formula (4) may be formed from a compound of formula (8) by reduction with a reducing agent (such as sodium borohydride, etc.). The reaction may be performed in a solvent (such as methanol, etc.) at a temperature typically ranging from 0° C. to 40° C.

Alternatively, a compound of formula (4), may be formed from a compound of formula (8) by reaction with a Grignard reagent (such as MeMgCl, MeMgBr, etc.). The reaction may be performed in a suitable solvent (such as diethyl ether, THF, etc.) at a temperature typically ranging from −78° C. to 40° C.

Alternatively, a compound of formula (4), may be formed from a compound of formula (8) by condensation with a sulfonyl hydrazide, such as 4-methylbenzenesulfonohydrazide, in a suitable solvent (such as 1,4-dioxane, etc.). The intermediate hydrazone may then be reacted with a boronic acid. The reaction may be performed in a suitable solvent (such as 1,4-dioxane, etc.) in the presence of a base (such as K₂CO₃, or Na₂CO₃, etc.), typically at reflux temperature according to procedure described in Barluenga J. et. al. Nat. Chem. 2009, 1, 494-499.

Alternatively, a compound of formula (4), in which R² is as defined in formula (I), may be formed by reacting a compound of formula (7) with a fluorinating reagent (such as DAST, etc.) in a suitable solvent (such as DCM, THF, etc.) at temperatures ranging from typically 0° C. to 60° C.

Alternatively, a compound of formula (4) may be formed from a compound of formula (7) by reaction with an alkyl halide (such as MeI, etc.) or another alkylating agent (such as an alkyl sulfonate, alkyl triflate, etc.). The reaction may be performed using a base (such as NaH, etc.) in a solvent (such as THF, 1,4-dioxane, etc.).

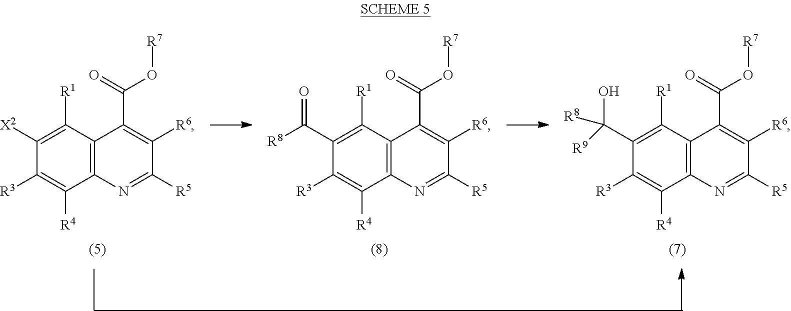

Scheme 5 illustrates synthetic routes to certain compounds of formula (7). A compound of formula (8), in which R⁸ is an alkyl group (such as a methyl group, etc.) may be formed by reacting a compound of formula (5) with a trialkyl(1-alkoxyvinyl)tin reagent (such as tributyl(1-ethoxyvinyl)tin, etc.). The reaction may be catalyzed by a Pd-reagent (such as PdCl₂(dppf)DCM, Pd(PPh₃)₄, etc.) in a suitable solvent (such as toluene, 1,4-dioxane, etc.) and at a temperature ranging from typically 80° C. to reflux.

Alternatively, a compound of formula (8), in which R⁸ is a hydrogen, may be formed by reacting a compound of formula (5) with formic acid and acetic acid anhydride. The reaction may be performed with a Pd-reagent (such as PdOAc₂, etc.) with a suitable ligand (such as butyl-1-adamantylphosphine, etc.) with NaHCO₃ and TEA, in a suitable solvent (such as DMF, etc.) and at a temperature ranging from typically 80° C. to 120° C.

A compound of formula (7), in which R⁸ and R⁹ are hydrogens, may be formed by reacting a compound of formula (5) with a (trialkylstannyl)methanol reagent (such as (tributylstannyl)methanol, etc.). The reaction may be catalyzed by a Pd-reagent (such as PdCl₂(dppf)DCM, Pd(PPh₃)₄, etc.) in a suitable solvent (such as toluene, 1,4-dioxane, etc.) and at a temperature ranging from typically 80° C. to reflux.

Alternatively, a compound of formula (7), in which R⁹ is a hydrogen, may be formed from a compound of formula (8) by reduction with a reducing agent (such as sodium borohydride, etc.). The reaction may be performed in a solvent (such as methanol, etc.) at a temperature typically ranging from 0° C. to 40° C.

Alternatively, a compound of formula (7), in which R⁹ is an alkyl group, may be formed from a compound of formula (8) by reaction with a Grignard reagent (such as MeMgCl, MeMgBr, etc.). The reaction may be performed in a suitable solvent (such as diethyl ether, THF, etc.) at a temperature typically ranging from −78° C. to 40° C.

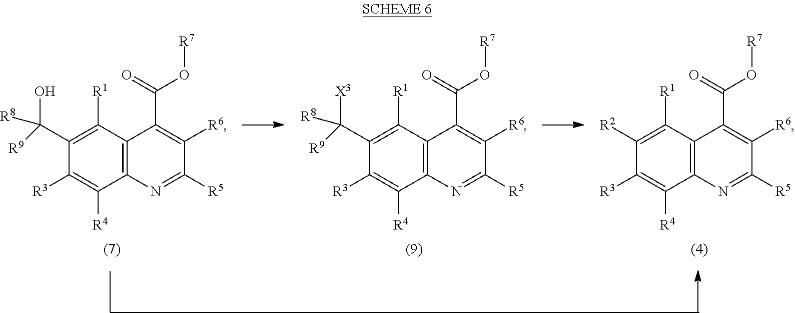

Scheme 6 illustrates synthetic routes to certain compounds of formula (4). A compound of formula (9) may be formed from a compound of formula (7) in which R⁸ and R⁹ are hydrogens or alkyl groups, by reaction with a suitable reagent (such as thionyl chloride, MsCl, etc.). The reaction may be performed in a suitable solvent (such as DCM, etc.), typically at temperatures ranging from 0° C. to 40° C.

A compound of formula (4) may be formed from a compound of formula (9) in which R⁸ and R⁹ are hydrogens or alkyl groups by reaction with an alcohol. The reaction may be performed in a suitable solvent (such as THF, DMF, etc.) in the presence of a base (such as NaH, etc.), typically at 0° C. to 40° C.

Alternatively, a compound of formula (4) may be formed from a compound of formula (9) in which R⁸ and R⁹ are hydrogens or alkyl groups by reaction with an amine. The reaction may be performed in a suitable solvent (such as MeCN, DMF, etc.) in the presence of a base (such as TEA, Cs₂CO₃, etc.) with or without additives (such as KI, etc.), typically at 0° C. to 80° C.

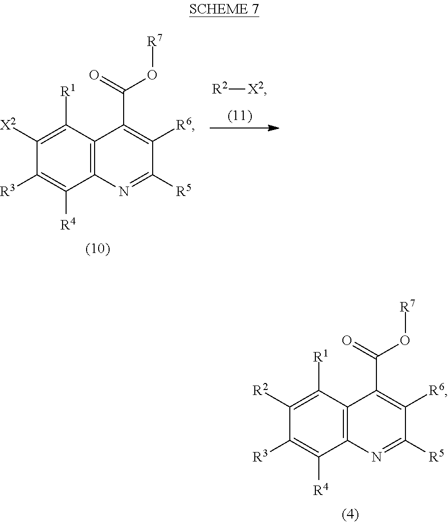

Scheme 7 illustrates synthetic routes to certain compounds of formula (4). A compound of formula (4) may be formed from a compound of formula (10) in which Y² is a boron derivative (such as boronic acid, boronic ester, trifluoroborate, etc.) by reaction with a reagent of formula (11) in which R² is as defined in formula (I) and X² is a leaving group as defined above. The reaction may be catalyzed by a Pd-reagent (such as PdCl₂(dppf)DCM, trans-bromo(N-succinimidyl)-bis(triphenylphosphine)palladium(II), etc.) in a suitable solvent (such as toluene, 1,4-dioxane, etc.) with or without water, and at a temperature ranging from typically 80° C. to 120° C.

Alternatively, a compound of formula (4) may be formed from a compound of formula (10) in which Y² is a boronic acid by reaction with a sulfonyl hydrazone, typically a tosylhydrazone, of an aldehyde or ketone. The reaction may be performed in a suitable solvent (such as 1,4-dioxane, etc.) in the presence of a base (such as K₂CO₃, Na₂CO₃, etc.) typically at reflux temperature according to procedure described in Barluenga J. et. al. Nat. Chem. 2009, 1, 494-499.

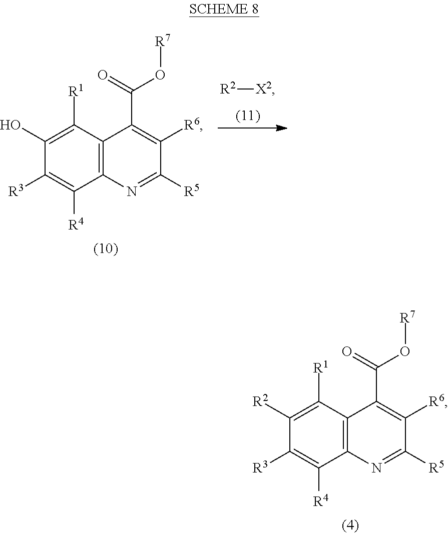

Scheme 8 illustrates synthetic routes to certain compounds of formula (4). A compound of formula (4), wherein R² is as defined in formula (I), may be formed from a compound of formula (12) by reaction with an alkyl halide, alkyl sulfonate or an alkyl triflate of formula (11). The reaction may be promoted by a base (such as Cs₂CO₃, K₂CO₃, etc.) in a suitable solvent (such as DMF, etc.) and at a temperature ranging from typically 20° C. to 100° C.

Alternatively, a compound of formula (4) may be formed from a compound of formula (12 by reaction with an alcohol of formula (11), wherein R² is as defined in formula (I) and X² is a hydroxyl group. The reaction may be performed in the presence of a phosphine (such as Ph₃P, etc.) and an azodicarboxylate (such as DIAD, etc.) in a suitable solvent (such as THF, etc.) and at a temperature ranging from typically 20° C. to 60° C.

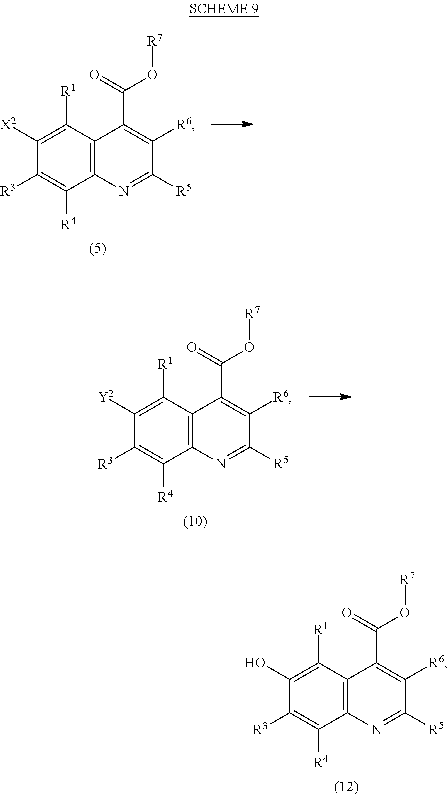

Scheme 9 illustrates synthetic routes to certain compounds of formula (12). A compound of formula (10) in which Y² is a boron derivative (such as boronic acid, boronic ester, trifluoroborate, etc.) may be formed from a compound of formula (5) in which X² is a leaving group as defined above, and a boron reagent (such as B₂(OH)₄, or B₂pin₂, etc.). The reaction may be catalyzed by a Pd-reagent (such as PdCl₂(dppf)DCM, etc.) and a base (such as KOAc, etc.) in a suitable solvent (such as toluene, 1,4-dioxane, EtOH, etc.) at a temperature ranging from typically 80° C. to 120° C.

A compound of formula (12) may be formed from a compound of formula (10) in which Y² is a boron derivative (such as boronic acid, boronic ester, etc.). The reaction may be promoted by a suitable reagent (such as NaBO₃, etc.) in a suitable solvent (such as THF/water mixture, etc.), typically at room temperature.

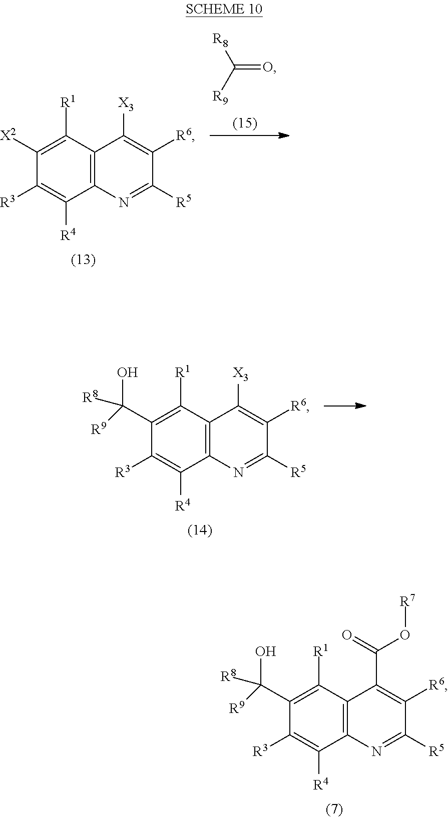

Scheme 10 illustrates synthetic routes to certain compounds of formula (7). A compound of formula (14) may be formed from a compound of formula (13), wherein X² and X³ are as defined above, and selected in such a way that X² may be selectively reacted with an organometallic reagent (such as BuLi, etc.) and then reacted with an aldehyde or ketone of formula (15), wherein R⁸ and R⁹ are as defined above. The reaction may be performed in a suitable solvent (such as THF, etc.) and typically performed at low temperatures form −78° C. to 0° C.

A compound of formula (7) may be formed by reacting a compound of formula (14) with carbon monoxide, typically at a pressure of 1 to 10 atm at a temperature ranging from typically 80° C. to 120° C. in a sealed vessel. The reaction may be catalyzed with a suitable Pd-reagent (e.g. Pd(dppf)Cl₂, etc.) in the presence of a base (e.g., TEA, etc.) in the presence of a suitable alcohol (such as MeOH, EtOH, etc.) in a suitable solvent, or using the alcohol as solvent.

It is understood that organic reactions described herein are performed according to laboratory practice known to person skilled in the art. It is understood that some of the reactions described herein may optionally be performed in different orders than laid out herein. It is understood that chiral isomers of compounds herein can be resolved at any stage in the synthetic process using chiral resolving agents described in the literature and known to person skilled in the art, or chiral chromatography methods described in the literature and known to person skilled in the art, or as described further in the Examples.

It is further understood that additional protective groups may optionally be needed in some of the steps described above, and it is further understood that a deprotection step therefore optionally may be performed, using method described in the literature and known to person skilled in the art. The protection and deprotection of functional groups is described in “Protective Groups in Organic Synthesis” 3^{rd} Ed, T. W. Greene and P. G. M. Wutz, Wiley-Interscience (1999), which publication is incorporated herein by reference.

### VIII. Numbered Embodiments

Embodiment 1. A compound having the structure of Formula (I):

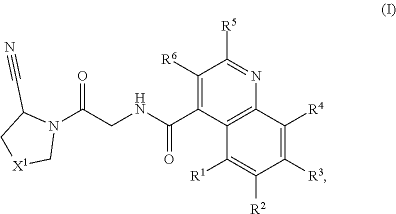

or a pharmaceutically acceptable salt thereof, wherein:
X¹ is selected from the group consisting of —S—, —S(O)—, and —S(O)₂—;
R¹ is selected from the group consisting of hydrogen, halogen, hydroxy, C₁₋₃-alkyl, and C₁₋₆-alkoxy;
R² is selected from the group consisting of
   (a) C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, cyano, C₁₋₆-alkyl, and halo-C₁₋₆-alkyl;
   (b) C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
   (c) phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy; and
   (d) a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
   (e) —OR⁷, wherein R⁷ is selected from the group consisting of C₁₋₆-alkyl, phenyl, and tetrahydropyranyl; wherein:
      (i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
      (ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
      (iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy;
R³ is selected from the group consisting of hydrogen, halogen, and C₁₋₃-alkyl;
R⁴ is selected from the group consisting of hydrogen, halogen, and C₁₋₃-alkyl;
R⁵ is selected from the group consisting of hydrogen, halogen, and C₁₋₃-alkyl; and
R⁶ is selected from the group consisting of hydrogen, halogen, and C₁₋₃-alkyl.

Embodiment 2. The compound of embodiment 1, or a pharmaceutically acceptable salt thereof, wherein the compound has the structure of Formula (II):

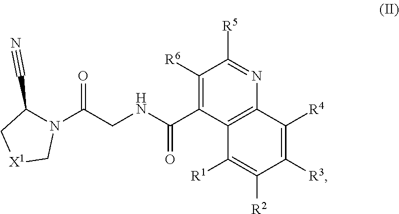

wherein X¹, R¹, R², R³, R⁴, R⁵, and R⁶ are as defined in embodiment 1.

Embodiment 3. The compound of embodiment 1 or 2, or a pharmaceutically acceptable salt thereof, wherein X¹ is —S—.

Embodiment 4. The compound of embodiment 1 or 2, or a pharmaceutically acceptable salt thereof, wherein X¹ is —S(O)—.

Embodiment 5. The compound of embodiment 1 or 2, or a pharmaceutically acceptable salt thereof, wherein X¹ is —S(O)₂—.

Embodiment 6. The compound of any of embodiments 1 to 5, or a pharmaceutically acceptable salt thereof, wherein R¹ is selected from the group consisting of hydrogen, halogen, and C₁₋₃-alkyl.

Embodiment 7. The compound of any of embodiments 1 to 5, or a pharmaceutically acceptable salt thereof, wherein R¹ is selected from the group consisting of hydrogen, chloro, fluoro, and methyl.

Embodiment 8. The compound of any of embodiments 1 to 5, or a pharmaceutically acceptable salt thereof, wherein R¹ is hydrogen.

Embodiment 9. The compound of any of embodiments 1 to 5, or a pharmaceutically acceptable salt thereof, wherein R¹ is chloro.

Embodiment 10. The compound of any of embodiments 1 to 5, or a pharmaceutically acceptable salt thereof, wherein R¹ is fluoro.

Embodiment 11. The compound of any of embodiments 1 to 5, or a pharmaceutically acceptable salt thereof, wherein R¹ is methyl.

Embodiment 12. The compound of any of embodiments 1 to 11, or a pharmaceutically acceptable salt thereof, wherein R³ is selected from the group consisting of hydrogen, chloro, fluoro, and methyl.

Embodiment 13. The compound of any of embodiments 1 to 11, or a pharmaceutically acceptable salt thereof, wherein R³ is hydrogen.

Embodiment 14. The compound of any of embodiments 1 to 11, or a pharmaceutically acceptable salt thereof, wherein R³ is chloro.

Embodiment 15. The compound of any of embodiments 1 to 11, or a pharmaceutically acceptable salt thereof, wherein R³ is fluoro.

Embodiment 16. The compound of any of embodiments 1 to 11, or a pharmaceutically acceptable salt thereof, wherein R³ is methyl.

Embodiment 17. The compound of any of embodiments 1 to 16, or a pharmaceutically acceptable salt thereof, wherein R⁴ is selected from the group consisting of hydrogen, halogen, and methyl.

Embodiment 18. The compound of any of embodiments 1 to 16, or a pharmaceutically acceptable salt thereof, wherein R⁴ is hydrogen.

Embodiment 19. The compound of any of embodiments 1 to 16, or a pharmaceutically acceptable salt thereof, wherein R⁴ is chloro.

Embodiment 20. The compound of any of embodiments 1 to 16, or a pharmaceutically acceptable salt thereof, wherein R⁴ is fluoro.

Embodiment 21. The compound of any of embodiments 1 to 16, or a pharmaceutically acceptable salt thereof, wherein R⁴ is methyl.

Embodiment 22. The compound of any of embodiments 1 to 21, or a pharmaceutically acceptable salt thereof, wherein R⁵ is selected from the group consisting of hydrogen, chloro, fluoro, and methyl.

Embodiment 23. The compound of any of embodiments 1 to 21, or a pharmaceutically acceptable salt thereof, wherein R⁵ is hydrogen.

Embodiment 24. The compound of any of embodiments 1 to 21, or a pharmaceutically acceptable salt thereof, wherein R⁵ is fluoro.

Embodiment 25. The compound of any of embodiments 1 to 21, or a pharmaceutically acceptable salt thereof, wherein R⁵ is chloro.

Embodiment 26. The compound of any of embodiments 1 to 21, or a pharmaceutically acceptable salt thereof, wherein R⁵ is methyl.

Embodiment 27. The compound of any of embodiments 1 to 26, or a pharmaceutically acceptable salt thereof, wherein R⁶ is selected from the group consisting of hydrogen, chloro, fluoro, and methyl.

Embodiment 28. The compound of any of embodiments 1 to 26, or a pharmaceutically acceptable salt thereof, wherein R⁶ is hydrogen.

Embodiment 29. The compound of any of embodiments 1 to 26, or a pharmaceutically acceptable salt thereof, wherein R⁶ is chloro.

Embodiment 30. The compound of any of embodiments 1 to 26, or a pharmaceutically acceptable salt thereof, wherein R⁶ is fluoro.

Embodiment 31. The compound of any of embodiments 1 to 26, or a pharmaceutically acceptable salt thereof, wherein R⁶ is methyl.

Embodiment 32. The compound of any of embodiments 1 to 5, or a pharmaceutically acceptable salt thereof, wherein one of the R¹, R³, R⁴, R⁵, and R⁶ substituents is selected from the group consisting of halogen and C₁₋₃-alkyl, and the remaining R¹, R³, R⁴, R⁵, and R⁶ substituents are all hydrogen.

Embodiment 33. The compound of embodiment 32, or a pharmaceutically acceptable salt thereof, wherein one of the R¹, R³, R⁴, R⁵, and R⁶ substituents is selected from the group consisting of chloro, fluoro, and methyl, and the remaining R¹, R³, R⁴, R⁵, and R⁶ substituents are all hydrogen.

Embodiment 34. The compound of embodiment 32, or a pharmaceutically acceptable salt thereof, wherein one of the R¹, R³, R⁴, R⁵, and R⁶ substituents is selected from the group consisting of chloro and fluoro, and the remaining R¹, R³, R⁴, R⁵, and R⁶ substituents are all hydrogen.

Embodiment 35. The compound of embodiment 32, or a pharmaceutically acceptable salt thereof, wherein one of the R¹, R³, R⁴, R⁵, and R⁶ substituents is methyl, and the remaining R¹, R³, R⁴, R⁵, and R⁶ substituents are all hydrogen.

Embodiment 36. The compound of any of embodiments 32 to 35, or a pharmaceutically acceptable salt thereof, wherein the compound has the structure of Formula (III-A).

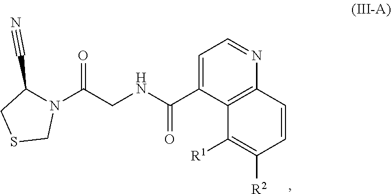

wherein R¹ and R² are as defined in embodiment 1.

Embodiment 37. The compound of any of embodiments 32 to 35, or a pharmaceutically acceptable salt thereof, wherein the compound has the structure of Formula (III-B):

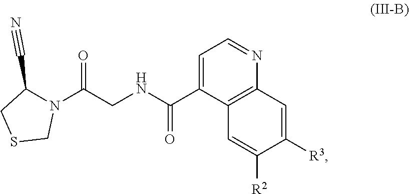

wherein R² and R³ are as defined in embodiment 1.

Embodiment 38. The compound of any of embodiments 32 to 35, or a pharmaceutically acceptable salt thereof, wherein the compound has the structure of Formula (III-C):

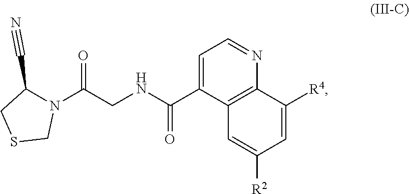

wherein R² and R⁴ are as defined in embodiment 1.

Embodiment 39. The compound of any of embodiments 32 to 35, or a pharmaceutically acceptable salt thereof, wherein the compound has the structure of Formula (III-D):

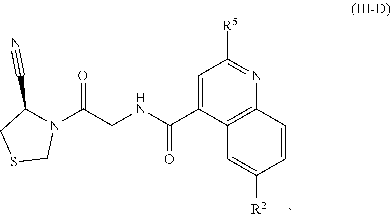

wherein R² and R⁵ are as defined in embodiment 1.

Embodiment 40. The compound of any of any of embodiments 32 to 35, or a pharmaceutically acceptable salt thereof, wherein the compound has the structure of Formula (III-E):

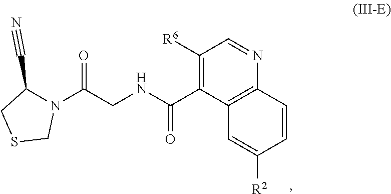

wherein R² and R⁶ are as defined in embodiment 1.

Embodiment 41. The compound of any of embodiments 1 to 5, or a pharmaceutically acceptable salt thereof, wherein at least two of the R¹, R³, R⁴, R⁵, and R⁶ substituents are independently selected from the group consisting of halogen and C₁₋₃-alkyl, and the remaining R¹, R³, R⁴, R⁵, and R⁶ substituents are all hydrogen.

Embodiment 42. The compound of embodiment 41, or a pharmaceutically acceptable salt thereof, wherein two of the R¹, R³, R⁴, R⁵, and R⁶ substituents are independently selected from the group consisting of chloro, fluoro, and methyl, and the remaining R¹, R³, R⁴, R⁵, and R⁶ substituents are all hydrogen.

Embodiment 43. The compound of embodiment 42, or a pharmaceutically acceptable salt thereof, wherein at least one of the R¹, R³, R⁴, R⁵, and R⁶ substituents is chloro.

Embodiment 44. The compound of embodiment 42, or a pharmaceutically acceptable salt thereof, wherein at least one of the R¹, R³, R⁴, R⁵, and R⁶ substituents is fluoro.

Embodiment 45. The compound of embodiment 42, or a pharmaceutically acceptable salt thereof, wherein at least one of the R¹, R³, R⁴, R⁵, and R⁶ substituents is methyl.

Embodiment 46. The compound of embodiment 1, or a pharmaceutically acceptable salt thereof, wherein the compound has the structure of Formula (IV):

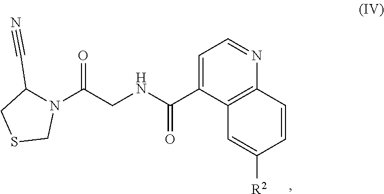

wherein R² is as defined in embodiment 1.

Embodiment 47. The compound of embodiment 1, or a pharmaceutically acceptable salt thereof, wherein the compound has the structure of Formula (IV-A):

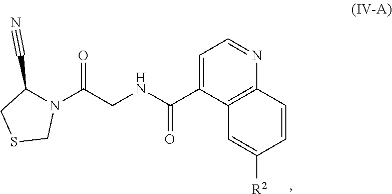

wherein R² is as defined in embodiment 1.

Embodiment 48. The compound of any of embodiments 1 to 47, or a pharmaceutically acceptable salt thereof, wherein R² is C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl.

Embodiment 49. The compound of any of embodiments 1 to 47, or a pharmaceutically acceptable salt thereof, wherein R² is C₁₋₃-alkyl, wherein the C₁₋₃-alkyl is optionally substituted with one or more substituents independently selected from the group consisting of halogen, hydroxy, cyano, C₃₋₆-cycloalkyl, halo-C₃₋₆-cycloalkyl, C₁₋₃-alkoxy, halo-C₁₋₃-alkoxy, C₃₋₆-cycloalkoxy, halo-C₃₋₆-cycloalkoxy, C₁₋₃-alkoxy-C₁₋₃-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₃-alkyl.

Embodiment 50. The compound of any of embodiments 1 to 47, or a pharmaceutically acceptable salt thereof, wherein R² is C₁₋₆-alkyl, wherein the C₁₋₆-alkyl is optionally substituted with one or more substituents independently selected from the group consisting of halogen, hydroxy, C₁₋₆-alkoxy, halo-C₁₋₆-alkoxy, C₃₋₆-cycloalkoxy, C₁₋₆-alkoxy-C₁₋₆-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl.

Embodiment 51. The compound of any of embodiments 1 to 47, or a pharmaceutically acceptable salt thereof, wherein R² is C₁₋₃-alkyl, wherein the C₁₋₃-alkyl is optionally substituted with one or more substituents independently selected from the group consisting of halogen, hydroxy, C₁₋₃-alkoxy, halo-C₁₋₃-alkoxy, C₃₋₆-cycloalkoxy, C₁₋₃-alkoxy-C₁₋₃-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₆-alkyl.

Embodiment 52. The compound of any of embodiments 1 to 47, or a pharmaceutically acceptable salt thereof, wherein R² is C₁₋₆-alkyl.

Embodiment 53. The compound of any of embodiments 1 to 47, or a pharmaceutically acceptable salt thereof, wherein R² is C₁₋₆-alkyl, wherein the C₁₋₆-alkyl is optionally substituted with one or more halogen.

Embodiment 54. The compound of any of embodiments 1 to 47, or a pharmaceutically acceptable salt thereof, wherein R² is C₁₋₆-alkyl, wherein the C₁₋₆-alkyl is optionally substituted with one or more fluoro.

Embodiment 55. The compound of any of embodiments 1 to 47, or a pharmaceutically acceptable salt thereof, wherein R² is C₁₋₆-alkyl, wherein the C₁₋₆-alkyl is optionally substituted with one or more hydroxy.

Embodiment 56. The compound of any of embodiments 1 to 47, or a pharmaceutically acceptable salt thereof, wherein R² is C₁₋₆-alkyl, wherein the C₁₋₆-alkyl is optionally substituted with one or more C₁₋₆-alkoxy.

Embodiment 57. The compound of any of embodiments 1 to 47, or a pharmaceutically acceptable salt thereof, wherein R² is C₁₋₆-alkyl, wherein the C₁₋₆-alkyl is optionally substituted with one or more halo-C₁₋₆-alkoxy.

Embodiment 58. The compound of any of embodiments 1 to 47, or a pharmaceutically acceptable salt thereof, wherein R² is C₁₋₆-alkyl, wherein the C₁₋₆-alkyl is optionally substituted with one or more C₃₋₆-cycloalkoxy.

Embodiment 59. The compound of any of embodiments 1 to 47, or a pharmaceutically acceptable salt thereof, wherein R² is C₁₋₆-alkyl, wherein the C₁₋₆-alkyl is optionally substituted with one or more C₁₋₆-alkoxy-C₁₋₆-alkoxy.

Embodiment 60. The compound of any of embodiments 1 to 47, or a pharmaceutically acceptable salt thereof, wherein R² is C₁₋₆-alkyl, wherein the C₁₋₆-alkyl is optionally substituted with one or more tetrahydrofuranyl, wherein the tetrahydrofuranyl is optionally substituted with one or more substituents independently selected from the group consisting of halogen, C₁₋₆-alkyl, and halo-C₁₋₆-alkyl.

Embodiment 61. The compound of any of embodiments 1 to 47, or a pharmaceutically acceptable salt thereof, wherein R² is C₁₋₆-alkyl, wherein the C₁₋₆-alkyl is optionally substituted with one or more tetrahydrofuranyloxy, wherein the tetrahydrofuranyloxy is optionally substituted with one or more substituents independently selected from the group consisting of halogen, C₁₋₆-alkyl, and halo-C₁₋₆-alkyl.

Embodiment 62. The compound of any of embodiments 1 to 47, or a pharmaceutically acceptable salt thereof, wherein R² is C₁₋₆-alkyl, wherein the C₁₋₆-alkyl is optionally substituted with one or more tetrahydropyranyl, wherein the tetrahydropyranyl is optionally substituted with one or more substituents independently selected from the group consisting of halogen, C₁₋₆-alkyl, and halo-C₁₋₆-alkyl.

Embodiment 63. The compound of any of embodiments 1 to 47, or a pharmaceutically acceptable salt thereof, wherein R² is C₁₋₆-alkyl, wherein the C₁₋₆-alkyl is optionally substituted with one or more morpholinyl, wherein the morpholinyl is optionally substituted with one or more substituents independently selected from the group consisting of halogen, C₁₋₆-alkyl, and halo-C₁₋₆-alkyl.

Embodiment 64. The compound of any of embodiments 1 to 47, or a pharmaceutically acceptable salt thereof, wherein R² is C₁₋₆-alkyl, wherein the C₁₋₆-alkyl is optionally substituted with one or more pyrazolyl, wherein the pyrazolyl is optionally substituted with one or more substituents independently selected from the group consisting of halogen, C₁₋₆-alkyl, and halo-C₁₋₆-alkyl.

Embodiment 65. The compound of any of embodiments 1 to 47, or a pharmaceutically acceptable salt thereof, wherein R² is C₁₋₆-alkyl, wherein the C₁₋₆-alkyl is optionally substituted with one or more isoxazolyl, wherein the isoxazolyl is optionally substituted with one or more substituents independently selected from the group consisting of halogen, C₁₋₆-alkyl, and halo-C₁₋₆-alkyl.

Embodiment 66. The compound of any of embodiments 1 to 47, or a pharmaceutically acceptable salt thereof, wherein R² is C₁₋₆-alkyl, wherein the C₁₋₆-alkyl is optionally substituted with one or more triazolyl, wherein the triazolyl is optionally substituted with one or more substituents independently selected from the group consisting of halogen, C₁₋₆-alkyl, and halo-C₁₋₆-alkyl.

Embodiment 67. The compound of any of embodiments 1 to 47, or a pharmaceutically acceptable salt thereof, wherein R² is C₁₋₆-alkyl, wherein the C₁₋₆-alkyl is optionally substituted with one or more pyridinyl, wherein the pyridinyl is optionally substituted with one or more substituents independently selected from the group consisting of halogen, C₁₋₆-alkyl, and halo-C₁₋₆-alkyl.

Embodiment 68. The compound of any of embodiments 1 to 47, or a pharmaceutically acceptable salt thereof, wherein R² is selected from the group consisting of methyl, fluoromethyl, difluoromethyl, trifluoromethyl, hydroxyethyl, hydroxypropyl, ethoxymethyl, difluoroethoxymethyl, methoxyethyl, dimethoxyethyl, difluoropropyl, trifluoropropyl, methoxypropyl, methoxyethoxymethyl, cyclopropoxymethyl, morpholinylmethyl, tetrahydrofuranylmethyl, tetrahydrofuranyloxymethyl, tetrahydropyranylmethyl, pyrazolylmethyl, dimethylpyrazolylmethyl, methylisoxazolylmethyl, methylisoxazolylethyl, methyltriazolylmethyl, dimethyltriazolylmethyl, dimethylpyridinylmethyl, methylpyridinylethyl, fluoropyridinylethyl, chloropyridinylethyl, and trifluoromethylpyridinylethyl.

Embodiment 69. The compound of embodiment 1, or a pharmaceutically acceptable salt thereof, wherein the compound is selected from the group consisting of:
N-(2-((R)-4-Cyanothiazolidin-3-yl)-2-oxoethyl)-6-((RS)-1-(3-fluoropyridin-2-yl)-ethyl)quinoline-4-carboxamide;
(R)—N-(2-(4-Cyanothiazolidin-3-yl)-2-oxoethyl)-6-(2-hydroxypropan-2-yl)quinoline-4-carboxamide;
(R)—N-(2-(4-Cyanothiazolidin-3-yl)-2-oxoethyl)-6-(trifluoromethyl)-quinoline-4-carboxamide;
(R)—N-(2-(4-Cyanothiazolidin-3-yl)-2-oxoethyl)-6-(2-methoxypropan-2-yl)quinoline-4-carboxamide;
(R)—N-(2-(4-Cyanothiazolidin-3-yl)-2-oxoethyl)-6-((2,5-dimethyl-2H-1,2,3-triazol-4-yl)-methyl)quinoline-4-carboxamide;
(R)—N-(2-(4-Cyanothiazolidin-3-yl)-2-oxoethyl)-6-(morpholino-methyl)quinoline-4-carboxamide;
N-(2-((R)-4-Cyanothiazolidin-3-yl)-2-oxoethyl)-6-((RS)-1-hydroxyethyl)quinoline-4-carboxamide;
N-(2-((R)-4-Cyanothiazolidin-3-yl)-2-oxoethyl)-6-((RS)-1-methoxyethyl)quinoline-4-carboxamide;
(R)—N-(2-(4-cyanothiazolidin-3-yl)-2-oxoethyl)-6-(ethoxymethyl)-quinoline-4-carboxamide;
(R)—N-(2-(4-cyanothiazolidin-3-yl)-2-oxoethyl)-6-methylquinoline-4-carboxamide;
(R)—N-(2-(4-cyanothiazolidin-3-yl)-2-oxoethyl)-6-(fluoromethyl)-quinoline-4-carboxamide;
N-(2-((R)-4-Cyanothiazolidin-3-yl)-2-oxoethyl)-6-((RS)-1,2-dimethoxyethyl)quinoline-4-carboxamide;
(R)—N-(2-(4-Cyanothiazolidin-3-yl)-2-oxoethyl)-6-((2-methoxyethoxy)-methyl)quinoline-4-carboxamide;
N-(2-((R)-4-Cyanothiazolidin-3-yl)-2-oxoethyl)-6-((((RS)-tetrahydrofuran-3-yl)oxy)-methyl)quinoline-4-carboxamide;
(R)—N-(2-(4-Cyanothiazolidin-3-yl)-2-oxoethyl)-6-((2,2-difluoroethoxy)methyl)quinoline-4-carboxamide;
(R)—N-(2-(4-Cyanothiazolidin-3-yl)-2-oxoethyl)-6-(cyclopropoxy-methyl)quinoline-4-carboxamide;
(R)—N-(2-(4-Cyanothiazolidin-3-yl)-2-oxoethyl)-6-((4-methylisoxazol-3-yl)methyl)-quinoline-4-carboxamide;
(R)—N-(2-(4-Cyanothiazolidin-3-yl)-2-oxoethyl)-6-((4,6-dimethylpyridin-3-yl)-methyl)quinoline-4-carboxamide;
(R)—N-(2-(4-Cyanothiazolidin-3-yl)-2-oxoethyl)-6-((1,3-dimethyl-1H-pyrazol-5-yl)-methyl)quinoline-4-carboxamide;
(R)—N-(2-(4-Cyanothiazolidin-3-yl)-2-oxoethyl)-6-((2-methyl-2H-1,2,3-triazol-4-yl)-methyl)quinoline-4-carboxamide;
(R)—N-(2-(4-Cyanothiazolidin-3-yl)-2-oxoethyl)-6-((5-methylisoxazol-3-yl)-methyl)quinoline-4-carboxamide;
N-(2-((R)-4-Cyanothiazolidin-3-yl)-2-oxoethyl)-6-((RS)-1-(5-methylisoxazol-3-yl)-ethyl)quinoline-4-carboxamide;
6-((RS)-1-(5-Chloropyridin-2-yl)ethyl)-N-(2-((R)-4-cyanothiazolidin-3-yl)-2-oxoethyl)quinoline-4-carboxamide;
N-(2-((R)-4-Cyanothiazolidin-3-yl)-2-oxoethyl)-6-((RS)-1-(3-methylisoxazol-5-yl)-ethyl)quinoline-4-carboxamide;
N-(2-((R)-4-Cyanothiazolidin-3-yl)-2-oxoethyl)-6-((RS)-1-(6-methylpyridin-3-yl)-ethyl)quinoline-4-carboxamide;
N-(2-((R)-4-Cyanothiazolidin-3-yl)-2-oxoethyl)-6-((RS)-1-(2-(trifluoromethyl)pyridin-4-yl)ethyl)quinoline-4-carboxamide;
(R)—N-(2-(4-Cyanothiazolidin-3-yl)-2-oxoethyl)-6-(2,2-difluoropropyl)quinoline-4-carboxamide;
(R)—N-(2-(4-Cyanothiazolidin-3-yl)-2-oxoethyl)-6-((tetrahydro-2H-pyran-4-yl)-methyl)quinoline-4-carboxamide;
(R)—N-(2-(4-Cyanothiazolidin-3-yl)-2-oxoethyl)-6-(3,3,3-trifluoropropyl)quinoline-4-carboxamide;
N-(2-((R)-4-Cyanothiazolidin-3-yl)-2-oxoethyl)-6-(((RS)-tetrahydrofuran-3-yl)-methyl)quinoline-4-carboxamide;
(R)—N-(2-(4-Cyanothiazolidin-3-yl)-2-oxoethyl)-6-(difluoromethyl)-quinoline-4-carboxamide;
(R)-6-((1H-Pyrazol-1-yl)methyl)-N-(2-(4-cyanothiazolidin-3-yl)-2-oxoethyl)quinoline-4-carboxamide;
(R)—N-(2-(4-cyanothiazolidin-3-yl)-2-oxoethyl)-6-(3-methoxypropyl)quinoline-4-carboxamide; and
(R)—N-(2-(4-cyanothiazolidin-3-yl)-2-oxoethyl)-6-(2-methoxyethyl)quinoline-4-carboxamide.

Embodiment 70. The compound of any of embodiments 1 to 47, or a pharmaceutically acceptable salt thereof, wherein R² is C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl.

Embodiment 71. The compound of any of embodiments 1 to 47, or a pharmaceutically acceptable salt thereof, wherein R² is C₃₋₆-cycloalkyl, wherein the C₃₋₆-cycloalkyl is optionally substituted with one or more substituents independently selected from the group consisting of halogen, hydroxy, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl.

Embodiment 72. The compound of any of embodiments 1 to 47, or a pharmaceutically acceptable salt thereof, wherein R² is C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl.

Embodiment 73. The compound of any of embodiments 1 to 47, or a pharmaceutically acceptable salt thereof, wherein R² is C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl.

Embodiment 74. The compound of any of embodiments 1 to 47, or a pharmaceutically acceptable salt thereof, wherein R² is C₃₋₆-cycloalkyl.

Embodiment 75. The compound of any of embodiments 1 to 47, or a pharmaceutically acceptable salt thereof, wherein R² is C₃₋₆-cycloalkyl, wherein the C₃₋₆-cycloalkyl is optionally substituted with one or more halogen.

Embodiment 76. The compound of any of embodiments 1 to 47, or a pharmaceutically acceptable salt thereof, wherein R² is C₃₋₆-cycloalkyl, wherein the C₃₋₆-cycloalkyl is optionally substituted with one or more cyano.

Embodiment 77. The compound of any of embodiments 1 to 47, or a pharmaceutically acceptable salt thereof, wherein R² is C₃₋₆-cycloalkyl, wherein the C₃₋₆-cycloalkyl is optionally substituted with one or more C₁₋₆-alkoxy.

Embodiment 78. The compound of any of embodiments 1 to 47, or a pharmaceutically acceptable salt thereof, wherein R² is C₃₋₆-cycloalkyl, wherein the C₃₋₆-cycloalkyl is optionally substituted with one or more isoxazolyl, wherein the isoxazolyl is optionally substituted with one or more substituents independently selected from the group consisting of halogen, C₁₋₆-alkyl, and halo-C₁₋₆-alkyl.

Embodiment 79. The compound of any of embodiments 1 to 47, or a pharmaceutically acceptable salt thereof, wherein R² is C₃₋₆-cycloalkyl, wherein the C₃₋₆-cycloalkyl is optionally substituted with one or more pyridinyl, wherein the pyridinyl is optionally substituted with one or more substituents independently selected from the group consisting of halogen, C₁₋₆-alkyl, and halo-C₁₋₆-alkyl.

Embodiment 80. The compound of any of embodiments 70 to 79, or a pharmaceutically acceptable salt thereof, wherein R² is optionally substituted cyclopropyl.

Embodiment 81. The compound of any of embodiments 70 to 79, or a pharmaceutically acceptable salt thereof, wherein R² is optionally substituted cyclobutyl.

Embodiment 82. The compound of any of embodiments 70 to 79, or a pharmaceutically acceptable salt thereof, wherein R² is optionally substituted cyclopentyl.

Embodiment 83. The compound of any of embodiments 70 to 79, or a pharmaceutically acceptable salt thereof, wherein R² is optionally substituted cyclohexyl.

Embodiment 84. The compound of any of embodiments 1 to 47, or a pharmaceutically acceptable salt thereof, wherein R² is selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl.

Embodiment 85. The compound of embodiment 1, or a pharmaceutically acceptable salt thereof, wherein the compound is selected from the group consisting of:
(R)—N-(2-(4-Cyanothiazolidin-3-yl)-2-oxoethyl)-6-(1-(2-(trifluoromethyl)pyridin-4-yl)-cyclopropyl)quinoline-4-carboxamide;
(R)—N-(2-(4-Cyanothiazolidin-3-yl)-2-oxoethyl)-6-(1-(6-methylpyridin-3-yl)-cyclopropyl)quinoline-4-carboxamide;
(R)—N-(2-(4-Cyanothiazolidin-3-yl)-2-oxoethyl)-6-(1-(3-fluoropyridin-2-yl)-cyclopropyl)quinoline-4-carboxamide;
(R)—N-(2-(4-Cyanothiazolidin-3-yl)-2-oxoethyl)-6-(1-(5-methylisoxazol-3-yl)-cyclopropyl)quinoline-4-carboxamide;
(R)-6-(1-(5-Chloropyridin-2-yl)cyclopropyl)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(1-(3-methylisoxazol-5-yl)-cyclopropyl)quinoline-4-carboxamide;
6-((1r,4R*)-4-Cyanocyclohexyl)-N-(2-((R)-4-cyanothiazolidin-3-yl)-2-oxoethyl)-quinoline-4-carboxamide;
6-((1r,4R*)-4-Cyanocyclohexyl)-N-(2-((R)-4-cyanothiazolidin-3-yl)-2-oxoethyl)-quinoline-4-carboxamide;
(R)-6-(1-Cyanocyclohexyl)-N-(2-(4-cyanothiazolidin-3-yl)-2-oxoethyl)quinoline-4-carboxamide;
N-(2-((R)-4-Cyanothiazolidin-3-yl)-2-oxoethyl)-6-((1r,3R*)-1-fluoro-3-methoxy-cyclobutyl)quinoline-4-carboxamide Isomer 1;
N-(2-((R)-4-Cyanothiazolidin-3-yl)-2-oxoethyl)-6-((1r,3R*)-1-fluoro-3-methoxy-cyclobutyl)quinoline-4-carboxamide Isomer 2;
(R)-6-(1-Cyanocyclopropyl)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(1-fluoro-cyclobutyl)quinoline-4-carboxamide;
(R)—N-(2-(4-Cyanothiazolidin-3-yl)-2-oxoethyl)-6-(1-ethoxycyclopropyl)quinoline-4-carboxamide;
(R)—N-(2-(4-Cyanothiazolidin-3-yl)-2-oxoethyl)-6-cyclopropylquinoline-4-carboxamide;
N-(2-((R)-4-Cyanothiazolidin-3-yl)-2-oxoethyl)-6-((1r,4R*)-4-methoxycyclohexyl)-quinoline-4-carboxamide Isomer 1; and
N-(2-((R)-4-Cyanothiazolidin-3-yl)-2-oxoethyl)-6-((1r,4R*)-4-methoxycyclohexyl)-quinoline-4-carboxamide Isomer 2.

Embodiment 86. The compound of any of embodiments 1 to 47, or a pharmaceutically acceptable salt thereof, wherein R² is phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy.

Embodiment 87. The compound of any of embodiments 1 to 47, or a pharmaceutically acceptable salt thereof, wherein R² is phenyl, wherein the phenyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, and C₁₋₃-alkoxy-C₁₋₃-alkoxy.

Embodiment 88. The compound of any of embodiments 1 to 47, or a pharmaceutically acceptable salt thereof, wherein R² is phenyl, wherein the phenyl is optionally substituted with one or more halogen.

Embodiment 89. The compound of any of embodiments 1 to 47, or a pharmaceutically acceptable salt thereof, wherein R² is phenyl, wherein the phenyl is optionally substituted with one or more C₁₋₆-alkyl.

Embodiment 90. The compound of any of embodiments 1 to 47, or a pharmaceutically acceptable salt thereof, wherein R² is phenyl, wherein the phenyl is optionally substituted with one or more halo-C₁₋₆-alkyl.

Embodiment 91. The compound of any of embodiments 1 to 47, or a pharmaceutically acceptable salt thereof, wherein R² is phenyl, wherein the phenyl is optionally substituted with one or more C₁₋₆-alkoxy.

Embodiment 92. The compound of any of embodiments 1 to 47, or a pharmaceutically acceptable salt thereof, wherein R² is phenyl, wherein the phenyl is optionally substituted with one or more halo-C₁₋₆-alkoxy.

Embodiment 93. The compound of any of embodiments 1 to 47, or a pharmaceutically acceptable salt thereof, wherein R² is phenyl, wherein the phenyl is optionally substituted with one or more C₁₋₆-alkoxy-C₁₋₆-alkoxy.

Embodiment 94. The compound of embodiment 1, or a pharmaceutically acceptable salt thereof, wherein the compound is (R)—N-(2-(4-cyanothiazolidin-3-yl)-2-oxoethyl)-6-(4-(3-methoxy-propoxy)-phenyl)quinoline-4-carboxamide.

Embodiment 95. The compound of any of embodiments 1 to 47, or a pharmaceutically acceptable salt thereof, wherein R² is a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 96. The compound of any of embodiments 1 to 47, or a pharmaceutically acceptable salt thereof, wherein R² is a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 97. The compound of any of embodiments 1 to 47, or a pharmaceutically acceptable salt thereof, wherein R² is a 5- or 6-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

Embodiment 98. The compound of any of embodiments 1 to 47, or a pharmaceutically acceptable salt thereof, wherein R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 99. The compound of any of embodiments 1 to 47, or a pharmaceutically acceptable salt thereof, wherein R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 100. The compound of any of embodiments 1 to 47, or a pharmaceutically acceptable salt thereof, wherein R² is a 5-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 101. The compound of any of embodiments 1 to 47, or a pharmaceutically acceptable salt thereof, wherein R² is a 5-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 102. The compound of any of embodiments 1 to 47, or a pharmaceutically acceptable salt thereof, wherein R² is a 6-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 103. The compound of any of embodiments 1 to 47, or a pharmaceutically acceptable salt thereof, wherein R² is a 6-membered ring heterocyclyl, wherein the heterocyclyl ring: (i) is a saturated, partially saturated, or completely unsaturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 104. The compound of any of embodiments 1 to 47, or a pharmaceutically acceptable salt thereof, wherein R² is a 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 105. The compound of any of embodiments 1 to 47, or a pharmaceutically acceptable salt thereof, wherein R² is a 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 106. The compound of any of embodiments 1 to 47, or a pharmaceutically acceptable salt thereof, wherein R² is selected from the group consisting of tetrahydrofuranyl, furanyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 107. The compound of any of embodiments 1 to 47, or a pharmaceutically acceptable salt thereof, wherein R² is selected from the group consisting of tetrahydrofuranyl, furanyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy.

Embodiment 108. The compound of any of embodiments 1 to 47, or a pharmaceutically acceptable salt thereof, wherein R² is tetrahydrofuranyl, wherein the tetrahydrofuranyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 109. The compound of any of embodiments 1 to 47, or a pharmaceutically acceptable salt thereof, wherein R² is tetrahydropyranyl, wherein the tetrahydropyranyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 110. The compound of any of embodiments 1 to 47, or a pharmaceutically acceptable salt thereof, wherein R² is dihydropyranyl, wherein the dihydropyranyl, is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 111. The compound of any of embodiments 1 to 47, or a pharmaceutically acceptable salt thereof, wherein R² is 1,4-dioxanyl, wherein the 1,4-dioxanyl is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 112. The compound of any of embodiments 1 to 47, or a pharmaceutically acceptable salt thereof, wherein R² is 3-oxabicyclo[4.1.0]heptane, wherein the 3-oxabicyclo[4.1.0]heptane is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

Embodiment 113. The compound of any of embodiments 1 to 47, or a pharmaceutically acceptable salt thereof, wherein R² is selected from the group consisting of tetrahydrofuranyl, furanyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane.

Embodiment 114. The compound of embodiment 1, or a pharmaceutically acceptable salt thereof, wherein the compound is selected from the group consisting of:
(R)—N-(2-(4-Cyanothiazolidin-3-yl)-2-oxoethyl)-6-(furan-3-yl)quinoline-4-carboxamide;
(R)—N-(2-(4-Cyanothiazolidin-3-yl)-2-oxoethyl)-6-(4-hydroxy-tetrahydro-2H-pyran-4-yl)-quinoline-4-carboxamide;
(R)—N-(2-(4-Cyanothiazolidin-3-yl)-2-oxoethyl)-6-(tetrahydro-2H-pyran-4-yl)quinoline-4-carboxamide;
(R)—N-(2-(4-Cyanothiazolidin-3-yl)-2-oxoethyl)-6-(4-methyl-tetrahydro-2H-pyran-4-yl)-quinoline-4-carboxamide;
(R)—N-(2-(4-Cyanothiazolidin-3-yl)-2-oxoethyl)-6-(3,6-dihydro-2H-pyran-4-yl)quinoline-4-carboxamide;
(R)—N-(2-(4-Cyanothiazolidin-3-yl)-2-oxoethyl)-6-(4-ethoxytetrahydro-2H-pyran-4-yl)-quinoline-4-carboxamide;
N-(2-((R)-4-Cyanothiazolidin-3-yl)-2-oxoethyl)-6-((RS)-tetrahydro-2H-pyran-2-yl)-quinoline-4-carboxamide;
N-(2-((R)-4-Cyanothiazolidin-3-yl)-2-oxoethyl)-6-((RS)-1,4-dioxan-2-yl)quinoline-4-carboxamide;
N-(2-((R)-4-Cyanothiazolidin-3-yl)-2-oxoethyl)-6-((RS)-tetrahydrofuran-2-yl)quinoline-4-carboxamide;
(R)—N-(2-(4-Cyanothiazolidin-3-yl)-2-oxoethyl)-6-(4-ethynyl-tetrahydro-2H-pyran-4-yl)-quinoline-4-carboxamide;
(R)-6-(4-Cyanotetrahydro-2H-pyran-4-yl)-N-(2-(4-cyanothiazolidin-3-yl)-2-oxoethyl)-quinoline-4-carboxamide;
N-(2-((R)-4-Cyanothiazolidin-3-yl)-2-oxoethyl)-6-(5,5-dimethyltetrahydrofuran-3-yl)-quinoline-4-carboxamide;
(R)—N-(2-(4-Cyanothiazolidin-3-yl)-2-oxoethyl)-6-(4-fluorotetrahydro-2H-pyran-4-yl)-quinoline-4-carboxamide;
(R)—N-(2-(4-Cyanothiazolidin-3-yl)-2-oxoethyl)-6-(4-methoxy-tetrahydro-2H-pyran-4-yl)quinoline-4-carboxamide;
N-(2-((R)-4-Cyanothiazolidin-3-yl)-2-oxoethyl)-6-((RS)-tetrahydrofuran-3-yl)quinoline-4-carboxamide;
N-(2-((R)-4-Cyanothiazolidin-3-yl)-2-oxoethyl)-6-((RS)-2,2-dimethyltetrahydro-2H-pyran-4-yl)quinoline-4-carboxamide; and
6-((1RS,6SR)-3-Oxabicyclo[4.1.0]heptan-6-yl)-N-(2-((R)-4-cyanothiazolidin-3-yl)-2-oxoethyl)quinoline-4-carboxamide.

Embodiment 115. The compound of any of embodiments 1 to 47, or a pharmaceutically acceptable salt thereof, wherein R² is —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
(ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
(iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

Embodiment 116. The compound of any of embodiments 1 to 47, or a pharmaceutically acceptable salt thereof, wherein R² is —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
(ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
(iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

Embodiment 117. The compound of any of embodiments 1 to 47, or a pharmaceutically acceptable salt thereof, wherein R² is —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
(ii) the tetrahydropyranyl is optionally substituted with one or C₁₋₆-alkyl.

Embodiment 118. The compound of any of embodiments 1 to 47, or a pharmaceutically acceptable salt thereof, wherein R² is —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
(ii) the tetrahydropyranyl is optionally substituted with one or C₁₋₃-alkyl.

Embodiment 119. The compound of any of embodiments 1 to 47, or a pharmaceutically acceptable salt thereof, wherein R² is —OR⁷, wherein R⁷ is C₁₋₆-alkyl, wherein the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy.

Embodiment 120. The compound of any of embodiments 1 to 47, or a pharmaceutically acceptable salt thereof, wherein R² is —OR⁷, wherein R⁷ is C₁₋₄-alkyl, wherein the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy.

Embodiment 121. The compound of any of embodiments 1 to 47, or a pharmaceutically acceptable salt thereof, wherein R² is —OR⁷, wherein R⁷ is C₁₋₄-alkyl, wherein the C₁₋₄-alkyl is optionally substituted with one or more substituents independently selected from the group consisting of fluoro.

Embodiment 122. The compound of any of embodiments 1 to 47, or a pharmaceutically acceptable salt thereof, wherein R² is —OR⁷, wherein R⁷ is phenyl, wherein the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

Embodiment 123. The compound of any of embodiments 1 to 47, or a pharmaceutically acceptable salt thereof, wherein R² is —OR⁷, wherein R⁷ phenyl.

Embodiment 124. The compound of any of embodiments 1 to 47, or a pharmaceutically acceptable salt thereof, wherein R² is —OR⁷, wherein R⁷ is tetrahydropyranyl; wherein the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

Embodiment 125. The compound of any of embodiments 1 to 47, or a pharmaceutically acceptable salt thereof, wherein R² is —OR⁷, wherein R⁷ tetrahydropyranyl; wherein the tetrahydropyranyl is optionally substituted with one or more C₁₋₃-alkyl.

Embodiment 126. The compound of any of embodiments 1 to 47, or a pharmaceutically acceptable salt thereof, wherein R² is —OR⁷, wherein R⁷ tetrahydropyranyl.

Embodiment 127. The compound of any of embodiments 1 to 47, or a pharmaceutically acceptable salt thereof, wherein R² is —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

Embodiment 128. The compound of embodiment 1, or a pharmaceutically acceptable salt thereof, wherein the compound is selected from the group consisting of:
(R)—N-(2-(4-Cyanothiazolidin-3-yl)-2-oxoethyl)-6-phenoxyquinoline-4-carboxamide;
(R)—N-(2-(4-Cyanothiazolidin-3-yl)-2-oxoethyl)-6-(2,2-difluoroethoxy)quinoline-4-carboxamide;
(R)—N-(2-(4-Cyanothiazolidin-3-yl)-2-oxoethyl)-6-(2-fluoro-2-methylpropoxy)quinoline-4-carboxamide;
(R)—N-(2-(4-Cyanothiazolidin-3-yl)-2-oxoethyl)-6-(2-methoxyethoxy)quinoline-4-carboxamide;
(R)—N-(2-(4-Cyanothiazolidin-3-yl)-2-oxoethyl)-6-(cyclopropyl-methoxy)quinoline-4-carboxamide;
(R)—N-(2-(4-Cyanothiazolidin-3-yl)-2-oxoethyl)-6-((tetrahydro-2H-pyran-4-yl)oxy)-quinoline-4-carboxamide;
(R)—N-(2-(4-Cyanothiazolidin-3-yl)-2-oxoethyl)-6-((1,3-difluoropropan-2-yl)oxy)-quinoline-4-carboxamide;
(R)—N-(2-(4-Cyanothiazolidin-3-yl)-2-oxoethyl)-6-((3,3-difluorocyclobutyl)-methoxy)quinoline-4-carboxamide;
N-(2-((R)-4-Cyanothiazolidin-3-yl)-2-oxoethyl)-6-(((RS)-2,2-dimethyltetrahydro-2H-pyran-4-yl)oxy)quinoline-4-carboxamide;
(R)-6-(Cyanomethoxy)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(2,2-difluoropropoxy)quinoline-4-carboxamide;
(R)—N-(2-(4-Cyanothiazolidin-3-yl)-2-oxoethyl)-6-propoxyquinoline-4-carboxamide; and
(R)—N-(2-(4-Cyanothiazolidin-3-yl)-2-oxoethyl)-6-(2-fluoroethoxy)-quinoline-4-carboxamide.

Embodiment 129. The compound of any of embodiments 1 to 47, or a pharmaceutically acceptable salt thereof, wherein R² is selected from the group consisting of:
(a) C₁₋₃-alkyl, wherein the C₁₋₃-alkyl is optionally substituted with one or more substituents independently selected from the group consisting of halogen, hydroxy, cyano, C₃₋₆-cycloalkyl, halo-C₃₋₆-cycloalkyl, C₁₋₃-alkoxy, halo-C₁₋₃-alkoxy, C₃₋₆-cycloalkoxy, halo-C₃₋₆-cycloalkoxy, C₁₋₃-alkoxy-C₁₋₃-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₃-alkyl;
(b) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(e) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
   (ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
   (iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

Embodiment 130. The compound of any of embodiments 1 to 47, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl; and
(b) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(c) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
   (ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
   (iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

Embodiment 131. The compound of any of embodiments 1 to 47, or a pharmaceutically acceptable salt thereof, wherein R² is selected from the group consisting of:
(a) C₁₋₆-alkyl, wherein the C₁₋₆-alkyl is optionally substituted with one or more substituents independently selected from the group consisting of halogen, hydroxy, C₁₋₆-alkoxy, halo-C₁₋₆-alkoxy, C₃₋₆-cycloalkoxy, C₁₋₆-alkoxy-C₁₋₆-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy; and
(e) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
   (ii) the tetrahydropyranyl is optionally substituted with one or C₁₋₆-alkyl.

Embodiment 132. The compound of any of embodiments 1 to 47, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(b) a 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy; and
(c) —OR⁷, wherein R⁷ is selected from the group consisting of C₁₋₆-alkyl, phenyl, and tetrahydropyranyl; wherein:
   (i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
   (ii) the tetrahydropyranyl is optionally substituted with one or C₁₋₆-alkyl.

Embodiment 133. The compound of any of embodiments 129 to 132, or a pharmaceutically acceptable salt thereof, wherein R² is a 4-membered ring heterocyclyl, wherein the heterocyclyl ring: (i) is a saturated or partially saturated monocyclic ring, (ii) has one oxygen ring atom with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

Embodiment 134. The compound of any of embodiments 129 to 132, or a pharmaceutically acceptable salt thereof, wherein R² is a 5-membered ring heterocyclyl, wherein the heterocyclyl ring: (i) is a saturated or partially saturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, and C₂₋₃-alkynyl, C₁₋₃-alkoxy.

Embodiment 135. The compound of any of embodiments 129 to 132, or a pharmaceutically acceptable salt thereof, wherein R² is a 6-membered ring heterocyclyl, wherein the heterocyclyl ring: (i) is a saturated or partially saturated monocyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

Embodiment 136. The compound of any of embodiments 129 to 132, or a pharmaceutically acceptable salt thereof, wherein R² is a 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy.

Embodiment 137. The compound of any of embodiments 1 to 47, or a pharmaceutically acceptable salt thereof, wherein R² is selected from the group consisting of:
(a) C₁₋₃-alkyl, wherein the C₁₋₃-alkyl is optionally substituted with one or more substituents independently selected from the group consisting of halogen, hydroxy, C₁₋₃-alkoxy, halo-C₁₋₃-alkoxy, C₃₋₆-cycloalkoxy, C₁₋₃-alkoxy-C₁₋₃-alkoxy, tetrahydrofuranyl, tetrahydrofuranyloxy, tetrahydropyranyl, morpholinyl, pyrazolyl, isoxazolyl, triazolyl, and pyridinyl; and wherein the tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 5-, 6-, or 7-membered ring heterocyclyl selected from the group consisting of tetrahydrofuranyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy;
(e) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
   (ii) the tetrahydropyranyl is optionally substituted with one or C₁₋₃-alkyl.

Embodiment 138. The compound of any of embodiments 1 to 47, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(b) a 5-, 6-, or 7-membered ring heterocyclyl selected from the group consisting of tetrahydrofuranyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(c) —OR⁷, wherein R⁷ is selected from the group consisting of C₁₋₄-alkyl, phenyl, and tetrahydropyranyl; wherein:
   (i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
   (ii) the tetrahydropyranyl is optionally substituted with one or C₁₋₃-alkyl.

Embodiment 139. The compound of any of embodiments 129 to 138, or a pharmaceutically acceptable salt thereof, wherein R² is optionally substituted cyclopropyl.

Embodiment 140. The compound of any of embodiments 129 to 138, or a pharmaceutically acceptable salt thereof, wherein R² is optionally substituted cyclobutyl.

Embodiment 141. The compound of any of embodiments 129 to 138, or a pharmaceutically acceptable salt thereof, wherein R² is optionally substituted cyclopentyl.

Embodiment 142. The compound of any of embodiments 129 to 138, or a pharmaceutically acceptable salt thereof, wherein R² is optionally substituted cyclohexyl.

Embodiment 143. The compound of any of embodiments 1 to 47, or a pharmaceutically acceptable salt thereof, wherein R² is selected from the group consisting of:
(a) optionally substituted C₁₋₃-alkyl selected from the group consisting of methyl, fluoromethyl, difluoromethyl, trifluoromethyl, hydroxyethyl, hydroxypropyl, ethoxymethyl, difluoroethoxymethyl, methoxyethyl, dimethoxyethyl, difluoropropyl, trifluoropropyl, methoxypropyl, methoxyethoxymethyl, cyclopropoxymethyl, morpholinylmethyl, tetrahydrofuranylmethyl, tetrahydrofuranyloxymethyl, tetrahydropyranylmethyl, pyrazolylmethyl, dimethylpyrazolylmethyl, methylisoxazolylmethyl, methylisoxazolylethyl, methyltriazolylmethyl, dimethyltriazolylmethyl, dimethylpyridinylmethyl, methylpyridinylethyl, fluoropyridinylethyl, chloropyridinylethyl, and trifluoromethylpyridinylethyl.
(b) optionally substituted C₃₋₆-cycloalkyl selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl;
(c) optionally substituted 5-, 6-, or 7-membered ring heterocyclyl selected from the group consisting of tetrahydrofuranyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane; and
(d) —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

Embodiment 144. The compound of any of embodiments 1 to 47, or a pharmaceutically acceptable salt thereof, wherein R² is selected from the group consisting of:
(a) optionally substituted C₃₋₆-cycloalkyl selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl;
(b) optionally substituted 5-, 6-, or 7-membered ring heterocyclyl selected from the group consisting of tetrahydrofuranyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane; and
(c) —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

Embodiment 145. The compound of any of embodiments 1 to 47, or a pharmaceutically acceptable salt thereof, wherein R² is selected from the group consisting of methoxycyclohexyl, tetrahydropyranyl, fluorobutoxy, and tetrahydropyranyloxy.

Embodiment 146. A pharmaceutical composition comprising a compound of any of embodiments 1 to 145, or a pharmaceutically acceptable salt thereof, and one or more pharmaceutically acceptable excipients.

Embodiment 147. A method of treating or preventing an FAP-mediated condition in a subject suffering from or susceptible to the FAP-mediated condition, the method comprising administering to the subject a therapeutically effective amount of a compound of any of embodiments 1 to 145, or a pharmaceutically acceptable salt thereof.

Embodiment 148. The method of embodiment 147, wherein the FAP-mediated condition is selected from the group consisting of liver disease, type 2 diabetes mellitus, cardiovascular conditions, obesity, obesity-related conditions, fibrosis, keloid disorder, inflammation, and cancer.

Embodiment 149. The method of embodiment 148, wherein the FAP-mediated condition is liver disease.

Embodiment 150. The method of embodiment 149, wherein the liver disease is nonalcoholic steatohepatitis.

Embodiment 151. The use of a compound of any of embodiments 1 to 145, or a pharmaceutically acceptable salt thereof, for the manufacture of a medicament for treating or preventing an FAP-mediated condition.

### VIX. Examples

The following descriptions of experiments, procedures, examples, and intermediates are intended to exemplify embodiments of the disclosure. They are in no way intended to be limiting. Other compounds of this disclosure may be prepared using the methods illustrated in these examples, either alone or in combination with techniques generally known in the art.

### A. General Conditions

Unless otherwise stated:
(i) operations were carried out at room temperature (rt), i.e., in the range 17 to 25° C., and under an atmosphere of an inert gas such as N₂;
(ii) where reactions refer to the use of a microwave reactor, one of the following microwave reactors was used: Biotage Initiator, Personal Chemistry Emrys Optimizer, Personal Chemistry Smith Creator, or CEM Explorer;
(iii) where reactions refer to the use of irradiation with LED, a commercial, standardized EvoluChem™ PhotoRedOx Box Photoreactor from HepatoChem equipped with a Kessil H150 blue LED (456 nm, 34 W) or a commercial standardized Photoreactor m2 from Penn Photon Devices equipped with a LED module (365 nm) was used;
(iv) in general, the course of reactions was followed by thin layer chromatography (TLC) and/or analytical high performance liquid chromatography (HPLC or UPLC) which was usually coupled to a mass spectrometer (LCMS);
(v) when necessary, organic solutions were dried over anhydrous MgSO₄ or Na₂SO₄, or by using ISOLUTE® Phase Separator, and work-up procedures were carried out using traditional phase separating techniques;
(vi) evaporations were carried out either by rotary evaporation in vacuo or in a Genevac HT-4/EZ-2 or Biotage V10;
(vii) unless otherwise stated, flash column chromatography was performed on straight phase silica, using either Merck Silica Gel (Art. 9385) or prep-packed cartridges such as Biotage® SNAP cartridges (40-63 μm silica, 4-330 g), Biotage® Sfär Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g), or Agela Flash Colum Silica-CS cartridges (80-330 g), or on reversed phase silica using Agela Technologies C-18, spherical cartridges (20-35 μm, 100 A, 80-330 g), manually or automated using a Grace Reveleris® X2 Flash system or similar system;
(viii) preparative reverse phase HPLC and preparative reverse phase SFC were performed using standard HPLC and SFC instruments, respectively, equipped with either a MS and/or UV triggered fraction collecting instrument, using either isocratic or a gradient of the mobile phase as described in the experimental section, and one of the following methods as described below:
(vii) preparative reverse phase HPLC and preparative reverse phase SFC were performed using standard HPLC and SFC instruments, respectively, equipped with either a MS and/or UV triggered fraction collecting instrument, using either isocratic or a gradient of the mobile phase as described in the experimental section, and one of the following methods as described below;
HPLC Prep Methods: PrepMethod A: The compound was purified by preparative HPLC on a Kromasil C8 column (10 μm, 250×50 mm ID) using a gradient of MeCN in H₂O/MeCN/FA (95/5/0.2) as mobile phase; PrepMethod B: The compound was purified by preparative HPLC on a XSelect CSH C18 OBD column (5 μm, 150×30 mm ID) using a gradient of MeCN in H₂O/TFA (0.05%) as mobile phase; PrepMethod C: The compound was purified by preparative HPLC on a Kromasil C8 column (10 μm, 250×20 mm ID) using a gradient of MeCN in H₂O/MeCN/FA (95/5/0.2) as mobile phase; PrepMethod D: The compound was purified by preparative HPLC on a XSelect CSH OBD column (5 μm, 150×30 mm ID) using a gradient of MeCN in H₂O/FA (0.1%) as mobile phase; PrepMethod E: The compound was purified by preparative HPLC on a XBridge C18 column (10 μm, 250×19 mm ID) using a gradient of MeCN in H₂O/MeCN/NH₃ (95/5/0.2) as mobile phase; PrepMethod F: The compound was purified by preparative HPLC on a Sunfire prep C18 column, (5 μm, 150×30 mm ID) using a gradient of MeCN in H₂O/FA (0.1%) as mobile phase; PrepMethod G: The compound was purified by preparative HPLC on a Waters Sunfire C18 ODB column (5 μm, 150×30 mm ID) using a gradient of MeCN in H₂O/FA (0.1 M) as mobile phase; PrepMethod H: The compound was purified by preparative HPLC on a XBridge Prep OBD C18 column (5 μm, 150×30 mm ID) using a gradient of MeCN in H₂O/NH₄HCO₃ (10 mM)+NH₃ (0.1%) as mobile phase; PrepMethod I: The compound was purified by preparative HPLC on a XBridge Prep OBD C18 column (5 μm, 150×30 mm ID) using a gradient of MeCN in H₂O/NH₄HCO₃ (10 mM) as mobile phase; PrepMethod J: The compound was purified by preparative HPLC on a XSelect CSH Prep C18 OBD column (5 μm, 250×19 mm ID) using a gradient of MeCN in H₂O/TFA (0.05%) as mobile phase; PrepMethod K: The compound was purified by preparative HPLC on a Sunfire C18 OBD Prep column (5 μm, 250×19 mm ID) using a gradient of MeCN in H₂O/NH₄HCO₃ (10 mM) as mobile phase; PrepMethod L: The compound was purified by preparative HPLC on a Kinetex EVO C18 column (5 μm, 150×30 mm ID) using a gradient of MeCN in H₂O/NH₄HCO₃ (10 mM) as mobile phase; PrepMethod M: The compound was purified by preparative HPLC on a XBridge C18 ODB column (5 μm, 150×30 mm ID) using a gradient of MeCN in H₂O/NH₃ (0.2%) as mobile phase; PrepMethod N: The compound was purified by preparative HPLC on a XSelect CSH Fluoro Phenyl column (5 μm, 150×30 mm ID) using a gradient of MeCN in H₂O/FA (0.1%) as mobile phase; PrepMethod O: The compound was purified by preparative HPLC on a XSelect CSH Fluoro Phenyl OBD column (5 μm, 250×19 mm ID) using a gradient of MeCN in H₂O/TFA (0.05%) as mobile phase; PrepMethod P: The compound was purified by preparative HPLC on a XSelect CSH Prep C18 OBD column (5 μm, 250×19 mm ID) using a gradient of MeCN in H₂O/TFA (0.1%) as mobile phase; PrepMethod Q: The compound was purified by preparative HPLC on a CHIRALPAK IA column (5 μm, 250×50 mm ID) using a gradient of EtOH in MTBE/NH₃-MeOH (0.5%, 2 M) as mobile phase; PrepMethod R: The compound was purified by preparative HPLC on a XSelect CSH OBD column (5 μm, 150×30 mm ID) using a gradient of MeCN in H₂O/TFA (0.1%) as mobile phase; PrepMethod S: The compound was purified by preparative HPLC on a XBridge Prep OBD C18 column (5 μm, 150×30 mm ID) using a gradient of MeCN in H₂O/FA (0.1%) as mobile phase; PrepMethod T: The compound was purified by preparative HPLC on a XSelect CSH C18 OBD column (5 μm, 150×30 mm ID) using a gradient of MeCN in H₂O/FA (0.1%) as mobile phase; PrepMethod U: The compound was purified by preparative HPLC on a CHIRALPAK IA-3 column (3 μm, 50×4.6 mm ID) using a gradient of EtOH in Hexane/DCM (3:1, 0.1% DEA) as mobile phase; PrepMethod V: The compound was purified by preparative HPLC on a XSelect CSH Prep C18 OBD column (5 μm, 250×19 mm ID) using a gradient of MeCN in H₂O/FA (0.1%) as mobile phase;
SFC Prep Methods: PrepMethod SFC-A: The compound was purified by preparative SFC on a Waters BEH column (5 μm, 250×30 mm ID) using EtOH/FA (20 mM) in CO₂ as mobile phase; PrepMethod SFC-B: The compound was purified by preparative SFC on a Phenomenex Luna Hilic column (5 μm, 250×30 mm ID) using EtOH/FA (20 mM) in CO₂ as mobile phase; PrepMethod SFC-C: The compound was purified by preparative SFC on a Phenomenex Luna Hilic column (5 μm, 250×30 mm ID) using MeOH/NH₃ (20 mM) in CO₂ as mobile phase;
Relevant fractions were collected, combined, and freeze-dried to give the purified compound, or relevant fractions were collected, combined, and concentrated at reduced pressure, extracted with DCM or EtOAc, and the organic phase was dried either over Na₂SO₄ or by using a phase-separator, and then concentrated at reduced pressure to give the purified compound;
(ix) chiral preparative chromatography was carried out using HPLC or SFC on a standard HPLC or SFC instruments, respectively, and using either isocratic or gradient run with mobile phase as described in the experimental section;
(x) yields, where present, are not necessarily the maximum attainable, and when necessary, reactions were repeated if a larger amount of the reaction product was required;
(xi) where certain compounds were obtained as an acid-addition salt (for example, a mono-hydrochloride salt or a di-hydrochloride salt), the stoichiometry of the salt was based on the number and nature of the basic groups in the compound, the exact stoichiometry of the salt was generally not determined, for example by means of elemental analysis data;
(xii) in general, the structures of the end-products of the Formula (I) were confirmed by nuclear magnetic resonance (NMR) and/or mass spectral techniques; proton NMR chemical shift values were measured on the delta scale using Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. In certain instances, protons can be masked or partially masked by solvent peaks and will therefore either be missing and not reported or reported as multiplets overlapping with solvent. The following abbreviations have been used (and derivatives thereof, e.g., dd, doublet of doublets, etc.): s, singlet; d, doublet; t, triplet; q, quartet; m, multiplet; br, broad; qn, quintet; and p, pentet. In some cases, the structures of the end-products of the Formula (I) might appear as rotamers in the NMR-spectrum, in which instances only peaks of the major rotamer are reported. Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported;
(xiii) intermediates were not necessarily fully purified but their structures and purity were assessed by TLC, analytical HPLC/UPLC, and/or NMR analysis and/or mass spectrometry;
(xiv) unless stated otherwise, compounds containing an asymmetric carbon and/or sulfur atom were not resolved;
(xv) in general, Examples and Intermediate compounds are named using ChemDraw Professional version 19.0.0.22 or 20.0.2.51 from PerkinElmer. ChemDraw Professional version 19.0.0.22 and 20.0.2.51 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.
   ChemDraw is optionally using labels in the graphical representation of stereocenters such as ‘&’ and ‘or’ to describe the configuration of the stereochemical centers present in the structure. In general, chemical structures of Examples and Intermediates containing the label ‘&’ at a stereocenter means the configuration of such Example or Intermediate at that stereocenter is a mixture of both (R) and (S); and a label ‘or’ means the configuration of such Example or Intermediate at that stereocenter is either (S) or (R). Absolute, unspecified, ‘&’, and ‘or’ stereocenters can all be present in a single structure.
   In general, for structures of Examples and Intermediates where all of the stereocenters are designated as ‘&’, the structure is named with a “rac-” prefix. For structures of Examples and Intermediates where all of the stereocenters are designated as ‘or’, the structure is named with a “rel-” prefix.
   In general, Examples and Intermediate compounds are named using the descriptors (RS) and (SR) to denote general ‘&’ centers for chemical structures with multiple chiral centers where only some are designated as ‘&’. The descriptors (R*) and (S*) are used to denote the general ‘or’ centers for chemical structures with multiple chiral centers where only some are designated as ‘or’.
   In general, the descriptors (r) and (s) are used to describe the absolute configuration of any pseudoasymmetric centers in the structures of Examples and Intermediates.
   In general, the label “Isomer 1” corresponds to the first eluted isomer, and “Isomer 2” corresponds to the second eluted isomer, on a given chiral HPLC column and eluent, and are used to distinguish two isomers containing one or more stereocenters with absolute unknown configuration at one or more stereocenters;
(xvi) where reactions refer to being degassed or purged, this can be performed for example by purging the reaction solvent with a constant flow of nitrogen for a suitable period of time (for example, 5 to 10 min);
(xvii) where reactions refer to applying an evacuate-refill cycle, this can be performed by evacuating the reaction vessel by applying vacuum to the reaction vessel and then refilling with an inert gas, typically N₂ or Argon. The process is generally repeated, typically three times; and
(xvii) in addition to the ones mentioned above, the following abbreviations have been used:
(xvii) in addition to the ones mentioned above, the following abbreviations have been used:

| | | | |
|---|---|---|---|
| Ala | Alanine | LC | Liquid chromatography |
| AMC | 7-Amino-4-methylcoumarin | LED | Light emitting diode |
| Aq | Aqueous | LiOtBu | Lithium tert-butoxide |
| Atm | Atmosphere | MeI | CH₃I |
| BINAP | 2,2′-Bis(diphenylphosphino)- | MeCN | Acetonitrile |
| | 1,1′-binaphthalene | MeOH | Methanol |
| Boc₂O | di-tert-Butyl dicarbonate | MS | Mass spectrometry |
| BSA | Bovine Serum Albumin | m/z | Mass spectrometry peak(s) |
| n-BuLi | n-Butyllithium | MW | Molecular weight |
| t-BuXPhos | di-tert-Butyl(2′,4′,6′- | N_{2 (g)} | Nitrogen in gas phase |
| | triisopropyl-[1,1′-biphenyl]-2- | NaHMDS | Hexamethyldisilazane |
| | yl)phosphane | | sodium salt |
| calcd | Calculated | NiBr₂•DME | Nickel(II) bromide 1,2- |
| cataCXium ® A | Mesylate[(di(1-adamantyl)-n- | | Dimethoxyethane complex |
| Pd G3 | butylphosphine)-2-(2′-amino- | NiBr₂•dtbbpy | [4,4′-Bis(1,1- |
| | 1,1′-biphenyl)]palladium(II), | | dimethylethyl)-2,2′- |
| | [(Di(1-adamantyl)- | | bipyridine] nickel (II) |
| | butylphosphine)-2-(2′-amino- | | dibromide |
| | 1,1′-biphenyl)]palladium(II) | NiBr₂ | Nickel(II) bromide 2- |
| | methanesulfonate | O(CH₂CH₂OCH₃)₂ | methoxyethyl ether |
| | [CAS No 1651823-59-4] | | complex |
| CLᵢₙₜ | Intrinsic clearance | NiCl₂•DME | Nickel(II) chloride 2- |
| Chaps | (3-((3-Cholamidopropyl) | | methoxyethyl ether |
| | dimethylammonio)-1- | | complex |
| | propanesulfonate) | NMR | Nuclear magnetic |
| CO_{(g)} | Carbonmonoxide in gas phase | | resonance |
| Conc | Concentrated | Pₐₚₚ | Apparent permeability |
| CR | Concentration response | | coefficient |
| DAST | Diethylaminosulfur | PBS | Phosphate Buffered Saline |
| | trifluoride | PCR | Polymeras chain reaction |
| DCC | N,N′- | Pd/C | Palladium on carbon |
| | Dicyclohexylcarbodiimide | PdCl₂ | Palladium dichloride |
| DCE | 1,2-Dichloroethane | Pd₂(dba)₃ | (Tris(dibenzylideneacetone) |
| DCM | Dichloromethane | | dipalladium(0) |
| Dess-Martin | 1,1,1-Tris(acetyloxy)-1,1- | Pd₂(dba)₃•CHCl₃ | (Tris(dibenzylideneacetone) |
| periodinane | dihydro-1,2-benziodoxol-3- | | dipalladium(0) chloroform |
| | (1H)-one | | adduct |
| DIAD | Diisopropyl azodicarboxylate | Pd(dppf)Cl₂ | 1,1′-Bis(diphenyl- |
| DIPEA | N-ethyl-N-isopropyl-propan- | | phosphino)ferrocene- |
| | 2-amine | | dichloropalladium(II) |
| DMA | N,N-Dimethylacetamide | Pd (dppf)Cl₂•DCM | [1,1′- |
| DMAP | 4-Dimethylaminopyridine | | Bis(diphenylphosphino)ferr |
| DME | Dimethyl ether | | ocene]dichloropalladium(II) |
| DMF | N,N-Dimethylformamide | | complex with |
| DMPU | N,N′-Dimethylpropylene-urea | | dichloromethane |
| DMSO | Dimethyl sulfoxide | Pd(dtbpf)Cl₂ | 1,1′-Bis(di-tert- |
| dppf | 1,1′-Ferrocenediyl- | | butylphosphino)ferrocene |
| | bis(diphenylphosphine) | | palladium dichloride |
| dtbbpy | 4-tert-Butyl-2-(4-tert- | Pd(OAc)₂ | Palladium acetate |
| | butylpyridin-2-yl)pyridine | Pd(PPh₃)₄ | Tetrakis(triphenyl- |
| EDTA | Ethylenediaminetetra- | | phosphine)-palladium(0) |
| | acetic acid | PREP | Prolyl oligopeptidase |
| EDC | 3-(Ethyliminomethylene- | Pro | Proline |
| | amino)-N,N-dimethyl-propan- | Royer ® Palladium | Pd/polyethylenimine/SiO₂ |
| | 1-amine; hydrochloride | Catalyst | |
| eq | Equivalent | rt | Room temperature |
| ESI | Electrospray ionization | RU | Response unit |
| Et₃N | Triethylamine | sat | Saturated |
| EtOAc | Ethyl acetate | SD | Standard deviation |
| EtOH | Ethanol | Selectfluor | 1-(chloromethyl)-4-fluoro- |
| Et₂Zn | Diethyl zinc | | 1,4-diazabicyclo[2.2.2]- |
| FA | Formic acid | | octane-1,4-diium |
| FAC | Final Assay Concentration | | tetrafluoroborate |
| FAP | Prolyl endopeptidase | SFC | Supercritical fluid |
| | fibroblast activation protein | | chromatography |
| Gly | Glycine | SPhos | Dicyclohexyl(2′,6′- |
| H_{2 (g)} | Hydrogen gas | | dimethoxy-[1,1′-biphenyl]- |
| HATU | (1-[Bis(dimethylamino)- | | 2-yl)phosphane |
| | methylene]-1H-1,2,3- | T3P | Propanephosphonic acid |
| | triazolo[4,5-b]pyridinium 3- | | anhydride |
| | oxide hexafluorophosphate | TBADT | Tetrakis(tetrabutyl- |
| hDPP7 | Human dipeptidylpeptidase 7 | | ammonium) decatungstate |
| hDPP8 | Human dipeptidylpeptidase 8 | TBME | tert-Butyl methyl ether |
| hDPP9 | Human dipeptidylpeptidase 9 | TCEP | Tris(2-carboxyethyl)- |
| HEPES | (4-(2-Hydroxyethyl)-1- | | phosphine hydrochloride |
| | piperazineethanesulfonic | TEA | Triethylamine |
| | acid) | TFA | Trifluoro acetic acid |
| hFAP | Human Prolyl endopeptidase | TFAA | Trifluoro acetic acid |
| | fibroblast activation protein | | anhydride |
| His | Histidine | THF | Tetrahydrofuran |
| HOBt | 1-Hydroxybenzotriazole; | TLC | Thin layer chromatography |
| | hydrate | TREAT-HF | Triethylamine |
| HPLC | High performance liquid | | trihydrofluoride |
| | chromatography | Tris HCl | Tris(hydroxymethyl)amino |
| HRMS | High resolution mass | | methane hydrochloride |
| | spectrometry | Triton X-100 | t-Octylphenoxypolyethoxy |
| IC₅₀ | Half-maximum inhibitory | | ethanol |
| | concentration | UPLC | Ultra performance liquid |
| ID | Inner diameter | | chromatography |
| Ir[dF(CF₃)ppy]₂ | [4,4′-Bis(1,1-dimethylethyl)- | UV | Ultraviolet |
| (dtbbpy)PF₆ | 2,2′-bipyridine- | w % | Weight percentage |
| | N1,N1′]bis[3,5-difluoro-2-[5- | XantPhos | (9,9-Dimethyl-9H- |
| | (trifluoromethyl)-2-pyridinyl- | | xanthene-4,5-diyl)- |
| | N]phenyl-C] | | bis(diphenylphosphane) |
| | Iridium(III) | XPhos | Dicyclohexyl(2′,4′,6′- |
| | hexafluorophosphate | | triisopropyl-[1,1′-biphenyl]- |
| | [CAS Number 870987-63-6] | | 2-yl)phosphane |
| KOAc | Potassium acetate | XPhos-Pd-G2 | Chloro(2- |
| KOtBu | Potassium tert-butoxide | | dicyclohexylphosphino- |
| | | | 2′,4′,6′-triisopropyl-1,1′- |
| | | | biphenyl)[2-(2′-amino-1,1′- |
| | | | biphenyl)]palladium(II), |
| | | | X-Phos aminobiphenyl |
| | | | palladium chloride |
| | | | precatalyst |
| | | | [CAS No 1310584-14-5] |
| | | Zn | Zinc |

UNITS

| | |
|---|---|
| A | Angström |
| C | Celcius |
| DA | Dalton |
| g | gram |
| h | hour(s) |
| M | Molar |
| MHz | megahertz |
| mg | milligram |
| min | minute(s) |
| mL | milliliter |
| mm | millimeter |
| mmol | millimole |
| μL | microliter |
| μm | micrometer |
| μmol | micromole |
| N | Normality |
| nm | nanometer |
| rpm | revolution per minute |
| v/v | volume by volume |
| W | watt |

### B. Intermediate Compounds

### Intermediate 1: tert-Butyl (R)-4-cyanothiazolidine-3-carboxylate

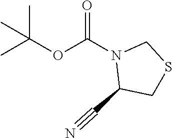

### Step a) tert-Butyl (R)-4-carbamoylthiazolidine-3-carboxylate

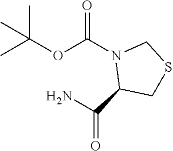

Boc₂O (18.6 mL, 80.2 mmol) was added to a stirred solution of tert-butyl (R)-4-carbamoylthiazolidine-3-carboxylate (17.0 g, 72.9 mmol) and pyridine (7.07 mL, 87.5 mmol) in EtOAc (170 mL) and the reaction mixture was stirred at rt for 3 h. Then, a solution of NH₃ (aq, 25%, 6 mL) was added dropwise and the mixture was stirred at rt overnight. The reaction mixture was diluted with EtOAc, the phases were separated and the organic phase was washed with sat NaCl, dried, filtered through a pad of silica gel, washed with EtOAc and evaporated to give the crude title compound (16.9 g, 100%) as a colorless oil, which was used directly in the next step.

### Step b) tert-Butyl (R)-4-cyanothiazolidine-3-carboxylate

TFAA (12.4 mL, 87.5 mmol) as a solution in EtOAc (20 mL) was added to a solution of crude tert-butyl (R)-4-carbamoylthiazolidine-3-carboxylate (16.9 g, 72.9 mmol) and pyridine (14.7 mL, 182 mmol) in EtOAc (150 mL) at rt. The mixture was stirred at rt for 4 h and then diluted with EtOAc, washed with HCl (1 M, aq), and sat NaHCO₃ (aq). The organic phase was dried, filtered through a pad of silica gel, washed with EtOAc, and evaporated to give a light yellow oil which solidified on standing. The crude solid material was suspended in heptane:EtOAc (4:1, 50 mL) and stirred at rt overnight. The solids were filtered off, washed with heptane:EtOAc (4:1), and dried to give title compound (12.0 g, 83%) as a colorless solid; ¹H NMR (400 MHz, CDCl₃) δ 5.20-4.79 (m, 1H), 4.60-4.53 (m, 1H), 4.53-4.36 (m, 1H), 3.40-3.18 (m, 2H), 1.51 (s, 9H).

### Intermediate 2: (R)-Thiazolidine-4-carbonitrile Hydrochloride

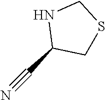

A solution of conc HCl (11 mL) in MeOH (140 mL) was added slowly to a solution of tert-butyl (R)-4-cyanothiazolidine-3-carboxylate Intermediate 1 (6.0 g, 28 mmol) in MeOH (140 mL) at rt. The clear colorless solution was stirred at rt for 2 h. Solvents were evaporated to give the title compound (4.22 g, 100%) as a colorless solid; ¹H NMR (400 MHz, CD₃OD) δ 4.90 (dd, 1H), 4.35-4.24 (m, 2H), 3.37-3.24 (m, 2H).

### Intermediate 3: tert-Butyl (R)-(2-(4-cyanothiazolidin-3-yl)-2-oxoethyl)carbamate

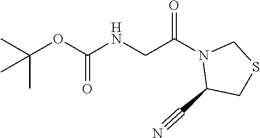 [SMILES: CC(C)(C)OC(=O)NCC(=O)N1CSC[C@H]1C#N]

DIPEA (19.6 mL, 112 mmol) was added to a suspension of (R)-thiazolidine-4-carbonitrile hydrochloride Intermediate 2 (4.22 g, 28 mmol), (tert-butoxycarbonyl)glycine (6.13 g, 35.0 mmol) and T3P (42 mL, 70 mmol, 50% solution in EtOAc) in EtOAc (120 mL). The mixture was heated at 60° C. for 4 h. The mixture was diluted with EtOAc, and sequentially washed with water, HCl (1 M, aq) and sat NaHCO₃ (aq). The organic phase was dried, filtered and evaporated. The residue was filtered through a pad of silica gel, washed with heptane:EtOAc (1:1) and evaporated to give an oil which was triturated with heptane:DCM to give the title compound (7.6 g, 100%) as an almost colorless solid; ¹H NMR (400 MHz, CDCl₃) δ 5.36-5.25 (m, 2H), 4.59-4.52 (m, 2H), 4.14-3.90 (m, 2H), 3.29 (d, 2H), 1.45 (s, 9H).

### Intermediate 4: (R)-3-Glycylthiazolidine-4-carbonitrile Hydrochloride

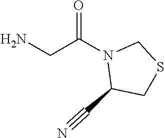 [SMILES: N#C[C@@H]1CSCN1C(=O)CN]

A solution of conc HCl (5.6 mL) in MeOH (140 mL) was added slowly to a solution of tert-butyl (R)-(2-(4-cyanothiazolidin-3-yl)-2-oxoethyl)carbamate Intermediate 3 (7.6 g, 28 mmol) in MeOH (140 mL), then the solution was stirred at rt overnight. The solvents were evaporated to give the title compound (5.8 g, 100%) as a colorless solid; ¹H NMR (400 MHz, CD₃OD) δ 5.34 (t, 1H), 4.72 (d, 1H), 4.62 (d, 1H), 4.11-3.94 (m, 2H), 3.41-3.36 (m, 2H).

### Intermediate 5: Methyl 6-acetylquinoline-4-carboxylate

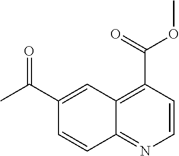 [SMILES: COC(=O)c1ccnc2ccc(C(C)=O)cc12]

Tributyl(1-ethoxyvinyl)stannane (1.63 g, 4.51 mmol) was added to methyl 6-bromoquinoline-4-carboxylate (1.0 g, 3.8 mmol) and Pd(dppf)Cl₂·DCM (0.307 g, 0.38 mmol) in degassed 1,4-dioxane (25 mL) at 20° C. under N₂ (g). The resulting mixture was stirred at 80° C. for 18 h. The mixture was diluted with MeCN (3 mL), ten drops of 3 N HCl(aq) was added and the stirring was continued for 30 min. The reaction mixture was diluted with water (10 mL) and extracted with EtOAc (3×10 mL). The organic layer was dried over Na₂SO₄, filtered and concentrated. The residue was purified by preparative TLC (pentane:EtOAc 1:1), to give the title compound (0.70 g, 81%) as a white solid; MS (ESI) m/z [M+H]⁺ 230.

### Intermediate 6: Methyl 6-(2-hydroxypropan-2-yl)quinoline-4-carboxylate

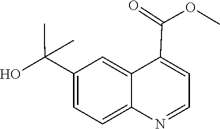 [SMILES: COC(=O)c1ccnc2ccc(C(C)(C)O)cc12]

CH₃MgCl (3 M in THF, 0.87 mL, 2.6 mmol) was added dropwise to a solution of methyl 6-acetylquinoline-4-carboxylate Intermediate 5 (0.30 g, 1.3 mmol) in THF (3 mL) at 0° C. and under an atmosphere of N₂ (g), and the reaction mixture was stirred at 0° C. for 3 h. The reaction was quenched with sat NH₄Cl (aq, 20 mL), and the mixture was extracted with DCM (3×20 mL). The combined organic layer was dried over Na₂SO₄, filtered and evaporated under reduced pressure. The residue was purified by preparative TLC (DCM:MeOH, 10:1), to give the title compound (0.20 g, 62%) as an orange gum; MS (ESI) m/z [M+H]⁺ 246.

### Intermediate 7: Methyl 6-(hydroxymethyl)quinoline-4-carboxylate

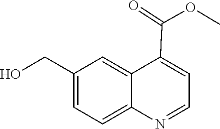 [SMILES: COC(=O)c1ccnc2ccc(CO)cc12]

(Tributylstannyl)methanol (1.45 g, 4.51 mmol) was added to a suspension of methyl 6-bromoquinoline-4-carboxylate (1.0 g, 3.8 mmol) and Pd(PPh₃)₄ (0.43 g, 0.38 mmol) in degassed toluene (25 mL) at 20° C. and under an atmosphere of N₂ (g), and the reaction mixture was stirred at 80° C. for 18 h. The solvent was removed under reduced pressure and the residue was purified by preparative TLC (EtOAc:pentane 3:2), to give the title compound (0.62 g, 76%) as a pale yellow solid; MS (ESI) m/z [M+H]⁺ 218.

### Intermediate 8: Methyl 6-formylquinoline-4-carboxylate

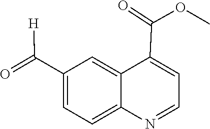 [SMILES: COC(=O)c1ccnc2ccc(C=O)cc12]

Dess-Martin periodinane (4.64 g, 10.9 mmol) was added to a solution of methyl 6-(hydroxymethyl)quinoline-4-carboxylate Intermediate 7 (950 mg, 4.37 mmol) in DCM (20 mL), and the reaction mixture was stirred at 25° C. for 2 h. The reaction mixture was concentrated in vacuo and the crude product was purified by preparative TLC (EtOAc:petroleum ether, 1:1) to give the title compound (850 mg, 90%) as yellow solid: MS (ESI) m/z [M+H]⁺ 215.

### Intermediate 9: Methyl 6-(chloromethyl)quinoline-4-carboxylate

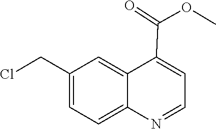 [SMILES: COC(=O)c1ccnc2ccc(CCl)cc12]

Methyl 6-(hydroxymethyl)quinoline-4-carboxylate Intermediate 7 (0.37 g, 1.7 mmol) was added to SOCl₂ (0.26 mL, 3.6 mmol) in DCM (18 mL) at 20° C. The resulting mixture was stirred at 20° C. for 16 h. The material was diluted with EtOAc and filtered through silica. The solvent was removed under reduced pressure to give the title compound (0.44 g) as a pale yellow solid: MS (ESI) m/z [M+H]⁺ 236.

### Intermediate 10: 6-((1H-Pyrazol-1-yl)methyl)quinoline-4-carboxylic Acid

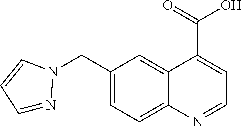 [SMILES: O=C(O)c1ccnc2ccc(Cn3cccn3)cc12]

Intermediate 9 (220 mg, 0.93 mmol) was added to a mixture of 1H-pyrazole (76 mg, 1.1 mmol), Cs₂CO₃ (1.52 g, 4.67 mmol) and KI (465 mg, 2.80 mmol) in MeCN (25 mL) at 20° C. The resulting mixture was stirred at 80° C. for 16 h. The reaction mixture was acidified with HCl (0.1 M) and volatiles were removed under reduced pressure. The dried solid was triturated with DCM (2×50 mL), filtered and concentrated under reduced pressure. The residue was purified by preparative HPLC PrepMethod F (gradient: 1-21%) to give the title compound (40 mg, 17%) as a white solid: MS (ESI) m/z [M+H]⁺ 254.

### Intermediate 11: tert-Butyl 6-(3-methoxypropyl)quinoline-4-carboxylate

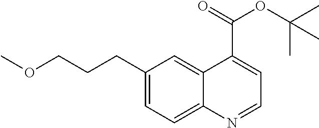 [SMILES: COCCCc1ccc2nccc(C(=O)OC(C)(C)C)c2c1]

NiBr₂ O(CH₂CH₂OCH₃)₂ (30 mg, 0.10 mmol) was added to a mixture of tert-butyl 6-bromoquinoline-4-carboxylate (WO 2019154886) (300 mg, 0.97 mmol), 1-bromo-3-methoxypropane (298 mg, 1.95 mmol), Zn (127 mg, 1.95 mmol), 4,4′-dimethoxy-2,2′-bipyridyl (21 mg, 0.10 mmol) and NaI (146 mg, 0.97 mmol) in DMPU (8 mL). The reaction was stirred at 80° C. for 2 h, diluted with EtOAc, and washed with water. The organic layer was dried over Na₂SO₄, filtered and evaporated under reduced pressure. The residue was purified by preparative TLC (EtOAc:petroleum ether; 1:5) to give the title compound (0.12 g, 41%) as a pale yellow gum: MS (ESI) m/z [M+H]⁺ 302.

### Intermediate 12: 6-(3-Methoxypropyl)quinoline-4-carboxylic Acid

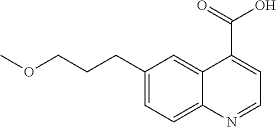 [SMILES: COCCCc1ccc2nccc(C(=O)O)c2c1]

tert-Butyl 6-(3-methoxypropyl)quinoline-4-carboxylate Intermediate 11 (160 mg, 0.53 mmol) was added to a mixture of TFA (4 mL) in DCM (10 mL) and stirred at rt for 2 h. Volatiles were removed under reduced pressure and the solid was further dried under vacuum to give the title compound (0.13 g) as a pale yellow gum: MS (ESI) m/z [M+H]⁺ 246.

### Intermediate 13: tert-Butyl 6-(2-methoxyethyl)quinoline-4-carboxylate

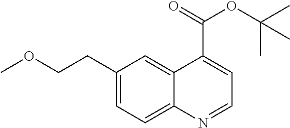 [SMILES: COCCc1ccc2nccc(C(=O)OC(C)(C)C)c2c1]

NiBr₂ O(CH₂CH₂OCH₃)₂ (45 mg, 0.15 mmol) was added to a mixture of tert-butyl 6-bromoquinoline-4-carboxylate (WO 2019154886) (300 mg, 0.97 mmol), 1-bromo-2-methoxyethane (271 mg, 1.95 mmol), Zn (127 mg, 1.95 mmol), 4,4′-dimethoxy-2,2′-bipyridyl (21 mg, 0.10 mmol) and NaI (146 mg, 0.97 mmol) in DMPU (10 mL). The reaction was stirred at 80° C. for 2 h, diluted with EtOAc, and washed with water. The organic layer was dried over Na₂SO₄, filtered and evaporated. The residue was purified by preparative TLC (EtOAc:petroleum ether; 1:4) to give the title compound (80 mg, 29%) as a pale yellow solid:

MS (ESI) m/z [M+H]⁺ 288.

### Intermediate 14: 6-(2-Methoxyethyl)quinoline-4-carboxylic Acid

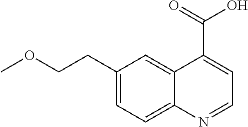 [SMILES: COCCc1ccc2nccc(C(=O)O)c2c1]

tert-Butyl 6-(2-methoxyethyl)quinoline-4-carboxylate Intermediate 13 (80 mg, 0.28 mmol) was added to a mixture of TFA (2 mL) in DCM (5 mL) and stirred at rt for 2 h. Volatiles were removed under reduced pressure and the solid was further dried under vacuum to give the title compound (64 mg, 99%) as a pale yellow gum: MS (ESI) m/z [M+H]⁺ 232.

### Intermediate 15: Methyl 6-(cyanomethyl)quinoline-4-carboxylate

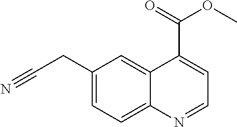 [SMILES: COC(=O)c1ccnc2ccc(CC#N)cc12]

Pd₂(dba)₃·CHCl₃ (194 mg, 0.19 mmol) was added in one portion to a suspension of methyl 6-bromoquinoline-4-carboxylate (500 mg, 1.88 mmol), 2-(trimethylsilyl)acetonitrile (277 mg, 2.44 mmol), ZnF₂ (136 mg, 1.32 mmol) and XantPhos (109 mg, 0.19 mmol) in DMF (8 mL) at 20° C. and under an atmosphere of N₂ (g), and the reaction mixture was stirred at 105° C. for 10 h. The reaction mixture was diluted with EtOAc (100 mL) and washed with sat brine (3×50 mL). The organic layer was dried over Na₂SO₄, filtered and evaporated in vacuo and the residue was purified by preparative TLC (EtOAc:petroleum ether, 1:2) to give the title compound (340 mg, 80%) as a yellow solid: MS (ESI) m/z [M+H]⁺ 227.

### Intermediate 16: Methyl 6-(1-cyanocyclopropyl)quinoline-4-carboxylate

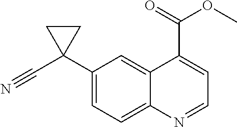 [SMILES: COC(=O)c1ccnc2ccc(C3(C#N)CC3)cc12]

NaH (32 mg, 1.3 mmol) was added portion wise to a solution of methyl 6-(cyanomethyl)quinoline-4-carboxylate Intermediate 15 (300 mg, 1.33 mmol) in DMF (2 mL) and the reaction mixture was stirred at rt for 1 h. 1,2-Dibromoethane (249 mg, 1.33 mmol) was added and the reaction mixture was stirred at 20° C. for 2 h. The reaction mixture was diluted with EtOAc (50 mL), and washed with water (2×20 mL). The organic layer was dried over Na₂SO₄, filtered and evaporated and the residue was purified by preparative

TLC (EtOAc:petroleum ether, 1:2) to give the title compound (200 mg, 60%) as a yellow solid: MS (ESI) m/z [M+H]⁺ 253.

### Intermediate 17: 6-(1-Cyanocyclopropyl)quinoline-4-carboxylic Acid

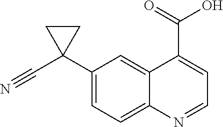 [SMILES: N#CC1(c2ccc3nccc(C(=O)O)c3c2)CC1]

A solution of methyl 6-(1-cyanocyclopropyl)quinoline-4-carboxylate Intermediate 16 (130 mg, 0.52 mmol) and LiOH (25 mg, 1.0 mmol) in THF (6 mL) and water (1 mL) was stirred at rt for 2 h. The solvent was removed under reduced pressure. The reaction mixture was acidified with 2 M HCl (aq) and the precipitate was collected by filtration, washed with water to give the crude title compound (70 mg, 57%) as a white solid. MS (ESI) m/z [M+H]⁺ 239.

### Intermediate 18: Methyl 6-(2-methoxyethoxy)quinoline-4-carboxylate

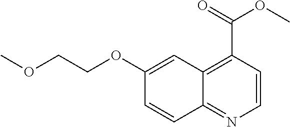

To a stirred solution of methyl 6-hydroxyquinoline-4-carboxylate (200 mg, 0.98 mmol) in DMF (5 mL) was added 1-bromo-2-methoxyethane (137 mg, 0.98 mmol) and Cs₂CO₃ (802 mg, 2.46 mmol). The resulting mixture was stirred at 25° C. for 2 h. The reaction mixture was filtered through Celite®. The solvent was removed under reduced pressure. The residue was purified by preparative TLC (petroleum ether:EtOAc, 2:1), to give the title compound (0.19 g, 73%) as a colourless oil; MS m/z (ESI), [M+H]⁺ 262.0.

### Intermediate 19: 6-(2-Methoxyethoxy)quinoline-4-carboxylic Acid

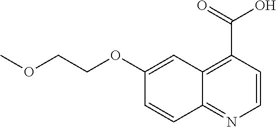 [SMILES: COCCOc1ccc2nccc(C(=O)O)c2c1]

LiOH (55 mg, 2.3 mmol) was added to methyl 6-(2-methoxyethoxy)quinoline-4-carboxylate Intermediate 18 (150 mg, 0.57 mmol) in THF (2 mL) and water (2 mL) at 25° C. The resulting suspension was stirred at 25° C. for 3 h. The reaction mixture was adjusted to pH 5 with 2 M HCl (aq). The solvent was removed under reduced pressure to give the crude product as a pale yellow solid; MS m/z (ESI), [M+H]⁺ 248.0.

### Intermediate 20: Methyl 6-(cyclopropylmethoxy)quinoline-4-carboxylate

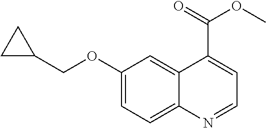 [SMILES: COC(=O)c1ccnc2ccc(OCC3CC3)cc12]

K₂CO₃ (340 mg, 2.46 mmol) was added to methyl 6-hydroxyquinoline-4-carboxylate (200 mg, 0.98 mmol) and (bromomethyl)cyclopropane (199 mg, 1.48 mmol) in DMF (3 mL) at 30° C. The resulting suspension was stirred under N₂ (g) at 60° C. for 3 h. The solvent was removed under reduced pressure. The residue was purified by preparative TLC (EtOAc:petroleum ether, 1:1) to give the title compound (197 mg, 78%) as a yellow solid; MS m/z (ESI), [M+H]⁺ 258.2.

### Intermediate 21: 6-(Cyclopropylmethoxy)quinoline-4-carboxylic Acid

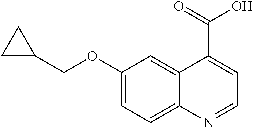 [SMILES: O=C(O)c1ccnc2ccc(OCC3CC3)cc12]

NaOH (148 mg, 3.7 mmol) was added to methyl 6-(cyclopropylmethoxy)quinoline-4-carboxylate Intermediate 20 (191 mg, 0.74 mmol) in MeOH (9 mL) and water (3 mL) at 25° C. The resulting solution was stirred at 25° C. for 1 h. The solvent was removed under reduced pressure. The residue was diluted with water (50 mL) and adjusted to pH 3 with 1 M HCl (aq). The aqueous phase was extracted with EtOAc (3×50 mL) and the organic layer was dried over Na₂SO₄, filtered and evaporated to give the title compound (173 mg, 96%) as a white solid that was used directly in the next step without further purification;

MS m/z (ESI), [M+H]⁺ 244.0.

### Intermediate 22: Methyl 6-((tetrahydro-2H-pyran-4-yl)oxy)quinoline-4-carboxylate

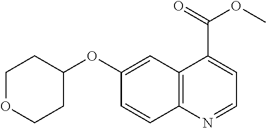 [SMILES: COC(=O)c1ccnc2ccc(OC3CCOCC3)cc12]

DIAD (574 μL, 2.95 mmol) was added slowly to methyl 6-hydroxyquinoline-4-carboxylate (120 mg, 0.59 mmol), tetrahydro-2H-pyran-4-ol (302 mg, 2.95 mmol) and triphenylphosphine (774 mg, 2.95 mmol) in THF (5 mL) at 60° C. The resulting suspension was stirred at 60° C. for 15 min. The solvent was removed under reduced pressure. The residue was purified by preparative TLC (EtOAc:Petroleum ether, 1:2), to give the crude title compound (1.1 g) as a yellow solid; MS m/z (ESI), [M+H]⁺ 288.2.

### Intermediate 23: 6-((Tetrahydro-2H-pyran-4-yl)oxy)quinoline-4-carboxylic Acid

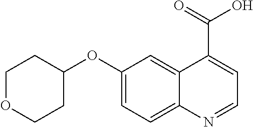 [SMILES: O=C(O)c1ccnc2ccc(OC3CCOCC3)cc12]

NaOH (762 mg, 19.1 mmol) was added to methyl 6-((tetrahydro-2H-pyran-4-yl)oxy)quinoline-4-carboxylate Intermediate 22 (1.10 g, 3.81 mmol) in MeOH (9 mL) and water (3 mL) at 25° C. The resulting solution was stirred at 25° C. for 1 h. The solvent was removed under reduced pressure. The residue was diluted with sat NaHCO₃ (aq, 50 mL) and extracted with EtOAc (3×50 mL). The aqueous layer was adjusted to pH 3 with 1 M HCl (aq) and then extracted with EtOAc (3×100 mL). The combined organic layer was dried over Na₂SO₄, filtered and evaporated to give the title compound (161 mg, 15%) as a white solid. The product was used directly in the next step without further purification; MS m/z (ESI), [M+H]⁺ 274.1.

### Intermediate 24: Methyl 6-((1,3-difluoropropan-2-yl)oxy)quinoline-4-carboxylate

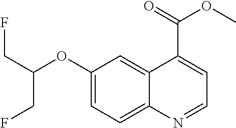 [SMILES: COC(=O)c1ccnc2ccc(OC(CF)CF)cc12]

DIAD (0.896 g, 4.43 mmol) was added dropwise to methyl 6-hydroxyquinoline-4-carboxylate (0.15 g, 0.74 mmol), 1,3-difluoropropan-2-ol (0.426 g, 4.43 mmol) and triphenylphosphine (1.16 g, 4.43 mmol) in THF (15 mL) at 60° C. The resulting solution was stirred at 60° C. for 15 min. The solvent was removed under reduced pressure. The crude product was purified by flash chromatography on silica, (gradient: 1-50% EtOAc in petroleum ether) to give crude title compound (1 g) as a pale yellow solid that was used in the next step; MS m/z (ESI), [M+H]⁺ 282.1.

### Intermediate 25: 6-((1,3-Difluoropropan-2-yl)oxy)quinoline-4-carboxylic Acid

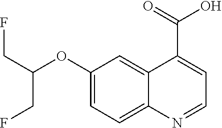 [SMILES: O=C(O)c1ccnc2ccc(OC(CF)CF)cc12]

NaOH (722 mg, 18.0 mmol) was added to methyl 6-((1,3-difluoropropan-2-yl)oxy)quinoline-4-carboxylate Intermediate 24 (1015 mg, 3.61 mmol) in MeOH (9 mL) and water (3 mL) at 25° C. The resulting solution was stirred under air at 25° C. for 1 h. The solvent was removed under reduced pressure. The residue was diluted with sat NaHCO₃ (aq, 50 mL) and extracted with EtOAc (3×50 mL). The aqueous layer was adjusted to pH 3 with 1 M HCl (aq) and extracted with EtOAc (3×50 mL). The organic layer was dried over Na₂SO₄, filtered and evaporated to give the title compound (174 mg, 18%) as a pale yellow solid that was used directly in the next step without further purification; MS m/z (ESI), [M+H]⁺ 268.0.

### Intermediate 26: Methyl 6-((3,3-difluorocyclobutyl)methoxy)quinoline-4-carboxylate

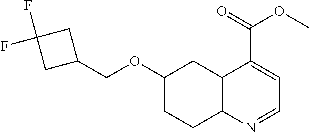 [SMILES: COC(=O)C1=CC=NC2CCC(OCC3CC(F)(F)C3)CC12]

Cs₂CO₃ (802 mg, 2.46 mmol) was added to methyl 6-hydroxyquinoline-4-carboxylate (200 mg, 0.98 mmol) and 3-(bromomethyl)-1,1-difluorocyclobutane (273 mg, 1.48 mmol) in DMF (3 mL) at 25° C. The resulting suspension was stirred under N₂ (g) at 55° C. for 3 h. The solvent was removed under reduced pressure. The residue was diluted with water (50 mL) and extracted with EtOAc (3×50 mL). The organic layer was dried over Na₂SO₄, filtered and evaporated. The residue was purified by preparative TLC (EtOAc:petroleum ether, 1:2), to give the title compound (132 mg, 43%) as a brown gum; MS m/z (ESI), [M+H]⁺ 308.0.

### Intermediate 27: 6-((3,3-Difluorocyclobutyl)methoxy)quinoline-4-carboxylic Acid

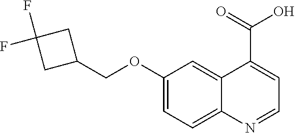 [SMILES: O=C(O)c1ccnc2ccc(OCC3CC(F)(F)C3)cc12]

NaOH (83 mg, 2.1 mmol) was added to methyl 6-((3,3-difluorocyclobutyl)methoxy)-quinoline-4-carboxylate Intermediate 26 (127 mg, 0.41 mmol) in MeOH (9 mL) and water (3 mL) at 25° C. The resulting solution was stirred at 25° C. for 1 h. The solvent was removed under reduced pressure and the mixture was diluted with water (50 mL). The pH was adjusted to pH 3 with 1 M HCl (aq). The aqueous phase was extracted with EtOAc (3×50 mL). The organic phase was dried over Na₂SO₄, filtered and evaporated to give the title compound (120 mg, 99%) as a white solid which was used directly in the next step without further purification; MS m/z (ESI), [M+H]⁺ 294.0.

### Intermediate 28: rac-Methyl (R)-6-((2,2-dimethyltetrahydro-2H-pyran-4-yl)oxy)quinoline-4-carboxylate

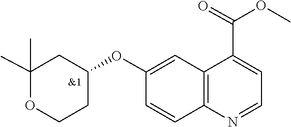

DIAD (718 μL, 3.69 mmol) was added slowly to methyl 6-hydroxyquinoline-4-carboxylate (150 mg, 0.74 mmol), rac-(R)-2,2-dimethyltetrahydro-2H-pyran-4-ol (WO 2012021591) (481 mg, 3.69 mmol) and triphenylphosphine (968 mg, 3.69 mmol) in THF (10 mL) at 60° C. The resulting suspension was stirred at 60° C. for 15 min. The solvent was removed under reduced pressure. The residue was triturated with Et₂O (2×15 mL) and filtered. The residue was purified by preparative TLC (EtOAc:petroleum ether, 1:2), to give the crude title compound (365 mg) as a yellow gum, that was used in the next step; MS m/z (ESI), [M+H]⁺ 316.0.

### Intermediate 29: rac-(R)-6-((2,2-Dimethyltetrahydro-2H-pyran-4-yl)oxy)quinoline-4-carboxylic Acid

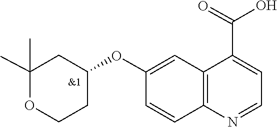 [SMILES: CC1(C)C[C@H](Oc2ccc3nccc(C(=O)O)c3c2)CCO1]

NaOH (224 mg, 5.60 mmol) was added to rac-methyl (R)-6-((2,2-dimethyltetrahydro-2H-pyran-4-yl)oxy)quinoline-4-carboxylate Intermediate 28 (353 mg, 1.12 mmol) in MeOH (9 mL) and water (3 mL) at 25° C. The resulting solution was stirred at 25° C. for 1 h. The solvent was removed under reduced pressure. The residue was diluted with sat NaHCO₃ (aq, 50 mL) and extracted with EtOAc (3×50 mL). The pH of the aqueous layer was adjusted to pH 3 with 1 M HCl (aq) and then extracted with EtOAc (3×50 mL). The organic layer was dried over Na₂SO₄, filtered and evaporated to give the title compound (194 mg, 57%) as a white solid that was directly used in the next step without further purification; MS m/z (ESI), [M+H]⁺ 302.2.

### Intermediate 30: tert-Butyl 6-(4,4,5,5-tetramethyl-1,3,2-dioxaborolan-2-yl)quinoline-4-carboxylate

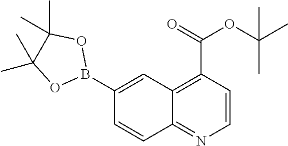 [SMILES: CC(C)(C)OC(=O)c1ccnc2ccc(B3OC(C)(C)C(C)(C)O3)cc12]

Pd(dppf)Cl₂ (137 mg, 0.19 mmol) was added to tert-butyl 6-bromoquinoline-4-carboxylate (WO 2019154886) (640 mg, 2.08 mmol), KOAc (549 mg, 5.59 mmol) and bis(pinacolato)diboron (711 mg, 2.80 mmol) in 1,4-dioxane (10 mL). The resulting solution was stirred under N₂ (g) at 80° C. for 3 h. The solvent was removed under reduced pressure. The mixture was diluted with EtOAc (50 mL) and washed sequentially with water (3×25 mL). The organic layer was dried over Na₂SO₄, filtered and evaporated. The residue was purified by preparative TLC (EtOAc:petroleum ether, 2:1), to give the title compound (540 mg, 73%) as a yellow solid; MS m/z (ESI), [M+H]⁺ 356.1.

### Intermediate 31: tert-Butyl 6-hydroxyquinoline-4-carboxylate

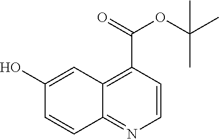 [SMILES: CC(C)(C)OC(=O)c1ccnc2ccc(O)cc12]

Sodium perborate tetrahydrate (458 mg, 2.98 mmol) was added to tert-butyl 6-(4,4,5,5-tetramethyl-1,3,2-dioxaborolan-2-yl)quinoline-4-carboxylate Intermediate 30 (580 mg, 1.63 mmol) in THF (8 mL) and water (4 mL). The resulting solution was stirred at 20° C. for 6 h. The solvent was removed under reduced pressure. The residue was purified by preparative TLC (EtOAc:petroleum ether, 1:2), to give the title compound (350 mg, 87%) as a yellow solid; MS m/z (ESI), [M+H]⁺ 246.0.

### Intermediate 32: tert-Butyl 6-(cyanomethoxy)quinoline-4-carboxylate

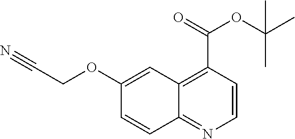 [SMILES: CC(C)(C)OC(=O)c1ccnc2ccc(OCC#N)cc12]

Bromoacetonitrile (257 mg, 2.14 mmol) was added to tert-butyl 6-hydroxyquinoline-4-carboxylate Intermediate 31 (350 mg, 1.43 mmol) and K₂CO₃ (493 mg, 3.57 mmol) in DMF (20 mL). The resulting solution was stirred under N₂ (g) at 60° C. for 6 h. The solvent was removed under reduced pressure. The residue was purified by preparative TLC (EtOAc:petroleum ether, 1:1), to give the title compound (320 mg, 79%) as a yellow solid; MS m/z (ESI), [M+H]⁺ 285.1.

### Intermediate 33: 6-(Cyanomethoxy)quinoline-4-carboxylic Acid

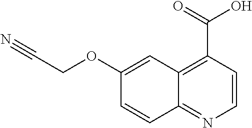 [SMILES: N#CCOc1ccc2nccc(C(=O)O)c2c1]

TFA (650 μL, 8.44 mmol) was added slowly to tert-butyl 6-(cyanomethoxy)quinoline-4-carboxylate Intermediate 32 (300 mg, 1.06 mmol) in DCM (3 mL). The resulting solution was stirred at 20° C. for 18 h. The solvent was removed under reduced pressure to give the crude title compound (300 mg, 83%); MS m/z (ESI), [M+H]⁺ 229.1.

### Intermediate 34: Methyl 6-(2-oxopropoxy)quinoline-4-carboxylate

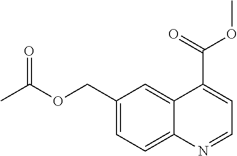 [SMILES: COC(=O)c1ccnc2ccc(COC(C)=O)cc12]

A solution of methyl 6-hydroxyquinoline-4-carboxylate (200 mg, 0.98 mmol), 1-bromopropan-2-one (270 mg, 1.97 mmol) and Cs₂CO₃ (641 mg, 1.97 mmol) in THF (8 mL) was stirred at 50° C. for 20 h. The solvent was removed under reduced pressure. The reaction mixture was diluted with EtOAc (25 mL), and washed with water (3×10 mL). The organic layer was dried over Na₂SO₄, filtered and evaporated. The crude was purified by preparative TLC (petroleum ether:EtOAc, 3:2), to give the title compound (220 mg, 86%) as a white solid; MS m/z (ESI), [M+H]⁺ 260.0.

### Intermediate 35: Methyl 6-(2,2-difluoropropoxy)quinoline-4-carboxylate

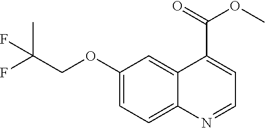 [SMILES: COC(=O)c1ccnc2ccc(OCC(C)(F)F)cc12]

DAST (561 μL, 4.24 mmol) was added to a solution of methyl 6-(2-oxopropoxy)quinoline-4-carboxylate Intermediate 34 (220 mg, 0.85 mmol) in DCM (10 mL) at 0° C. The reaction was stirred at 25° C. for 20 h. The solvent was removed under reduced pressure. The residue was purified by preparative TLC (petroleum ether:EtOAc, 3:2), to give the title compound (180 mg, 75%) as a white solid; MS m/z (ESI), [M+H]⁺ 282.1.

### Intermediate 36: 6-(2,2-Difluoropropoxy)quinoline-4-carboxylic Acid

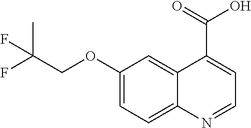 [SMILES: CC(F)(F)COc1ccc2nccc(C(=O)O)c2c1]

A solution of methyl 6-(2,2-difluoropropoxy)quinoline-4-carboxylate Intermediate 35 (170 mg, 0.60 mmol) and LiOH (29 mg, 1.2 mmol) in MeOH (6 mL) and water (1 mL) was stirred at 25° C. for 3 h. The solvent was removed under reduced pressure. The mixture was diluted with water (10 mL) and pH adjusted to pH 6 with 1 M HCl (aq). The precipitate was collected by filtration, washed with water (25 mL) and dried under vacuum to give the title compound (150 mg, 93%) as a white solid that was used without further purification; MS m/z (ESI), [M+H]⁺ 268.0.

### Intermediate 37: rac-(R)-1-(4-Chloroquinolin-6-yl)-1-(3-fluoropyridin-2-yl)ethan-1-ol

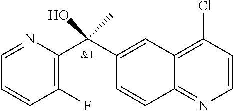 [SMILES: C[C@](O)(c1ccc2nccc(Cl)c2c1)c1ncccc1F]

n-BuLi (4.95 mL, 12.4 mmol, 2.5 M in hexane) was added slowly to 6-bromo-4-chloroquinoline (2.0 g, 8.3 mmol) in THF (20 mL) under N₂ (g) at −78° C. The resulting solution was stirred under N₂ (g) at −78° C. for 3 h. 1-(3-fluoropyridin-2-yl)ethan-1-one (1.15 g, 8.25 mmol) was added slowly, over a period of 15 min, to the stirred mixture warmed to −40° C. The resulting solution was stirred under N₂ (g) at −40° C. for 2 h. The reaction mixture was quenched with sat NH₄Cl (aq, 20 mL) and extracted with EtOAc (3×50 mL). The organic layer was dried over Na₂SO₄, filtered and evaporated. The crude product was purified by flash chromatography on C18 (gradient: 40-45% MeCN in water) to give the title compound (687 mg, 27%) as a brown gum after evaporation of pure fractions; MS m/z (ESI), [M+H]⁺ 303.2.

### Intermediate 38: rac-Methyl (R)-6-(1-(3-fluoropyridin-2-yl)-1-hydroxyethyl)quinoline-4-carboxylate

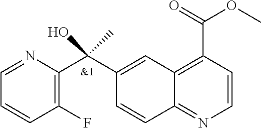 [SMILES: COC(=O)c1ccnc2ccc([C@](C)(O)c3ncccc3F)cc12]

Pd(OAc)₂ (54 mg, 0.24 mmol) and Pd(dppf)Cl₂ (88 mg, 0.12 mmol) was added to a mixture of rac-(R)-1-(4-chloroquinolin-6-yl)-1-(3-fluoropyridin-2-yl)ethan-1-ol Intermediate 37 (727 mg, 2.40 mmol), TEA (1.0 mL, 7.2 mmol) and dppf (200 mg, 0.36 mmol) in MeOH (5 mL) under N₂ (g). The resulting mixture was stirred at 100° C. for 12 h under CO (g) atmosphere (20 atm). The solvent was removed under reduced pressure. The residue was diluted with water (50 mL) and extracted with EtOAc (3×50 mL). The organic layer was dried over Na₂SO₄, filtered and evaporated. The crude product was purified by flash chromatography on silica (gradient: 50-80% EtOAc in petroleum ether) to give the title compound (537 mg, 68%) as a brown gum after evaporation of pure fractions; MS m/z (ESI), [M+H]⁺ 327.2.

### Intermediate 39: rac-Methyl (R)-6-(1-(3-fluoropyridin-2-yl)ethyl)quinoline-4-carboxylate

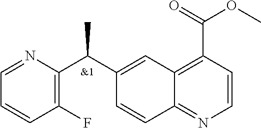 [SMILES: COC(=O)c1ccnc2ccc([C@H](C)c3ncccc3F)cc12]

rac-Methyl (R)-6-(1-(3-fluoropyridin-2-yl)-1-hydroxyethyl)quinoline-4-carboxylate Intermediate 38 (120 mg, 0.37 mmol) was added to triethylsilane (5 mL) and TFA (5 mL) at 13° C. The resulting solution was stirred under N₂ (g) at 100° C. for 2 days. Further triethylsilane (1 mL) and TFA (1 mL) was added and the reaction was stirred for 1 day at 100° C. The solvent was removed under reduced pressure. The residue was diluted with sat NaHCO₃ (aq, 50 mL) and extracted with EtOAc (3×100 mL). The organic layer was dried over Na₂SO₄, filtered and evaporated. The crude product was purified by flash chromatography on C18, (gradient: 40-60% MeCN in water) to give the title compound (12 mg, 10%) as a yellow gum after evaporation of pure fractions; MS m/z (ESI), [M+H]⁺ 311.1.

### Intermediate 40: rac-(R)-6-(1-(3-Fluoropyridin-2-yl)ethyl)quinoline-4-carboxylic Acid

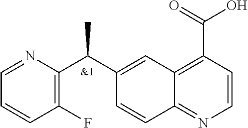 [SMILES: C[C@@H](c1ccc2nccc(C(=O)O)c2c1)c1ncccc1F]

NaOH (32 mg, 0.79 mmol) was added to rac-methyl (R)-6-(1-(3-fluoropyridin-2-yl)ethyl)quinoline-4-carboxylate Intermediate 39 (49 mg, 0.16 mmol) in MeOH (3 mL) and water (1 mL) at 15° C. The resulting solution was stirred under N₂ (g) at 15° C. for 1 h. The solvent was removed under reduced pressure. The mixture was diluted with water (10 mL) and the pH adjusted to pH 3 with 1 M HCl (aq). The aqueous phase was extracted with EtOAc (3×50 mL). The organic layer was dried over Na₂SO₄, filtered and evaporated to give the crude title compound (100 mg) as a colorless gum that was used directly in the next step; MS m/z (ESI), [M+H]⁺ 297.2.

### Intermediate 41: 2,2′-(Cyclopropane-1,1-diyl)bis(4,4,5,5-tetramethyl-1,3,2-dioxaborolane)

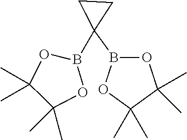

Bromocyclopropane (7.0 g, 58 mmol) and 4,4,4′,4′,5,5,5′,5′-octamethyl-2,2′-bi(1,3,2-dioxaborolane) (14.1 g, 55.6 mmol) in THF (50 mL) was added dropwise, over a period of 30 min, to lithium 2,2,6,6-tetramethylpiperidin-1-ide (11.9 g, 81.0 mmol) in THF (100 mL), under N₂ (g) and cooled to −78° C. The resulting solution was stirred at −80° C. for 1 h. The reaction mixture was poured into sat NaHCO₃ (aq, 300 mL) and extracted with EtOAc (3×200 mL). The organic layer was dried over Na₂SO₄, filtered and evaporated. The crude product was purified by flash chromatography on silica (gradient 0-2% EtOAc in petroleum ether) to give the title compound (7.2 g, 42%) as a white solid after evaporation of pure fractions; ¹H NMR (300 MHz, CDCl₃): δ 1.20 (s, 24H), 0.78 (s, 4H).

### Intermediate 42: 4-Chloro-6-(1-(4,4,5,5-tetramethyl-1,3,2-dioxaborolan-2-yl)cyclopropyl)quinoline

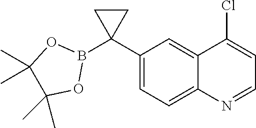 [SMILES: CC1(C)OB(C2(c3ccc4nccc(Cl)c4c3)CC2)OC1(C)C]

cataCXium® A Pd G3 (0.557 g, 0.77 mmol) was added to 2,2′-(cyclopropane-1,1-diyl)bis(4,4,5,5-tetramethyl-1,3,2-dioxaborolane) Intermediate 41 (5.0 g, 17 mmol) and 6-bromo-4-chloroquinoline (5.36 g, 22.1 mmol) and Cs₂CO₃ (11.1 g, 34.0 mmol) in 1,4-dioxane (20 mL) and water (2 mL) at 20° C. The resulting solution was stirred under N₂ (g) at 100° C. for 20 h. The reaction mixture was diluted with EtOAc, dried over Na₂SO₄, filtered and evaporated to afford a brown gum. The crude product was purified by flash chromatography on silica, (gradient 2-20% EtOAc in petroleum ether) to give the title compound (3.8 g, 67%) as a pale yellow oil which solidified when standing; MS m/z (ESI), [M+H]⁺ 330.3.

### Intermediate 43: Potassium (1-(4-chloroquinolin-6-yl)cyclopropyl)-trifluoroborate

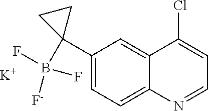

KHF₂ (5.40 g, 69.2 mmol) was added to 4-chloro-6-(1-(4,4,5,5-tetramethyl-1,3,2-dioxaborolan-2-yl)cyclopropyl)quinoline Intermediate 42 (3.8 g, 12 mmol) in MeOH (20 mL) at 20° C. The resulting solution was stirred under N₂ (g) at 80° C. for 14 h. The reaction mixture was evaporated. The crude white solid was triturated with petroleum ether:TBME, 1:1 to give a solid. The precipitate was collected by filtration, washed with hot MeCN (100 mL). The filtrate was concentrated to give the title compound (2.3 g, 64%) as a yellow solid that was used without further purification; MS m/z (ESI), [M+H]⁺ 269.9.

### Intermediate 44: 4-Chloro-6-(1-(2-(trifluoromethyl)pyridin-4-yl)cyclopropyl)-quinoline

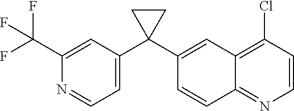 [SMILES: FC(F)(F)c1cc(C2(c3ccc4nccc(Cl)c4c3)CC2)ccn1]

cataCXium® A Pd G3 (57 mg, 0.08 mmol) was added to 4-bromo-2-(trifluoromethyl)pyridine (263 mg, 1.16 mmol), potassium (1-(4-chloroquinolin-6-yl)cyclopropyl)trifluoroborate Intermediate 43 (300 mg, 0.97 mmol) and Cs₂CO₃ (632 mg, 1.94 mmol) in 1,4-dioxane (10 mL) and water (1 mL) at 20° C. The resulting solution was stirred under N₂ (g) at 100° C. for 14 h. The reaction mixture was dried over Na₂SO₄, and filtered through Celite®. The filter cake was washed with DCM (20 mL). The solvent was removed under reduced pressure. The residue was purified by preparative TLC (EtOAc:petroleum ether, 1:1), to give the title compound (256 mg, 76%) as a pale yellow gum. MS m/z (ESI), [M+H]⁺ 349.0.

### Intermediate 45: Methyl 6-(1-(2-(trifluoromethyl)pyridin-4-yl)cyclopropyl)-quinoline-4-carboxylate

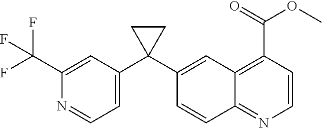 [SMILES: COC(=O)c1ccnc2ccc(C3(c4ccnc(C(F)(F)F)c4)CC3)cc12]

TEA (318 μL, 2.28 mmol) was added to 4-chloro-6-(1-(2-(trifluoromethyl)pyridin-4-yl)cyclopropyl)quinoline Intermediate 44 (265 mg, 0.76 mmol), Pd(OAc)₂ (17 mg, 0.08 mmol), Pd(dppf)Cl₂ (28 mg, 0.04 mmol) and dppf (63 mg, 0.11 mmol) in MeOH (5 mL) at 13° C. The resulting solution was stirred at 100° C. for 12 h under CO (g) atmosphere (20 atm). The solvent was removed under reduced pressure. The residue was diluted with water (50 mL) and extracted with EtOAc (3×50 mL). The organic layer was dried over Na₂SO₄, filtered and evaporated. The residue was purified by preparative TLC (EtOAc:petroleum ether, 1:1), to give the title compound (161 mg, 56%) as a brown gum; MS m/z (ESI), [M+H]⁺ 373.1.

### Intermediate 46: 6-(1-(2-(Trifluoromethyl)pyridin-4-yl)cyclopropyl)quinoline-4-carboxylic Acid

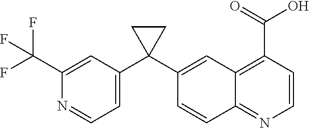 [SMILES: O=C(O)c1ccnc2ccc(C3(c4ccnc(C(F)(F)F)c4)CC3)cc12]

NaOH (86 mg, 2.2 mmol) was added to methyl 6-(1-(2-(trifluoromethyl)pyridin-4-yl)cyclopropyl)quinoline-4-carboxylate Intermediate 45 (160 mg, 0.43 mmol) in MeOH (6 mL) and water (2 mL) at 15° C. The resulting solution was stirred at 15° C. for 1 h. The solvent was removed under reduced pressure. The residue was diluted with water (10 mL) and the pH was adjusted to pH 3 with 1 M HCl (aq). The aqueous phase was extracted with EtOAc (3×50 mL) and the organic layer was dried over Na₂SO₄, filtered and evaporated to give the crude title compound (158 mg) as a orange solid that was used directly in the next step without further purification; MS m/z (ESI), [M+H]⁺ 359.0.

### Intermediate 47: 6-(Furan-3-yl)quinoline-4-carboxylic Acid

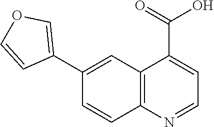 [SMILES: O=C(O)c1ccnc2ccc(-c3ccoc3)cc12]

6-Bromoquinoline-4-carboxylic acid (250 mg, 0.99 mmol), furan-3-ylboronic acid (111 mg, 0.99 mmol), Pd(dppf)Cl₂ (65 mg, 0.10 mmol) and Cs₂CO₃ (969 mg, 2.98 mmol) were mixed in 1,4-dioxane (6 mL) and water (1.5 mL). The mixture was bubbled with N₂ (g) for 10 min, then stirred under N₂ (g) at rt for 19 h. HCl (3.8 M, 783 μL, 2.98 mmol) and DMSO was added (5 mL) and the 1,4-dioxane was evaporated. The mixture was filtered through a syringe filter and purified by preparative HPLC, PrepMethod A (gradient 5-40%) to give the title compound (137 mg, 58%) as a solid after evaporation of solvents and co-evaporation with EtOH (1×); MS m/z (ESI), [M+H]⁺ 240.0.

### Intermediate 48: 4-(4-Chloroquinolin-6-yl)tetrahydro-2H-pyran-4-ol

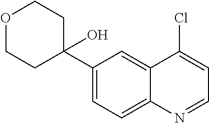 [SMILES: OC1(c2ccc3nccc(Cl)c3c2)CCOCC1]

6-Bromo-4-chloroquinoline (10 g, 41 mmol) was dissolved, under an argon atmosphere, in THF (200 mL), in a dried two necked flask and cooled to −70° C. n-BuLi (2.5 M in hexane, 24.7 mL, 61.9 mmol) was slowly added dropwise so that the internal temperature did not exceed −65° C. The reaction mixture was stirred for 1 h at −70° C. Tetrahydro-4H-pyran-4-one (6.19 g, 61.9 mmol) in THF (10 mL) was slowly added dropwise so that the internal temperature did not exceed −65° C. The reaction solution was stirred for 1 h at −70° C. and then warmed to rt and stirred for 3 h. The reaction mixture was poured into sat NH₄Cl (100 mL) and extracted with EtOAc (4×100 mL). The organic layer was dried over Na₂SO₄, filtered and evaporated. The crude product was purified by flash chromatography on silica (gradient: 10-50% EtOAc in petroleum ether) to give the title compound (3.5 g, 32%) as a pale yellow solid; MS m/z (ESI), [M+H]⁺ 264.0.

### Intermediate 49: Methyl 6-(4-hydroxytetrahydro-2H-pyran-4-yl)quinoline-4-carboxylate

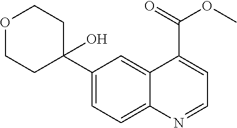 [SMILES: COC(=O)c1ccnc2ccc(C3(O)CCOCC3)cc12]

Pd(OAc)₂ (0.17 g, 0.76 mmol) and Pd(dppf)Cl₂·DCM (0.31 g, 0.38 mmol) was added to a mixture of 4-(4-chloroquinolin-6-yl)tetrahydro-2H-pyran-4-ol Intermediate 48 (2.0 g, 7.6 mmol) dppf (0.63 g, 1.1 mmol) and TEA (3.2 mL, 23 mmol) in MeOH (20 mL) under N₂ (g). The resulting mixture was stirred at 100° C. for 12 h under CO (g) atmosphere (10 atm). The solvent was removed under reduced pressure. The reaction mixture was diluted with EtOAc (50 mL), and washed sequentially with water (3×25 mL). The organic layer was dried over Na₂SO₄, filtered and evaporated. The crude product was purified by flash chromatography on silica (gradient: 10-50% EtOAc in petroleum ether) to give the title compound (1.6 g, 73%) as a pale yellow solid; MS m/z (ESI), [M+H]⁺ 288.1.

### Intermediate 50: 6-(4-Hydroxytetrahydro-2H-pyran-4-yl)quinoline-4-carboxylic Acid

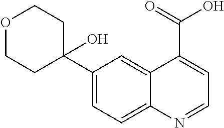 [SMILES: O=C(O)c1ccnc2ccc(C3(O)CCOCC3)cc12]

A solution of methyl 6-(4-hydroxytetrahydro-2H-pyran-4-yl)quinoline-4-carboxylate Intermediate 49 (180 mg, 0.63 mmol) and LiOH (45 mg, 1.9 mmol) in MeOH (8 mL) and water (2 mL) was stirred at 30° C. for 2 h. The solvent was removed under reduced pressure. The mixture was diluted with water (10 mL) and the pH was adjusted to pH 6 with 1 M HCl. The reaction mixture was diluted with water (20 mL) and extracted with EtOAc (3×25 mL). The organic layer was dried over Na₂SO₄, filtered and evaporated to give the title compound (100 mg, 58%) as an orange solid that was used directly in the next step; MS m/z (ESI), [M+H]⁺ 274.1.

### Intermediate 51: 6-(2-Hydroxypropan-2-yl)quinoline-4-carboxylic Acid

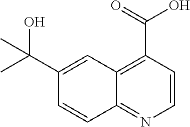 [SMILES: CC(C)(O)c1ccc2nccc(C(=O)O)c2c1]

A solution of methyl 6-(2-hydroxypropan-2-yl)quinoline-4-carboxylate Intermediate 6 (80 mg, 0.33 mmol) and LiOH (16 mg, 0.65 mmol) in THF (4 mL) and water (0.5 mL) was stirred at rt for 1 h. The solvent was removed under reduced pressure. The mixture was acidified with 2 M HCl and diluted with EtOAc, then washed with water. The organic layer was dried over Na₂SO₄, filtered and evaporated to give the title compound (60 mg, 80%) that was used directly in the next step without further purification; MS m/z (ESI), [M+H]⁺ 232.0.

### Intermediate 52: 6-(Trifluoromethyl)quinoline-4-carboxylic Acid

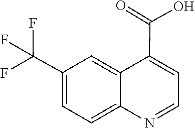 [SMILES: O=C(O)c1ccnc2ccc(C(F)(F)F)cc12]

A mixture of 4-bromo-6-(trifluoromethyl)quinoline (150 mg, 0.54 mmol), Pd(OAc)₂ (12 mg, 0.05 mmol), XantPhos (47 mg, 0.08 mmol), DCC (224 mg, 1.09 mmol), TEA (0.227 mL, 1.63 mmol) and acetic formic anhydride (335 mg, 3.80 mmol) was stirred at 100° C. for 2 h. The solvent was removed under reduced pressure. The mixture was diluted with EtOAc, and washed with water. The aqueous layer was evaporated to give the title compound (80 mg, 61%) as a pale yellow product that was used directly in the next step without further purification; MS m/z (ESI), [M+H]⁺ 242.0.

### Intermediate 53: Ethyl 6-(4,4,5,5-tetramethyl-1,3,2-dioxaborolan-2-yl)quinoline-4-carboxylate

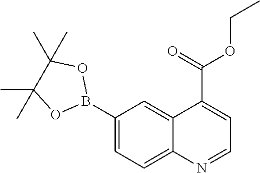 [SMILES: CCOC(=O)c1ccnc2ccc(B3OC(C)(C)C(C)(C)O3)cc12]

Ethyl 6-bromoquinoline-4-carboxylate (1.0 g, 3.6 mmol), 4,4,4′,4′,5,5,5′,5′-octamethyl-2,2′-bi(1,3,2-dioxaborolane) (0.997 g, 3.93 mmol), KOAc (0.701 g, 7.14 mmol) and Pd(dppf)Cl₂·DCM (0.146 g, 0.18 mmol) were added to a microwave vial. The mixture was flushed with N₂ (g), 1,4-dioxane (12 mL) was added and the mixture bubbled with N₂ (g) for 10 min. The vial was capped and heated in an oil-bath at 80° C. overnight. After cooling to rt, the reaction was diluted with EtOAc (100 mL) and washed with water (2×) and brine (2×), dried (MgSO₄) and evaporated. The residue was purified by automated flash chromatography (25 g silica column, gradient: 5-50% of EtOAc in heptane over 25 column volumes, wavelength: 240 nm) to give the title compound (1.13 g, 97%) as a syrup after evaporation of solvents and kept under vacuum for 3 days. The compound solidified upon standing; MS m/z (ESI), [M+H]⁺ 328.2.

### Intermediate 54: Ethyl 6-((2,5-dimethyl-2H-1,2,3-triazol-4-yl)methyl)quinoline-4-carboxylate

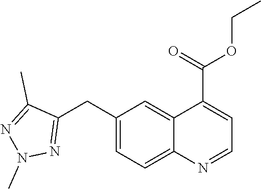 [SMILES: CCOC(=O)c1ccnc2ccc(Cc3nn(C)nc3C)cc12]

Ethyl 6-(4,4,5,5-tetramethyl-1,3,2-dioxaborolan-2-yl)quinoline-4-carboxylate Intermediate 53 (200 mg, 0.61 mmol), 4-(bromomethyl)-2,5-dimethyl-2H-1,2,3-triazole (WO 2007096576) (128 mg, 0.67 mmol), Na₂CO₃ (259 mg, 2.45 mmol) and trans-bromo(N-succinimidyl)-bis(triphenylphosphine)palladium(II) (25 mg, 0.03 mmol) were added to a microwave vial. Toluene (5 mL) and water (1 mL) were added. The mixture was bubbled with N₂ (g) for 10 min. The vial was capped and heated in a microwave oven at 100° C. for 2 h. DCM and water were added, the mixture stirred, filtered through a phase separator and evaporated. The residue was purified by automated flash chromatography (10 g Biotage® KP-SIL column, gradient: 20-100% EtOAc in heptane over 40 column volumes, wavelength: 245 nm) to give the title compound (96 mg, 50%); MS m/z (ESI), [M+H]⁺ 311.3.

### Intermediate 55: tert-Butyl 6-(tetrahydro-2H-pyran-4-yl)quinoline-4-carboxylate

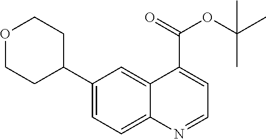 [SMILES: CC(C)(C)OC(=O)c1ccnc2ccc(C3CCOCC3)cc12]

(4-(tert-Butoxycarbonyl)quinolin-6-yl)boronic acid Intermediate 169 (814 mg, 2.98 mmol) was added to 4-methyl-N′-(tetrahydro-4H-pyran-4-ylidene)benzenesulfonohydrazide (J Org Chem. 2014 79, 328) (400 mg, 1.49 mmol) and K₂CO₃ (824 mg, 5.96 mmol) in 1,4-dioxane (150 mL) at 20° C. The resulting solution was stirred at 110° C. for 15 h. The solvent was removed under reduced pressure. The mixture was poured into sat NaHCO₃ (aq, 150 mL) and extracted with EtOAc (3×150 mL). The organic layer was dried over Na₂SO₄, filtered and evaporated. The residue was purified by preparative TLC (petroleum ether:EtOAc, 2:1), to give the title compound (100 mg, 21%) as a pale yellow gum; MS m/z (ESI), [M+H]⁺ 314.1.

### Intermediate 56: 4-Carboxy-6-(tetrahydro-2H-pyran-4-yl)quinolin-1-ium 2,2,2-trifluoroacetate

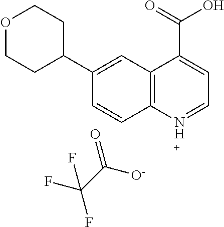 [SMILES: O=C(O)c1ccnc2ccc(C3CCOCC3)cc12 ; O=C([O-])C(F)(F)F]

TFA (1.0 mL, 13 mmol) was added to tert-butyl 6-(tetrahydro-2H-pyran-4-yl)quinoline-4-carboxylate Intermediate 55 (99 mg, 0.32 mmol) in DCM (2 mL) at 20° C. The resulting solution was stirred at 20° C. for 16 h. The solvent was removed under reduced pressure to give the title compound containing 3.3 eq TFA (0.234 g, 99%) as a brown gum that was used directly in the next step without further purification; MS m/z (ESI), [M+H]⁺ 258.2.

### Intermediate 57: tert-Butyl 6-(4-methyltetrahydro-2H-pyran-4-yl)quinoline-4-carboxylate

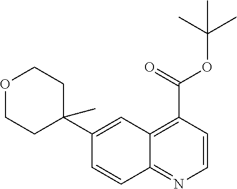 [SMILES: CC(C)(C)OC(=O)c1ccnc2ccc(C3(C)CCOCC3)cc12]

1,3-Di-tert-butyl-1H-imidazol-3-ium chloride (33 mg, 0.15 mmol) was added to tert-butyl 6-bromoquinoline-4-carboxylate (WO 2019154886) (200 mg, 0.65 mmol), 4-bromo-4-methyltetrahydro-2H-pyran (J Am Chem Soc. 2015 137, 11562) (89 mg, 0.50 mmol), nickel(II) (Z)-4-oxopent-2-en-2-olate (26 mg, 0.10 mmol), magnesium chloride (71 mg, 0.75 mmol), Zn (65 mg, 1.0 mmol) and DMAP (73 mg, 0.60 mmol) in DMA (8 mL) at 20° C. The resulting suspension was stirred under N₂ (g) at 25° C. for 18 h. The reaction mixture was diluted with EtOAc (60 mL) and filtered through Celite®. The organic layers were combined and washed with brine (2×50 mL). The organic layer was dried over Na₂SO₄, filtered and evaporated to give an yellow oil. The residue was purified by preparative TLC (petroleum ether:EtOAc, 4:1), to give the crude title compound as a pale yellow oil. The crude was further purified by preparative HPLC PrepMethod B to give the title compound (25 mg, 15%) as a yellow oil; MS m/z (ESI), [M+H]⁺ 328.1.

### Intermediate 58: 6-(4-Methyltetrahydro-2H-pyran-4-yl)quinoline-4-carboxylic Acid

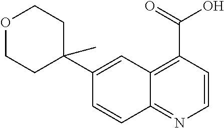 [SMILES: CC1(c2ccc3nccc(C(=O)O)c3c2)CCOCC1]

TFA (0.50 mL, 6.5 mmol) was added to tert-butyl 6-(4-methyltetrahydro-2H-pyran-4-yl)quinoline-4-carboxylate Intermediate 57 (25 mg, 0.08 mmol) in DCM (5 mL) at 20° C. The resulting solution was stirred at 20° C. for 15 h. The solvent was removed under reduced pressure to give the title compound (20 mg, 97%) as a yellow gum. The product was used directly in the next step without further purification; MS m/z (ESI), [M+H]⁺ 272.3.

### Intermediate 59: 6-(3,6-dihydro-2H-Pyran-4-yl)quinoline-4-carboxylic Acid

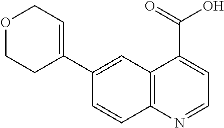 [SMILES: O=C(O)c1ccnc2ccc(C3=CCOCC3)cc12]

6-Bromoquinoline-4-carboxylic acid (100 mg, 0.40 mmol), 2-(3,6-dihydro-2H-pyran-4-yl)-4,4,5,5-tetramethyl-1,3,2-dioxaborolane (ACS Med Chem Lett. 2016 7, 666) (92 mg, 0.44 mmol), Pd(dtbpf)Cl₂ (26 mg, 0.04 mmol) and Cs₂CO₃ (388 mg, 1.19 mmol) were mixed in 1,4-dioxane (3 mL) and water (0.75 mL). The mixture was bubbled with N₂ (g) for 10 min, then stirred under N₂ (g) at rt overnight. DMSO was added (2 mL) and the 1,4-dioxane evaporated. The mixture was filtered through a syringe filter and purified by preparative HPLC; PrepMethod C (gradient 0-30%). The MeCN was evaporated and the remaining water phase freeze dried to give the title compound (30 mg, 29%) as a solid; MS m/z (ESI), [M+H]⁺ 256.1.

### Intermediate 60: 6-(4-Ethoxytetrahydro-2H-pyran-4-yl)quinoline-4-carboxylic Acid

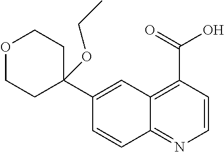 [SMILES: CCOC1(c2ccc3nccc(C(=O)O)c3c2)CCOCC1]

NaH (56 mg, 1.4 mmol, 60% wt) was added to a stirred solution of methyl 6-(4-hydroxytetrahydro-2H-pyran-4-yl)quinoline-4-carboxylate Intermediate 49 (200 mg, 0.70 mmol) in DMF (2 mL) cooled to 0° C. The resulting suspension was stirred at 10° C. for 30 min. Ethyl iodide (139 μL, 1.74 mmol) was added slowly to the suspension above at 10° C. The resulting solution was stirred at 10° C. for 1 h. Further ethyl iodide (139 μL, 1.74 mmol) was added and the suspension was stirred at 10° C. for 1 h. NaOH (56 mg, 1.4 mmol) was added slowly to the mixture above at 10° C. The resulting solution was stirred at 10° C. for 1 h. The reaction mixture was adjusted to pH ˜6 with 1 M HCl (aq), and filtered through Celite®, The filtrate was purified directly by preparative HPLC PrepMethod D (gradient 10-35%) to give the title compound (130 mg, 62%) as a white solid; MS m/z (ESI), [M+H]⁺ 302.1.

### Intermediate 61: Methyl 6-(morpholinomethyl)quinoline-4-carboxylate

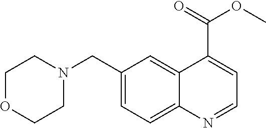 [SMILES: COC(=O)c1ccnc2ccc(CN3CCOCC3)cc12]

Morpholine (222 μL, 2.55 mmol) was added to methyl 6-(chloromethyl)quinoline-4-carboxylate Intermediate 9 (200 mg, 0.85 mmol), TEA (591 μL, 4.24 mmol) and KI (282 mg, 1.70 mmol) in MeCN (18 mL) at 20° C. The resulting mixture was stirred at 20° C. for 15 h. The reaction mixture was diluted with DCM (100 mL) and washed sequentially with sat NaHCO₃ (aq, 3×100 mL). The organic layer was dried over Na₂SO₄, filtered and evaporated. The residue was purified by preparative TLC (EtOAc:petroleum ether, 1:1) to give the title compound (0.15 g, 61%) as a pale yellow oil; MS m/z (ESI), [M+H]⁺ 287.0.

### Intermediate 62: 6-(Morpholinomethyl)quinoline-4-carboxylic Acid

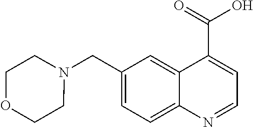 [SMILES: O=C(O)c1ccnc2ccc(CN3CCOCC3)cc12]

Methyl 6-(morpholinomethyl)quinoline-4-carboxylate Intermediate 61 (120 mg, 0.42 mmol) was added to LiOH (240 mg, 10.0 mmol) in water (6 mL) and MeOH (18 mL) at 20° C. The resulting mixture was stirred at 20° C. for 16 h. The reaction mixture was adjusted to pH 3 with 1 M HCl. The solution was filtered and dried over Na₂SO₄. The solvent was removed under reduced pressure to give the title compound (100 mg, 88%) as a pale yellow solid that was used directly in the next step without further purification; MS m/z (ESI), [M+H]⁺ 272.9.

### Intermediate 63: rac-Methyl (R)-6-(1-hydroxyethyl)quinoline-4-carboxylate

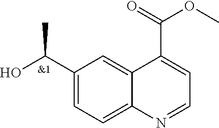 [SMILES: COC(=O)c1ccnc2ccc([C@H](C)O)cc12]

NaBH₄ (27 mg, 0.65 mmol) was added to a solution of methyl 6-acetylquinoline-4-carboxylate Intermediate 5 (300 mg, 1.31 mmol) in MeOH (5 mL), and the reaction mixture was stirred at 25° C. for 2 h. The reaction mixture was concentrated in vacuo and the crude product was purified by preparative TLC (EtOAc:pentane, 1:1) to give the title compound (200 mg, 66%) as a yellow solid; MS (ESI) m/z [M+H]⁺ 232.

### Intermediate 64: rac-(R)-6-(1-Hydroxyethyl)quinoline-4-carboxylic Acid

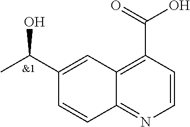 [SMILES: C[C@@H](O)c1ccc2nccc(C(=O)O)c2c1]

A solution rac-methyl (R)-6-(1-hydroxyethyl)quinoline-4-carboxylate Intermediate 63 (130 mg, 0.56 mmol) and LiOH (27 mg, 1.1 mmol) in THF (6 mL) and water (1 mL) was stirred at rt for 1 h. The solvent was removed under reduced pressure. The reaction mixture was acidified with 0.1 M HCl, diluted with EtOAc and washed with water. The organic layer was dried over Na₂SO₄, filtered and evaporated to give the title compound (85 mg, 69%) that was used directly in the next step without further purification; MS m/z (ESI), [M+H]⁺ 218.3.

### Intermediate 65: Methyl 6-(ethoxymethyl)quinoline-4-carboxylate

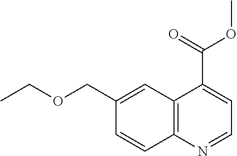 [SMILES: CCOCc1ccc2nccc(C(=O)OC)c2c1]

Cs₂CO₃ (918 mg, 2.82 mmol) was added to methyl 6-bromoquinoline-4-carboxylate (300 mg, 1.13 mmol), potassium (ethoxymethyl)trifluoroborate (374 mg, 2.25 mmol), Pd(OAc)₂ (25 mg, 0.11 mmol) in 1,4-dioxane (10 mL) and water (1 mL) at 25° C. The resulting suspension was stirred at 100° C. for 16 h under N₂ (g). The solvent was removed under reduced pressure. The residue was diluted with water (50 mL), extracted with EtOAc (3×50 mL), the organic layer was dried over Na₂SO₄, filtered and evaporated. The residue was purified by preparative TLC (EtOAc:petroleum ether 1:2), to afford the title compound (0.165 g, 60%) as a brown gum; MS (ESI) m/z [M+H]⁺ 246.

### Intermediate 66: 6-(Ethoxymethyl)quinoline-4-carboxylic Acid

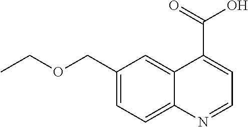 [SMILES: CCOCc1ccc2nccc(C(=O)O)c2c1]

NaOH (130 mg, 3.26 mmol) was added to methyl 6-(ethoxymethyl)quinoline-4-carboxylate Intermediate 65 (160 mg, 0.65 mmol) in MeOH (9 mL) and water (3 mL) at 25° C. The resulting solution was stirred at 25° C. for 1 h under N₂ (g). The solvent was removed under reduced pressure. The residue was diluted with water (50 mL) and adjusted to pH 3 using 1 M HCl (aq). The aqueous phase was extracted with EtOAc (3×50 mL), the organic layer was dried over Na₂SO₄, filtered and evaporated to afford the title compound (0.133 g, 88%) as a pale yellow solid; MS (ESI) m/z [M+H]⁺ 232.1.

### Intermediate 67: Methyl 6-(fluoromethyl)quinoline-4-carboxylate

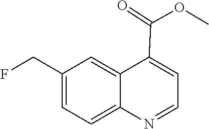 [SMILES: COC(=O)c1ccnc2ccc(CF)cc12]

DAST (0.365 mL, 2.76 mmol) was added to methyl 6-(hydroxymethyl)quinoline-4-carboxylate Intermediate 7 (300 mg, 1.38 mmol) in DCM (10 mL) at rt. The resulting mixture was stirred at rt for 2 h. The solvent was removed under reduced pressure and the residue was purified by preparative TLC (pentane:EtOAc, 2:1), to afford the title compound (200 mg, 66%) as a pale yellow solid; MS (ESI) m/z [M+H]⁺ 220.

### Intermediate 68: 6-(Fluoromethyl)quinoline-4-carboxylic Acid

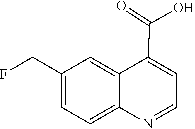 [SMILES: O=C(O)c1ccnc2ccc(CF)cc12]

A solution of methyl 6-(fluoromethyl)quinoline-4-carboxylate Intermediate 67 (160 mg, 0.73 mmol) and LiOH (35 mg, 1.5 mmol) in THF (5 mL) and water (1 mL) was stirred at rt for 1 h. The solvent was removed under reduced pressure. The reaction mixture was diluted with H₂O (5 mL) and acidified with 0.1 M HCl. The precipitate was collected by filtration and washed with water to provide the title compound (80 mg, 53%) as a white solid; MS (ESI) m/z [M+H]⁺ 206.

### Intermediate 69: Methyl 6-((2-methoxyethoxy)methyl)quinoline-4-carboxylate and

### Intermediate 70: rac-methyl (R)-6-(1,2-dimethoxyethyl)quinoline-4-carboxylate

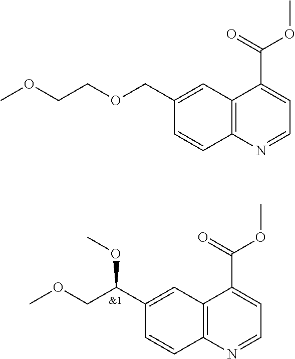

A mixture of methyl 6-bromoquinoline-4-carboxylate (133 mg, 0.5 mmol), NiBr₂·dtbbpy (12 mg, 0.03 mmol) and TBADT (17 mg, 5.0 μmol) in a 5 mL microwave vial was brought into a glove box. MeCN (5 mL) was added. The resulting solution was then added to a 5 mL microwave vial containing K₃PO₄ (117 mg, 0.55 mmol). 1,2-dimethoxyethane (0.26 mL, 2.5 mmol) was added to the resulting suspension and the vial was then capped and brought out from the glove box and placed in a photoreactor and irradiated at 365 nm for 18 h. The cap was removed and the mixture was stirred open to air for 15 min and then filtered and concentrated. The resulting residue was purified using preparative HPLC, PrepMethod E, (gradient 15-55%) to give a mixture of the title compounds (46 mg, 34%); MS (ESI) m/z [M+H]⁺ 276.1.

### Intermediate 71: 6-((2-Methoxyethoxy)methyl)quinoline-4-carboxylic Acid and

### Intermediate 72: rac-(R)-6-(1,2-dimethoxyethyl)quinoline-4-carboxylic Acid

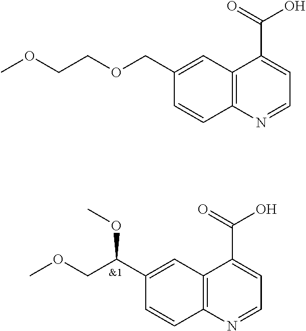 [SMILES: COCCOCc1ccc2nccc(C(=O)O)c2c1 ; COC[C@@H](OC)c1ccc2nccc(C(=O)O)c2c1]

Aq LiOH (574 μL, 0.57 mmol, 1 M) was added to a mixture of 6-((2-methoxyethoxy)-methyl)quinoline-4-carboxylic acid Intermediate 69 and rac-methyl (R)-6-(1,2-dimethoxyethyl)quinoline-4-carboxylate Intermediate 70 (79 mg, 0.29 mmol) in THF (2.3 mL). The reaction was stirred at rt overnight and then concentrated. The residue was purified by preparative HPLC, PrepMethod C, (gradient: 0-30%) to give a mixture of the title compounds Intermediate 71 and Intermediate 72 (60 mg, 80%); MS (ESI) m/z [M+H]⁺ 262.2.

### Intermediate 73: rac-Methyl (R)-6-(tetrahydro-2H-pyran-2-yl)quinoline-4-carboxylate

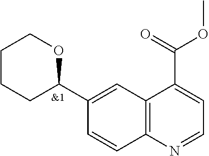 [SMILES: COC(=O)c1ccnc2ccc([C@H]3CCCCO3)cc12]

A solution of methyl 6-bromoquinoline-4-carboxylate (133 mg, 0.5 mmol), TBADT (17 mg, 5.0 μmol)) and NiBr₂·dtbbpy (12 mg, 0.03 mmol) in deoxygenated MeCN (5 mL) in a microwave vial (2-5 mL size) was added to a vial containing K₃PO₄ (117 mg, 0.55 mmol) (which had been crushed with a mortar and pestle and dried by heating with a heat gun under vacuum in a microwave vial) under N₂ (g). The resulting suspension was bubbled with N₂ (g) for 10 min with cooling at 0° C. Degassed tetrahydro-2H-pyran (0.25 mL, 2.5 mmol) was then added and the reaction vial was placed in a photoreactor and irradiated at 365 nm for 18 h. The reaction mixture was stirred open to air for 15 min and then filtered through a syringe filter. The filter was washed several times with EtOAc. The resulting solution was concentrated to and the residue was purified by straight phase flash chromatography on silica (gradient: 25-50% of EtOAc in heptane) to give the title compound (20 mg, 15%) as a thin film;

MS (ESI) m/z [M+H]⁺ 272.1.

### Intermediate 74: rac-Methyl (R)-6-(1,4-dioxan-2-yl)quinoline-4-carboxylate

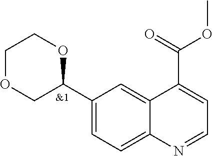 [SMILES: COC(=O)c1ccnc2ccc([C@H]3COCCO3)cc12]

A solution of methyl 6-bromoquinoline-4-carboxylate (133 mg, 0.5 mmol), TBADT (17 mg, 5.0 μmol)) and NiBr₂·dtbbpy (12 mg, 0.03 mmol) in deoxygenated MeCN (5 mL) was added under N₂ (g) to a vial containing K₃PO₄ (117 mg, 0.55 mmol) which had been crushed with a mortar and pestle and dried by heating with a heat gun under vacuum. The resulting suspension was bubbled with N₂ (g) for 10 min with cooling at 0° C. Degassed 1,4-dioxane (0.21 mL, 2.5 mmol) was then added and the reaction vial was placed in a photoreactor and irradiated at 365 nm for 18 h. The reaction mixture was stirred open to air for 15 min, filtered and the filter was washed several times with EtOAc. The combined filtrates were concentrated and the residue was purified by flash chromatography using a gradient of 25-50% EtOAc in heptane to give the title compound (45 mg, 33%) as a yellow film;

MS (ESI) m/z [M+H]⁺ 274.0.

### Intermediate 75: rac-Methyl (R)-6-(tetrahydrofuran-2-yl)quinoline-4-carboxylate

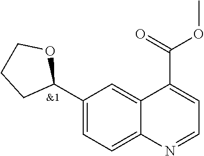 [SMILES: COC(=O)c1ccnc2ccc([C@H]3CCCO3)cc12]

A solution of methyl 6-bromoquinoline-4-carboxylate (106 mg, 0.4 mmol), TBADT (13 mg, 4.0 μmol)) and NiBr₂·dtbbpy (10 mg, 0.02 mmol) in deoxygenated MeCN (4 mL) was added to a vial containing K₃PO₄ (93 mg, 0.44 mmol) under N₂ (g). The resulting suspension was bubbled with N₂ (g) for 10 min with cooling at 0° C. THF (0.16 mL, 2.0 mmol) was then added and the reaction vial was placed in a photoreactor and irradiated at 365 nm for 18 h. The reaction mixture was then filtered through a syringe filter and the filter was washed several times with EtOAc. The combined filtrates were concentrated and the residue was purified by preparative HPLC, PrepMethod E, (gradient: 15-55%) to give the title compound (26 mg, 25%) as a yellow film; MS (ESI) m/z [M+H]⁺ 258.1.

### Intermediate 76: rac-(R)-6-(Tetrahydrofuran-2-yl)quinoline-4-carboxylic Acid

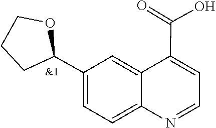 [SMILES: O=C(O)c1ccnc2ccc([C@H]3CCCO3)cc12]

Aq LiOH (505 μL, 0.51 mmol, 1 M) was added to a solution rac-methyl (R)-6-(tetrahydrofuran-2-yl)quinoline-4-carboxylate Intermediate 75 (65 mg, 0.25 mmol) in THF (2 mL). The mixture was stirred at rt for 2 h and was then concentrated. The residue was purified by preparative HPLC, PrepMethod C, (gradient: 0-30%) to give the title compound (43 mg, 70%) as a white solid; MS (ESI) m/z [M+H]⁺ 244.0.

### Intermediate 77: rac-(R)-6-(((Tetrahydrofuran-3-yl)oxy)methyl)quinoline-4-carboxylic Acid

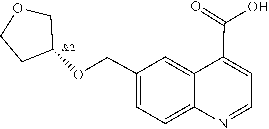 [SMILES: O=C(O)c1ccnc2ccc(CO[C@@H]3CCOC3)cc12]

A solution of NaH (38 mg, 0.95 mmol; 60% wt in oil) and tetrahydrofuran-3-ol (84 mg, 0.95 mmol) in DMF (5 mL) was stirred at 0° C. for 10 min. Then methyl 6-(chloromethyl)-quinoline-4-carboxylate Intermediate 9 (75 mg, 0.32 mmol) was added. The reaction was stirred at 0° C. for 1 h. Water (1 mL) was added and the reaction was stirred at 0° C. for 30 min. The solvent was removed under reduced pressure. The residue was diluted with water. The mixture was adjusted to pH 6 with 0.1 M HCl and diluted with EtOAc (20 mL), and washed with water (3×10 mL). The organic layer was dried over Na₂SO₄, filtered and concentrated to afford the title compound (70 mg, 80%); MS (ESI) m/z [M+H]⁺ 274.

### Intermediate 78: 6-((2,2-Difluoroethoxy)methyl)quinoline-4-carboxylic Acid

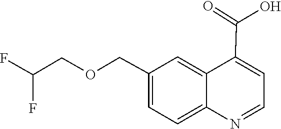 [SMILES: O=C(O)c1ccnc2ccc(COCC(F)F)cc12]

2,2-Difluoroethan-1-ol (89 mg, 1.1 mmol) and methyl 6-(chloromethyl)quinoline-4-carboxylate Intermediate 9 (85 mg, 0.36 mmol) were treated in a similar manner as described for compound Intermediate 77 to give the title compound (90 mg, 93%);

MS (ESI) m/z [M+H]⁺ 268.

### Intermediate 79: 6-((4-Methylisoxazol-3-yl)methyl)quinoline-4-carboxylic Acid

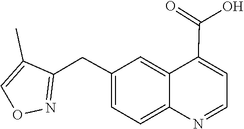 [SMILES: Cc1conc1Cc1ccc2nccc(C(=O)O)c2c1]

In a microwave vial was placed 6-bromoquinoline-4-carboxylic acid (83 mg, 0.33 mmol), 4,4,4′,4′,5,5,5′,5′-octamethyl-2,2′-bi(1,3,2-dioxaborolane) (92 mg, 0.36 mmol), KOAc (97 mg, 0.99 mmol), Pd(dppf)Cl₂·DCM (40 mg, 0.05 mmol) and 1,4-dioxane (2 mL). The vial was capped, evacuated and back-filled with N₂ (g) three times. The vial was then heated at 120° C. for 30 min in a microwave reactor. The vial was cooled to rt and 3-(bromomethyl)-4-methylisoxazole (58 mg, 0.33 mmol), Pd(dtbpf)Cl₂ (32 mg, 0.05 mmol) and aq K₂CO₃ (659 μL, 1.32 mmol, 2 M) were added. The reaction mixture was stirred under N₂ (g) at rt overnight. The mixture was partitioned between water and EtOAc and the pH of the aqueous phase was adjusted to weakly acidic by 2 M HCl (aq). The organic phase was recovered, and the aqueous phase was extracted once more with EtOAc. The combined organic phase was evaporated and the compound was purified by preparative HPLC, PrepMethod C, (gradient: 5-40%) to give the title compound (17 mg, 19%); MS (ESI) m/z [M+H]⁺ 269.

### Intermediate 80: Ethyl 6-((4,6-dimethylpyridin-3-yl)methyl)quinoline-4-carboxylate

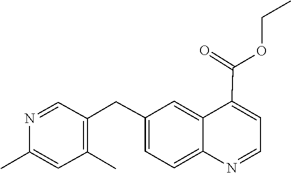 [SMILES: CCOC(=O)c1ccnc2ccc(Cc3cnc(C)cc3C)cc12]

A vial was charged with ethyl 6-(4,4,5,5-tetramethyl-1,3,2-dioxaborolan-2-yl)quinoline-4-carboxylate Intermediate 53 (200 mg, 0.61 mmol), 5-(bromomethyl)-2,4-dimethylpyridine (135 mg, 0.67 mmol), Na₂CO₃ (259 mg, 2.45 mmol) and trans-bromo(N-succinimidyl)-bis(triphenylphosphine)palladium(II) (25 mg, 0.03 mmol). Toluene (5 mL) and water (1 mL) were added, and the mixture was bubbled with N₂ (g) for 10 min. The vial was capped and heated at 100° C. for 2 h. The reaction mixture was partitioned between DCM and water and the layers were separated using a phase-separator. The organic layer was concentrated and the residue was purified by straight phase flash chromatography on silica (gradient: 20-100% of EtOAc in heptane) to give the title compound (50 mg, 26%); MS (ESI) m/z [M+H]⁺ 321.4.

### Intermediate 81: Ethyl 6-((1,3-dimethyl-1H-pyrazol-5-yl)methyl)quinoline-4-carboxylate

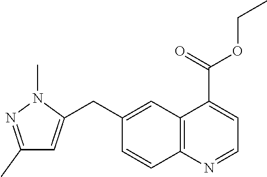 [SMILES: CCOC(=O)c1ccnc2ccc(Cc3cc(C)nn3C)cc12]

The title compound was prepared in a similar manner as described for Intermediate 80 using ethyl 6-(4,4,5,5-tetramethyl-1,3,2-dioxaborolan-2-yl)quinoline-4-carboxylate Intermediate 53 (190 mg, 0.58 mmol) and 5-(bromomethyl)-1,3-dimethyl-1H-pyrazole (121 mg, 0.64 mmol) to give the title compound (80 mg, 45%); MS (ESI) m/z [M+H]⁺ 310.3.

### Intermediate 82: Ethyl 6-((2-methyl-2H-1,2,3-triazol-4-yl)methyl)quinoline-4-carboxylate

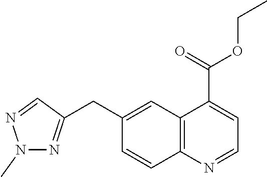 [SMILES: CCOC(=O)c1ccnc2ccc(Cc3cnn(C)n3)cc12]

The title compound was prepared in a similar manner as described for Intermediate 80 using ethyl 6-(4,4,5,5-tetramethyl-1,3,2-dioxaborolan-2-yl)quinoline-4-carboxylate Intermediate 53 (190 mg, 0.58 mmol) and 4-(bromomethyl)-2-methyl-2H-1,2,3-triazole (112 mg, 0.64 mmol) to give the title compound (83 mg, 48%); MS (ESI) m/z [M+H]⁺ 297.2.

### Intermediate 83: Ethyl 6-((5-methylisoxazol-3-yl)methyl)quinoline-4-carboxylate

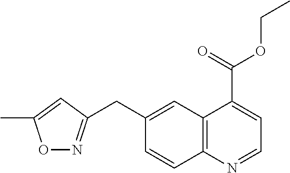 [SMILES: CCOC(=O)c1ccnc2ccc(Cc3cc(C)on3)cc12]

The title compound was prepared in a similar manner as described for Intermediate 80 using ethyl 6-(4,4,5,5-tetramethyl-1,3,2-dioxaborolan-2-yl)quinoline-4-carboxylate Intermediate 53 (95 mg, 0.29 mmol) and 3-(bromomethyl)-5-methylisoxazole (51 mg, 0.29 mmol) with additional 3-(bromomethyl)-5-methylisoxazole (51 mg, 0.29 mmol) and trans-bromo(N-succinimidyl)bis(triphenylphosphine)palladium(II) (12 mg, 0.01 mmol) added after 2 h and a total reaction time of 3 h, to give the title compound (43 mg, 50%); MS (ESI) m/z [M+H]⁺ 297.3.

### Intermediate 84: 2-Methyl-5-(1-(4,4,5,5-tetramethyl-1,3,2-dioxaborolan-2-yl)cyclopropyl)pyridine

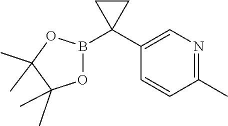

A vial was charged with 5-bromo-2-methylpyridine (293 mg, 1.70 mmol), 2,2′-(cyclopropane-1,1-diyl)bis(4,4,5,5-tetramethyl-1,3,2-dioxaborolane) (250 mg, 0.85 mmol), cataCXium® A Pd G3 (31 mg, 0.04 mmol) and Cs₂CO₃ (831 mg, 2.55 mmol). 1,4-Dioxane (15 mL) and water (1.5 mL) were added, and the mixture was degassed by bubbling N₂ (g) for 10 min. The mixture was stirred at 100° C. overnight. The reaction mixture was partitioned between DCM and water and the layers were separated using a phase separator. The organic layer was concentrated and the residue was purified by straight phase flash chromatography on silica (gradient: 10-50% of EtOAc in heptane) to give the title compound (31 mg, 14%) as a solid; MS (ESI) m/z [M+H]⁺ 260.1.

### Intermediate 85: Potassium trifluoro(1-(6-methylpyridin-3-yl)cyclopropyl)-borate

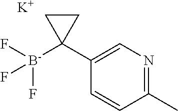

KHF₂ (65 mg, 0.84 mmol) was added to a solution of 2-methyl-5-(1-(4,4,5,5-tetramethyl-1,3,2-dioxaborolan-2-yl)cyclopropyl)pyridine Intermediate 84 (31 mg, 0.12 mmol) in MeOH (1.5 mL) and the resulting mixture was heated at reflux for 3.5 h. After cooling to rt the mixture was concentrated and the residue was triturated with 20% diethyl ether in heptane (2 mL) for 15 min, then with ether (2×2 mL). The remaining solids were stirred briefly in hot MeCN (5 mL) during which the product dissolved. The mixture was filtered, and the solids were washed with 2 mL of hot MeCN. The combined filtrates were concentrated to give the title compound (25 mg, 87%) as a white semi solid; MS (ESI) m/z [M−K]⁻ 200.1.

### Intermediate 86: Ethyl 6-(1-(6-methylpyridin-3-yl)cyclopropyl)quinoline-4-carboxylate

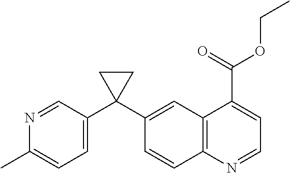 [SMILES: CCOC(=O)c1ccnc2ccc(C3(c4ccc(C)nc4)CC3)cc12]

A mixture of ethyl 6-bromoquinoline-4-carboxylate (56 mg, 0.20 mmol), potassium trifluoro(1-(6-methylpyridin-3-yl)cyclopropyl)borate Intermediate 85 (25 mg, 0.10 mmol), cataCXium® A Pd G3 (4 mg, 5 μmol) and Cs₂CO₃ (102 mg, 0.31 mmol) in a mixture of toluene (3 mL) and water (0.3 mL) was degassed by bubbling N₂ (g) for 10 min and the mixture was heated at 95° C. for 17 h. After cooling to rt, the reaction mixture was partitioned between DCM and water and the layers were separated using a phase separator. The organic layer was concentrated. The residue was purified by straight phase flash chromatography on silica (gradient: 20-100% of EtOAc in heptane) to give the title compound (8 mg, 23%); MS (ESI) m/z [M+H]⁺ 333.3.

### Intermediate 87: Methyl 6-(2-methoxypropan-2-yl)quinoline-4-carboxylate

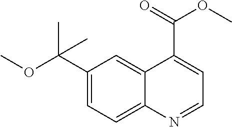

NaH (60% in oil, 24 mg, 0.61 mmol) was added to a solution of methyl 6-(2-hydroxypropan-2-yl)quinoline-4-carboxylate Intermediate 6 (100 mg, 0.41 mmol) in DMF (1 mL), and the reaction mixture was stirred at 20° C. for 30 min. CH₃I (51 μL, 0.82 mmol) was added to the reaction mixture and it was stirred at rt for 16 h. An additional batch was prepared as described above using (70 mg, 0.29 mmol) of Intermediate 6. The reaction mixtures from both batches were combined and the reaction was quenched with water (10 mL) and extracted with EtOAc (3×10 mL). The combined organic layer was dried over Na₂SO₄, filtered and evaporated. The residue was purified by preparative TLC (DCM:MeOH, 10:1), to give the title compound (70 mg, 48%) as a brown gum; MS (ESI) m/z [M+H]⁺ 260.1.

### Intermediate 88: 4-Chloro-6-(1-(3-fluoropyridin-2-yl)cyclopropyl)quinoline

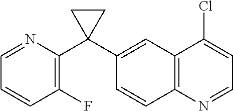 [SMILES: Fc1cccnc1C1(c2ccc3nccc(Cl)c3c2)CC1]

CataCXium® A Pd G3 (24 mg, 0.03 mmol) was added to potassium (1-(4-chloroquinolin-6-yl)cyclopropyl)trifluoroborate Intermediate 43 (205 mg, 0.66 mmol), 2-bromo-3-fluoropyridine (140 mg, 0.79 mmol) and Cs₂CO₃ (432 mg, 1.32 mmol) in 1,4-dioxane (10 mL) and water (1 mL) at 20° C. The resulting solution was stirred at 100° C. for 15 h under N₂ (g). The reaction mixture was diluted with EtOAc, and the organic layer was dried over Na₂SO₄, filtered and evaporated to afford yellow gum. The residue was purified by preparative TLC (EtOAc:petroleum ether 1:2), to give the title compound (59 mg, 30%) as a pale yellow gum; MS (ESI) m/z [M+H]⁺ 299.

### Intermediate 89: Methyl 6-(1-(3-fluoropyridin-2-yl)cyclopropyl)quinoline-4-carboxylate

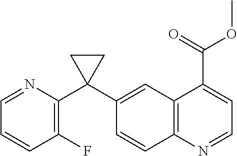 [SMILES: COC(=O)c1ccnc2ccc(C3(c4ncccc4F)CC3)cc12]

Pd(OAc)₂ (6.5 mg, 0.03 mmol) and Pd(dppf)Cl₂ (21 mg, 0.03 mmol) were added to a mixture of 4-chloro-6-(1-(3-fluoropyridin-2-yl)cyclopropyl)quinoline Intermediate 88 (43 mg, 0.14 mmol), TEA (40 μL, 0.29 mmol) and dppf (16 mg, 0.03 mmol) in MeOH (5 mL) under N₂ (g). The resulting mixture was stirred at 100° C. for 12 h under 20 atm CO (g). The solvent was removed under reduced pressure. The residue was diluted with water (50 mL) and extracted with EtOAc (3×50 mL). The organic layer was dried over Na₂SO₄, filtered and concentrated. The residue was purified by preparative TLC (EtOAc:petroleum ether 1:1) to give the title compound (0.040 g, 86%) as a brown gum; MS (ESI) m/z [M+H]⁺ 349.

### Intermediate 90: 6-(1-(3-Fluoropyridin-2-yl)cyclopropyl)quinoline-4-carboxylic Acid

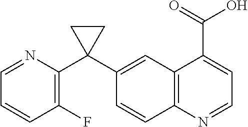 [SMILES: O=C(O)c1ccnc2ccc(C3(c4ncccc4F)CC3)cc12]

NaOH (33 mg, 0.82 mmol) was added to methyl 6-(1-(3-fluoropyridin-2-yl)cyclopropyl)-quinoline-4-carboxylate Intermediate 89 (53 mg, 0.16 mmol) in MeOH (6 mL) and water (1 mL) at 15° C. The resulting solution was stirred at 15° C. for 1 h. The solvent was removed under reduced pressure. The residue was diluted with water (10 mL), adjusted to pH 3 using 1 M HCl (aq) and extracted with EtOAc (3×50 mL). The combined organic layer was dried over Na₂SO₄, filtered and concentrated to give the title compound (72 mg) as an orange solid which was used in the next step directly without further purification;

MS (ESI) m/z [M+H]⁺ 309.

### Intermediate 91: tert-Butyl 6-(1-(5-methylisoxazol-3-yl)vinyl)quinoline-4-carboxylate

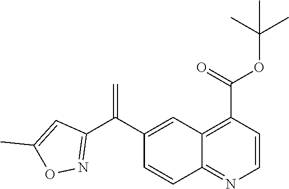 [SMILES: C=C(c1ccc2nccc(C(=O)OC(C)(C)C)c2c1)c1cc(C)on1]

A solution of 1-(5-methylisoxazol-3-yl)ethan-1-one (200 mg, 1.60 mmol) and 4-methylbenzenesulfonohydrazide (435 mg, 2.34 mmol) in 1,4-dioxane (20 mL) was stirred at 80° C. for 1 h. tert-Butyl 6-bromoquinoline-4-carboxylate (WO2019154886) (400 mg, 1.30 mmol), XantPhos (113 mg, 0.19 mmol), LiOtBu (312 mg, 3.89 mmol) and Pd₂(dba)₃ (59 mg, 0.06 mmol) were added to the reaction mixture. The mixture was heated at 100° C. with stirring for 1 h. The reaction mixture was diluted with EtOAc (30 mL) and washed with water (3×10 mL). The organic layer was dried over Na₂SO₄, filtered and concentrated. The residue was purified by preparative TLC (petroleum ether:EtOAc 3:1), to give the title compound (0.18 g, 41%) as a pale yellow solid; MS (ESI) m/z [M+H]⁺ 339.

### Intermediate 92: tert-Butyl 6-(1-(5-methylisoxazol-3-yl)cyclopropyl)quinoline-4-carboxylate

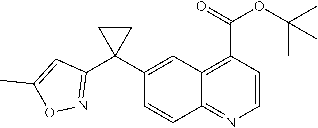 [SMILES: Cc1cc(C2(c3ccc4nccc(C(=O)OC(C)(C)C)c4c3)CC2)no1]

A solution of potassium 2-methylpropan-2-olate (133 mg, 1.19 mmol) and trimethylsulfoxonium iodide (262 mg, 1.19 mmol) in MeCN (10 mL) was stirred at 60° C. for 1 h. tert-Butyl 6-(1-(5-methylisoxazol-3-yl)vinyl)quinoline-4-carboxylate Intermediate 91 (200 mg, 0.59 mmol) was added and the reaction was stirred at 30° C. for 10 h. The reaction mixture was diluted with EtOAc (30 mL) and washed with water (3×15 mL). The organic layer was dried over Na₂SO₄, filtered and evaporated. The residue was purified by preparative TLC (petroleum ether:EtOAc, 3:1), to give the title compound (0.15 g, 72%) as a pale yellow solid; MS (ESI) m/z [M+H]⁺ 351.

### Intermediate 93: rac-tert-Butyl (R)-6-(1-(5-methylisoxazol-3-yl)ethyl)quinoline-4-carboxylate

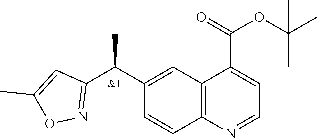 [SMILES: Cc1cc([C@@H](C)c2ccc3nccc(C(=O)OC(C)(C)C)c3c2)no1]

A mixture of tert-butyl 6-(1-(5-methylisoxazol-3-yl)vinyl)quinoline-4-carboxylate Intermediate 91 (90 mg, 0.27 mmol) and Lindlar catalyst (57 mg, 0.03 mmol) in EtOAc (6 mL) was stirred under an atmosphere of H₂ (g) at 2 atm and 30° C. for 5 h. The mixture was filtered, and the filtrate was concentrated under vacuum. The residue was purified by preparative TLC (petroleum ether:EtOAc 3:1) to give the title compound (40 mg, 44%) as a pale yellow solid;

MS (ESI) m/z [M+H]⁺ 339.

### Intermediate 94: rac-(R)-6-(1-(5-methylisoxazol-3-yl)ethyl)quinoline-4-carboxylic Acid

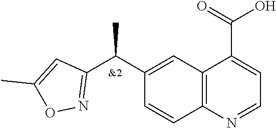 [SMILES: Cc1cc([C@@H](C)c2ccc3nccc(C(=O)O)c3c2)no1]

The title compound was prepared in a similar manner as described for Example 45, step A starting from rac-tert-butyl (R)-6-(1-(5-methylisoxazol-3-yl)ethyl)quinoline-4-carboxylate Intermediate 93 (140 mg, 0.41 mmol) to give the title compound (0.21 g, 100%) as a pale yellow gum that was used in the next step directly without further purification;

MS (ESI) m/z [M+H]⁺ 283.

### Intermediate 95: Methyl 6-(1-(4,4,5,5-tetramethyl-1,3,2-dioxaborolan-2-yl)cyclopropyl)quinoline-4-carboxylate

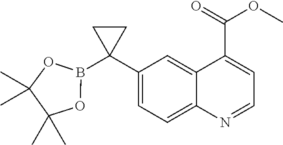

The title compound was prepared in a similar manner as described for Intermediate 42 starting from 2,2′-(cyclopropane-1,1-diyl)bis(4,4,5,5-tetramethyl-1,3,2-dioxaborolane) (3.00 g, 10.2 mmol) and methyl 6-bromoquinoline-4-carboxylate (2.85 g, 10.7 mmol). Purification by straight phase flash chromatography on silica (gradient: 2-30% EtOAc in petroleum ether) to give the title compound (1.3 g, 36%) as a pale yellow gum;

MS (ESI) m/z [M+H]⁺ 354.3.

### Intermediate 96: Potassium trifluoro(1-(4-(methoxycarbonyl)quinolin-6-yl)cyclopropyl)borate

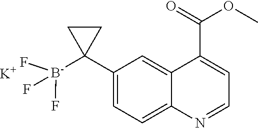 [SMILES: [K+] ; COC(=O)c1ccnc2ccc(C3([B-](F)(F)F)CC3)cc12]

Methyl 6-(1-(4,4,5,5-tetramethyl-1,3,2-dioxaborolan-2-yl)cyclopropyl)quinoline-4-carboxylate Intermediate 95 (1.3 g, 3.7 mmol) was reacted in a similar manner as described in the preparation of Intermediate 43 to give the title compound (0.60 g, 49%) as a white solid; MS (ESI) m/z [M−K]⁻ 294.0.

### Intermediate 97: Methyl 6-(1-(5-chloropyridin-2-yl)cyclopropyl)quinoline-4-carboxylate

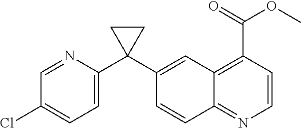 [SMILES: COC(=O)c1ccnc2ccc(C3(c4ccc(Cl)cn4)CC3)cc12]

CataCXium A Pd G3 (68 mg, 0.09 mmol) was added to 5-chloro-2-iodopyridine (334 mg, 1.40 mmol), potassium trifluoro(1-(4-(methoxycarbonyl)quinolin-6-yl)cyclopropyl)borate Intermediate 96 (310 mg, 0.93 mmol), butyldi(1-adamantyl)phosphine (34 mg, 0.09 mmol) and Cs₂CO₃ (606 mg, 1.86 mmol) in 1,4-dioxane (5 mL) and water (1 mL) at 20° C. The resulting solution was stirred at 80° C. for 15 h under N₂ (g). The solvent was removed under reduced pressure. The reaction mixture was diluted with DCM and washed with water (2×10 mL). The organic layer was dried over Na₂SO₄, filtered and concentrated. The residue was purified by preparative HPLC, PrepMethod O (gradient 47-57%), to give the title compound (11 mg, 3.5%) as a white solid; MS (ESI) m/z [M+H]⁺ 339.

### Intermediate 98: tert-Butyl 6-(1-(5-chloropyridin-2-yl)vinyl)quinoline-4-carboxylate

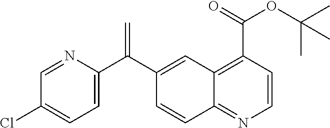 [SMILES: C=C(c1ccc2nccc(C(=O)OC(C)(C)C)c2c1)c1ccc(Cl)cn1]

A solution of 1-(5-chloropyridin-2-yl)ethan-1-one (150 mg, 0.96 mmol) and 4-methylbenzenesulfonohydrazide (269 mg, 1.45 mmol) in 1,4-dioxane (8 mL) was stirred at 80° C. for 1 h. Then tert-butyl 6-bromoquinoline-4-carboxylate (WO 2019154886) (297 mg, 0.96 mmol), Pd₂(dba)₃ (88 mg, 0.10 mmol), XPhos (138 mg, 0.29 mmol) and LiOtBu (232 mg, 2.89 mmol) was added under N₂ (g). The reaction was stirred at 110° C. for 2 h. The reaction mixture was diluted with EtOAc and washed with water. The organic layer was dried over Na₂SO₄, filtered and concentrated. The residue was purified by preparative TLC (petroleum ether:EtOAc 2:1), to afford the title compound (0.15 g, 42%) as a pale yellow solid;

MS (ESI) m/z [M+H]⁺ 367.

### Intermediate 99: rac-tert-Butyl (R)-6-(1-(5-chloropyridin-2-yl)ethyl)quinoline-4-carboxylate

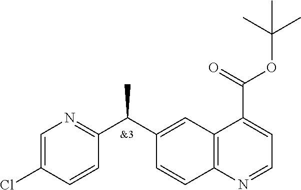 [SMILES: C[C@@H](c1ccc2nccc(C(=O)OC(C)(C)C)c2c1)c1ccc(Cl)cn1]

The title compound was prepared in a similar manner as described for Intermediate 93 using tert-butyl 6-(1-(5-chloropyridin-2-yl)vinyl)quinoline-4-carboxylate Intermediate 98 (150 mg, 0.41 mmol) to give the title compound (0.11 g, 73%) as a pale yellow gum; MS (ESI) m/z [M+H]⁺ 369.

### Intermediate 100: rac-(R)-6-(1-(5-chloropyridin-2-yl)ethyl)quinoline-4-carboxylic Acid

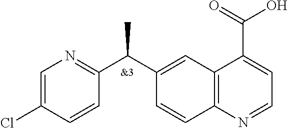 [SMILES: C[C@@H](c1ccc2nccc(C(=O)O)c2c1)c1ccc(Cl)cn1]

The title compound was prepared in a similar manner as described for Example 45, step A starting from tert-butyl 6-(1-(5-chloropyridin-2-yl)ethyl)quinoline-4-carboxylate Intermediate 99 (150 mg, 0.41 mmol) to give the title compound (0.205 g, 100%) as a pale yellow gum; MS (ESI) m/z [M+H]⁺ 313.

### Intermediate 101: (Z)-4-Methyl-N′-(1-(3-methylisoxazol-5-yl)ethylidene)-benzenesulfonohydrazide

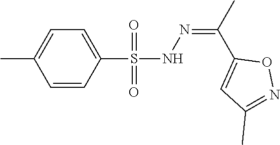 [SMILES: C/C(=N/NS(=O)(=O)c1ccc(C)cc1)c1cc(C)no1]

4-Methylbenzenesulfonohydrazide (3.44 g, 18.5 mmol) was added to 1-(3-methylisoxazol-5-yl)ethan-1-one (2.1 g, 17 mmol) in DCE (60 mL) at rt. The resulting mixture was stirred at 80° C. for 16 h. The solvent was removed under reduced pressure and the residue was purified by straight phase flash chromatography on silica (gradient: 20-50% EtOAc in petroleum ether) to give the title compound (4.6 g, 93%) as a white solid;

MS (ESI) m/z [M+H]⁺ 294.

### Intermediate 102: tert-Butyl 6-(1-(3-methylisoxazol-5-yl)vinyl)quinoline-4-carboxylate

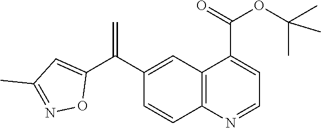 [SMILES: C=C(c1ccc2nccc(C(=O)OC(C)(C)C)c2c1)c1cc(C)no1]

Pd₂(dba)₃ (15 mg, 0.02 mmol), XPhos (15 mg, 0.03 mmol) and LiOtBu (286 mg, 3.57 mmol) were added to a solution of tert-butyl 6-bromoquinoline-4-carboxylate (WO 2019154886) (500 mg, 1.62 mmol) and (Z)-4-methyl-N-(1-(3-methylisoxazol-5-yl)ethylidene)-benzenesulfonohydrazide Intermediate 101 (476 mg, 1.62 mmol) in 1,4-dioxane (20 mL). The resulting solution was stirred at 70° C. for 4 h under N₂ (g). The solvent was removed under reduced pressure. The residue was purified by preparative TLC (petroleum ether:EtOAc 2:1), to afford the title compound (0.386 g, 71%) as a pale yellow solid; MS (ESI) m/z [M+H]⁺ 337.2.

### Intermediate 103: 6-(1-(3-methylisoxazol-5-yl)vinyl)quinoline-4-carboxylic Acid

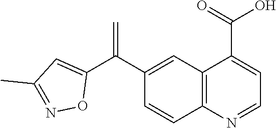 [SMILES: C=C(c1ccc2nccc(C(=O)O)c2c1)c1cc(C)no1]

Pd₂(dba)₃ (21 mg, 0.02 mmol), XPhos (22 mg, 0.05 mmol) and LiOtBu (397 mg, 4.96 mmol) were added to a solution of methyl 6-bromoquinoline-4-carboxylate (600 mg, 2.25 mmol) and (Z)-4-methyl-N-(1-(3-methylisoxazol-5-yl)ethylidene)benzenesulfonohydrazide Intermediate 101 (661 mg, 2.25 mmol) in 1,4-dioxane (20 mL). The resulting solution was stirred at 70° C. for 24 h under N₂ (g). The solvent was removed under reduced pressure. The residue was purified by preparative HPLC (petroleum ether:EtOAc 2:1), to afford the title compound (0.386 g, 71%) as a brown solid; MS (ESI) m/z [M+H]⁺ 281.0.

### Intermediate 104: rac-(R)-6-(1-(3-Methylisoxazol-5-yl)ethyl)quinoline-4-carboxylic Acid

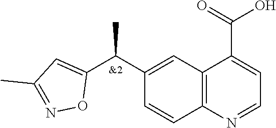 [SMILES: Cc1cc([C@@H](C)c2ccc3nccc(C(=O)O)c3c2)on1]

6-(1-(3-Methylisoxazol-5-yl)vinyl)quinoline-4-carboxylic acid Intermediate 103 (300 mg, 1.07 mmol) and 10% Pd/C (2.33 g, 2.14 mmol) in THF (30 mL) was stirred under an atmosphere of H₂ (g) at 25° C. for 12 h. The reaction mixture was filtered through Celite® and the filtrate was concentrated to give the title compound (0.30 g, 99%) as a yellow solid which was used in the next step directly without further purification; MS (ESI) m/z [M+H]⁺ 283.

### Intermediate 105: tert-Butyl 6-(1-(3-methylisoxazol-5-yl)cyclopropyl)quinoline-4-carboxylate

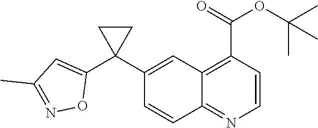 [SMILES: Cc1cc(C2(c3ccc4nccc(C(=O)OC(C)(C)C)c4c3)CC2)on1]

Dimethylmethanesulfinic iodide (343 mg, 1.56 mmol) was added to tert-butyl 6-(1-(3-methylisoxazol-5-yl)vinyl)quinoline-4-carboxylate Intermediate 102 (350 mg, 1.04 mmol) and KOtBu (175 mg, 1.56 mmol) in MeCN (5 mL) at 25° C. The resulting suspension was stirred at 25° C. for 12 h. The solvent was removed under reduced pressure and the residue was purified by preparative TLC (petroleum ether:EtOAc 2:1) to give the title compound (0.287 g, 79%) as an oil which solidified on standing; MS (ESI) m/z [M+H]⁺ 351.2.

### Intermediate 106: rac-Methyl (R)-6-(1-(6-methylpyridin-3-yl)ethyl)quinoline-4-carboxylate

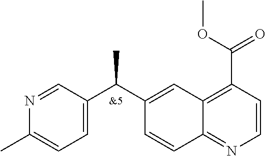 [SMILES: COC(=O)c1ccnc2ccc([C@H](C)c3ccc(C)nc3)cc12]

A solution of methyl 6-acetylquinoline-4-carboxylate Intermediate 5 (200 mg, 0.87 mmol) and 4-methylbenzenesulfonohydrazide (171 mg, 0.92 mmol) in 1,4-dioxane (10 mL) was stirred at 80° C. for 1 h. (6-Methylpyridin-3-yl)boronic acid (179 mg, 1.31 mmol) and Na₂CO₃ (139 mg, 1.31 mmol) were added to the reaction mixture. The system was refluxed at 100° C. with stirring for 12 h. The reaction mixture was diluted with EtOAc (30 mL) and washed with water (3×15 mL). The organic layer was dried over Na₂SO₄, filtered and concentrated. The residue was purified by preparative TLC (petroleum ether:EtOAc 1:1), to give the title compound (0.13 g, 49%) as a white solid; MS (ESI) m/z [M+H]⁺ 307.

### Intermediate 107: rac-(R)-6-(1-(6-Methylpyridin-3-yl)ethyl)quinoline-4-carboxylic Acid

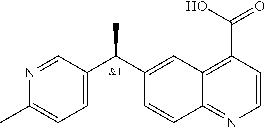 [SMILES: Cc1ccc([C@@H](C)c2ccc3nccc(C(=O)O)c3c2)cn1]

A solution of rac-methyl (R)-6-(1-(6-methylpyridin-3-yl)ethyl)quinoline-4-carboxylate Intermediate 106 (120 mg, 0.39 mmol) and LiOH (28 mg, 1.2 mmol) in MeOH (5 mL) and water (1 mL) was stirred at 30° C. for 2 h. The solvent was removed under reduced pressure. The residue was diluted with water (10 mL), and adjusted to pH 6 using 1 M HCl(aq). The mixture was diluted with EtOAc (30 mL) and the organic phase was extracted with water (3×10 mL). The aqueous layers were concentrated to give the title compound (0.10 g, 87%) as a pale yellow solid which was used in the next step directly without further purification;

MS (ESI) m/z [M+H]⁺ 293.

### Intermediate 108: rac-Methyl (R)-6-(1-(2-(trifluoromethyl)pyridin-4-yl)ethyl)quinoline-4-carboxylate

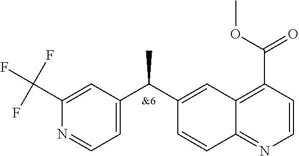 [SMILES: COC(=O)c1ccnc2ccc([C@H](C)c3ccnc(C(F)(F)F)c3)cc12]

A solution of methyl 6-acetylquinoline-4-carboxylate Intermediate 5 (200 mg, 0.87 mmol) and 4-methylbenzenesulfonohydrazide (171 mg, 0.92 mmol) in 1,4-dioxane (10 mL) was stirred at 80° C. for 1 h. (2-(Trifluoromethyl)pyridin-4-yl)boronic acid (250 mg, 1.31 mmol) and Na₂CO₃ (139 mg, 1.31 mmol) was added to the reaction mixture. The system was refluxed at 100° C. with stirring for 12 h. The mixture was filtered, and the filtrate was concentrated under vacuum. The residue was dissolved in EtOAc and the organic phase was washed with water. The organic layer was dried over Na₂SO₄, filtered and concentrated. The residue was purified by preparative TLC (petroleum ether:EtOAc 1:1) to give the title compound (0.13 g, 41%) as a white solid; MS (ESI) m/z [M+H]⁺ 361.

### Intermediate 109: rac-(R)-6-(1-(2-(Trifluoromethyl)pyridin-4-yl)ethyl)quinoline-4-carboxylic Acid

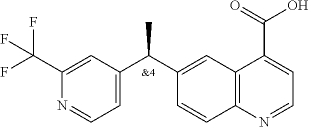 [SMILES: C[C@H](c1ccnc(C(F)(F)F)c1)c1ccc2nccc(C(=O)O)c2c1]

A solution of rac-methyl (R)-6-(1-(2-(trifluoromethyl)pyridin-4-yl)ethyl)quinoline-4-carboxylate Intermediate 108 (120 mg, 0.33 mmol) and LiOH (24 mg, 1.0 mmol) in MeOH (5 mL) and water (1 mL) was stirred at rt for 2 h. The solvent was removed under reduced pressure. The residue was diluted with water and the mixture was adjusted to pH 6. The precipitate was collected by filtration and washed with water to provide the title compound (0.105 g, 91%) as a white solid; MS (ESI) m/z [M+H]⁺ 347.

### Intermediate 110: Methyl 6-(prop-1-en-2-yl)quinoline-4-carboxylate

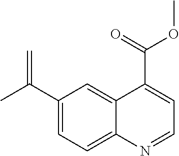 [SMILES: C=C(C)c1ccc2nccc(C(=O)OC)c2c1]

Pd(dppf)Cl₂·DCM (61 mg, 0.08 mmol) was added to methyl 6-bromoquinoline-4-carboxylate (400 mg, 1.50 mmol), 4,4,5,5-tetramethyl-2-(prop-1-en-2-yl)-1,3,2-dioxaborolane (547 mg, 3.01 mmol) and K₂CO₃ (416 mg, 3.01 mmol) in 1,4-dioxane (15 mL) and water (3 mL) at rt. The resulting suspension was stirred at 85° C. for 2 h. The solvent was removed under reduced pressure. The residue taken up in EtOAc (25 mL) and washed with water (2×20 mL). The organic layer was dried over Na₂SO₄, filtered and concentrated. The residue was purified by preparative TLC (petroleum ether:EtOAc, 3:1) to afford the title compound (0.30 g, 88%) as a pale yellow solid; MS (ESI) m/z [M+H]⁺ 228.1.

### Intermediate 111: Methyl 6-(2,2-difluoropropyl)quinoline-4-carboxylate

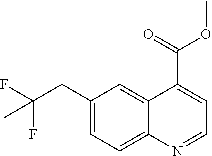 [SMILES: COC(=O)c1ccnc2ccc(CC(C)(F)F)cc12]

1-Iodo-4-methylbenzene (29 mg, 0.13 mmol) and methyl 6-(prop-1-en-2-yl)quinoline-4-carboxylate Intermediate 110 (300 mg, 1.32 mmol) were dissolved in chloroform (3 mL) and added to a mixture of HF-Pyridine (0.96 mL, 11 mmol, 70%) and TREAT-HF (2.04 mL, 12.5 mmol) at rt. The reaction mixture was stirred for 2 min, followed by addition of Selectfluor (701 mg, 1.98 mmol). The resulting solution was stirred at rt for 15 h. The reaction mixture was poured into sat NaHCO₃ (aq), extracted with DCM (3×50 mL), the organic layer was dried over Na₂SO₄, filtered and concentrated under reduced pressure. The residue was purified by preparative TLC (petroleum ether:EtOAc, 3:1) to afford the title compound (0.28 g, 80%) as a grey solid; MS (ESI) m/z [M+H]⁺ 266.1.

### Intermediate 112: 6-(2,2-Difluoropropyl)quinoline-4-carboxylic Acid

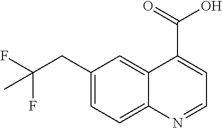 [SMILES: CC(F)(F)Cc1ccc2nccc(C(=O)O)c2c1]

A mixture of methyl 6-(2,2-difluoropropyl)quinoline-4-carboxylate Intermediate 111 (230 mg, 0.87 mmol) and LiOH (42 mg, 1.7 mmol) in MeOH (10 mL) and water (2 mL) was stirred at 30° C. for 2 h. The solvent was removed under reduced pressure. Water (10 mL) was added to the residue and the pH was adjusted to pH 6 with HCl (1 M). The precipitate was collected by filtration, washed with water (50 mL) and dried under vacuum to afford the title compound (0.19 g, 87%) as a white solid; MS (ESI) m/z [M+H]⁺ 252.1.

### Intermediate 113: rac-tert-Butyl (R)-6-(4-cyanocyclohex-1-en-1-yl)quinoline-4-carboxylate

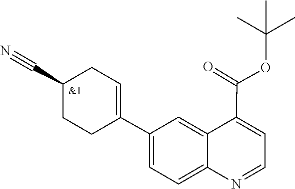 [SMILES: CC(C)(C)OC(=O)c1ccnc2ccc(C3=CC[C@H](C#N)CC3)cc12]

Pd(dppf)Cl₂ (71 mg, 0.10 mmol) was added to a mixture of tert-butyl 6-bromoquinoline-4-carboxylate (WO 2019154886) (300 mg, 0.97 mmol), rac-(R)-4-(4,4,5,5-tetramethyl-1,3,2-dioxaborolan-2-yl)cyclohex-3-ene-1-carbonitrile (340 mg, 1.46 mmol) and K₂CO₃ (269 mg, 1.95 mmol) in 1,4-dioxane (10 mL) under N₂. The reaction was heated at 80° C. for 2 h. The solvent was removed under reduced pressure. The residue was dissolved in EtOAc (30 mL) and washed with water (3×10 mL). The organic layer was dried over Na₂SO₄, filtered and concentrated. Purified by preparative TLC (petroleum ether:EtOAc, 2:1) to afford the title compound (0.25 g, 77%) as a yellow solid; MS (ESI) m/z [M+H]⁺ 335.2.

### Intermediate 114: tert-Butyl 6-((1r,4SR)-4-cyanocyclohexyl)quinoline-4-carboxylate

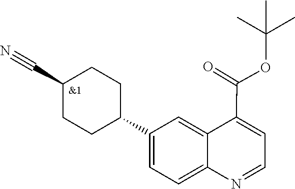 [SMILES: CC(C)(C)OC(=O)c1ccnc2ccc([C@H]3CC[C@H](C#N)CC3)cc12]

rac-tert-Butyl (R)-6-(4-cyanocyclohex-1-en-1-yl)quinoline-4-carboxylate Intermediate 113 (260 mg, 0.78 mmol) and Pd/C (83 mg, 0.08 mmol) in EtOAc (8 mL) was stirred under an atmosphere of hydrogen at rt for 15 h. The reaction mixture was filtered, and the filtrate was concentrated under reduced pressure. The residue was purified by preparative TLC (petroleum ether:EtOAc, 4:1) to afford the title compound (0.11 g, 42%) as a yellow solid;

MS (ESI) m/z [M+H]⁺ 337.2.

### Intermediate 115: 6-((1r,4SR)-4-Cyanocyclohexyl)quinoline-4-carboxylic Acid

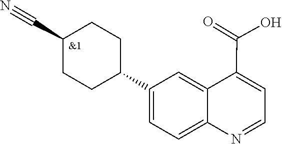 [SMILES: N#C[C@H]1CC[C@H](c2ccc3nccc(C(=O)O)c3c2)CC1]

A mixture of tert-butyl 6-((1r,4SR)-4-cyanocyclohexyl)quinoline-4-carboxylate Intermediate 114 (110 mg, 0.33 mmol) in TFA (3 mL) and DCM (10 mL) was stirred at 30° C. for 3 h. The solvent was removed under reduced pressure. The solid was dried under vacuum to afford the title compound (0.165 g, 99%) as a dark yellow solid. Used as such in the next step;

MS (ESI) m/z [M+H]⁺ 281.1.

### Intermediate 116: 6-((1r,4RS)-4-Cyanocyclohexyl)-N-(2-((R)-4-cyanothiazolidin-3-yl)-2-oxoethyl)quinoline-4-carboxamide

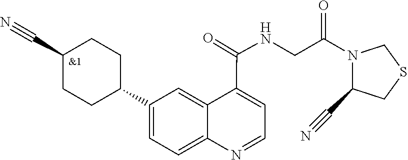 [SMILES: N#C[C@@H]1CSCN1C(=O)CNC(=O)c1ccnc2ccc([C@H]3CC[C@H](C#N)CC3)cc12]

A solution of 6-((1r,4SR)-4-cyanocyclohexyl)quinoline-4-carboxylic acid Intermediate 115 (80 mg, 0.29 mmol), (R)-3-glycylthiazolidine-4-carbonitrile hydrochloride Intermediate 4 (77 mg, 0.37 mmol), EDC (109 mg, 0.57 mmol), HOBt (77 mg, 0.57 mmol) and DIPEA (249 μL, 1.43 mmol) in MeCN (4 mL) and EtOAc (4 mL) was stirred at 50° C. for 2 h. The solvent was removed under reduced pressure. The residue was dissolved in EtOAc (25 mL) and washed with water (2×15 mL). The organic layer was dried over Na₂SO₄, filtered and concentrated under reduced pressure. The residue was purified by preparative TLC (petroleum ether:EtOAc, 1:100) to afford the title compound (0.10 g, 810%) as a white solid;

MS (ESI) m/z [M+H]⁺ 434.2.

### Intermediate 117: tert-Butyl 6-((tetrahydro-2H-pyran-4-yl)methyl)quinoline-4-carboxylate

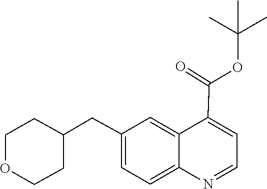 [SMILES: CC(C)(C)OC(=O)c1ccnc2ccc(CC3CCOCC3)cc12]

NiBr₂·DME (30 mg, 0.10 mmol) was added to a mixture of tert-butyl 6-bromoquinoline-4-carboxylate (WO 2019154886) (300 mg, 0.97 mmol), 4-(bromomethyl)tetrahydro-2H-pyran (349 mg, 1.95 mmol), Zn (127 mg, 1.95 mmol), NaI (146 mg, 0.97 mmol) and 4-4′-dimethoxy-2-2′-bipyridine (63 mg, 0.29 mmol) in DMPU (10 mL) under N₂ (g). The reaction mixture was stirred at 80° C. for 2 h. The reaction mixture was diluted with EtOAc (25 mL) and washed with water (3×10 mL). The organic layer was dried over Na₂SO₄, filtered and concentrated under reduced pressure. The residue was purified by preparative TLC (petroleum ether:EtOAc, 4:1), to afford the title compound (40 mg, 13%) as a yellow solid; MS (ESI) m/z [M+H]⁺ 328.1.

### Intermediate 118: 6-((Tetrahydro-2H-pyran-4-yl)methyl)quinoline-4-carboxylic Acid

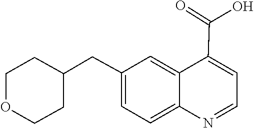 [SMILES: O=C(O)c1ccnc2ccc(CC3CCOCC3)cc12]

A mixture of tert-butyl 6-((tetrahydro-2H-pyran-4-yl)methyl)quinoline-4-carboxylate Intermediate 117 (40 mg, 0.12 mmol) in TFA (1 mL) and DCM (2 mL) was stirred ar rt for 2 h. The solvent was removed under reduced pressure to afford the title compound (33 mg, 100%) as a yellow solid. Used as such in the next step; MS (ESI) m/z [M+H]⁺ 272.1.

### Intermediate 119: Methyl 6-(4-formyltetrahydro-2H-pyran-4-yl)quinoline-4-carboxylate

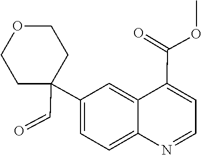 [SMILES: COC(=O)c1ccnc2ccc(C3(C=O)CCOCC3)cc12]

Pd(OAc)₂ (40 mg, 0.18 mmol) was added to methyl 6-bromoquinoline-4-carboxylate (0.60 g, 2.3 mmol), SPhos (93 mg, 0.23 mmol), tetrahydro-2H-pyran-4-carbaldehyde (0.309 g, 2.71 mmol) and Cs₂CO₃ (0.882 g, 2.71 mmol) in 1,4-dioxane (20 mL) at 0° C. The reaction mixture was stirred at 100° C. for 30 h. The solvent was removed under reduced pressure. The residue was purified by preparative TLC (petroleum ether:EtOAc, 2:1) to afford the title compound (0.40 g, 59%) as a yellow oil which solidified on standing;

MS (ESI) m/z [M+H]⁺ 300.1.

### Intermediate 120: 6-(4-Ethynyltetrahydro-2H-pyran-4-yl)quinoline-4-carboxylic Acid

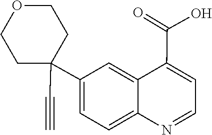 [SMILES: C#CC1(c2ccc3nccc(C(=O)O)c3c2)CCOCC1]

Methyl 6-(4-formyltetrahydro-2H-pyran-4-yl)quinoline-4-carboxylate Intermediate 119 (0.550 g, 1.84 mmol) was dissolved in MeOH (6 mL) and added to dimethyl (1-diazo-2-oxopropyl)phosphonate (1.06 g, 5.51 mmol) and K₂CO₃ (1.27 g, 9.19 mmol) in MeOH (30 mL) at 0° C. The reaction mixture was stirred at rt for 15 h. The solvent was removed under reduced pressure. The crude product was purified by preparative HPLC, PrepMethod F (gradient 13-23%) to afford the title compound (0.15 g, 29%) as a white solid; MS (ESI) m/z [M+H]⁺ 282.1.

### Intermediate 121: tert-Butyl 6-(3,3,3-trifluoropropyl)quinoline-4-carboxylate

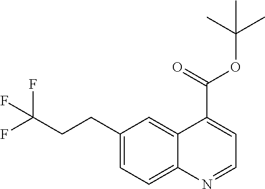 [SMILES: CC(C)(C)OC(=O)c1ccnc2ccc(CCC(F)(F)F)cc12]

NiBr₂·DME (45 mg, 0.15 mmol) was added to a mixture of tert-butyl 6-bromoquinoline-4-carboxylate (WO 2019154886) (300 mg, 0.97 mmol), 3-bromo-1,1,1-trifluoropropane (345 mg, 1.95 mmol), Zn (127 mg, 1.95 mmol), NaI (146 mg, 0.97 mmol) and 4-4′-dimethoxy-2-2′-bipyridine (63 mg, 0.29 mmol) in DMPU (10 mL) under N₂ (g). The reaction mixture was stirred at 80° C. for 2 h. The reaction mixture was diluted with EtOAc (25 mL) and washed with water (3×10 mL). The organic layer was dried over Na₂SO₄, filtered and concentrated under reduced pressure. The residue was purified by preparative TLC (petroleum ether:EtOAc, 3:1) to afford the title compound (0.15 g, 47%) as a pale yellow solid; MS (ESI) m/z [M+H]⁺ 326.2.

### Intermediate 122: 6-(3,3,3-Trifluoropropyl)quinoline-4-carboxylic Acid

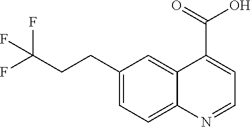 [SMILES: O=C(O)c1ccnc2ccc(CCC(F)(F)F)cc12]

A mixture of tert-butyl 6-(3,3,3-trifluoropropyl)quinoline-4-carboxylate Intermediate 121 (150 mg, 0.46 mmol) in TFA (2 mL) and DCM (8 mL) was stirred at 30° C. for 2 h. The solvent was removed under reduced pressure to afford the title compound (0.124 g, 100%) as a pale yellow solid, which was used in the next step without further purification;

MS (ESI) m/z [M+H]⁺ 270.1.

### Intermediate 123: 6-(4-Cyanotetrahydro-2H-pyran-4-yl)quinoline-4-carboxylic Acid

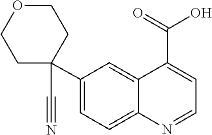 [SMILES: N#CC1(c2ccc3nccc(C(=O)O)c3c2)CCOCC1]

NaHMDS (1.07 mL, 2.14 mmol, 2 M in THF) was added to tert-butyl 6-bromoquinoline-4-carboxylate (WO 2019154886) (300 mg, 0.97 mmol), tetrahydro-2H-pyran-4-carbonitrile (130 mg, 1.17 mmol), Pd(OAc)₂ (4.4 mg, 0.02 mmol) and rac-BINAP (12 mg, 0.02 mmol) in 1,4-dioxane (5 mL) at 30° C. The resulting suspension was stirred at 90° C. for 3 h under N₂ (g). The solvent was removed under reduced pressure. The residue was first purified by preparative TLC (petroleum ether:EtOAc, 2:1), followed by purification by preparative HPLC, PrepMethod F (gradient 13-23%) to afford the title compound (21 mg, 7%) as a white solid; MS (ESI) m/z [M+H]⁺ 283.0.

### Intermediate 124: 6-(1-Cyanocyclohexyl)quinoline-4-carboxylic Acid

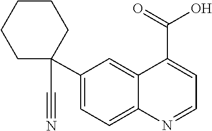 [SMILES: N#CC1(c2ccc3nccc(C(=O)O)c3c2)CCCCC1]

Pd(OAc)₂ (4.4 mg, 0.02 mmol) was added to tert-butyl 6-bromoquinoline-4-carboxylate (WO 2019154886) (300 mg, 0.97 mmol), cyclohexanecarbonitrile (128 mg, 1.17 mmol), rac-BINAP (12 mg, 0.02 mmol) and NaHMDS (1.07 mL, 2.14 mmol, 2 M in THF) in 1,4-dioxane (8 mL) at rt. The resulting suspension was stirred at 90° C. for 3 h under N₂ (g). The solvent was removed under reduced pressure. The crude was purified by preparative HPLC, PrepMethod D (gradient 27-38%) to afford the title compound (21 mg, 8%) as a pale yellow solid; MS (ESI) m/z [M+H]⁺ 281.1.

### Intermediate 125: (Z)—N′-(5,5-Dimethyldihydrofuran-3(2H)-ylidene)-4-methylbenzenesulfonohydrazide

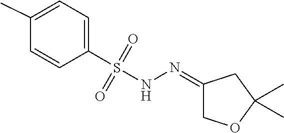

4-Methylbenzenesulfonohydrazide (587 mg, 3.15 mmol) was added to 5,5-dimethyl-dihydrofuran-3(2H)-one (300 mg, 2.63 mmol) in EtOH (20 mL, 99.5%) at rt. The resulting mixture was stirred at 80° C. for 4 h. The solvent was removed under reduced pressure to afford a pale yellow gum. The residue was triturated with petroleum ether:EtOAc (3:1) to give a solid which was dried under vacuum to give the title compound (0.50 g, 67%) as a white solid. Used as such in the next step; MS (ESI) m/z [M+H]⁺ 283.2.

### Intermediate 126: 6-(5,5-Dimethyl-2,5-dihydrofuran-3-yl)quinoline-4-carboxylic Acid

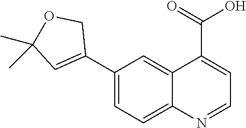 [SMILES: CC1(C)C=C(c2ccc3nccc(C(=O)O)c3c2)CO1]

LiOtBu (566 mg, 7.07 mmol) was added to tert-butyl 6-bromoquinoline-4-carboxylate (WO 2019154886) (991 mg, 3.21 mmol), (Z)—N-(5,5-dimethyldihydrofuran-3(2H)-ylidene)-4-methylbenzenesulfonohydrazide Intermediate 125 (908 mg, 3.21 mmol), Pd₂(dba)₃ (59 mg, 0.06 mmol) and XPhos (61 mg, 0.13 mmol) in 1,4-dioxane (15 mL) at rt. The reaction mixture was stirred at 80° C. for 16 h under N₂ (g). The reaction was incomplete, so the temperature was increased to 90° C. and the reaction mixture was stirred for a further 22 h. The reaction mixture was filtered through Celite®, and the filter cake was washed with DCM (10 mL). The filtrate was concentrated and the residue was first purified by straight phase flash chromatography on silica (gradient: 10-50% MeOH in DCM), followed by purification by preparative HPLC, PrepMethod F (gradient 14-23%) to afford the title compound (0.186 g, 22%) as a pale yellow solid; MS (ESI) m/z [M+H]⁺ 270.0.

### Intermediate 127: Methyl 6-(5,5-dimethyl-2,5-dihydrofuran-3-yl)quinoline-4-carboxylate

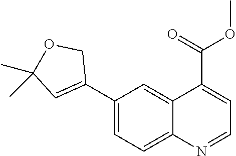

SOCl₂ (379 mg, 3.18 mmol) was added to 6-(5,5-dimethyl-2,5-dihydrofuran-3-yl)quinoline-4-carboxylic acid Intermediate 126 (172 mg, 0.64 mmol) in MeOH (10 mL). The reaction mixture was stirred at rt for 16 h under N₂ (g). The reaction was incomplete and more SOCl₂ (379 mg, 3.18 mmol) was added and the mixture was stirred at rt for a further 16 h. The solvent was removed under reduced pressure, to afford the title compound (0.237 g, 98%) as a yellow gum. The product was used in the next step without further purification;

MS (ESI) m/z [M+H]⁺ 284.0.

### Intermediate 128: rac-methyl (R)-6-(5,5-dimethyltetrahydrofuran-3-yl)quinoline-4-carboxylate

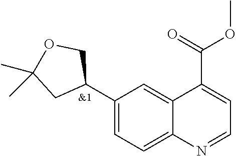

Royer® Palladium Catalyst (100 mg, 0.09 mmol) was added to methyl 6-(5,5-dimethyl-2,5-dihydrofuran-3-yl)quinoline-4-carboxylate Intermediate 127 (197 mg, 0.70 mmol) in EtOAc (10 mL) at rt. The system was evacuated and back-filled with hydrogen 5×. The resulting suspension was stirred at rt for 16 h under H₂ (g). The reaction mixture was filtered through Celite®, and the filter cake was washed with MeOH (100 mL). The filtrate was concentrated under reduced pressure and the residue was purified by preparative TLC (petroleum ether:EtOAc, 2:1) to afford the title compound (56 mg, 28%) as a colourless gum;

MS (ESI) m/z [M+H]⁺ 286.0.

### Intermediate 129: rac-(R)-6-(5,5-dimethyltetrahydrofuran-3-yl)quinoline-4-carboxylic Acid

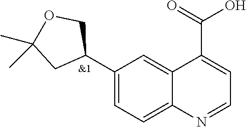 [SMILES: CC1(C)C[C@H](c2ccc3nccc(C(=O)O)c3c2)CO1]

NaOH (35 mg, 0.88 mmol) was added to methyl 6-(5,5-dimethyltetrahydrofuran-3-yl)quinoline-4-carboxylate Intermediate 128 (50 mg, 0.18 mmol) in MeOH (3 mL) and water (1 mL) and the reaction mixture was stirred at rt for 1 h under air. The reaction mixture was diluted with water (10 mL) and adjusted to pH 3 with HCl (1 M). The aq. phase was extracted with EtOAc (3×50 mL). The combined organic layers were dried over Na₂SO₄, filtered and concentrated under reduced pressure to afford the title compound (41 mg, 85%) as a colourless gum. The product was used in the next step without further purification; MS (ESI) m/z [M+H]⁺ 272.0.

### Intermediate 130: 1-(4-Chloroquinolin-6-yl)-3-methoxycyclobutan-1-ol

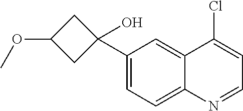 [SMILES: COC1CC(O)(c2ccc3nccc(Cl)c3c2)C1]

6-Bromo-4-chloroquinoline (1.0 g, 4.1 mmol) was dissolved in THF (25 mL) in a dried two necked flask under an argon atmosphere, cooled to −70° C., and n-BuLi (2.5 mL, 6.2 mmol, 2.5 M in hexane) were slowly added dropwise so that the internal temperature did not exceed −65° C. The reaction mixture was stirred for 1 h at −70° C. Subsequently, 3-methoxycyclobutan-1-one (0.619 g, 6.19 mmol) in THF (3 mL) was slowly added dropwise so that the internal temperature did not exceed −65° C. The reaction mixture was stirred further for 1 h at −70° C. and subsequently warmed to rt. It was stirred for 3 h at 30° C. The reaction mixture was quenched with sat aq NH₄Cl (25 mL), extracted with EtOAc (3×50 mL), the organic layer was dried over Na₂SO₄, filtered and concentrated under reduced pressure. The residue was purified by preparative TLC (petroleum ether:EtOAc, 2:1) to afford the title compound (0.30 g, 28%) as a white solid; MS (ESI) m/z [M+H]⁺ 264.1.

### Intermediate 131: Methyl 6-(1-hydroxy-3-methoxycyclobutyl)quinoline-4-carboxylate

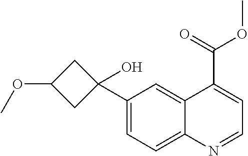

A mixture of 1-(4-chloroquinolin-6-yl)-3-methoxycyclobutan-1-ol Intermediate 130 (300 mg, 1.14 mmol), Pd(OAc)₂ (26 mg, 0.11 mmol), Pd(dppf)Cl₂ (42 mg, 0.06 mmol), dppf (95 mg, 0.17 mmol) and TEA (476 μL, 3.41 mmol) in MeOH (15 mL) under an atmosphere of CO (g) at 10 atm was stirred at 100° C. for 12 h. The solid was filtered off and the filtrate was concentrated. The residue was taken up in EtOAc (30 mL) and washed with water (3×10 mL). The organic layer was dried over Na₂SO₄, filtered and concentrated under reduced pressure. The residue was purified by preparative TLC (petroleum ether:EtOAc, 1:1), to afford the title compound (0.23 g, 70%) as a pale yellow solid; MS (ESI) m/z [M+H]⁺ 288.1.

### Intermediate 132: Methyl 6-(1-fluoro-3-methoxycyclobutyl)quinoline-4-carboxylate

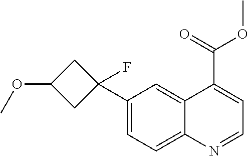

A solution of DAST (212 μL, 1.60 mmol) in DCM (1 mL) was added dropwise to a stirred solution of methyl 6-(1-hydroxy-3-methoxycyclobutyl)quinoline-4-carboxylate Intermediate 131 (230 mg, 0.80 mmol) in DCM (10 mL) at 0° C. The reaction mixture was stirred at rt for 2 h. The solvent was removed under reduced pressure. The residue was purified by preparative TLC (petroleum ether:EtOAc, 2:1), to afford the title compound (0.17 g, 73%) as a pale yellow solid; MS (ESI) m/z [M+H]⁺ 290.1.

### Intermediate 133: 6-(1-Fluoro-3-methoxycyclobutyl)quinoline-4-carboxylic Acid

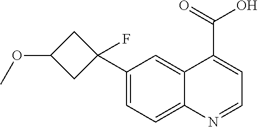 [SMILES: COC1CC(F)(c2ccc3nccc(C(=O)O)c3c2)C1]

A mixture of methyl 6-(1-fluoro-3-methoxycyclobutyl)quinoline-4-carboxylate Intermediate 132 (170 mg, 0.59 mmol) and NaOH (71 mg, 1.8 mmol) in MeOH (8 mL) and water (2 mL) was stirred at 30° C. for 2 h. The solvent was removed under reduced pressure and water (10 mL) was added. The pH was adjusted to pH 6 with 1 M HCl. The precipitate was collected by filtration, washed with water (30 mL) and dried under vacuum to afford the title compound (0.15 g, 93%) as a pale yellow solid, which was used without further purification;

MS (ESI) m/z [M+H]⁺ 276.1.

### Intermediate 134: Methyl 6-(4-fluorotetrahydro-2H-pyran-4-yl)quinoline-4-carboxylate

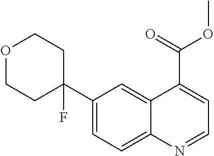 [SMILES: COC(=O)c1ccnc2ccc(C3(F)CCOCC3)cc12]

Methyl 6-(4-hydroxytetrahydro-2H-pyran-4-yl)quinoline-4-carboxylate Intermediate 49 (190 mg, 0.66 mmol) was dissolved in anhydrous DCM (10 mL) and cooled to −40° C. under N₂ (g), DAST (175 μL, 1.32 mmol) was added dropwise and the resulting mixture was stirred at rt for 1 h. The reaction was quenched with sat NaHCO₃ (aq, 25 mL) and extracted with DCM (3×25 mL). The organic layers were combined and washed with water (2×15 mL). The organic layer was dried over Na₂SO₄, filtered and concentrated under reduced pressure. The residue was purified by preparative TLC (DCM:MeOH, 10:1), to afford the title compound (0.19 g, 99%) as a beige oil which solidified on standing; MS (ESI) m/z [M+H]⁺ 290.1.

### Intermediate 135: 6-(4-Fluorotetrahydro-2H-pyran-4-yl)quinoline-4-carboxylic Acid

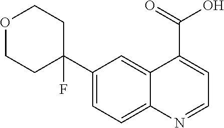 [SMILES: O=C(O)c1ccnc2ccc(C3(F)CCOCC3)cc12]

Methyl 6-(4-fluorotetrahydro-2H-pyran-4-yl)quinoline-4-carboxylate Intermediate 134 (170 mg, 0.59 mmol) was dissolved in MeOH (6 mL) and cooled to 0° C. A solution of NaOH (94 mg, 2.4 mmol) in water (2 mL) was added slowly. The reaction mixture was stirred at rt for 1 h, followed by dilution with water (20 mL), and the pH was adjusted to pH 6 with HCl (2 M). The mixture was concentrated under reduced pressure to afford the title compound (0.23 g, 142%) as a white solid, which was used in the next step directly without further purification; MS (ESI) m/z [M+H]⁺ 276.1.

### Intermediate 136: (4-(Methoxycarbonyl)quinolin-6-yl)boronic Acid

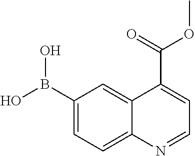 [SMILES: COC(=O)c1ccnc2ccc(B(O)O)cc12]

XPhos (14 mg, 0.03 mmol) and XPhos-Pd-G2 (12 mg, 0.02 mmol) were added to methyl 6-bromoquinoline-4-carboxylate (200 mg, 0.75 mmol), B₂(OH)₄ (202 mg, 2.25 mmol) and KOAc (221 mg, 2.25 mmol) in EtOH (15 mL, 99.5%) at rt. The resulting suspension was stirred at 80° C. for 12 h under N₂ (g). The reaction mixture was poured into brine (150 mL), extracted with EtOAc (3×100 mL), the combined organic layer was dried over Na₂SO₄, filtered and concentrated under reduced pressure to afford the title compound (0.17 g, 98%) as a brown solid. The product was used in the next step directly without further purification;

MS (ESI) m/z [M+H]⁺ 232.1.

### Intermediate 137: (E)-4-Methyl-N′-((tetrahydrofuran-3-yl)methylene)-benzenesulfonohydrazide

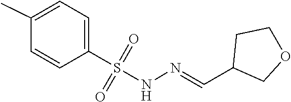 [SMILES: Cc1ccc(S(=O)(=O)N/N=C/C2CCOC2)cc1]

4-Methylbenzenesulfonohydrazide (1.12 g, 5.99 mmol) was added to tetrahydrofuran-3-carbaldehyde (50% in water) (1.0 g, 5.0 mmol) in EtOH (10 mL, 99.5%) at rt. The reaction mixture was stirred at 80° C. for 15 h. The reaction mixture was poured into brine (150 mL), extracted with EtOAc (3×100 mL), the combined organic layer was dried over Na₂SO₄, filtered and concentrated under reduced pressure to afford the title compound (0.80 g, 60%) as a pale yellow gum. The product was used in the next step directly without further purification; MS (ESI) m/z [M+H]⁺ 269.1.

### Intermediate 138: rac-Methyl (R)-6-((tetrahydrofuran-3-yl)methyl)quinoline-4-carboxylate

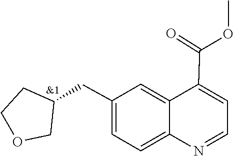 [SMILES: COC(=O)c1ccnc2ccc(C[C@H]3CCOC3)cc12]

K₂CO₃ (479 mg, 3.47 mmol) was added to (4-(methoxycarbonyl)quinolin-6-yl)boronic acid Intermediate 136 (801 mg, 3.47 mmol) and (E)-4-methyl-N-((tetrahydrofuran-3-yl)methylene)benzenesulfonohydrazide Intermediate 137 (620 mg, 2.31 mmol) in 1,4-dioxane (15 mL) at rt. The resulting solution was stirred at 110° C. for 15 h. The reaction mixture was poured into brine (150 mL), extracted with EtOAc (3×75 mL), the combined organic layer was dried over Na₂SO₄, filtered and concentrated under reduced pressure. The residue was purified by preparative TLC (petroleum ether:EtOAc, 1:1) to afford the title compound (0.10 g, 16%) as a pale yellow gum; MS (ESI) m/z [M+H]⁺ 272.1.

### Intermediate 139: rac-(R)-6-((Tetrahydrofuran-3-yl)methyl)quinoline-4-carboxylic Acid

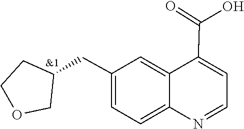 [SMILES: O=C(O)c1ccnc2ccc(C[C@H]3CCOC3)cc12]

LiOH (44 mg, 1.8 mmol) was added to rac-methyl (R)-6-((tetrahydrofuran-3-yl)methyl)quinoline-4-carboxylate Intermediate 138 (100 mg, 0.37 mmol) in MeOH (15 mL) and water (5 mL) at rt. The resulting solution was stirred at rt for 15 h. The reaction mixture was poured into water (150 mL), acidified with HCl (2 M) and extracted with DCM:MeOH (10:1) (5×100 mL). The combined organic layer was dried over Na₂SO₄, filtered and concentrated under reduced pressure to afford the title compound (90 mg, 95%) as a white solid. The product was used in the next step directly without further purification; MS (ESI) m/z [M+H]⁺ 258.2.

### Intermediate 140: 6-(4-Methoxytetrahydro-2H-pyran-4-yl)quinoline-4-carboxylic Acid

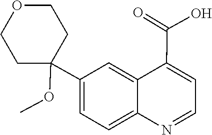 [SMILES: COC1(c2ccc3nccc(C(=O)O)c3c2)CCOCC1]

NaH (84 mg, 2.1 mmol, 60% wt) was added to a solution of methyl 6-(4-hydroxytetrahydro-2H-pyran-4-yl)quinoline-4-carboxylate Intermediate 49 (200 mg, 0.70 mmol) in DMF (10 mL). The reaction mixture was stirred at rt for 30 min, followed by addition of MeI (198 mg, 1.39 mmol). The reaction was stirred at rt for 2 h. LiOH (33 mg, 1.4 mmol) and water (2 mL) was added and the reaction mixture was stirred at rt for 2 h. The solvent was removed under reduced pressure. Water (5 mL) and EtOAc (25 mL) were added, and the organic layer was washed with water (3×10 mL), dried over Na₂SO₄, filtered and concentrated under reduced pressure to afford the title compound (0.18 g, 90%) as a brown solid. The product was used in the next step directly without further purification; MS (ESI) m/z [M+H]⁺ 288.1.

### Intermediate 141: rac-Ethyl (R)-6-(tetrahydrofuran-3-yl)quinoline-4-carboxylate

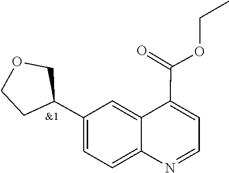 [SMILES: CCOC(=O)c1ccnc2ccc([C@H]3CCOC3)cc12]

### Step a) Preparation of Precatalyst Stock Solution

A vial was charged with NiCl₂·DME (3.0 mg, 0.014 mmol) and dtbbpy (3.7 mg, 0.014 mmol). The vial was sealed, evacuated and filled with N₂ (g) before DME (3 mL) was added. The precatalyst stock solution was stirred and bubbled with N₂ (g) for 10 min and then placed in an ultrasound bath for 5 min.

### Step b) Preparation of Intermediate 141

An oven dried vial was charged with Ir[dF(CF₃)ppy]₂(dtbbpy)PF₆ (10 mg, 8.9 μmol), rac-(R)-3-bromotetrahydrofuran (202 mg, 1.34 mmol), ethyl 6-bromoquinoline-4-carboxylate (250 mg, 0.89 mmol), tris(trimethylsilyl)silane (413 μL, 1.34 mmol) and Na₂CO₃ (189 mg, 1.78 mmol). The vial was sealed, DME (5 mL) (degassed by bubbling with N₂ (g) for 10 min) was added and the vial was evacuated and filled with N₂ (g). A precatalyst stock solution prepared as described in Step a) (0.981 mL) was transferred into the reaction vial. The reaction mixture was bubbled with N₂ (g) and stirred for 10 min, then sealed with parafilm. The reaction was stirred and irradiated with a 34 W blue LED lamp under a fan overnight. DCM and water were added, the mixture was stirred, filtered through a phase separator and concentrated. The residue was purified by straight phase flash chromatography on silica (gradient: 10-60% EtOAc in heptane) to afford the title compound (36 mg, 15%); MS (ESI) m/z [M+H]⁺ 272.3.

### Intermediate 142: rac-(R)-6-(Tetrahydrofuran-3-yl)quinoline-4-carboxylic Acid

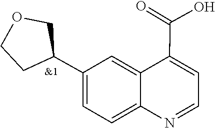 [SMILES: O=C(O)c1ccnc2ccc([C@H]3CCOC3)cc12]

rac-Ethyl (R)-6-(tetrahydrofuran-3-yl)quinoline-4-carboxylate Intermediate 141 (36 mg, 0.13 mmol) was dissolved in MeOH (2 mL) and aq NaOH (265 μL, 0.27 mmol, 1 M) was added. The reaction was stirred at 50° C. for 20 min. HCl (87 μL, 0.33 mmol, 3.8 M) was added to the reaction mixture and the mixture was concentrated under reduced pressure and co-evaporated with EtOH (2×) and MeCN to afford the title compound (32 mg, 100%). The product was used in the next step directly without further purification; MS (ESI) m/z [M+H]⁺ 244.0.

### Intermediate 143: rac-Ethyl (R)-6-(2,2-dimethyltetrahydro-2H-pyran-4-yl)quinoline-4-carboxylate

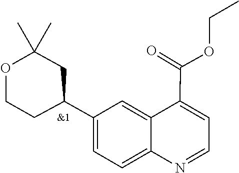 [SMILES: CCOC(=O)c1ccnc2ccc([C@H]3CCOC(C)(C)C3)cc12]

An oven dried vial was charged with Ir[dF(CF₃)ppy]₂(dtbbpy)PF₆ (10 mg, 8.9 μmol), rac-(R)-4-bromo-2,2-dimethyltetrahydro-2H-pyran (258 mg, 1.34 mmol), ethyl 6-bromoquinoline-4-carboxylate (250 mg, 0.89 mmol), tris(trimethylsilyl)silane (413 μL, 1.34 mmol) and Na₂CO₃ (189 mg, 1.78 mmol). The vial was sealed, DME (5 mL) (degassed by bubbling with N₂ (g) for 10 min) was added, evacuated and filled with N₂ (g). A precatalyst stock solution prepared as described for Intermediate 141 Step a) (0.98 mL) was transferred into the reaction vial. The reaction mixture was bubbled with N₂ (g) and stirred for 10 min, then sealed with parafilm. The reaction was stirred and irradiated with a 34 W blue LED lamp under a fan overnight. DCM and water were added, the mixture was stirred, filtered through a phase separator and concentrated under reduced pressure. The residue was purified by straight phase flash chromatography on silica (gradient: 5-50% EtOAc in heptane) to afford the title compound (44 mg, 16%). The product was used in the next step directly without further purification; MS (ESI) m/z [M+H]⁺ 314.3.

### Intermediate 144: rac-(R)-6-(2,2-Dimethyltetrahydro-2H-pyran-4-yl)quinoline-4-carboxylic Acid

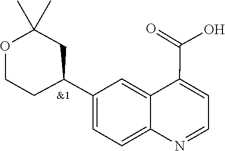 [SMILES: CC1(C)C[C@@H](c2ccc3nccc(C(=O)O)c3c2)CCO1]

rac-Ethyl (R)-6-(2,2-dimethyltetrahydro-2H-pyran-4-yl)quinoline-4-carboxylate Intermediate 143 (44 mg, 0.15 mmol) was dissolved in MeOH (2 mL) and aq NaOH (294 μL, 0.29 mmol, 1 M) was added. The reaction was stirred at 50° C. for 20 min. HCl (97 μL, 0.37 mmol, 3.8 M) was added to the reaction mixture and the mixture was concentrated under reduced pressure and co-evaporated with EtOH (2×) and MeCN to afford the title compound (42 mg, 100%). The product was used in the next step directly without further purification;

MS (ESI) m/z [M+H]⁺ 286.3.

### Intermediate 145: Potassium rac-((1R,6R)-3-oxabicyclo[4.1.0]heptan-6-yl)trifluoroborate

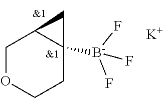 [SMILES: F[B-](F)(F)[C@]12CCOC[C@H]1C2 ; [K+]]

KHF₂ (1.04 g, 13.3 mmol) was added to a solution of rac-2-((1R,6R)-3-oxabicyclo[4.1.0]heptan-6-yl)-4,4,5,5-tetramethyl-1,3,2-dioxaborolane (425 mg, 1.90 mmol) in MeOH (12 mL) and the reaction mixture was refluxed for 4 h. After cooling to rt the mixture was concentrated under reduced pressure and the residue was triturated with 20% diethyl ether in heptane (25 mL) for 1 h. The mixture was filtered, and the solids were washed with diethyl ether (15 mL). The solids were stirred briefly in hot MeCN (60 mL), during which the product dissolved. Filtered to remove inorganic salts. The filtrate was concentrated to give the title compound (0.366 g, 95%) as a white solid; 1H NMR (500 MHz, CD₃OD) δ 3.94-3.80 (m, 2H), 3.48 (ddd, 1H), 3.18 (td, 1H), 1.89 (dt, 1H), 1.53 (ddd, 1H), 0.74-0.67 (m, 1H), 0.53 (dd, 1H), 0.06 (s, 1H),

### Intermediate 146: rac-Ethyl 6-((1R,6S)-3-oxabicyclo[4.1.0]heptan-6-yl)quinoline-4-carboxylate

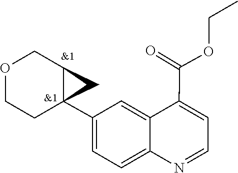 [SMILES: CCOC(=O)c1ccnc2ccc([C@]34CCOC[C@H]3C4)cc12]

A vial was charged with ethyl 6-bromoquinoline-4-carboxylate (148 mg, 0.53 mmol), potassium rac-((1R,6R)-3-oxabicyclo[4.1.0]heptan-6-yl)trifluoroborate Intermediate 145 (54 mg, 0.26 mmol), cataCXium® A Pd G3 (9.6 mg, 0.01 mmol) and Cs₂CO₃ (259 mg, 0.79 mmol). Toluene (6 mL) and water (0.6 mL) were added and the mixture was degassed by bubbling N₂ (g) for 10 min. The mixture was heated at 90° C. for 3 h, then stirred at rt overnight. The reaction mixture was diluted with DCM and water, stirred, filtered through a phase separator and concentrated under reduced pressure. The residue was purified by straight phase flash chromatography on silica (gradient: 10-60% EtOAc in heptane) to afford the title compound (27 mg, 34%). The product was used in the next step directly without further purification; MS (ESI) m/z [M+H]⁺ 298.3.

### Intermediate 147: rac-6-((1R,6S)-3-Oxabicyclo[4.1.0]heptan-6-yl)quinoline-4-carboxylic Acid

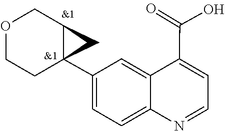 [SMILES: O=C(O)c1ccnc2ccc([C@]34CCOC[C@H]3C4)cc12]

rac-Ethyl 6-((1R,6S)-3-oxabicyclo[4.1.0]heptan-6-yl)quinoline-4-carboxylate Intermediate 146 (27 mg, 0.09 mmol) was dissolved in MeOH (1 mL) and aq NaOH (272 μL, 0.27 mmol, 1 M) was added. The reaction was stirred at 50° C. for 20 min. HCl (84 μL, 0.32 mmol, 3.8 M) was added to the reaction mixture and the mixture was concentrated under reduced pressure and co-evaporated with EtOH (2×) to afford the title compound (24 mg, 100%). The product was used in the next step directly without further purification;

MS (ESI) m/z [M+H]⁺ 270.1.

### Intermediate 148: Methyl 6-(difluoromethyl)quinoline-4-carboxylate

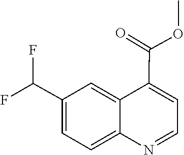 [SMILES: COC(=O)c1ccnc2ccc(C(F)F)cc12]

Methyl 6-formylquinoline-4-carboxylate Intermediate 8 (180 mg, 0.84 mmol) in DCM (5 mL) was cooled in an ice-bath and DAST (0.22 mL, 1.7 mmol) was added over a period of 1 min. The reaction mixture was stirred at rt for 12 h. The solvent was removed under reduced pressure. The residue was purified by preparative TLC (petroleum ether:EtOAc, 3:1) to afford the title compound (100 mg, 50%) as a white solid; MS (ESI) m/z [M+H]⁺ 238.0.

### Intermediate 149: 6-(Difluoromethyl)quinoline-4-carboxylic Acid

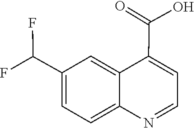 [SMILES: O=C(O)c1ccnc2ccc(C(F)F)cc12]

A mixture of methyl 6-(difluoromethyl)quinoline-4-carboxylate Intermediate 148 (100 mg, 0.42 mmol) and LiOH (20 mg, 0.84 mmol) in THF (3 mL) and water (0.5 mL) was stirred at rt for 1 h. The solvent was removed under reduced pressure. The reaction mixture was acidified with HCl (0.1 M) and the precipitate was collected by filtration, washed with water to afford the title compound (60 mg, 64%) as a white solid; MS (ESI) m/z [M+H]⁺ 224.2.

### Intermediate 150: 6-(4-(3-Methoxypropoxy)phenyl)quinoline-4-carboxylic Acid

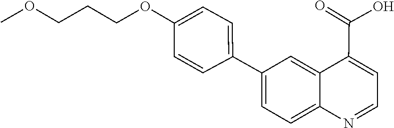 [SMILES: COCCCOc1ccc(-c2ccc3nccc(C(=O)O)c3c2)cc1]

A mixture of 6-bromoquinoline-4-carboxylic acid (130 mg, 0.52 mmol), (4-(3-methoxypropoxy)phenyl)boronic acid (108 mg, 0.52 mmol), Cs₂CO₃ (336 mg, 1.03 mmol) and Pd(dtbpf)Cl₂ (50 mg, 0.08 mmol) in 1,4-dioxane:water (4:1) (5.16 mL) was stirred under N₂ (g) for 2 h. The 1,4-dioxane was removed under reduced pressure. The water phase was diluted with water and acidified to pH 2 using HCl (1 M). The precipitate was filtered off, washed with diethyl ether and dried under vacuum to afford the title compound (50 mg, 29%);

MS (ESI) m/z [M+H]⁺ 338.3.

### Intermediate 151: Methyl 6-(2-fluoro-2-methylpropoxy)quinoline-4-carboxylate

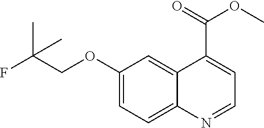 [SMILES: COC(=O)c1ccnc2ccc(OCC(C)(C)F)cc12]

1-Bromo-2-fluoro-2-methylpropane (7.63 g, 49.2 mmol) and Cs₂CO₃ (40.1 g, 123 mmol) was added to a stirred solution of methyl 6-hydroxyquinoline-4-carboxylate (10 g, 49 mmol) in DMF (200 mL) and the reaction mixture was stirred at 100° C. for 48 h. The reaction mixture was filtered through Celite®, and the solvent was removed under reduced pressure to give the title compound (10 g, 73%) as a brown solid. The product was used in the next step without further purification; MS m/z (ESI), [M+H]⁺ 278.2.

### Intermediate 152: 6-(2-Fluoro-2-methylpropoxy)quinoline-4-carboxylic Acid

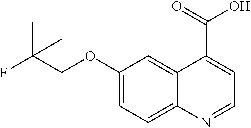 [SMILES: CC(C)(F)COc1ccc2nccc(C(=O)O)c2c1]

LiOH (1.73 g, 72.1 mmol) and water (50 mL) was added to a solution of methyl 6-(2-fluoro-2-methylpropoxy)quinoline-4-carboxylate Intermediate 151 (10 g, 36 mmol) in THF (50 mL) and the reaction mixture was stirred at 25° C. for 4 h. The reaction mixture was diluted with water (100 mL) the pH was adjusted to pH 5 with HCl (2 M), and the resulting mixture was filtered through Celite®. The solids were washed with water (100 mL) and the crude product was purified by reversed phase flash chromatography on a C18 column (gradient: 0-40% of MeCN in water) to give the title compound (2.2 g, 23%) as a grey solid: MS m/z (ESI) [M+H]⁺ 264.2.

### Intermediate 153: Methyl 6-(2,2-difluoroethoxy)quinoline-4-carboxylate

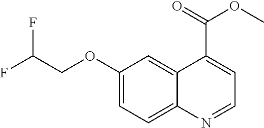 [SMILES: COC(=O)c1ccnc2ccc(OCC(F)F)cc12]

2-Bromo-1,1-difluoroethane (143 mg, 0.98 mmol) and Cs₂CO₃ (802 mg, 2.46 mmol) was added to a stirred solution of methyl 6-hydroxyquinoline-4-carboxylate (200 mg, 0.98 mmol) in DMF (5 mL) and the reaction mixture was stirred at 25° C. for 12 h. The solvent was removed under reduced pressure and the residue was purified by preparative TLC (petroleum ether:EtOAc; 2:1), to give the title compound (0.17 g, 65%) as a white solid: MS (ESI) m/z [M+H]⁺ 268.0.

### Intermediate 154: 6-(2,2-Difluoroethoxy)quinoline-4-carboxylic Acid

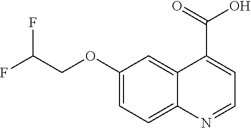 [SMILES: O=C(O)c1ccnc2ccc(OCC(F)F)cc12]

A solution of methyl 6-(2,2-difluoroethoxy)quinoline-4-carboxylate Intermediate 153 (150 mg, 0.56 mmol) and LiOH (67 mg, 2.8 mmol) in THF (2 mL) and water (2 mL) was stirred at 25° C. for 3 h. The pH of the reaction mixture was adjusted to 5 using HCl (2 M), and the solvent was removed under reduced pressure to give the title compound (0.13 g, 91%) as a white solid. The product was used in the next step without further purification: MS (ESI) m/z [M+H]⁺ 253.9.

### Intermediate 155: Methyl 6-propoxyquinoline-4-carboxylate

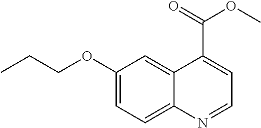 [SMILES: CCCOc1ccc2nccc(C(=O)OC)c2c1]

K₂CO₃ (340 mg, 2.46 mmol) was added to a solution of methyl 6-hydroxyquinoline-4-carboxylate (200 mg, 0.98 mmol) and 1-iodopropane (251 mg, 1.48 mmol) in DMF (3 mL) at 30° C., and the reaction mixture was stirred at 60° C. for 16 h. The solvent was removed under reduced pressure and the crude product was purified by preparative TLC (EtOAc:petroleum ether, 1:1), to give the title compound (166 mg, 69%) as a brown gum: MS (ESI) m/z [M+H]⁺ 246.2.

### Intermediate 156: 6-Propoxyquinoline-4-carboxylic Acid

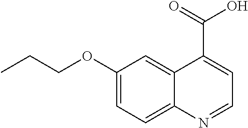 [SMILES: CCCOc1ccc2nccc(C(=O)O)c2c1]

NaOH (131 mg, 3.28 mmol) was added to a solution of methyl 6-propoxyquinoline-4-carboxylate Intermediate 155 (161 mg, 0.66 mmol) in MeOH (9 mL) and water (3 mL) at 25° C. and the reaction mixture was stirred at 25° C. for 1 h. The solvent was removed under reduced pressure and the pH of the reaction mixture was adjusted to 3 with HCl (1 M). The reaction mixture was diluted with water (50 mL) and washed with EtOAc (3×50 mL). The combined organic layer was dried over Na₂SO₄, filtered and evaporated to give the title compound (149 mg, 98%) as a pale yellow solid: MS (ESI) m/z [M+H]⁺ 232.

### Intermediate 157: Methyl 6-(2-fluoroethoxy)quinoline-4-carboxylate

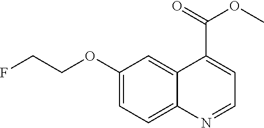 [SMILES: COC(=O)c1ccnc2ccc(OCCF)cc12]

Cs₂CO₃ (802 mg, 2.46 mmol) was added to methyl 6-hydroxyquinoline-4-carboxylate (200 mg, 0.98 mmol) and 1-bromo-2-fluoroethane (187 mg, 1.48 mmol) in DMF (3 mL) at 30° C. and the reaction mixture was stirred at 60° C. for 3 h. The solvent was removed under reduced pressure and the reaction mixture was diluted with water (20 mL) and washed with EtOAc (3×50 mL). The combined organic layer was dried over Na₂SO₄, filtered and evaporated to give the title compound (0.159 g, 65%) as a pale yellow solid: MS (ESI) m/z [M+H]⁺ 250.

### Intermediate 158: 6-(2-Fluoroethoxy)quinoline-4-carboxylic Acid

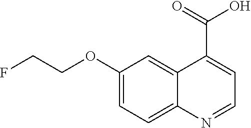 [SMILES: O=C(O)c1ccnc2ccc(OCCF)cc12]

NaOH (123 mg, 3.07 mmol) was added to a solution of methyl 6-(2-fluoroethoxy)quinoline-4-carboxylate Intermediate 157 (153 mg, 0.61 mmol) in MeOH (9 mL) and water (3 mL) at 25° C. and the reaction mixture was stirred at 25° C. for 1 h. The solvent was removed under reduced pressure and the reaction mixture was diluted with EtOAc (50 mL) and washed with sat NaHCO₃ (aq, 3×100 mL). The pH of the aqueous phase was adjusted to 3 with HCl (1 M) and extracted with EtOAc (3×100 mL). The combined organic layer was dried over Na₂SO₄, filtered and evaporated to give the title compound (0.133 g, 92%) as a pale yellow solid: MS (ESI) m/z [M+H]⁺ 236.2.

### Intermediate 159: 1-(4-Chloroquinolin-6-yl)cyclobutan-1-ol

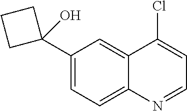 [SMILES: OC1(c2ccc3nccc(Cl)c3c2)CCC1]

6-Bromo-4-chloroquinoline (1.0 g, 4.1 mmol) was dissolved in THF (20 mL) in a dried two necked flask under an argon atmosphere and cooled to −70° C. n-BuLi (2.47 mL, 6.19 mmol) were slowly added dropwise so that the internal temperature did not exceed −65° C. The reaction mixture was stirred at −70° C. for 1 h. A solution of cyclobutanone (0.43 g, 6.2 mmol) in THF (3 mL) were slowly added dropwise so that the internal temperature did not exceed −65° C. The reaction solution was stirred at −70° C. for 1 h and subsequently warmed to rt, and then stirred at rt for 3 h. The reaction mixture was quenched with ice, extracted with EtOAc, and the organic layer was dried over Na₂SO₄, filtered and evaporated. The residue was purified by preparative TLC (petroleum ether:EtOAc, 1:1), to give the title compound (0.35 g, 36%) as a pale yellow solid: MS (ESI) m/z [M+H]⁺ 234.

### Intermediate 160: Methyl 6-(1-hydroxycyclobutyl)quinoline-4-carboxylate

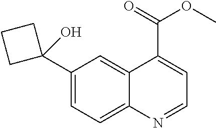 [SMILES: COC(=O)c1ccnc2ccc(C3(O)CCC3)cc12]

Pd(OAc)₂ (31 mg, 0.14 mmol) and Pd(dppf)Cl₂·DCM (56 mg, 0.07 mmol) was added to a mixture of 1-(4-chloroquinolin-6-yl)cyclobutan-1-ol Intermediate 159 (320 mg, 1.37 mmol) and Et₃N (573 μL, 4.11 mmol) in MeOH (15 mL) under a N₂ (g) atmosphere. The resulting mixture was stirred under CO (g) atmosphere (10 atm) at 100° C. for 12 h. The reaction mixture was filtered, and the filtrate was concentrated under vacuum. The crude product was diluted with EtOAc, and washed with water, and the organic layer was dried over Na₂SO₄, filtered and evaporated. The crude product was purified by preparative TLC (petroleum ether:EtOAc, 1:1), to give the title compound (0.27 g, 77%) as a pale yellow solid: MS (ESI) m/z [M+H]⁺ 258.

### Intermediate 161: Methyl 6-(1-fluorocyclobutyl)quinoline-4-carboxylate

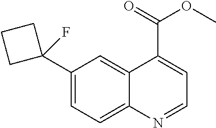 [SMILES: COC(=O)c1ccnc2ccc(C3(F)CCC3)cc12]

A solution of DAST (277 μL, 2.10 mmol) in DCM (1 mL) was added dropwise to a stirred solution of methyl 6-(1-hydroxycyclobutyl)quinoline-4-carboxylate Intermediate 160 (270 mg, 1.05 mmol) in DCM (10 mL) at 0° C., and the reaction mixture was stirred at rt for 2 h. The solvent was removed under reduced pressure and the crude product was purified by preparative TLC (petroleum ether:EtOAc, 2:1), to give the title compound (0.20 g, 73%) as a pale yellow solid: MS (ESI) m/z [M+H]⁺ 260.

### Intermediate 162: 6-(1-Fluorocyclobutyl)quinoline-4-carboxylic Acid

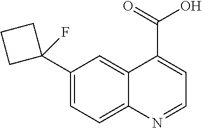 [SMILES: O=C(O)c1ccnc2ccc(C3(F)CCC3)cc12]

A solution of methyl 6-(1-fluorocyclobutyl)quinoline-4-carboxylate Intermediate 161 (200 mg, 0.77 mmol) and LiOH (55 mg, 2.3 mmol) in MeOH (8 mL) and water (2 mL) was stirred at rt for 2 h. The solvent was removed under reduced pressure. The reaction mixture was diluted with water, and the pH was adjusted to 6. The precipitate was collected by filtration, washed with water and dried under vacuum to give the title compound (0.18 g, 95%) as a white solid: MS (ESI) m/z [M+H]⁺ 246.

### Intermediate 163: Methyl 6-(1-ethoxyvinyl)quinoline-4-carboxylate

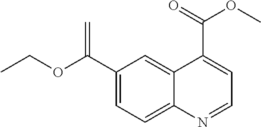 [SMILES: C=C(OCC)c1ccc2nccc(C(=O)OC)c2c1]

A solution of methyl 6-bromoquinoline-4-carboxylate (400 mg, 1.50 mmol), tributyl(1-ethoxyvinyl)stannane (651 mg, 1.80 mmol) and Pd(dppf)Cl₂·DCM (123 mg, 0.15 mmol) in 1,4-dioxane (15 mL) was stirred at 80° C. for 15 h. The solvent was removed under reduced pressure and the crude solid was dried under vacuum. The crude product was purified by preparative TLC (pentane:EtOAc, 4:1), to give the title compound (300 mg, 78%) as a grey solid: MS (ESI) m/z [M+H]⁺ 258.

### Intermediate 164: Methyl 6-(1-ethoxycyclopropyl)quinoline-4-carboxylate

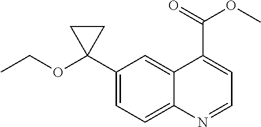 [SMILES: CCOC1(c2ccc3nccc(C(=O)OC)c3c2)CC1]

A solution of CH₂I₂ (1.04 g, 3.89 mmol) in DCM (2 mL) was added to a solution of Et₂Zn (1.60 g, 1.94 mmol, 15 wt % in hexane) in DCM (10 mL) at −78° C. and under a N₂ (g) atmosphere and the reaction mixture was stirred at −15° C. for 30 min. A solution of TFA (0.150 mL, 1.94 mmol) in DCM (2 mL) was added and the reaction mixture was stirred for 30 min. A solution of methyl 6-(1-ethoxyvinyl)quinoline-4-carboxylate Intermediate 163 (250 mg, 0.97 mmol) in DCM (2 mL) was added and the reaction was stirred at rt for 3 h. The solvent was removed under reduced pressure and the crude product was purified by preparative TLC (pentane:EtOAc, 4:1), to give the title compound (60 mg, 23%) as a white solid: MS (ESI) m/z [M+H]⁺ 272.

### Intermediate 165: 6-(1-Ethoxycyclopropyl)quinoline-4-carboxylic Acid

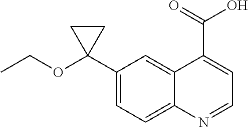 [SMILES: CCOC1(c2ccc3nccc(C(=O)O)c3c2)CC1]

A solution of methyl 6-(1-ethoxycyclopropyl)quinoline-4-carboxylate Intermediate 164 (60 mg, 0.22 mmol) and LiOH (11 mg, 0.44 mmol) in THF (3 mL) and water (0.5 mL) was stirred at rt for 1 h. The solvent was removed under reduced pressure and the reaction mixture was acidified with HCl (2 M). The reaction mixture was diluted with EtOAc and washed with water. The organic layer was dried over Na₂SO₄, filtered and evaporated to give the title compound (45 mg, 79%) which was used in the next step without further purification: MS (ESI) m/z [M+H]⁺ 258.

### Intermediate 166: Methyl 6-cyclopropylquinoline-4-carboxylate

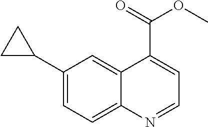 [SMILES: COC(=O)c1ccnc2ccc(C3CC3)cc12]

Pd(dppf)Cl₂ (49 mg, 0.07 mmol) was added to methyl 6-bromoquinoline-4-carboxylate (180 mg, 0.68 mmol), cyclopropylboronic acid (87 mg, 1.0 mmol) and K₂CO₃ (187 mg, 1.35 mmol) in 1,4-dioxane (5 mL) under N₂ (g) atmosphere and the reaction mixture was stirred at 80° C. for 4 h. The reaction mixture was filtered, and the filtrate was concentrated under vacuum. The crude product was purified by preparative TLC (petroleum ether:EtOAc, 1:1), to give the title compound (120 mg, 78%) as a white solid; MS (ESI) m/z [M+H]⁺ 228.

### Intermediate 167: 6-Cyclopropylquinoline-4-carboxylic Acid

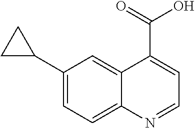 [SMILES: O=C(O)c1ccnc2ccc(C3CC3)cc12]

A solution of methyl 6-cyclopropylquinoline-4-carboxylate Intermediate 166 (120 mg, 0.53 mmol) and LiOH (25 mg, 1.1 mmol) in THF (5 mL) and water (1 mL) was stirred at rt for 1 h. The solvent was removed under reduced pressure and the reaction mixture was acidified with HCl (0.1 M). The reaction mixture was diluted with EtOAc and washed with water. The organic layer was dried over Na₂SO₄, filtered and evaporated to give the title compound (80 mg, 71%) which was used without further purification: MS (ESI) m/z [M+H]⁺ 214.

### Intermediate 168: N′-(4-Methoxycyclohexylidene)-4-methylbenzene-sulfonohydrazide

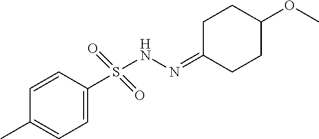

4-Methylbenzenesulfonohydrazide (799 mg, 4.29 mmol) was added to a solution of 4-methoxycyclohexan-1-one (500 mg, 3.90 mmol) in MeCN (20 mL) and the reaction mixture was stirred at 60° C. for 16 h. The solvent was removed under reduced pressure and the crude product was purified by straight phase flash chromatography on silica (gradient: 30-50% EtOAc in petroleum ether) to give the title compound (1.1 g, 95%) as a pale yellow solid: MS (ESI) m/z [M+H]⁺ 297.0.

### Intermediate 169: (4-(tert-Butoxycarbonyl)quinolin-6-yl)boronic Acid

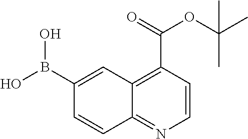 [SMILES: CC(C)(C)OC(=O)c1ccnc2ccc(B(O)O)cc12]

XPhos (31 mg, 0.06 mmol) and XPhos-Pd-G2 (26 mg, 0.03 mmol) were added to a solution of tert-butyl 6-bromoquinoline-4-carboxylate (WO 2019154886) (500 mg, 1.62 mmol), B₂(OH)₄ (436 mg, 4.87 mmol) and KOAc (478 mg, 4.87 mmol) in EtOH (20 mL, 99.5%) at 20° C. The reaction mixture was stirred at 80° C. for 12 h under an atmosphere of N₂ (g). The reaction mixture was poured into sat brine (150 mL), extracted with EtOAc (3×100 mL), and the combined organic layers were dried over Na₂SO₄, filtered and evaporated to give the title compound (0.51 g) as a crude as a brown solid: MS (ESI) m/z [M+H]⁺ 274.2.

### Intermediate 170: tert-Butyl 6-((1r,4SR)-4-methoxycyclohexyl)quinoline-4-carboxylate

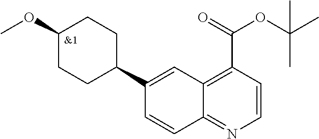 [SMILES: CO[C@H]1CC[C@@H](c2ccc3nccc(C(=O)OC(C)(C)C)c3c2)CC1]

(4-(tert-Butoxycarbonyl)quinolin-6-yl)boronic acid Intermediate 169 (507 mg, 1.86 mmol) was added to a solution of N-(4-methoxycyclohexylidene)-4-methylbenzenesulfonohydrazide Intermediate 168 (250 mg, 0.84 mmol) and K₂CO₃ (933 mg, 6.75 mmol) in 1,4-dioxane (80 mL) at 20° C., and the reaction mixture was stirred at 110° C. for 15 h. The reaction mixture was poured into sat brine (150 mL), extracted with EtOAc (3×100 mL), and the combined organic layers were dried over Na₂SO₄, filtered and evaporated. The crude product was purified by preparative TLC (EtOAc:petroleum ether, 1:2), to give the title compound (84 mg, 29%) as a pale yellow gum: MS (ESI) m/z [M+H]⁺ 342.3.

### Intermediate 171: 6-((1r,4SR)-4-Methoxycyclohexyl)quinoline-4-carboxylic Acid

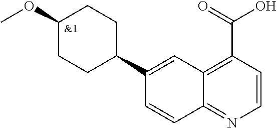 [SMILES: CO[C@H]1CC[C@@H](c2ccc3nccc(C(=O)O)c3c2)CC1]

TFA (2.0 mL, 26 mmol) was added to tert-butyl 6-(4-methoxycyclohexyl)quinoline-4-carboxylate Intermediate 170 (205 mg, 0.60 mmol) in DCM (2 mL) and the reaction mixture was stirred at 20° C. for 16 h. The solvent was removed under reduced pressure to give the title compound (0.375 g) as a brown gum: MS (ESI) m/z [M+H]⁺ 286.

### Intermediate 172: N-(2-((R)-4-Cyanothiazolidin-3-yl)-2-oxoethyl)-6-((1r,4RS)-4-methoxycyclohexyl)quinoline-4-carboxamide

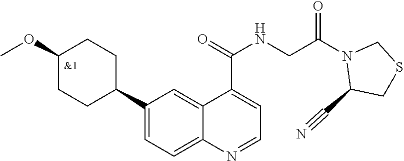 [SMILES: CO[C@H]1CC[C@@H](c2ccc3nccc(C(=O)NCC(=O)N4CSC[C@H]4C#N)c3c2)CC1]

DIPEA (4.5 mL, 26 mmol) was added to a solution of 6-(4-methoxycyclohexyl)quinoline-4-carboxylic acid Intermediate 171 (365 mg, 1.28 mmol), (R)-3-glycylthiazolidine-4-carbonitrile hydrochloride Intermediate 4 (398 mg, 1.92 mmol), HOBt (1.73 g, 12.8 mmol) and EDC (2.45 g, 12.8 mmol) in MeCN (6.0 mL) and EtOAc (6 mL) at 20° C., and the reaction mixture was stirred at 50° C. for 2 h under N₂ (g). The solvent was removed under reduced pressure, and the residue was diluted with sat NaHCO₃ (aq, 200 mL) and washed with EtOAc (4×200 mL). The combined organic layer was dried over Na₂SO₄, filtered, and evaporated to afford crude product.

### Intermediate 173: 6-Phenoxyquinoline-4-carboxylic Acid

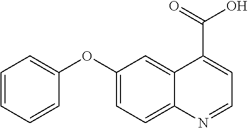 [SMILES: O=C(O)c1ccnc2ccc(Oc3ccccc3)cc12]

### Step a) Ethyl 6-phenoxyquinoline-4-carboxylate

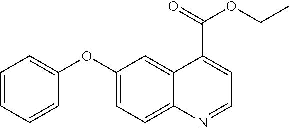 [SMILES: CCOC(=O)c1ccnc2ccc(Oc3ccccc3)cc12]

A mixture of ethyl 6-bromoquinoline-4-carboxylate (155 mg, 0.55 mmol), t-BuXPhos (7 mg, 0.02 mmol), K₃PO₄ (292 mg, 1.37 mmol), Pd(OAc)₂ (2.5 mg, 11 μmol) and phenol (62 mg, 0.66 mmol) in toluene (1.8 mL) was purged with N₂ (g) The reaction vessel was capped and heated at 90° C. overnight. The reaction mixture was diluted with EtOAc and washed sequentially with water and brine. The organic phase was dried using a phase-separator and concentrated at reduced pressure, to give the subtitle compound (158 mg, 98%): MS (ESI) m/z [M+H]⁺ 294.2.

### Step b) 6-Phenoxyquinoline-4-carboxylic Acid

1 M NaOH (aq, 0.539 mL, 0.54 mmol) was added to a solution of ethyl 6-phenoxyquinoline-4-carboxylate (158 mg, 0.54 mmol) in MeOH (0.4 ml) and the reaction mixture was heated at 60° C. for 2 h. 3.8 M HCl (aq, 0.14 mL, 0.54 mmol) was added and the mixture was concentrated under reduced pressure to give the crude title compound (125 mg) as a white solid: MS (ESI) m/z [M+H]⁺ 266.1.

### Intermediate 174: rac-Methyl (R)-6-(1-methoxyethyl)quinoline-4-carboxylate

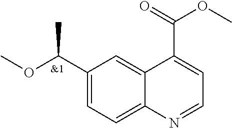

NaH (41.5 mg, 1.04 mmol) was added to rac-methyl (R)-6-(1-hydroxyethyl)quinoline-4-carboxylate Intermediate 63 (120 mg, 0.52 mmol) in DMF (3 mL) cooled to 0° C. The resulting mixture was stirred at 0° C. for 10 min. Then iodomethane (110 mg, 0.78 mmol) was added, and the resulting mixture was stirred at rt for 1 h. The solvent was removed under reduced pressure and the residue was purified by preparative TLC (petroleum ether:EtOAc, 1:1), to afford the title compound (80 mg, 63%) as a colourless solid. MS (ESI) m/z [M+H]⁺ 246.0.

### Intermediate 175: rac-(R)-6-(1-Methoxyethyl)quinoline-4-carboxylic Acid

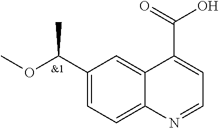 [SMILES: CO[C@@H](C)c1ccc2nccc(C(=O)O)c2c1]

LiOH (12 mg, 0.49 mmol) was added to a solution of rac-methyl (R)-6-(1-methoxyethyl)quinoline-4-carboxylate Intermediate 174 (30 mg, 0.12 mmol) in MeOH (0.9 mL) and water (0.3 mL) and the reaction mixture was stirred at 20° C. for 3 h. The solvent was removed under reduced pressure and the residue was acidified with 2 M HCl (aq) to pH˜6-7. The aqueous phase was extracted with DCM, and concentrated in vacuo to give the crude title compound (30 mg) as pale yellow solid; MS (ESI) m/z [M+H]⁺ 232

### Intermediate 176: 6-(2-Methoxypropan-2-yl)quinoline-4-carboxylic Acid

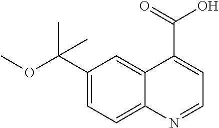 [SMILES: COC(C)(C)c1ccc2nccc(C(=O)O)c2c1]

LiOH (9 mg, 0.39 mmol) was added to a solution of methyl 6-(2-methoxypropan-2-yl)quinoline-4-carboxylate Intermediate 87 (50 mg, 0.19 mmol) in MeOH (0.9 mL) and water (0.3 mL) and the reaction mixture was stirred at 20° C. for 4 h. The reaction mixture was acidified with 2 M HCl (aq) to pH˜5-6, and the reaction mixture was concentrated to dryness to give the crude title compound (60 mg) as pale yellow solid; MS (ESI) m/z [M+H]⁺ 246.

### C. Final Compounds

### Example 1: (R)—N-(2-(4-Cyanothiazolidin-3-yl)-2-oxoethyl)-6-phenoxyquinoline-4-carboxamide

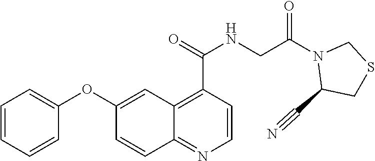 [SMILES: N#C[C@@H]1CSCN1C(=O)CNC(=O)c1ccnc2ccc(Oc3ccccc3)cc12]

To a suspension of 6-phenoxyquinoline-4-carboxylic acid Intermediate 173 (46 mg, 0.17 mmol), HOBt (27 mg, 0.17 mmol) and EDC (42 mg, 0.22 mmol) in EtOAc (1 mL) and MeCN (1 mL) was added crude (R)-3-glycylthiazolidine-4-carbonitrile hydrochloride Intermediate 4 (30 mg, 0.14 mmol) and DIPEA (0.076 mL, 0.43 mmol) to give a milky suspension that was stirred at rt overnight. The mixture was diluted with EtOAc, washed with sat NaHCO₃ (aq) and brine, dried, filtered and evaporated. The residue was purified by preparative HPLC, PrepMethod G (gradient: 5-90%) to give the title compound (17 mg, 27%); HRMS (ESI) m/z [M+H]⁺ calcd for C₂₂H₁₉N₄O₃S: 419.1172, found: 419.1174; ¹H NMR (600 MHz, DMSO-d₆) 9.07 (t, 1H), 8.93 (d, 1H), 8.12 (d, 1H), 7.89 (d, 1H), 7.60 (d, 1H), 7.55 (dd, 1H), 7.47-7.41 (m, 2H), 7.21 (t, 1H), 7.14-7.09 (m, 2H), 5.31 (dd, 1H), 4.84 (d, 1H), 4.68 (d, 1H), 4.28-4.24 (m, 2H), 3.43-3.36 (m, overlapping with solvent peak).

### Example 2: (R)—N-(2-(4-Cyanothiazolidin-3-yl)-2-oxoethyl)-6-(2,2-difluoroethoxy)quinoline-4-carboxamide

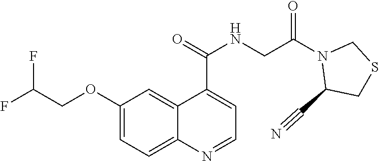 [SMILES: N#C[C@@H]1CSCN1C(=O)CNC(=O)c1ccnc2ccc(OCC(F)F)cc12]

HATU (293 mg, 0.77 mmol) and DIPEA (359 μL, 2.05 mmol) were added to a solution of 6-(2,2-difluoroethoxy)quinoline-4-carboxylic acid Intermediate 154 (130 mg, 0.51 mmol) and (R)-3-glycylthiazolidine-4-carbonitrile hydrochloride Intermediate 4 (88 mg, 0.51 mmol) in DMF (5 mL) and the reaction mixture was stirred at 25° C. for 2 h. The solvent was removed under reduced pressure and the crude product was purified by preparative TLC (DCM:MeOH, 19:1) followed by purification by preparative HPLC, PrepMethod H (gradient: 23-43%) to give the title compound (76 mg, 36%); HRMS (ESI) m/z [M+H]⁺ calcd for C₁₈H₁₇F₂N₄O₃S: 407.0984, found: 407.0982; ¹H NMR (300 MHz, DMSO-d₆) δ 9.11 (t, 1H) 8.85 (d, 1H) 8.03 (d, 1H) 7.97 (d, 1H) 7.60-7.50 (m, 2H) 6.47 (tt, 1H) 5.32 (dd, 1H) 4.90 (d, 1H) 4.71 (d, 1H) 4.47 (td, 2H) 4.33 (d, 2H) 3.46-3.34 (m, 2H).

### Example 3: (R)—N-(2-(4-Cyanothiazolidin-3-yl)-2-oxoethyl)-6-(2-fluoro-2-methylpropoxy)quinoline-4-carboxamide

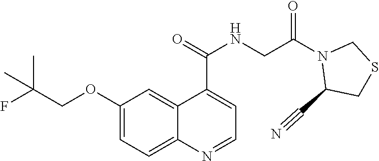 [SMILES: CC(C)(F)COc1ccc2nccc(C(=O)NCC(=O)N3CSC[C@H]3C#N)c2c1]

To a stirred solution of 6-(2-fluoro-2-methylpropoxy)quinoline-4-carboxylic acid Intermediate 152 (2.0 g, 7.6 mmol) in DMF (10 mL) was added (R)-3-glycylthiazolidine-4-carbonitrile hydrochloride Intermediate 4 (1.58 g, 7.60 mmol), T3P (19.3 g, 30.4 mmol, 50% in EtOAc), DIPEA (5.3 mL, 30 mmol) at 25° C. under air. The resulting solution was stirred at 25° C. for 2 h. The reaction mixture was diluted with water (100 mL) and extracted with DCM (2×200 mL). The organic layer was dried over Na₂SO₄, filtered and evaporated. The residue was purified by flash chromatography on silica (gradient: 0-5% MeOH in DCM). Fractions containing product were combined and evaporated to afford crude product which was further purified by preparative TLC (DCM:MeOH, 20:1), to give the title compound (1.78 g, 56%); HRMS (ESI) m/z [M+H]⁺ calcd for C₂₀H₂₂FN₄O₃S: 417.1390, found: 417.1392; ¹H NMR (300 MHz, DMSO-d₆) δ 9.11 (t, 1H), 8.82 (d, 1H), 8.05-7.92 (m, 2H), 7.57-7.45 (m, 2H), 5.33 (dd, 1H), 4.91 (d, 1H), 4.72 (d, 1H), 4.34 (d, 2H), 4.21 (d, 2H) 3.48-3.34 (m, 2H), 1.50 (d, 6H).

### Example 4: (R)—N-(2-(4-Cyanothiazolidin-3-yl)-2-oxoethyl)-6-(2-methoxyethoxy)quinoline-4-carboxamide

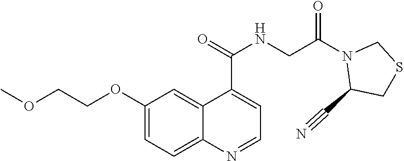 [SMILES: COCCOc1ccc2nccc(C(=O)NCC(=O)N3CSC[C@H]3C#N)c2c1]

To a stirred solute-on of 6-(2-methoxyethoxy)quinoline-4-carboxylic acid Intermediate 19 (120 mg, 0.49 mmol) in DMF (5 mL) was added (R)-3-glycylthiazolidine-4-carbonitrile hydrochloride Intermediate 4 (101 mg, 0.49 mmol), DIPEA (339 μL, 1.94 mmol), HATU (277 mg, 0.73 mmol). The resulting solution was stirred at 25° C. for 2 h. The solvent was removed under reduced pressure. The residue was purified by preparative TLC (DCM:MeOH, 19:1) to afford crude product, followed by purification by preparative HPLC, PrepMethod H (gradient 17-37%) to give the title compound (41 mg, 21%); HRMS (ESI) m/z [M+H]⁺ calcd for C₁₉H₂₁N₄O₄S: 401.1278, found: 401.1272; ¹H NMR (300 MHz, DMSO-d₆) δ 9.08 (t, 1H), 8.80 (d, 1H), 8.05-7.82 (m, 2H), 7.61-7.34 (m, 2H), 5.32 (dd, 1H), 4.89 (d, 1H), 4.70 (d, 1H), 4.44-4.12 (m, 4H), 3.86-3.61 (m, 2H), 3.50-3.20 (m, 5H).

### Example 5: (R)—N-(2-(4-Cyanothiazolidin-3-yl)-2-oxoethyl)-6-(cyclopropyl-methoxy)quinoline-4-carboxamide

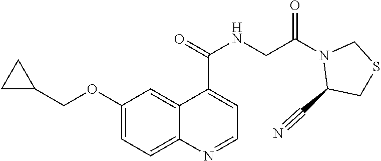 [SMILES: N#C[C@@H]1CSCN1C(=O)CNC(=O)c1ccnc2ccc(OCC3CC3)cc12]

DIPEA (2.34 mL, 13.4 mmol) was added to 6-(cyclopropylmethoxy)quinoline-4-carboxylic acid Intermediate 21 (163 mg, 0.67 mmol), (R)-3-glycylthiazolidine-4-carbonitrile hydrochloride Intermediate 4 (209 mg, 1.01 mmol), HOBt (905 mg, 6.70 mmol) and EDC (1.29 g, 6.70 mmol) in MeCN (6 mL) and EtOAc (6 mL) at 25° C. The resulting solution was stirred at 25° C. for 16 h under N₂ (g). The solvent was removed under reduced pressure. The residue was diluted with sat NaHCO₃ (aq, 250 mL) and extracted with EtOAc (3×250 mL). The organic layers were combined and washed sequentially with water (3×50 mL). The organic layer was dried over Na₂SO₄, filtered and evaporated to afford crude product that was purified by preparative HPLC, PrepMethod F (gradient 30-40%) to give the title compound (134 mg, 50%); HRMS (ESI) m/z [M+H]⁺ calcd for C₂₀H₂₁N₄O₃S: 397.1328, found: 397.1336; ¹H NMR (400 MHz, DMSO-d₆) δ 9.08 (t, 1H), 8.80 (d, 1H), 7.98 (d, 1H), 7.85 (d, 1H), 7.53-7.44 (m, 2H), 5.33 (dd, 1H), 4.90 (d, 1H), 4.71 (d, 1H), 4.32 (d, 2H), 4.07-3.99 (m, 2H), 3.59-3.33 (m, overlapping with solvent peak), 1.37-1.22 (m, 1H), 0.65-0.56 (m, 2H), 0.45-0.34 (m, 2H).

### Example 6: (R)—N-(2-(4-Cyanothiazolidin-3-yl)-2-oxoethyl)-6-((tetrahydro-2H-pyran-4-yl)oxy)quinoline-4-carboxamide

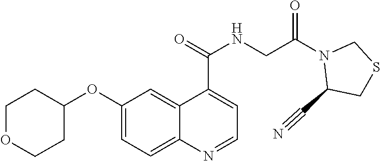 [SMILES: N#C[C@@H]1CSCN1C(=O)CNC(=O)c1ccnc2ccc(OC3CCOCC3)cc12]

DIPEA (997 μL, 5.71 mmol) was added to 6-((tetrahydro-2H-pyran-4-yl)oxy)quinoline-4-carboxylic acid Intermediate 23 (156 mg, 0.57 mmol), (R)-3-glycylthiazolidine-4-carbonitrile hydrochloride Intermediate 4 (178 mg, 0.86 mmol), HOBt (771 mg, 5.71 mmol) and EDC (1.09 g, 5.71 mmol) in MeCN (6 mL) and EtOAc (6 mL) at 25° C. The resulting solution was stirred under N₂ (g) at 25° C. for 16 h. The solvent was removed under reduced pressure. The residue was diluted with sat NaHCO₃ (aq, 250 mL) and extracted with EtOAc (3×250 mL). The organic layers were combined, washed sequentially with water (3×50 mL), dried over Na₂SO₄, filtered and evaporated to give crude product that was purified by preparative HPLC, PrepMethod F (gradient 19-30%) to give the title compound (126 mg, 51%); HRMS (ESI) m/z [M+H]⁺ calcd for C₂₁H₂₃N₄O₄S: 427.1434, found: 427.1436; ¹H NMR (400 MHz, DMSO-d₆) δ 9.11 (t, 1H), 8.79 (d, 1H), 8.01-7.95 (m, 2H), 7.50-7.42 (m, 2H), 5.34 (dd, 1H), 4.94-4.82 (m, 2H), 4.70 (d, 1H), 4.31 (d, 2H), 3.97-3.83 (m, 2H), 3.68-3.56 (m, 2H), 3.48-3.33 (m, overlapping with solvent peak), 2.17-2.06 (m, 2H), 1.71-1.59 (m, 2H).

### Example 7: (R)—N-(2-(4-Cyanothiazolidin-3-yl)-2-oxoethyl)-6-((1,3-difluoropropan-2-yl)oxy)quinoline-4-carboxamide

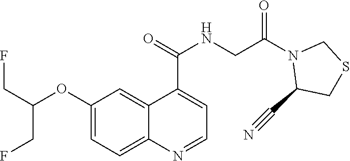 [SMILES: N#C[C@@H]1CSCN1C(=O)CNC(=O)c1ccnc2ccc(OC(CF)CF)cc12]

DIPEA (536 μL, 3.07 mmol) was added to 6-((1,3-difluoropropan-2-yl)oxy)quinoline-4-carboxylic acid Intermediate 25 (164 mg, 0.61 mmol), (R)-3-glycylthiazolidine-4-carbonitrile hydrochloride Intermediate 4 (191 mg, 0.92 mmol), HOBt (829 mg, 6.14 mmol) and EDC (1.18 g, 6.14 mmol) in MeCN (6 mL) and EtOAc (6 mL) at 25° C. The resulting solution was stirred at 25° C. for 16 h under N₂ (g). The solvent was removed under reduced pressure. The residue was diluted with sat NaHCO₃ (aq, 200 mL), extracted with EtOAc (3×200 mL). The organic layers were combined, washed sequentially with water (3×50 mL), dried over Na₂SO₄, filtered and evaporated to give crude product that was purified by preparative HPLC, PrepMethod D (gradient 24-34%) to give the title compound (145 mg, 56%); HRMS (ESI) m/z [M+H]⁺ calcd for C₁₉H₁₉F₂N₄O₃S: 421.1140, found: 421.1156; ¹H NMR (300 MHz, DMSO-d₆) δ 9.13 (t, 1H), 8.85 (d, 1H), 8.16 (d, 1H), 8.04 (d, 1H), 7.60-7.49 (m, 2H), 5.38 (dd, 1H), 5.32-5.07 (m, 1H), 5.01-4.56 (m, 6H), 4.35 (d, 2H), 3.49-3.33 (m, overlapping with solvent peak).

### Example 8: (R)—N-(2-(4-Cyanothiazolidin-3-yl)-2-oxoethyl)-6-((3,3-difluorocyclobutyl)methoxy)quinoline-4-carboxamide

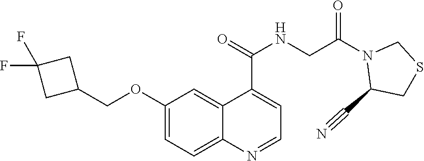 [SMILES: N#C[C@@H]1CSCN1C(=O)CNC(=O)c1ccnc2ccc(OCC3CC(F)(F)C3)cc12]

DIPEA (66 μL, 0.38 mmol) was added to 6-((3,3-difluorocyclobutyl)methoxy)quinoline-4-carboxylic acid Intermediate 27 (110 mg, 0.38 mmol), (R)-3-glycylthiazolidine-4-carbonitrile hydrochloride Intermediate 4 (78 mg, 0.38 mmol), HOBt (51 mg, 0.38 mmol) and EDC (72 mg, 0.38 mmol) in MeCN (6 mL) and EtOAc (6 mL) at 25° C. The resulting solution was stirred under N₂ (g) at 25° C. for 16 h. The solvent was removed under reduced pressure. The residue was diluted with sat NaHCO₃ (aq, 200 mL) and extracted with EtOAc (3×200 mL). The organic layers were combined, washed sequentially with water (3×50 mL), dried over Na₂SO₄, filtered and evaporated to give the crude product that was purified by preparative HPLC, PrepMethod D (gradient 30-40%) to give the title compound (86 mg, 51%); HRMS (ESI) m/z [M+H]⁺ calcd for C₂₁H₂₁F₂N₄O₃S: 447.1296, found: 447.1296; ¹H NMR (300 MHz, DMSO-d₆) δ 9.13 (t, 1H), 8.82 (d, 1H), 8.04-7.93 (m, 2H), 7.57-7.43 (m, 2H), 5.33 (dd, 1H), 4.91 (d, 1H), 4.71 (d, 1H), 4.45-4.28 (m, 2H), 4.25 (d, 2H), 3.76-3.33 (m, overlapping with solvent peak), 2.88-2.50 (m, overlapping with solvent peak).

### Example 9: N-(2-((R)-4-Cyanothiazolidin-3-yl)-2-oxoethyl)-6-(((RS)-2,2-dimethyltetrahydro-2H-pyran-4-yl)oxy)quinoline-4-carboxamide

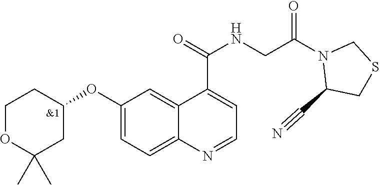 [SMILES: CC1(C)C[C@@H](Oc2ccc3nccc(C(=O)NCC(=O)N4CSC[C@H]4C#N)c3c2)CCO1]

DIPEA (548 μL, 3.14 mmol) was added to rac-(R)-6-((2,2-dimethyltetrahydro-2H-pyran-4-yl)oxy)quinoline-4-carboxylic acid Intermediate 29 (189 mg, 0.63 mmol), (R)-3-glycylthiazolidine-4-carbonitrile hydrochloride Intermediate 4 (195 mg, 0.94 mmol), HOBt (848 mg, 6.27 mmol) and EDC (1.20 mg, 6.27 mmol) in MeCN (6 mL) and EtOAc (6 mL) at 25° C. The resulting solution was stirred under N₂ (g) at 50° C. for 16 h. The solvent was removed under reduced pressure. The residue was diluted with sat NaHCO₃ (aq, 200 mL) and extracted with EtOAc (3×200 mL). The organic layers were combined, washed sequentially with water (3×50 mL), dried over Na₂SO₄, filtered and evaporated to give the crude product that was purified by preparative HPLC, PrepMethod D (gradient 21-31%) to give the title compound (0.124 g, 44%); HRMS (ESI) m/z [M+H]⁺ calcd for C₂₃H₂₇N₄O₄S: 455.1748, found: 455.1730; ¹H NMR (400 MHz, DMSO-d₆) δ 9.18-9.09 (m, 1H), 8.80 (d, 1H), 8.01-7.91 (m, 2H), 7.50-7.40 (m, 2H), 5.34-5.26 (m, 1H), 5.04-4.94 (m, 1H), 4.91 (dd, 1H), 4.73 (dd, 1H), 4.46-4.19 (m, 2H), 3.89-3.69 (m, 2H), 3.60-3.33 (m, overlapping with solvent peak), 2.22-2.10 (m, 1H), 2.08-1.95 (m, 1H), 1.59-1.39 (m, 2H), 1.30 (s, 3H), 1.23 (s, 3H).

### Example 10: (R)-6-(Cyanomethoxy)-N-(2-(4-cyanothiazolidin-3-yl)-2-oxoethyl)quinoline-4-carboxamide

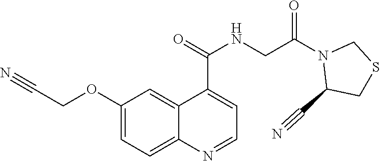 [SMILES: N#CCOc1ccc2nccc(C(=O)NCC(=O)N3CSC[C@H]3C#N)c2c1]

EDC (504 mg, 2.63 mmol), DIPEA (2.3 mL, 13 mmol) and HOBt (355 mg, 2.63 mmol) were added to 6-(cyanomethoxy)quinoline-4-carboxylic acid Intermediate 33 (300 mg, 1.31 mmol) and (R)-3-glycylthiazolidine-4-carbonitrile hydrochloride Intermediate 4 (338 mg, 1.97 mmol) in EtOAc (2 mL) and MeCN (2 mL). The resulting solution was stirred at 20° C. for 18 h. The reaction mixture was diluted with EtOAc (20 mL) and washed sequentially with sat NaHCO₃ (aq, 3×20 mL). The organic layer was dried over Na₂SO₄, filtered and evaporated. The residue was purified by preparative TLC (MeOH:DCM, 1:40), to give crude product. The product was purified by preparative HPLC PrepMethod D (gradient (21-31%) to give the title compound (8 mg, 4%) after freeze drying; HRMS (ESI) m/z [M+H]⁺ calcd for C₁₈H₁₆N₅O₃S: 382.0968, found: 382.0960; ¹H NMR (400 MHz, CD₃OD) δ 8.86 (d, 1H), 8.21 (d, 1H), 8.08 (d, 1H), 7.65 (d, 1H), 7.58 (dd, 1H), 5.39 (dd, 1H), 5.25-5.21 (m, 2H), 4.86-4.77 (m, overlapping with solvent peak), 4.51-4.35 (m, 2H), 3.45-3.35 (m, 2H).

### Example 11: (R)—N-(2-(4-Cyanothiazolidin-3-yl)-2-oxoethyl)-6-(2,2-difluoropropoxy)quinoline-4-carboxamide

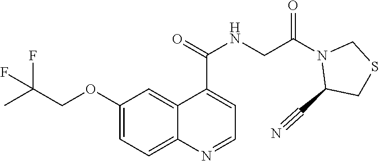 [SMILES: CC(F)(F)COc1ccc2nccc(C(=O)NCC(=O)N3CSC[C@H]3C#N)c2c1]

A solution of 6-(2,2-difluoropropoxy)quinoline-4-carboxylic acid Intermediate 36 (80 mg, 0.30 mmol), (R)-3-glycylthiazolidine-4-carbonitrile hydrochloride Intermediate 4 (93 mg, 0.45 mmol), EDC (86 mg, 0.45 mmol), HOBt (61 mg, 0.45 mmol) and DIPEA (261 μL, 1.50 mmol) in MeCN (5 mL) and EtOAc (5 mL) was stirred at 50° C. for 1 h. The solvent was removed under reduced pressure. The mixture was diluted with EtOAc (25 mL) and washed with water (3×10 mL). The organic layer was dried over Na₂SO₄, filtered and evaporated to give the crude product which was purified by preparative HPLC, PrepMethod D (gradient 26-37%) to give the title compound (75 mg, 59%); HRMS (ESI) m/z [M+H]⁺ calcd for C₁₉H₁₉F₂N₄O₃S: 421.1140, found: 421.1142; ¹H NMR (400 MHz, CD₃OD) δ 8.82 (d, 1H), 8.08-7.99 (m, 2H), 7.62 (d, 1H), 7.57 (dd, 1H), 5.36 (dd, 1H), 4.90-4.86 (m, overlapping with solvent peak), 4.81 (d, 1H), 4.53-4.46 (m, 4H), 3.49-3.36 (m, 2H), 1.83 (t, 3H).

### Example 12: N-(2-((R)-4-Cyanothiazolidin-3-yl)-2-oxoethyl)-6-((RS)-1-(3-fluoropyridin-2-yl)ethyl)quinoline-4-carboxamide

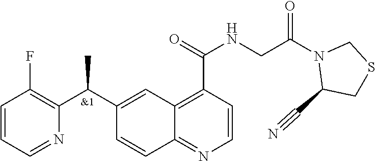 [SMILES: C[C@@H](c1ccc2nccc(C(=O)NCC(=O)N3CSC[C@H]3C#N)c2c1)c1ncccc1F]

DIPEA (360 μL, 2.06 mmol) was added to rac-(R)-6-(1-(3-fluoropyridin-2-yl)ethyl)quinoline-4-carboxylic acid Intermediate 40 (65 mg, 0.10 mmol), (R)-3-glycylthiazolidine-4-carbonitrile hydrochloride Intermediate 4 (43 mg, 0.21 mmol), HOBt (139 mg, 1.03 mmol) and EDC (198 mg, 1.03 mmol) in MeCN (5 mL) and EtOAc (5 mL) at 15° C. The resulting solution was stirred under N₂ (g) at 15° C. for 16 h. The solvent was removed under reduced pressure. The residue was diluted with sat NaHCO₃ (aq, 100 mL) and extracted with EtOAc (3×100 mL). The organic layer was dried over Na₂SO₄, filtered and evaporated to give crude product that was purified by preparative HPLC, PrepMethod D (gradient 25-40%) to give the title compound (20 mg, 43%) after evaporation of relevant fractions; HRMS (ESI) m/z [M+H]⁺ calcd for C₂₃H₂₁FN₅O₂S: 450.1394, found: 450.1378; ¹H NMR (300 MHz, CDCl₃) δ 8.92 (d, 1H), 8.50-8.38 (m, 1H), 8.36-8.25 (m, 1H), 8.20-8.08 (m, 1H), 7.82 (dt, 1H), 7.55 (d, 1H), 7.40-7.30 (m, 1H), 7.24-7.14 (m, 1H), 7.13-6.97 (m, 1H), 5.41-5.29 (m, 1H), 4.83 (q, 1H), 4.77-4.64 (m, 2H), 4.61-4.46 (m, 1H), 4.45-4.30 (m, 1H), 3.37 (d, 2H), 1.83 (d, 3H).

### Example 13: (R)—N-(2-(4-Cyanothiazolidin-3-yl)-2-oxoethyl)-6-(1-(2-(trifluoromethyl)pyridin-4-yl)cyclopropyl)quinoline-4-carboxamide

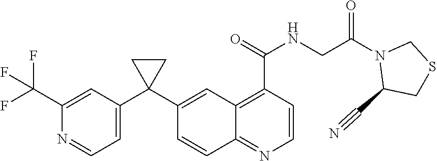 [SMILES: N#C[C@@H]1CSCN1C(=O)CNC(=O)c1ccnc2ccc(C3(c4ccnc(C(F)(F)F)c4)CC3)cc12]

DIPEA (590 μL, 3.38 mmol) was added to 6-(1-(2-(trifluoromethyl)pyridin-4-yl)cyclopropyl)quinoline-4-carboxylic acid Intermediate 46 (121 mg, 0.34 mmol), (R)-3-glycylthiazolidine-4-carbonitrile hydrochloride Intermediate 4 (140 mg, 0.68 mmol) and HATU (385 mg, 1.01 mmol) in MeCN (5 mL) and EtOAc (5 mL) at 15° C. The resulting solution was stirred under N₂ (g) at 50° C. for 2 h. The solvent was removed under reduced pressure. The residue was diluted with sat NaHCO₃ (aq, 200 mL) and extracted with EtOAc (4×200 mL). The organic layers were combined and washed with water (3×50 mL). The organic layer was dried over Na₂SO₄, filtered and evaporated. The crude product was purified by preparative HPLC, PrepMethod D (gradient 35-50%) to give the title compound (54 mg, 31%); HRMS (ESI) m/z [M+H]⁺ calcd for C₂₅H₂₁F₃N₅O₂S: 512.1362, found: 512.1366; ¹H NMR (300 MHz, CDCl₃) δ 8.99 (d, 1H), 8.58 (d, 1H), 8.36-8.23 (m, 2H), 7.74 (dd, 1H), 7.66 (d, 1H), 7.39 (s, 1H), 7.24-7.06 (m, 2H), 5.33 (t, 1H), 4.72-4.60 (m, 2H), 4.56-4.28 (m, 2H), 3.38 (d, 2H), 1.73-1.62 (m, 2H), 1.58-1.48 (m, 2H).

### Example 14: (R)—N-(2-(4-Cyanothiazolidin-3-yl)-2-oxoethyl)-6-(furan-3-yl)quinoline-4-carboxamide

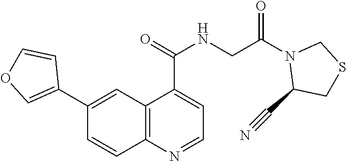 [SMILES: N#C[C@@H]1CSCN1C(=O)CNC(=O)c1ccnc2ccc(-c3ccoc3)cc12]

6-(Furan-3-yl)quinoline-4-carboxylic acid Intermediate 47 (15 mg, 0.06 mmol), HATU (36 mg, 0.09 mmol) and DIPEA (44 μL, 0.25 mmol) were mixed in MeCN (0.5 mL) and EtOAc (0.5 mL). (R)-3-Glycylthiazolidine-4-carbonitrile HCl Intermediate 4 (16 mg, 0.08 mmol) was added, and the reaction stirred at rt for 1 h. DCM (4 mL) and NaHCO₃ (aq, 3 mL) were added. The mixture was stirred and filtered through a phase separator with more DCM and evaporated to afford the crude product which was purified by preparative SFC, PrepMethod SFC-A (gradient 30-35%) to give the title compound (15 mg, 61%); HRMS (ESI) m/z [M+H]⁺ calcd for C₂₀H₁₇N₄O₃S: 393.1016, found: 393.1006; ¹H NMR (600 MHz, DMSO-d₆) 9.13 (t, 1H), 8.88 (d, 1H), 8.70 (d, 1H), 8.38 (s, 1H), 8.09 (dd, 1H), 8.04 (d, 1H), 7.75 (t, 1H), 7.50 (d, 1H), 7.18 (d, 1H), 5.37 (dd, 1H), 4.88 (d, 1H), 4.70 (d, 1H), 4.39-4.27 (m, 2H), 3.44-3.37 (m, overlapping with solvent peak).

### Example 15: (R)—N-(2-(4-Cyanothiazolidin-3-yl)-2-oxoethyl)-6-(4-hydroxy-tetrahydro-2H-pyran-4-yl)quinoline-4-carboxamide

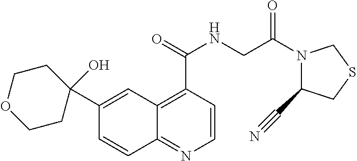 [SMILES: N#C[C@@H]1CSCN1C(=O)CNC(=O)c1ccnc2ccc(C3(O)CCOCC3)cc12]

A solution of 6-(4-hydroxytetrahydro-2H-pyran-4-yl)quinoline-4-carboxylic acid Intermediate 50 (100 mg, 0.37 mmol), (R)-3-glycylthiazolidine-4-carbonitrile hydrochloride Intermediate 4 (114 mg, 0.55 mmol), EDC (140 mg, 0.73 mmol), HOBt (99 mg, 0.73 mmol) and DIPEA (320 μL, 1.83 mmol) in MeCN (4 mL) and EtOAc (4 mL) was stirred at 45° C. for 5 h. The solvent was removed under reduced pressure. The residue was dissolved in EtOAc (30 mL) and washed sequentially with water (3×15 mL). The organic layer was dried over Na₂SO₄, filtered and evaporated to give the crude product which was purified by preparative HPLC, PrepMethod D (gradient 5-30%) to give the title compound (40 mg, 25%); HRMS (ESI) m/z [M+H]⁺ calcd for C₂₁H₂₃N₄O₄S: 427.1434, found: 427.1448; ¹H NMR (300 MHz, CD₃OD) δ 8.90 (d, 1H), 8.55-8.47 (m, 1H), 8.13-7.99 (m, 2H), 7.65 (d, 1H), 5.37 (dd, 1H), 4.87-4.74 (m, overlapping with solvent peak), 4.50-4.36 (m, 2H), 4.07-3.79 (m, 4H), 3.47-3.34 (m, 2H), 2.44-2.18 (m, 2H), 1.88-1.70 (m, 2H).

### Example 16: (R)—N-(2-(4-Cyanothiazolidin-3-yl)-2-oxoethyl)-6-(2-hydroxypropan-2-yl)quinoline-4-carboxamide

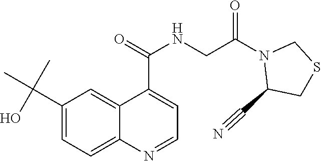 [SMILES: CC(C)(O)c1ccc2nccc(C(=O)NCC(=O)N3CSC[C@H]3C#N)c2c1]

A solution of 6-(2-hydroxypropan-2-yl)quinoline-4-carboxylic acid Intermediate 51 (60 mg, 0.26 mmol), (R)-3-glycylthiazolidine-4-carbonitrile hydrochloride Intermediate 4 (108 mg, 0.52 mmol), EDC (75 mg, 0.39 mmol), HOBt (60 mg, 0.39 mmol) and DIPEA (0.227 mL, 1.30 mmol) in DMF (5 mL) was stirred at rt for 3 h. The solvent was removed under reduced pressure. The residue was purified by preparative TLC (DCM:MeOH, 10:1) to give crude product, that was further purified by preparative HPLC, PrepMethod I (gradient 25-45%) to give the title compound (40 mg, 40%); HRMS (ESI) m/z [M+H]⁺ calcd for C₁₉H₂₁N₄O₃S: 385.1328, found: 385.1312; ¹H NMR (300 MHz, CD₃OD) δ 8.89 (d, 1H), 8.46 (dd, 1H), 8.11-7.98 (m, 2H), 7.65 (d, 1H), 5.37 (dd, 1H), 4.87-4.74 (m, overlapping with solvent peak), 4.52-4.33 (m, 2H), 3.52-3.33 (m, overlapping with solvent peak), 1.65 (s, 6H).

### Example 17: (R)—N-(2-(4-Cyanothiazolidin-3-yl)-2-oxoethyl)-6-(trifluoromethyl)-quinoline-4-carboxamide

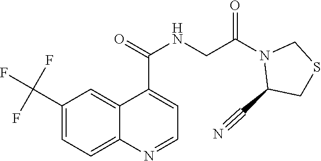 [SMILES: N#C[C@@H]1CSCN1C(=O)CNC(=O)c1ccnc2ccc(C(F)(F)F)cc12]

A solution of 6-(trifluoromethyl)quinoline-4-carboxylic acid Intermediate 52 (80 mg, 0.33 mmol), (R)-3-glycylthiazolidine-4-carbonitrile hydrochloride Intermediate 4 (138 mg, 0.66 mmol), EDC (95 mg, 0.50 mmol), HOBt (76 mg, 0.50 mmol) and DIPEA (0.29 mL, 1.7 mmol) in DMF (5 mL) was stirred at rt for 3 h. The solvent was removed under reduced pressure. The residue was purified by preparative TLC (DCM:MeOH, 20:1) to give crude product that was further purified by preparative HPLC PrepMethod I (gradient 30-40%) to give the title compound (45 mg, 34%); HRMS (ESI) m/z [M+H]⁺ calcd for C₁₇H₁₄F₃N₄O₂S: 395.0784, found: 395.0794; ¹H NMR (300 MHz, CD₃OD) δ 9.10 (d, 1H), 8.87-8.77 (m, 1H), 8.28 (d, 1H), 8.04 (dd, 1H), 7.82 (d, 1H), 5.39 (dd, 1H), 4.87-4.69 (m, overlapping with solvent peak), 4.54-4.33 (m, 2H), 3.53-3.32 (m, overlapping with solvent peak).

### Example 18: (R)—N-(2-(4-Cyanothiazolidin-3-yl)-2-oxoethyl)-6-(2-methoxypropan-2-yl)quinoline-4-carboxamide

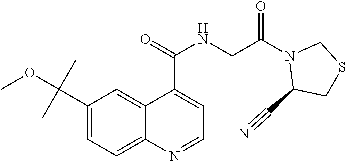 [SMILES: COC(C)(C)c1ccc2nccc(C(=O)NCC(=O)N3CSC[C@H]3C#N)c2c1]

A solution of 6-(2-methoxypropan-2-yl)quinoline-4-carboxylic acid Intermediate 176 (70 mg, 0.29 mmol), (R)-3-glycylthiazolidine-4-carbonitrile hydrochloride Intermediate 4 (119 mg, 0.57 mmol), EDC (82 mg, 0.43 mmol), HOBt (66 mg, 0.43 mmol) and DIPEA (0.25 mL, 1.4 mmol) in DMF (5 mL) was stirred at rt for 3 h. The solvent was removed under reduced pressure. The residue was purified by preparative TLC (DCM:MeOH, 20:1), to give the title compound (45 mg, 39%); HRMS (ESI) m/z [M+H]⁺ calcd for C₂₀H₂₃N₄O₃S: 399.1486, found: 399.1482; ¹H NMR (300 MHz, CD₃OD) δ 8.92 (d, 1H), 8.41 (d, 1H), 8.09 (d, 1H), 7.96 (dd, 1H), 7.66 (d, 1H), 5.37 (dd, 1H), 4.87-4.68 (m, overlapping with solvent peak), 4.52-4.33 (m, 2H), 3.52-3.30 (m, overlapping with solvent peak), 3.11 (s, 3H), 1.64 (s, 6H).

### Example 19: (R)—N-(2-(4-Cyanothiazolidin-3-yl)-2-oxoethyl)-6-((2,5-dimethyl-2H-1,2,3-triazol-4-yl)methyl)quinoline-4-carboxamide

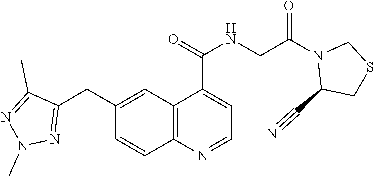 [SMILES: Cc1nn(C)nc1Cc1ccc2nccc(C(=O)NCC(=O)N3CSC[C@H]3C#N)c2c1]

### Step a) 6-((2,5-Dimethyl-2H-1,2,3-triazol-4-yl)methyl)quinoline-4-carboxylic Acid

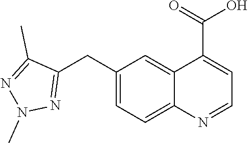 [SMILES: Cc1nn(C)nc1Cc1ccc2nccc(C(=O)O)c2c1]

Ethyl 6-((2,5-dimethyl-2H-1,2,3-triazol-4-yl)methyl)quinoline-4-carboxylate Intermediate 54 (95 mg, 0.31 mmol) was dissolved in MeOH (4 mL) and aq NaOH (612 μL, 0.61 mmol, 1 M) was added. The reaction was stirred at 50° C. for 20 min, then cooled to rt. HCl (201 μL, 0.77 mmol, 3.8 M) was added and the mixture was evaporated and dried azeotropically with EtOH (2×) and MeCN (1×) to give the crude title compound that was used as such in the next step; MS m/z (ESI), [M+H]⁺ 283.3.

### Step b) 6-((2,5-Dimethyl-2H-1,2,3-triazol-4-yl)methyl)quinoline-4-carboxylic Acid

6-((2,5-Dimethyl-2H-1,2,3-triazol-4-yl)methyl)quinoline-4-carboxylic acid (75 mg, 0.27 mmol), HATU (152 mg, 0.40 mmol) and DIPEA (186 μL, 1.06 mmol) were dissolved in MeCN (2 mL) and EtOAc (2 mL). (R)-3-glycylthiazolidine-4-carbonitrile hydrochloride Intermediate 4 (66 mg, 0.32 mmol) was added and the reaction stirred at rt for 3 h. The reaction mixture was diluted with DCM (10 mL) and NaHCO₃ (aq, 7 mL) stirred and filtered through a phase separator with more DCM and evaporated. The residue was purified by preparative HPLC PrepMethod C (gradient 5-50%) to give the title compound (52 mg, 44%). HRMS (ESI) m/z [M+H]⁺ calcd for C₂₁H₂₂N₇O₂S: 436.1550, found: 436.1546; ¹H NMR (500 MHz, DMSO-d₆) δ 9.11 (t, 1H), 8.96 (d, 1H), 8.20 (d, 1H), 8.02 (d, 1H), 7.65 (dd, 1H), 7.58 (d, 1H), 5.36 (dd, 1H), 4.89 (d, 1H), 4.72 (d, 1H), 4.32 (d, 2H), 4.12 (s, 2H), 4.00 (s, 3H), 3.42-3.35 (m, overlapping with solvent peak), 2.16 (s, 3H).

### Example 20: (R)—N-(2-(4-Cyanothiazolidin-3-yl)-2-oxoethyl)-6-(tetrahydro-2H-pyran-4-yl)quinoline-4-carboxamide

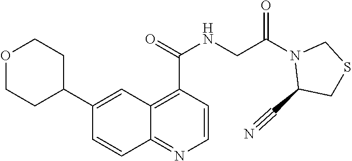 [SMILES: N#C[C@@H]1CSCN1C(=O)CNC(=O)c1ccnc2ccc(C3CCOCC3)cc12]

DIPEA (1.05 mL, 5.99 mmol) was added to 4-carboxy-6-(tetrahydro-2H-pyran-4-yl)quinolin-1-ium 2,2,2-trifluoroacetate Intermediate 56 (224 mg, 0.30 mmol, containing 3.3 eq TFA), (R)-3-glycylthiazolidine-4-carbonitrile hydrochloride Intermediate 4 (62 mg, 0.30 mmol), HOBt (405 mg, 3.00 mmol) and EDC (574 mg, 3.00 mmol) in MeCN (5 mL) and EtOAc (5 mL) at 20° C. The resulting solution was stirred under N₂ (g) at 20° C. for 16 h. The solvent was removed under reduced pressure. The residue was diluted with sat NaHCO₃ (aq, 250 mL) and extracted with EtOAc (3×250 mL). The organic layers were combined and washed with water (3×50 mL), dried over Na₂SO₄, filtered and evaporated to give crude product that was purified by preparative HPLC, PrepMethod D (gradient 10-40%) to give the title compound (30 mg, 24%); HRMS (ESI) m/z [M+H]⁺ calcd for C₂₁H₂₃N₄O₃S: 411.1486, found: 411.1466; ¹H NMR (300 MHz, DMSO-d₆) δ 9.10 (t, 1H), 8.94 (d, 1H), 8.19 (d, 1H), 8.04 (d, 1H), 7.79 (dd, 1H), 7.55 (d, 1H), 5.36 (dd, 1H), 4.91 (d, 1H), 4.73 (d, 1H), 4.34 (d, 2H), 4.05-3.95 (m, 2H), 3.55-3.36 (m, 4H), 3.08-2.91 (m, 1H), 1.89-1.68 (m, 4H).

### Example 21: (R)—N-(2-(4-Cyanothiazolidin-3-yl)-2-oxoethyl)-6-(4-methyl-tetrahydro-2H-pyran-4-yl)quinoline-4-carboxamide

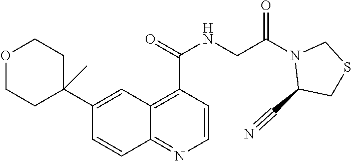 [SMILES: CC1(c2ccc3nccc(C(=O)NCC(=O)N4CSC[C@H]4C#N)c3c2)CCOCC1]

EDC (42 mg, 0.22 mmol) was added slowly to 6-(4-methyltetrahydro-2H-pyran-4-yl)quinoline-4-carboxylic acid Intermediate 58 (20 mg, 0.07 mmol), (R)-3-glycylthiazolidine-4-carbonitrile hydrochloride Intermediate 4 (31 mg, 0.15 mmol), HOBt (30 mg, 0.22 mmol) and TEA (103 μL, 0.74 mmol) in MeCN (5 mL) and EtOAc (5 mL) at 20° C. The resulting solution was stirred at 20° C. for 15 h. The reaction mixture was poured into sat NaHCO₃ (aq, 100 mL) and extracted with EtOAc (3×50 mL). The organic layer was dried over Na₂SO₄, filtered and evaporated to give crude product as a yellow gum. The crude was purified by preparative HPLC PrepMethod J (gradient 20-31%) to give the title compound (13 mg, 21%); HRMS (ESI) m/z [M+H]⁺ calcd for C₂₂H₂₅N₄O₃S: 425.1642, found: 425.1642; ¹H NMR (300 MHz, CD₃OD) δ 8.31 (d, 1H), 8.08 (d, 1H), 7.83-7.73 (m, 2H), 7.68-7.59 (m, 1H), 5.46-5.32 (m, 1H), 4.89-4.76 (m, overlapping with solvent peak), 4.42 (d, 2H), 3.95-3.79 (m, 2H), 3.74-3.57 (m, 2H), 3.50-3.26 (m, overlapping with solvent peak), 2.64-2.45 (m, 2H), 2.01-1.81 (m, 2H), 1.43 (s, 3H).

### Example 22: (R)—N-(2-(4-Cyanothiazolidin-3-yl)-2-oxoethyl)-6-(3,6-dihydro-2H-pyran-4-yl)quinoline-4-carboxamide

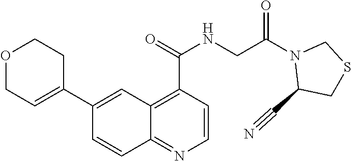 [SMILES: N#C[C@@H]1CSCN1C(=O)CNC(=O)c1ccnc2ccc(C3=CCOCC3)cc12]

6-(3,6-Dihydro-2H-pyran-4-yl)quinoline-4-carboxylic acid Intermediate 59 (30 mg, 0.12 mmol), HATU (67 mg, 0.18 mmol) and DIPEA (62 μL, 0.35 mmol) were mixed in DMF (1 mL). (R)-3-glycylthiazolidine-4-carbonitrile hydrochloride Intermediate 4 (29 mg, 0.14 mmol) was added and the reaction stirred at rt overnight. EtOAc (8 mL) and NaHCO₃ (aq, 4 mL) were added, the mixture was stirred and the phases separated. The organic phase was washed with aqueous NaCl (3×). The combined aqueous phases was extracted with EtOAc (2×). The organic phases were combined, dried over MgSO₄ and evaporated to give crude product which was purified by preparative SFC, PrepMethod SFC-B (gradient 5-90%) to give the title compound (17 mg, 35%); HRMS (ESI) m/z [M+H]⁺ calcd for C₂₁H₂₁N₄O₃S: 409.1328, found: 409.1336; ¹H NMR (600 MHz, DMSO-d₆) 9.11 (t, 1H), 8.93 (d, 1H), 8.49-8.43 (m, 1H), 8.07-7.97 (m, 2H), 7.54 (d, 1H), 6.59-6.53 (m, 1H), 5.33 (dd, 1H), 4.90 (d, 1H), 4.72 (d, 1H), 4.41-4.24 (m, 4H), 3.88 (t, 2H), 3.43-3.35 (m, overlapping with solvent peak), 2.63-2.57 (m, 2H).

### Example 23: (R)—N-(2-(4-Cyanothiazolidin-3-yl)-2-oxoethyl)-6-(4-ethoxytetrahydro-2H-pyran-4-yl)quinoline-4-carboxamide

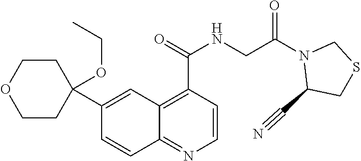 [SMILES: CCOC1(c2ccc3nccc(C(=O)NCC(=O)N4CSC[C@H]4C#N)c3c2)CCOCC1]

TEA (509 μL, 3.65 mmol) was added to a stirred suspension of 6-(4-ethoxytetrahydro-2H-pyran-4-yl)quinoline-4-carboxylic acid Intermediate 60 (110 mg, 0.37 mmol), (R)-3-glycylthiazolidine-4-carbonitrile hydrochloride Intermediate 4 (152 mg, 0.73 mmol), HOBt (247 mg, 1.83 mmol) and EDC (350 mg, 1.83 mmol) in MeCN (10 mL) and EtOAc (10 mL) at 10° C. The resulting suspension was stirred at 50° C. for 2 h. The solvent was removed under reduced pressure. The residue was partitioned between sat NaHCO₃ (aq, 50 mL) and EtOAc (100 mL). The aqueous layer was extracted with EtOAc (5×100 mL). The organic layers were combined and washed with water (3×50 mL). The aqueous layers were combined and extracted with EtOAc (3×20 mL). All organic layers were combined, dried over Na₂SO₄, filtered and evaporated. The crude product was purified by preparative HPLC PrepMethod D (gradient 15-30%) to give the title compound (140 mg, 84%); HRMS (ESI) m/z [M+H]⁺ calcd for C₂₃H₂₇N₄O₄S: 455.1748, found: 455.1754; ¹H NMR (300 MHz, DMSO-d₆) 9.22 (t, 1H), 9.06 (d, 1H), 8.47-8.36 (m, 1H), 8.15 (d, 1H), 7.99 (dd, 1H), 7.68 (d, 1H), 5.34 (dd, 1H), 4.92 (d, 1H), 4.73 (d, 1H), 4.38-4.27 (m, 2H), 3.87-3.69 (m, 4H), 3.48-3.31 (m, 2H), 3.05 (q, 2H), 2.15-1.94 (m, 4H), 1.08 (t, 3H).

### Example 24: (R)—N-(2-(4-Cyanothiazolidin-3-yl)-2-oxoethyl)-6-(morpholino-methyl)quinoline-4-carboxamide

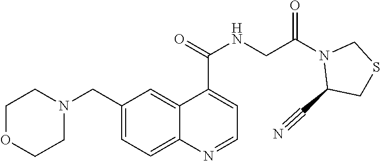 [SMILES: N#C[C@@H]1CSCN1C(=O)CNC(=O)c1ccnc2ccc(CN3CCOCC3)cc12]

DIPEA (1.46 mL, 8.37 mmol) was added to 6-(morpholinomethyl)quinoline-4-carboxylic acid Intermediate 62 (114 mg, 0.42 mmol), (R)-3-glycylthiazolidine-4-carbonitrile hydrochloride Intermediate 4 (174 mg, 0.84 mmol), HOBt (566 mg, 4.19 mmol) and EDC (803 mg, 4.19 mmol) in MeCN (5 mL) and EtOAc (5 mL) at 20° C. The resulting solution was stirred under N₂ (g) at 20° C. for 16 h. The solvent was removed under reduced pressure. The residue was diluted with sat NaHCO₃ (aq, 100 mL) and extracted with EtOAc (5×100 mL). The organic layers were combined and washed with water (3×50 mL), dried over Na₂SO₄, filtered and evaporated. The crude product was purified by preparative HPLC PrepMethod D (gradient 2-10%) to give the title compound (6 mg, 3%). HRMS (ESI) m/z [M+H]⁺ calcd for C₂₁H₂₄N₅O₃S: 426.1594, found: 426.1596; ¹H NMR (400 MHz, DMSO-d₆) δ 9.09 (t, 1H), 8.95 (d, 1H), 8.20 (d, 1H), 8.05 (d, 1H), 7.80 (dd, 1H), 7.56 (d, 1H), 5.36 (dd, 1H), 4.89 (d, 1H), 4.72 (d, 1H), 4.33 (d, 2H), 3.67 (s, 2H), 3.63-3.56 (m, 4H), 3.45-3.30 (m, overlapping with solvent peak), 2.45-2.37 (m, 4H).

### Example 25: N-(2-((R)-4-Cyanothiazolidin-3-yl)-2-oxoethyl)-6-((RS)-1-hydroxyethyl)quinoline-4-carboxamide

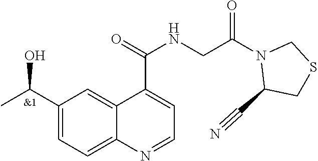 [SMILES: C[C@@H](O)c1ccc2nccc(C(=O)NCC(=O)N3CSC[C@H]3C#N)c2c1]

A solution of rac-(R)-6-(1-hydroxyethyl)quinoline-4-carboxylic acid Intermediate 64 (85 mg, 0.39 mmol), (R)-3-glycylthiazolidine-4-carbonitrile hydrochloride Intermediate 4 (163 mg, 0.78 mmol), EDC (113 mg, 0.59 mmol), HOBt (90 mg, 0.59 mmol) and DIPEA (0.342 mL, 1.96 mmol) in DMF (6 mL) was stirred at rt for 3 h. The solvent was removed under reduced pressure. The residue was purified by preparative TLC (DCM:MeOH, 10:1) to give crude product that was purified by preparative HPLC PrepMethod K (gradient 10-25%) to give the title compound (65 mg, 44%); HRMS (ESI) m/z [M+H]⁺ calcd for C₁₈H₁₉N₄O₃S: 371.1172, found: 371.1180; ¹H NMR (300 MHz, DMSO-d₆) δ 9.09 (t, 1H), 8.94 (d, 1H), 8.25-8.18 (m, 1H), 8.04 (d, 1H), 7.83 (dd, 1H), 7.55 (d, 1H), 5.46-5.32 (m, 2H), 4.98-4.84 (m, 2H), 4.72 (d, 1H), 4.33 (d, 2H), 3.46-3.31 (m, overlapping with solvent peak), 1.41 (d, 3H).

### Example 26: N-(2-((R)-4-Cyanothiazolidin-3-yl)-2-oxoethyl)-6-((RS)-1-methoxyethyl)quinoline-4-carboxamide

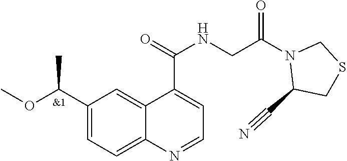 [SMILES: CO[C@@H](C)c1ccc2nccc(C(=O)NCC(=O)N3CSC[C@H]3C#N)c2c1]

A solution of rac-(R)-6-(1-methoxyethyl)quinoline-4-carboxylic acid Intermediate 175 (55 mg, 0.24 mmol), (R)-3-glycylthiazolidine-4-carbonitrile hydrochloride Intermediate 4 (99 mg, 0.48 mmol), EDC (68 mg, 0.36 mmol), HOBt (55 mg, 0.36 mmol) and DIPEA (0.208 mL, 1.19 mmol) in DMF (6 mL) was stirred at rt for 3 h. The solvent was removed under reduced pressure. The residue was purified by preparative TLC (DCM:MeOH, 20:1) and then by preparative HPLC PrepMethod S (gradient 22-32%) to give the title compound (15 mg, 16%); HRMS (ESI) m/z [M+H]⁺ calcd for C₁₉H₂₁N₄O₃S: 385.1328, found: 385.1348; ¹H NMR (300 MHz, CD₃OD) δ 8.92 (d, 1H), 8.33-8.27 (m, 1H), 8.10 (d, 1H), 7.84 (dd, 1H), 7.68 (d, 1H), 5.38 (dd, 1H), 4.88-4.68 (m, overlapping with solvent peak), 4.58 (q, 1H), 4.52-4.37 (m, 2H), 3.52-3.32 (m, overlapping with solvent peak), 3.27 (d, 3H), 1.49 (d, 3H).

### Example 27: (R)—N-(2-(4-cyanothiazolidin-3-yl)-2-oxoethyl)-6-(ethoxymethyl)-quinoline-4-carboxamide

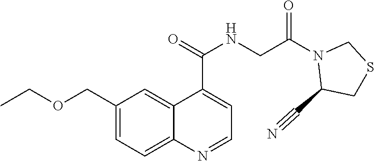 [SMILES: CCOCc1ccc2nccc(C(=O)NCC(=O)N3CSC[C@H]3C#N)c2c1]

DIPEA (483 μL, 2.76 mmol) was added to 6-(ethoxymethyl)quinoline-4-carboxylic acid Intermediate 66 (128 mg, 0.55 mmol), (R)-3-glycylthiazolidine-4-carbonitrile hydrochloride Intermediate 4 (172 mg, 0.83 mmol), HOBt (747 mg, 5.53 mmol) and EDC (1.06 g, 5.53 mmol) in a mixture of MeCN (6 mL) and EtOAc (6 mL) at 20° C. The resulting solution was stirred at 20° C. for 16 h under N₂ (g). The solvent was removed under reduced pressure. The residue was diluted with sat NaHCO₃ (aq, 200 mL) and extracted with EtOAc (3×200 mL). The combined organic layer was washed with water (3×50 mL), dried over Na₂SO₄, filtered and concentrated. The residue was purified by preparative HPLC, PrepMethod F, (gradient: 24-34%) to afford the title compound (0.104 g, 49%); HRMS (ESI) m/z [M+H]⁺ calcd for C₁₉H₂₁N₄O₃S: 385.1328, found: 385.1334; ¹H NMR (300 MHz, DMSO-d₆) δ 9.13 (t, 1H), 8.98 (d, 1H), 8.30-8.21 (d, 1H), 8.08 (d, 1H), 7.78 (dd, 1H), 7.59 (d, 1H), 5.37 (dd, 1H), 4.91 (d, 1H), 4.73 (d, 1H), 4.65 (s, 2H), 4.34 (d, 2H), 3.56 (q, 2H), 3.47-3.36 (m, overlapping with solvent peak), 1.20 (t, 3H).

### Example 28: (R)—N-(2-(4-cyanothiazolidin-3-yl)-2-oxoethyl)-6-methylquinoline-4-carboxamide

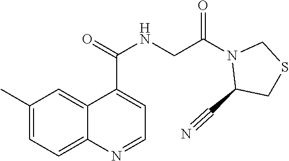 [SMILES: Cc1ccc2nccc(C(=O)NCC(=O)N3CSC[C@H]3C#N)c2c1]

A solution of 6-methylquinoline-4-carboxylic acid (110 mg, 0.59 mmol), (R)-3-glycylthiazolidine-4-carbonitrile hydrochloride Intermediate 4 (244 mg, 1.18 mmol), EDC (169 mg, 0.88 mmol), HOBt (135 mg, 0.88 mmol) and DIPEA (0.51 mL, 2.9 mmol) in DMF (6 mL) was stirred at rt for 3 h. The solvent was removed under reduced pressure and the residue was purified by preparative TLC (DCM:MeOH, 20:1) and further purified by preparative HPLC, PrepMethod L, (gradient: 22-32%) to give the title compound (45 mg, 23%); HRMS (ESI) m/z [M+H]⁺ calcd for C₁₇H₁₇N₄O₂S: 341.1066, found: 341.1064; ¹H NMR (300 MHz, CD₃OD) δ 8.85 (d, 1H), 8.16 (s, 1H), 7.98 (d, 1H), 7.68 (dd, 1H), 7.62 (d, 1H), 5.37 (dd, 1H), 4.86-4.74 (m, overlapping with solvent peak), 4.40 (d, 2H), 3.52-3.34 (m, overlapping with solvent peak), 2.56 (s, 3H).

### Example 29: (R)—N-(2-(4-cyanothiazolidin-3-yl)-2-oxoethyl)-6-(fluoromethyl)-quinoline-4-carboxamide

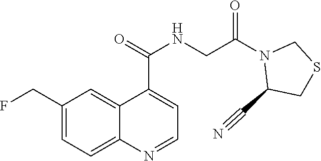 [SMILES: N#C[C@@H]1CSCN1C(=O)CNC(=O)c1ccnc2ccc(CF)cc12]

A solution of 6-(fluoromethyl)quinoline-4-carboxylic acid Intermediate 68 (80 mg, 0.39 mmol), (R)-3-glycylthiazolidine-4-carbonitrile hydrochloride Intermediate 4 (162 mg, 0.78 mmol), EDC (112 mg, 0.58 mmol), HOBt (90 mg, 0.58 mmol) and DIPEA (0.340 mL, 1.95 mmol) in DMF (5 mL) was stirred at rt for 3 h. The solvent was removed under reduced pressure and the residue was purified by preparative TLC (DCM:MeOH, 10:1) and further purified by preparative HPLC, PrepMethod I, (gradient: 22-32%) to afford the title compound (40 mg, 29%); HRMS (ESI) m/z [M+H]⁺ calcd for C₁₇H₁₆FN₄O₂S: 359.0972, found: 359.0960; ¹H NMR (300 MHz, CD₃OD) δ 8.96 (d, 1H), 8.44-8.38 (m, 1H), 8.13 (d, 1H), 7.87 (dd, 1H), 7.70 (d, 1H), 5.60 (d, 2H), 5.38 (dd, 1H), 4.85-4.68 (m, overlapping with solvent peak), 4.42 (d, 2H), 3.52-3.33 (m, overlapping with solvent peak).

### Example 30: N-(2-((R)-4-Cyanothiazolidin-3-yl)-2-oxoethyl)-6-((RS)-1,2-dimethoxyethyl)quinoline-4-carboxamide

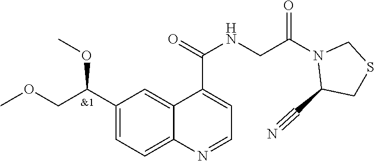 [SMILES: COC[C@@H](OC)c1ccc2nccc(C(=O)NCC(=O)N3CSC[C@H]3C#N)c2c1]

To a mixture of rac-(R)-6-(1,2-dimethoxyethyl)quinoline-4-carboxylic acid Intermediate 71 and 6-((2-methoxyethoxy)methyl)quinoline-4-carboxylic acid Intermediate 72 (60 mg, 0.23 mmol), HOBt (47 mg, 0.34 mmol) and EDC (88 mg, 0.46 mmol) in EtOAc (1.5 mL) and MeCN (1.5 mL) was added (R)-3-glycylthiazolidine-4-carbonitrile hydrochloride Intermediate 4 (72 mg, 0.34 mmol) followed by DIPEA (180 μL, 1.03 mmol). The mixture was stirred at rt for 6 h. Additional EDC (70 mg), HOBt (30 mg) and DIPEA (130 μL) was added and the mixture was stirred overnight at rt. The reaction was then diluted with EtOAc and sat NaHCO₃ (aq). The aqueous layer was extracted using EtOAc and the combined organic layers were dried using a phase separator and concentrated. The resulting residue was purified by preparative SFC, PrepMethod SFC-C (gradient 20-25%) to give the title compounds Example 30 (38 mg, 39%); HRMS (ESI) m/z [M+H]⁺ calcd for C₂₀H₂₃N₄O₄S: 415.1434, found: 415.1440; ¹H NMR (500 MHz, CDCl₃) δ 8.87 (d, 1H), 8.19 (d, 1H), 8.11 (d, 1H), 7.74 (dd, 1H), 7.47 (d, 1H), 7.32 (t, 1H), 5.26-5.23 (m, 1H), 4.64 (s, 2H), 4.55 (dd, 1H), 4.51-4.42 (m, 1H), 4.30 (dt, 1H), 3.68-3.60 (dd, 1H), 3.49 (dd, 1H), 3.36 (s, 3H), 3.32-3.28 (m, 5H).

### Example 31: (R)—N-(2-(4-Cyanothiazolidin-3-yl)-2-oxoethyl)-6-((2-methoxyethoxy)-methyl)quinoline-4-carboxamide

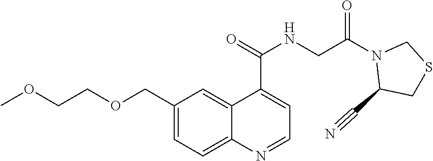 [SMILES: COCCOCc1ccc2nccc(C(=O)NCC(=O)N3CSC[C@H]3C#N)c2c1]

Synthesized as a mixture with Example 30 and separated (see Example 30). Example 31 (29 mg, 31%); HRMS (ESI) m/z [M+H]⁺ calcd for C₂₀H₂₃N₄O₄S: 415.1434, found: 415.1438; ¹H NMR (500 MHz, CDCl₃) δ 8.88 (d, 1H), 8.21-8.16 (m, 1H), 8.09 (d, 1H), 7.77 (dd, 1H), 7.47 (d, 1H), 7.33-7.24 (m, overlapping with solvent peak), 5.24 (t, 1H), 4.72 (s, 2H), 4.63 (s, 2H), 4.44 (dd, 1H), 4.29 (dd, 1H), 3.69-3.62 (m, 2H), 3.60-3.55 (m, 2H), 3.37 (s, 3H), 3.33-3.28 (m, 2H).

### Example 32: N-(2-((R)-4-Cyanothiazolidin-3-yl)-2-oxoethyl)-6-((RS)-tetrahydro-2H-pyran-2-yl)quinoline-4-carboxamide

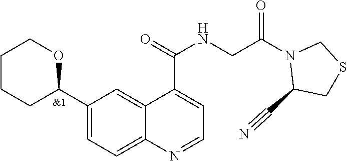 [SMILES: N#C[C@@H]1CSCN1C(=O)CNC(=O)c1ccnc2ccc([C@H]3CCCCO3)cc12]

### Step a) rac-(R)-6-(Tetrahydro-2H-pyran-2-yl)quinoline-4-carboxylic Acid

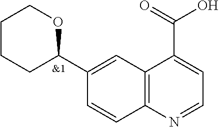 [SMILES: O=C(O)c1ccnc2ccc([C@H]3CCCCO3)cc12]

Aq LiOH (133 μL, 0.13 mmol, 1 M) was added to a solution of rac-methyl (R)-6-(tetrahydro-2H-pyran-2-yl)quinoline-4-carboxylate Intermediate 73 (18 mg, 0.07 mmol) in THF (0.53 mL). The reaction was stirred at rt for 2 h and then treated with 1 M HCl (0.4 mL) and concentrated. The resulting residue was treated with MeOH, filtered and concentrated to afford the crude title compound (20 mg) as a yellow film which was used directly in the next step; MS (ESI) m/z [M+H]⁺ 258.1.

### Step b) N-(2-((R)-4-Cyanothiazolidin-3-yl)-2-oxoethyl)-6-((RS)-tetrahydro-2H-pyran-2-yl)quinoline-4-carboxamide

(R)-3-Glycylthiazolidine-4-carbonitrile hydrochloride Intermediate 4 (31 mg, 0.15 mmol) was added to a suspension of the crude rac-(R)-6-(tetrahydro-2H-pyran-2-yl)quinoline-4-carboxylic acid (18 mg, 0.07 mmol), HOBt (18 mg, 0.14 mmol) and EDC (29 mg, 0.15 mmol) in EtOAc (313 μL) and MeCN (313 μL). DIPEA (132 μL, 0.76 mmol) was added and the mixture was stirred at rt overnight. The reaction mixture was concentrated, and the resulting residue was dissolved in EtOAc and sat NaHCO₃ (aq). The aqueous layer was extracted using EtOAc (3×) and the combined organic layers were dried using a phase separator and concentrated. The residue was purified by preparative HPLC, PrepMethod M, (gradient: 5-95%), to give the title compound (14 mg, 57% calculated over two steps); HRMS (ESI) m/z [M+H]⁺ calcd for C₂₁H₂₃N₄O₃S: 411.1486, found: 411.1478; ¹H NMR (600 MHz, DMSO-d₆) δ 9.09 (t, 1H), 8.96 (d, 1H), 8.24 (dd, 1H), 8.05 (d, 1H), 7.79 (dd, 1H), 7.57 (d, 1H), 5.36 (dd, 1H), 4.90 (d, 1H), 4.73 (d, 1H), 4.54-4.48 (m, 1H), 4.36-4.32 (m, 2H), 4.11-4.05 (m, 1H), 3.60 (td, 1H), 3.44-3.38 (m, overlapping with solvent peak), 1.95-1.87 (m, 2H), 1.75-1.47 (m, 4H).

### Example 33: N-(2-((R)-4-Cyanothiazolidin-3-yl)-2-oxoethyl)-6-((RS)-1,4-dioxan-2-yl)quinoline-4-carboxamide

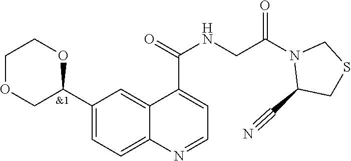 [SMILES: N#C[C@@H]1CSCN1C(=O)CNC(=O)c1ccnc2ccc([C@H]3COCCO3)cc12]

### Step a) rac-(R)-6-(1,4-Dioxan-2-yl)quinoline-4-carboxylic Acid

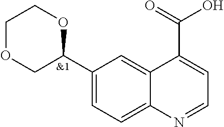 [SMILES: O=C(O)c1ccnc2ccc([C@H]3COCCO3)cc12]

rac-Methyl (R)-6-(1,4-dioxan-2-yl)quinoline-4-carboxylate Intermediate 74 (45 mg, 0.16 mmol) was treated as described for Example 32 Step a) to afford crude rac-(R)-6-(1,4-dioxan-2-yl)quinoline-4-carboxylic acid which was used directly in the next step without further purification; MS (ESI) m/z [M+H]⁺ 259.98.

### Step b) N-(2-((R)-4-Cyanothiazolidin-3-yl)-2-oxoethyl)-6-((RS)-1,4-dioxan-2-yl)quinoline-4-carboxamide

The title compound was synthesized in an analogous manner to Example 32 starting from rac-(R)-6-(1,4-dioxan-2-yl)quinoline-4-carboxylic acid from Step a). Purification by preparative HPLC, PrepMethod G, (gradient 5-95%) to give the title compound (20 mg, 29%); HRMS (ESI) m/z [M+H]⁺ calcd for C₂₀H₂₁N₄O₄S: 413.1278, found: 413.1278; ¹H NMR (600 MHz, DMSO-d₆) δ 9.11 (t, 1H), 8.99 (d, 1H), 8.34-8.30 (m, 1H), 8.07 (d, 1H), 7.83 (dd, 1H), 7.59 (d, 1H), 5.37 (dd, 1H), 4.90 (dd, 1H), 4.78 (dd, 1H), 4.73 (d, 1H), 4.41-4.30 (m, 2H), 3.98-3.92 (m, 2H), 3.83 (td, 1H), 3.80-3.75 (m, 1H), 3.69-3.62 (m, 1H), 3.45-3.37 (m, overlapping with solvent peak).

### Example 34: N-(2-((R)-4-Cyanothiazolidin-3-yl)-2-oxoethyl)-6-((RS)-tetrahydrofuran-2-yl)quinoline-4-carboxamide

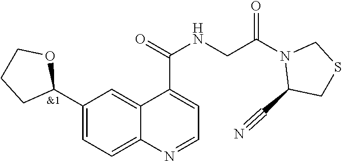 [SMILES: N#C[C@@H]1CSCN1C(=O)CNC(=O)c1ccnc2ccc([C@H]3CCCO3)cc12]

(R)-3-Glycylthiazolidine-4-carbonitrile hydrochloride Intermediate 4 (48 mg, 0.23 mmol) was added to a suspension of rac-(R)-6-(tetrahydrofuran-2-yl)quinoline-4-carboxylic acid Intermediate 76 (43 mg, 0.18 mmol), HOBt (33 mg, 0.21 mmol) and EDC (51 mg, 0.27 mmol) in EtOAc (1 mL) and MeCN (1 mL). DIPEA (123 μL, 0.71 mmol) was added and the mixture was stirred at rt for 6 h. Additional (R)-3-glycylthiazolidine-4-carbonitrile hydrochloride Intermediate 4 (10 mg), HOBt (7 mg), EDC (10 mg) and DIPEA (25 μL) was added. The reaction was stirred overnight and then concentrated and diluted with EtOAc and sat NaHCO₃ (aq). The aqueous layer was extracted using EtOAc and the combined organic layers were dried using a phase separator and concentrated. The residue was purified by preparative HPLC, PrepMethod G, (gradient: 5-95%) to give the title compound (26 mg, 38%); HRMS (ESI) m/z [M+H]⁺ calcd for C₂₀H₂₁N₄O₃S: 397.1328, found: 397.1306; ¹H NMR (600 MHz, DMSO-d₆) δ 9.10 (t, 1H), 8.96 (d, 1H), 8.26 (t, 1H), 8.06 (d, 1H), 7.77 (dd, 1H), 7.57 (dd, 1H), 5.37-5.33 (m, 1H), 5.00 (t, 1H), 4.90 (dd, 1H), 4.73 (dd, 1H), 4.40-4.28 (m, 2H), 4.11-4.04 (m, 1H), 3.90-3.85 (m, 1H), 3.47-3.27 (m, overlapping with solvent peak), 2.44-2.36 (m, 1H), 2.06-1.95 (m, 2H), 1.82-1.73 (m, 1H).

### Example 35: N-(2-((R)-4-Cyanothiazolidin-3-yl)-2-oxoethyl)-6-((((RS)-tetrahydrofuran-3-yl)oxy)methyl)quinoline-4-carboxamide

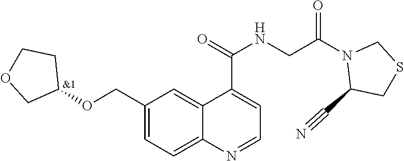 [SMILES: N#C[C@@H]1CSCN1C(=O)CNC(=O)c1ccnc2ccc(CO[C@H]3CCOC3)cc12]

A solution of rac-6-(((tetrahydrofuran-3-yl)oxy)methyl)quinoline-4-carboxylic acid Intermediate 77 (70 mg, 0.26 mmol), (R)-3-glycylthiazolidine-4-carbonitrile hydrochloride Intermediate 4 (80 mg, 0.38 mmol), EDC (74 mg, 0.38 mmol), HOBt (52 mg, 0.38 mmol) and DIPEA (224 μL, 1.28 mmol) in MeCN (5 mL) and EtOAc (5 mL) was stirred at 50° C. for 1 h. The solvent was removed under reduced pressure. The residue was dissolved in EtOAc (25 mL) and washed with water (3×10 mL). The organic layer was dried over Na₂SO₄, filtered and concentrated. The residue was purified by preparative HPLC, PrepMethod N (gradient: 13-23%), to give the title compound (30 mg, 28%); HRMS (ESI) m/z [M+H]⁺ calcd for C₂₁H₂₃N₄O₄S: 427.1434, found: 427.1440; ¹H NMR (400 MHz, CD₃OD) δ 9.15 (d, 1H), 8.61-8.55 (m, 1H), 8.20 (d, 1H), 8.07 (dd, 1H), 7.98 (d, 1H), 5.41-5.33 (m, 1H), 4.88-4.70 (m, overlapping with solvent peak), 4.47 (s, 2H), 4.41-4.33 (m, 1H), 4.01-3.89 (m, 2H), 3.88-3.78 (m, 2H), 3.55-3.34 (m, 2H), 2.23-2.02 (m, 2H).

### Example 36: (R)—N-(2-(4-Cyanothiazolidin-3-yl)-2-oxoethyl)-6-((2,2-difluoroethoxy)methyl)quinoline-4-carboxamide

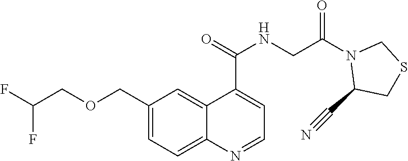 [SMILES: N#C[C@@H]1CSCN1C(=O)CNC(=O)c1ccnc2ccc(COCC(F)F)cc12]

The title compound was prepared from 6-((2,2-difluoroethoxy)methyl)quinoline-4-carboxylic acid Intermediate 78 (90 mg, 0.34 mmol) in a manner analogous to Example 35. The compound was purified by preparative HPLC, PrepMethod F, (gradient: 28-38%) to give the title compound (92 mg, 65%); HRMS (ESI) m/z [M+H]⁺ calcd for C₁₉H₁₉F₂N₄O₃S: 421.1140, found: 421.1154; ¹H NMR (300 MHz, CD₃OD) δ 8.94 (d, 1H), 8.42-8.34 (m, 1H), 8.10 (d, 1H), 7.85 (dd, 1H), 7.68 (d, 1H), 6.04 (tt, 1H), 5.37 (dd, 1H) 4.87-4.76 (m, overlapping with solvent peak), 4.51-4.32 (m, 2H), 3.79 (td, 2H), 3.54-3.34 (m, 2H).

### Example 37: (R)—N-(2-(4-Cyanothiazolidin-3-yl)-2-oxoethyl)-6-(cyclopropoxy-methyl)quinoline-4-carboxamide

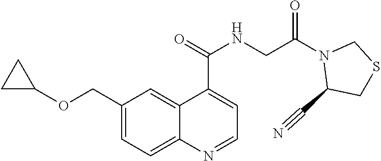 [SMILES: N#C[C@@H]1CSCN1C(=O)CNC(=O)c1ccnc2ccc(COC3CC3)cc12]

### Step a) 6-(Cyclopropoxymethyl)quinoline-4-carboxylic Acid

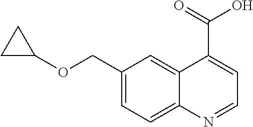 [SMILES: O=C(O)c1ccnc2ccc(COC3CC3)cc12]

A solution of NaH (54 mg, 1.4 mmol; 60% wt in oil) and cyclopropanol (59 mg, 1.0 mmol) in DMF (4 mL) was stirred at 0° C. for 10 min. Methyl 6-(chloromethyl)quinoline-4-carboxylate Intermediate 9 (80 mg, 0.34 mmol) was added and the reaction was stirred at 0° C. for 1 h. Water (1 mL) was added and the solution was stirred at rt for 30 min. The solvent was removed under reduced pressure. The residue was diluted with water and the solution mixture was adjusted to pH 6. The solution was diluted with EtOAc (25 mL), and washed sequentially with water (3×10 mL). The organic layer was dried over Na₂SO₄, filtered and concentrated to give the crude title compound (120 mg); MS (ESI) m/z [M+H]⁺ 244.

### Step b) (R)—N-(2-(4-Cyanothiazolidin-3-yl)-2-oxoethyl)-6-(cyclopropoxymethyl)-quinoline-4-carboxamide

A solution of 6-(cyclopropoxymethyl)quinoline-4-carboxylic acid from Step a) (85 mg, 0.18 mmol), (R)-3-glycylthiazolidine-4-carbonitrile hydrochloride Intermediate 4 (94 mg, 0.45 mmol), EDC (134 mg, 0.70 mmol), HOBt (94 mg, 0.70 mmol) and DIPEA (305 μL, 1.75 mmol) in MeCN (5 mL) and EtOAc (5 mL) was stirred at 50° C. for 2 h. The solvent was removed under reduced pressure. The residue was dissolved in EtOAc (25 mL) and washed sequentially with water (2×10 mL). The organic layer was dried over Na₂SO₄, filtered and concentrated. The residue was purified by preparative HPLC, PrepMethod D (gradient: 21-31%) to give the title compound (48 mg, 35%). HRMS (ESI) m/z [M+H]⁺ calcd for C₂₀H₂₁N₄O₃S: 397.1328, found: 397.1320; ¹H NMR (300 MHz, CD₃OD) δ 8.92 (d, 1H), 8.38-8.33 (m, 1H), 8.08 (d, 1H), 7.83 (dd, 1H), 7.68 (d, 1H), 5.38 (dd, 1H), 4.87-4.74 (m, 4H), 4.51-4.33 (d, 2H), 3.51-3.36 (m, 3H), 0.71-0.60 (m, 2H), 0.60-0.47 (m, 2H).

### Example 38: (R)—N-(2-(4-Cyanothiazolidin-3-yl)-2-oxoethyl)-6-((4-methylisoxazol-3-yl)methyl)quinoline-4-carboxamide

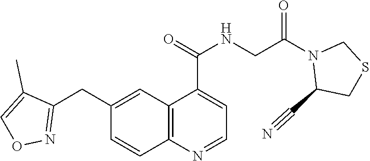 [SMILES: Cc1conc1Cc1ccc2nccc(C(=O)NCC(=O)N3CSC[C@H]3C#N)c2c1]

HATU (30 mg, 0.08 mmol) and DIPEA (44 μL, 0.25 mmol) were added to a suspension of 6-((4-methylisoxazol-3-yl)methyl)quinoline-4-carboxylic acid Intermediate 79 (17 mg, 0.06 mmol) and (R)-3-glycylthiazolidine-4-carbonitrile hydrochloride Intermediate 4 (13 mg, 0.06 mmol) in EtOAc (0.25 mL) and MeCN (0.25 mL). The solution was stirred at rt overnight. The reaction mixture was evaporated and the residue was purified by preparative HPLC, PrepMethod G (gradient: 5-95%) to give the title compound (7.4 mg, 28%); HRMS (ESI) m/z [M+H]⁺ calcd for C₂₁H₂₀N₅O₃S: 422.1282, found: 422.1254; ¹H NMR (600 MHz, DMSO-d₆) δ 9.10 (t, 1H), 8.97 (d, 1H), 8.58 (d, 1H), 8.22 (d, 1H), 8.05 (d, 1H), 7.67 (dd, 1H), 7.58 (d, 1H), 5.36 (dd, 1H), 4.89 (d, 1H), 4.73 (d, 1H), 4.32 (d, 2H), 4.21-4.18 (m, 2H), 3.46-3.35 (m, overlapping with solvent peak), 1.88 (s, 3H).

### Example 39: (R)—N-(2-(4-Cyanothiazolidin-3-yl)-2-oxoethyl)-6-((4,6-dimethylpyridin-3-yl)methyl)quinoline-4-carboxamide

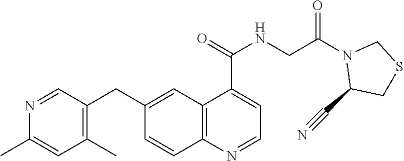 [SMILES: Cc1cc(C)c(Cc2ccc3nccc(C(=O)NCC(=O)N4CSC[C@H]4C#N)c3c2)cn1]

### Step a) 6-((4,6-Dimethylpyridin-3-yl)methyl)quinoline-4-carboxylic Acid

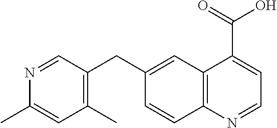 [SMILES: Cc1cc(C)c(Cc2ccc3nccc(C(=O)O)c3c2)cn1]

Ethyl 6-((4,6-dimethylpyridin-3-yl)methyl)quinoline-4-carboxylate Intermediate 80 (50 mg, 0.16 mmol) was dissolved in MeOH (2.5 mL) and aq NaOH (312 μL, 0.31 mmol, 1 M) was added. The reaction was stirred at 50° C. for 20 min and was then cooled to rt. HCl (103 μL, 0.39 mmol, 3.8 M) was added and the mixture evaporated and co-evaporated with EtOH (2×) and then with MeCN to give the crude subtitle compound that was used as such in the next step.

### Step b) (R)—N-(2-(4-Cyanothiazolidin-3-yl)-2-oxoethyl)-6-((4,6-dimethylpyridin-3-yl)methyl)quinoline-4-carboxamide

The resulting crude 6-((4,6-dimethylpyridin-3-yl)methyl)quinoline-4-carboxylic acid (42 mg) was mixed with HATU (82 mg, 0.22 mmol) and DIPEA (100 μL, 0.57 mmol) in 1:1 MeCN/EtOAc (2 mL). (R)-3-Glycylthiazolidine-4-carbonitrile hydrochloride Intermediate 4 (36 mg, 0.17 mmol) was added and the reaction mixture was stirred at rt for 3 h. The reaction mixture was partitioned between DCM (10 mL) and NaHCO₃ (aq, 7 mL), the layers were separated using a phase separator and the organic layer was concentrated. The residue was purified by preparative HPLC, PrepMethod C (gradient: 0-40%) to give the title compound (23 mg, 33% calculated over two steps); HRMS (ESI) m/z [M+H]⁺ calcd for C₂₄H₂₄N₅O₂S: 446.1646, found: 446.1632; ¹H NMR (500 MHz, DMSO-d₆) δ 9.09 (t, 1H), 8.97 (d, 1H), 8.69 (s, 1H), 8.21 (s, 1H), 8.06 (d, 1H), 7.78 (s, 1H), 7.71 (d, 1H), 7.61-7.56 (m, 1H), 5.35 (dd, 1H), 4.88 (d, 1H), 4.71 (d, 1H), 4.37 (s, 2H), 4.34-4.22 (m, 2H), 3.49-3.41 (m, overlapping with solvent peak), 2.66 (s, 3H), 2.45 (s, 3H).

### Example 40: (R)—N-(2-(4-Cyanothiazolidin-3-yl)-2-oxoethyl)-6-((1,3-dimethyl-1H-pyrazol-5-yl)methyl)quinoline-4-carboxamide

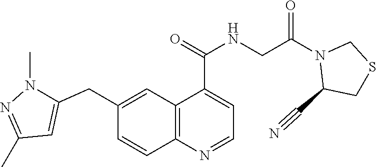 [SMILES: Cc1cc(Cc2ccc3nccc(C(=O)NCC(=O)N4CSC[C@H]4C#N)c3c2)n(C)n1]

The title compound was prepared as described for Example 39 using ethyl ((1,3-dimethyl-1H-pyrazol-5-yl)methyl)quinoline-4-carboxylate Intermediate 81 (80 mg, 0.26 mmol). Purification by preparative HPLC, PrepMethod C (gradient: 5-55%) to give the title compound (63 mg, 56% calculated over two steps); HRMS (ESI) m/z [M+H]⁺ calcd for C₂₂H₂₃N₆O₂S: 435.1598, found: 435.1602; ¹H NMR (500 MHz, DMSO-d₆) δ 9.13 (t, 1H), 8.99 (d, 1H), 8.27 (s, 1H), 8.06 (d, 1H), 7.69 (d, 1H), 7.61 (d, 1H), 5.81 (s, 1H), 5.36 (dd, 1H), 4.89 (d, 1H), 4.72 (d, 1H), 4.33 (d, 2H), 4.17 (s, 2H), 3.68 (s, 3H), 3.42-3.31 (m, overlapping with solvent peak), 2.07 (s, 3H).

### Example 41: (R)—N-(2-(4-Cyanothiazolidin-3-yl)-2-oxoethyl)-6-((2-methyl-2H-1,2,3-triazol-4-yl)methyl)quinoline-4-carboxamide

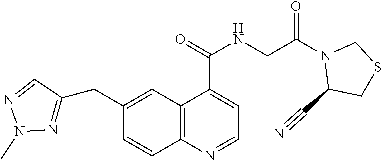 [SMILES: Cn1ncc(Cc2ccc3nccc(C(=O)NCC(=O)N4CSC[C@H]4C#N)c3c2)n1]

The title compound was prepared as described for Example 39 using ethyl 6-((2-methyl-2H-1,2,3-triazol-4-yl)methyl)quinoline-4-carboxylate Intermediate 82 (80 mg, 0.27 mmol). Purification by preparative HPLC, PrepMethod C (gradient: 5-50%) to give the title compound (40 mg, 35% calculated over two steps); HRMS (ESI) m/z [M+H]⁺ calcd for C₂₀H₂₀N₇O₂S: 422.1394, found: 422.1388; ¹H NMR (500 MHz, DMSO-d₆) δ 9.15 (t, 1H), 9.01 (d, 1H), 8.27-8.23 (m, 1H), 8.05 (d, 1H), 7.75 (dd, 1H), 7.64 (d, 1H), 7.61 (s, 1H), 5.37 (dd, 1H), 4.90 (d, 1H), 4.73 (d, 1H), 4.34 (d, 2H), 4.20 (s, 2H), 4.08 (s, 3H), 3.44-3.29 (m, overlapping with solvent peak).

### Example 42: (R)—N-(2-(4-Cyanothiazolidin-3-yl)-2-oxoethyl)-6-((5-methylisoxazol-3-yl)methyl)quinoline-4-carboxamide

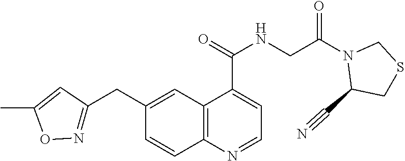 [SMILES: Cc1cc(Cc2ccc3nccc(C(=O)NCC(=O)N4CSC[C@H]4C#N)c3c2)no1]

The title compound was prepared as described for Example 39 using ethyl 6-((5-methylisoxazol-3-yl)methyl)quinoline-4-carboxylate Intermediate 83 (43 mg, 0.15 mmol). Purification by preparative HPLC, PrepMethod C (gradient: 10-60%) to give the title compound (31 mg, 51% calculated over two steps); HRMS (ESI) m/z [M+H]⁺ calcd for C₂₁H₂₀N₅ O₃S: 422.1282, found: 422.1268; ¹H NMR (500 MHz, DMSO-d₆) δ 9.18-9.08 (m, 1H), 9.01-8.94 (m, 1H), 8.26 (s, 1H), 8.05 (dd, 1H), 7.75-7.69 (m, 1H), 7.63-7.56 (m, 1H), 6.18-6.09 (m, 1H), 5.39-5.34 (m, 1H), 4.90 (d, 1H), 4.72 (d, 1H), 4.34 (d, 2H), 4.15-4.12 (m, 2H), 3.46-3.39 (m, overlapping with solvent peak), 2.33 (s, 3H).

### Example 43: (R)—N-(2-(4-Cyanothiazolidin-3-yl)-2-oxoethyl)-6-(1-(6-methylpyridin-3-yl)cyclopropyl)quinoline-4-carboxamide

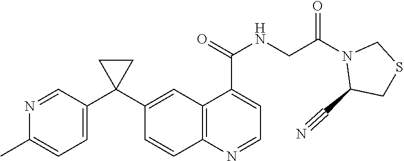 [SMILES: Cc1ccc(C2(c3ccc4nccc(C(=O)NCC(=O)N5CSC[C@H]5C#N)c4c3)CC2)cn1]

### Step a) 6-(1-(6-Methylpyridin-3-yl)cyclopropyl)quinoline-4-carboxylic Acid

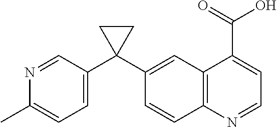 [SMILES: Cc1ccc(C2(c3ccc4nccc(C(=O)O)c4c3)CC2)cn1]

Ethyl 6-(1-(6-methylpyridin-3-yl)cyclopropyl)quinoline-4-carboxylate Intermediate 86 (8 mg, 0.02 mmol) was dissolved in MeOH (1 mL) and aq NaOH (72 μL, 0.07 mmol, 1 M) was added. The reaction was stirred at 50° C. for 20 min, then cooled to rt. HCl (22 μL, 0.08 mmol, 3.8 M) was added and the mixture evaporated and co-evaporated with EtOH (2×) give the crude subtitle compound that was used as such in the next step.

### Step b) (R)—N-(2-(4-Cyanothiazolidin-3-yl)-2-oxoethyl)-6-(1-(6-methylpyridin-3-yl)cyclopropyl)quinoline-4-carboxamide

Crude 6-(1-(6-methylpyridin-3-yl)cyclopropyl)quinoline-4-carboxylic acid was mixed with HATU (15 mg, 0.04 mmol) and DIPEA (18 μL, 0.11 mmol) in 1:1 MeCN/EtOAc (0.8 mL). (R)-3-Glycylthiazolidine-4-carbonitrile hydrochloride Intermediate 4 (6.6 mg, 0.03 mmol) was added and the reaction mixture was stirred at rt for 3 h. The reaction mixture was partitioned between DCM (4 mL) and sat NaHCO₃ (aq, 3 mL), the layers were separated using a phase separator and the organic layer was concentrated. The residue was purified by preparative HPLC, PrepMethod G (gradient: 5-95%) to give the title compound (1 mg, 10% calculated over two steps); HRMS (ESI) m/z [M+H]⁺ calcd for C₂₅H₂₄N₅O₂S: 458.1646, found: 458.1648.

### Example 44: (R)—N-(2-(4-Cyanothiazolidin-3-yl)-2-oxoethyl)-6-(1-(3-fluoropyridin-2-yl)cyclopropyl)quinoline-4-carboxamide

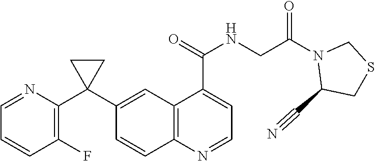 [SMILES: N#C[C@@H]1CSCN1C(=O)CNC(=O)c1ccnc2ccc(C3(c4ncccc4F)CC3)cc12]

DIPEA (646 μL, 3.70 mmol) was added to 6-(1-(3-fluoropyridin-2-yl)cyclopropyl)quinoline-4-carboxylic acid Intermediate 90 (57 mg, 0.18 mmol), (R)-3-glycylthiazolidine-4-carbonitrile hydrochloride Intermediate 4 (77 mg, 0.37 mmol), HOBt (250 mg, 1.85 mmol) and EDC (354 mg, 1.85 mmol) in MeCN (3 mL) and EtOAc (3 mL) at 15° C. The resulting solution was stirred at 50° C. for 2 h under N₂ (g). The solvent was removed under reduced pressure and the residue was diluted with sat NaHCO₃ (aq, 200 mL) and extracted with EtOAc (3×200 mL). The organic layers were combined and washed with water (3×50 mL), dried over Na₂SO₄, filtered and concentrated. The residue was purified by preparative HPLC, PrepMethod D (gradient: 25-40%) to give the title compound (19 mg, 22%); HRMS (ESI) m/z [M+H]⁺ calcd for C₂₄H₂₁FN₅O₂S: 462.1394, found: 462.1390; ¹H NMR (300 MHz, DMSO-d₆) δ 9.07 (t, 1H), 8.92 (d, 1H), 8.43 (dt, 1H), 8.16 (d, 1H), 7.98 (d, 1H), 7.69-7.51 (m, 3H), 7.43 (dt, 1H), 5.35 (dd, 1H), 4.89 (d, 1H), 4.72 (d, 1H), 4.31 (d, 2H), 3.47-3.35 (m, overlapping with solvent peak), 1.62-1.43 (m, 4H).

### Example 45: (R)—N-(2-(4-Cyanothiazolidin-3-yl)-2-oxoethyl)-6-(1-(5-methylisoxazol-3-yl)cyclopropyl)quinoline-4-carboxamide

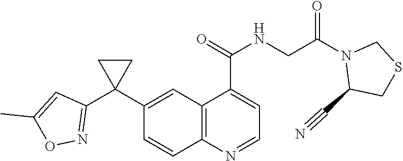 [SMILES: Cc1cc(C2(c3ccc4nccc(C(=O)NCC(=O)N5CSC[C@H]5C#N)c4c3)CC2)no1]

### Step a) 6-(1-(5-methylisoxazol-3-yl)cyclopropyl)quinoline-4-carboxylic Acid

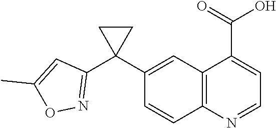 [SMILES: Cc1cc(C2(c3ccc4nccc(C(=O)O)c4c3)CC2)no1]

A solution of tert-butyl 6-(1-(5-methylisoxazol-3-yl)cyclopropyl)quinoline-4-carboxylate Intermediate 92 (140 mg, 0.40 mmol) in 1:2 TFA/DCM (6 mL) was stirred at 30° C. for 5 h. The solvent was removed under reduced pressure to give the title compound as a TFA-salt (0.20 g) that was used in the next step directly without further purification;

MS (ESI) m/z [M+H]⁺ 295.

### Step b) (R)—N-(2-(4-Cyanothiazolidin-3-yl)-2-oxoethyl)-6-(1-(5-methylisoxazol-3-yl)cyclopropyl)quinoline-4-carboxamide

A solution of 6-(1-(5-methylisoxazol-3-yl)cyclopropyl)quinoline-4-carboxylic acid from Step a) (130 mg), (R)-3-glycylthiazolidine-4-carbonitrile hydrochloride Intermediate 4 (138 mg, 0.66 mmol), EDC (169 mg, 0.88 mmol), HOBt (119 mg, 0.88 mmol) and DIPEA (386 μL, 2.21 mmol) in EtOAc (5 mL) and MeCN (5 mL) was stirred at 50° C. for 3 h. The solvent was removed under reduced pressure. The residue was dissolved in EtOAc (30 mL) and washed sequentially with water (3×15 mL). The organic layer was dried over Na₂SO₄, filtered and concentrated. The residue was purified by preparative HPLC, PrepMethod D (gradient: 25-35%) to afford the title compound (70 mg, 60% calculated over two steps); HRMS (ESI) m/z [M+H]⁺ calcd for C₂₃H₂₂N₅O₃S: 448.1438, found: 448.1448; ¹H NMR (300 MHz, CD₃OD) δ 8.91 (d, 1H), 8.51-8.35 (m, 1H), 8.05 (d, 1H), 7.84 (dd, 1H), 7.66 (d, 1H), 5.90 (s, 1H), 5.43-5.30 (m, 1H), 4.85-4.68 (m, overlapping with solvent peak), 4.50-4.31 (m, 2H), 3.52-3.34 (m, overlapping with solvent peak), 2.34 (s, 3H), 1.61-1.41 (m, 4H).

### Example 46: N-(2-((R)-4-Cyanothiazolidin-3-yl)-2-oxoethyl)-6-((RS)-1-(5-methylisoxazol-3-yl)ethyl)quinoline-4-carboxamide

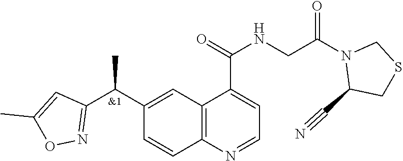 [SMILES: Cc1cc([C@@H](C)c2ccc3nccc(C(=O)NCC(=O)N4CSC[C@H]4C#N)c3c2)no1]

The title compound was prepared in a similar manner as described for Example 45, Step b), starting from 6-(1-(5-methylisoxazol-3-yl)ethyl)quinoline-4-carboxylic acid TFA Intermediate 94 (200 mg, 0.39 mmol). Purification by preparative HPLC, PrepMethod D (gradient: 25-35%) to give the title compound (70 mg, 41%); HRMS (ESI) m/z [M+H]⁺ calcd for C₂₂H₂₂N₅O₃S: 436.1438, found: 436.1452; ¹H NMR (300 MHz, CD₃OD) δ 8.91 (d, 1H), 8.47-8.37 (m, 1H), 8.04 (d, 1H), 7.74 (dt, 1H), 7.64 (t, 1H), 6.11 (d, 1H), 5.45-5.33 (m, 1H), 4.89-4.66 (m, overlapping with solvent peak), 4.55-4.33 (m, 3H), 3.47-3.36 (m, 2H), 2.35 (s, 3H), 1.75 (d, 3H).

### Example 47: (R)-6-(1-(5-Chloropyridin-2-yl)cyclopropyl)-N-(2-(4-cyanothiazolidin-3-yl)-2-oxoethyl)quinoline-4-carboxamide

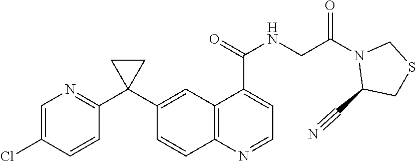 [SMILES: N#C[C@@H]1CSCN1C(=O)CNC(=O)c1ccnc2ccc(C3(c4ccc(Cl)cn4)CC3)cc12]

### Step a) 6-(1-(5-Chloropyridin-2-yl)cyclopropyl)quinoline-4-carboxylic Acid

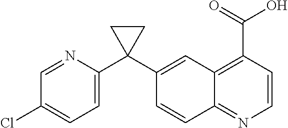 [SMILES: O=C(O)c1ccnc2ccc(C3(c4ccc(Cl)cn4)CC3)cc12]

LiOH (25 mg, 1.0 mmol) was added to methyl 6-(1-(5-chloropyridin-2-yl)cyclopropyl)quinoline-4-carboxylate Intermediate 97 (70 mg, 0.21 mmol) in 1:1 THF/water (6 mL) at 20° C. The resulting solution was stirred at 25° C. for 4 h. The reaction mixture was adjusted to pH 4 with 2 M HCl (aq). The solvent was removed under reduced pressure to give the crude title compound; MS (ESI) m/z [M+H]⁺ 325.0.

### Step b) (R)-6-(1-(5-Chloropyridin-2-yl)cyclopropyl)-N-(2-(4-cyanothiazolidin-3-yl)-2-oxoethyl)quinoline-4-carboxamide

(R)-3-Glycylthiazolidine-4-carbonitrile hydrochloride Intermediate 4 (26 mg, 0.12 mmol) and DIPEA (65 μL, 0.37 mmol) were added to a suspension of crude 6-(1-(5-chloropyridin-2-yl)cyclopropyl)quinoline-4-carboxylic acid from Step a) (40 mg), EDC (35 mg, 0.18 mmol) and HOBt (25 mg, 0.18 mmol) in a mixture of 1,4-dioxane (1 mL) and DCM (1 mL). The resulting suspension was stirred at 25° C. for 16 h. The solvent was removed under reduced pressure and the residue was purified by preparative HPLC, PrepMethod H (gradient: 34-54%) to give the title compound (5 mg, 5%); HRMS (ESI) m/z [M+H]⁺ calcd for C₂₄H₂₁ClN₅O₂S: 478.1098, found: 478.1084; ¹H NMR (300 MHz, CDCl₃) δ 8.98 (d, 1H), 8.44 (d, 1H), 8.34-8.25 (m, 1H), 8.15 (d, 1H), 7.80 (dd, 1H), 7.55 (d, 1H), 7.38 (dd, 1H), 7.09-6.94 (m, 1H), 6.77 (d, 1H), 5.30 (t, 1H), 4.71-4.63 (m, 2H), 4.60-4.45 (m, 1H), 4.44-4.21 (m, 1H), 3.43-3.30 (m, 2H), 1.84-1.66 (m, 2H), 1.55-1.40 (m, 2H).

### Example 48: 6-((RS)-1-(5-Chloropyridin-2-yl)ethyl)-N-(2-((R)-4-cyanothiazolidin-3-yl)-2-oxoethyl)quinoline-4-carboxamide

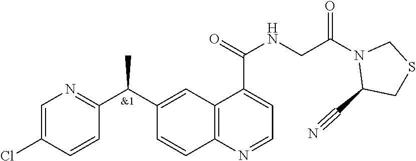 [SMILES: C[C@@H](c1ccc2nccc(C(=O)NCC(=O)N3CSC[C@H]3C#N)c2c1)c1ccc(Cl)cn1]

A solution of 6-(1-(5-chloropyridin-2-yl)ethyl)quinoline-4-carboxylic acid Intermediate 100 (110 mg, 0.35 mmol), (R)-3-glycylthiazolidine-4-carbonitrile hydrochloride Intermediate 4 (110 mg, 0.53 mmol), EDC (135 mg, 0.70 mmol), HOBt (95 mg, 0.70 mmol) and DIPEA (307 μL, 1.76 mmol) in 1:1 EtOAc/MeCN (10 mL) was stirred at 50° C. for 1.5 h. The solvent was removed under reduced pressure. The residue was dissolved in EtOAc (25 mL) and washed sequentially with water (3×10 mL). The organic layer was dried over Na₂SO₄, filtered and concentrated. The residue was purified by preparative HPLC, PrepMethod O (gradient: 24-35%) to give the title compound (45 mg, 28%) as a pale yellow gum; HRMS (ESI) m/z [M+H]⁺ calcd for C₂₃H₂₁ClN₅O₂S: 466.1098, found: 466.1102; ¹H NMR (300 MHz, CD₃OD) δ 9.14 (d, 1H), 8.75-8.65 (m, 1H), 8.54-8.44 (m, 1H), 8.21-8.05 (m, 2H), 8.03-7.94 (m, 1H), 7.89-7.77 (m, 1H), 7.56 (dd, 1H), 5.39 (dd, 1H), 4.87-4.72 (m, overlapping with solvent peak), 4.71-4.59 (m, 1H), 4.47 (s, 2H), 3.55-3.35 (m, 2H), 1.84 (dd, 3H).

### Example 49: N-(2-((R)-4-Cyanothiazolidin-3-yl)-2-oxoethyl)-6-((RS)-1-(3-methylisoxazol-5-yl)ethyl)quinoline-4-carboxamide

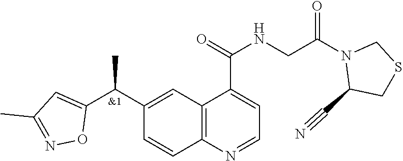 [SMILES: Cc1cc([C@@H](C)c2ccc3nccc(C(=O)NCC(=O)N4CSC[C@H]4C#N)c3c2)on1]

To a stirred solution of rac-(R)-6-(1-(3-methylisoxazol-5-yl)ethyl)quinoline-4-carboxylic acid Intermediate 104 (200 mg, 0.71 mmol) in DMF (5 mL) was added HATU (404 mg, 1.06 mmol), (R)-3-glycylthiazolidine-4-carbonitrile hydrochloride Intermediate 4 (147 mg, 0.71 mmol), and DIPEA (495 μL, 2.83 mmol). The resulting solution was stirred at 25° C. for 2 h. The solvent was removed under reduced pressure. The residue was purified by preparative TLC (DCM:MeOH, 19:1) and further purified by preparative HPLC, PrepMethod P (gradient: 40-50%) to give the title compound (34 mg, 11%); HRMS (ESI) m/z [M+H]⁺ calcd for C₂₂H₂₂N₅O₃S: 436.1438, found: 436.1442; ¹H NMR (300 MHz, DMSO-d₆) δ 9.14 (m, 1H), 8.96 (d, 1H), 8.42-8.25 (m, 1H), 8.05 (d, 1H), 7.72 (dt, 1H), 7.57 (dd, 1H), 6.22 (d, 1H), 5.41-5.28 (m, 1H), 4.89 (d, 1H), 4.71 (d, 1H), 4.57-4.44 (m, 1H), 4.32 (d, 2H), 3.49-3.28 (m, 2H), 2.17 (s, 3H), 1.66 (d, 3H).

### Example 50: (R)—N-(2-(4-Cyanothiazolidin-3-yl)-2-oxoethyl)-6-(1-(3-methylisoxazol-5-yl)cyclopropyl)quinoline-4-carboxamide

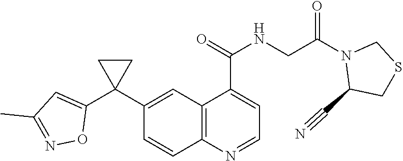 [SMILES: Cc1cc(C2(c3ccc4nccc(C(=O)NCC(=O)N5CSC[C@H]5C#N)c4c3)CC2)on1]

### Step a) 6-(1-(3-Methylisoxazol-5-yl)cyclopropyl)quinoline-4-carboxylic Acid

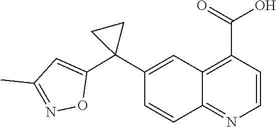 [SMILES: Cc1cc(C2(c3ccc4nccc(C(=O)O)c4c3)CC2)on1]

TFA (185 μL, 2.40 mmol) was added to a stirred solution of tert-butyl 6-(1-(3-methylisoxazol-5-yl)cyclopropyl)quinoline-4-carboxylate Intermediate 105 (168 mg, 0.48 mmol) in DCM (3 mL). The resulting solution was stirred at 25° C. for 24 h. The solvent was removed under reduced pressure and the crude product was purified by C18-flash chromatography, (gradient: 0-30% MeCN in water) to give the title compound (97 mg, 69%); MS (ESI) m/z [M+H]⁺ 295.1.

### Step b) (R)—N-(2-(4-Cyanothiazolidin-3-yl)-2-oxoethyl)-6-(1-(3-methylisoxazol-5-yl)cyclopropyl)quinoline-4-carboxamide

(R)-3-Glycylthiazolidine-4-carbonitrile hydrochloride Intermediate 4 (85 mg, 0.41 mmol), 50% T3P in EtOAc (173 mg, 0.27 mmol) and DIPEA (48 μL, 0.27 mmol) was added to a stirred solution of 6-(1-(3-methylisoxazol-5-yl)cyclopropyl)quinoline-4-carboxylic acid from Step a) (80 mg) in DMF (2 mL). The resulting solution was stirred at 25° C. for 2 h. The solvent was removed under reduced pressure and the residue was purified by preparative TLC (DCM:MeOH, 19:1) followed by preparative HPLC, PrepMethod H (gradient: 26-46%) to give the title compound (52 mg, 29% calculated over two steps). HRMS (ESI) m/z [M+H]⁺ calcd for C₂₃H₂₂N₅O₃S: 448.1438, found: 448.1410; ¹H NMR (300 MHz, DMSO-d₆) δ 9.12 (t, 1H), 8.98 (d, 1H), 8.37 (d, 1H), 8.07 (d, 1H), 7.81 (dd, 1H), 7.59 (d, 1H), 5.93 (s, 1H), 5.36 (dd, 1H), 4.89 (d, 1H), 4.72 (d, 1H), 4.32 (d, 2H), 3.50-3.33 (m, overlapping with solvent peak), 2.12 (s, 3H), 1.64-1.38 (m, 4H).

### Example 51: N-(2-((R)-4-Cyanothiazolidin-3-yl)-2-oxoethyl)-6-((RS)-1-(6-methylpyridin-3-yl)ethyl)quinoline-4-carboxamide

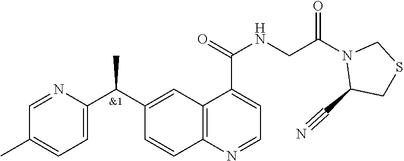 [SMILES: Cc1ccc([C@@H](C)c2ccc3nccc(C(=O)NCC(=O)N4CSC[C@H]4C#N)c3c2)nc1]

A solution of rac-6-(1-(6-methylpyridin-3-yl)ethyl)quinoline-4-carboxylic acid Intermediate 107 (100 mg, 0.34 mmol), (R)-3-glycylthiazolidine-4-carbonitrile hydrochloride Intermediate 4 (107 mg, 0.51 mmol), EDC (131 mg, 0.68 mmol), HOBt (92 mg, 0.68 mmol) and DIPEA (299 μL, 1.71 mmol) in EtOAc (5 mL) and MeCN (5 mL) was stirred at 50° C. for 2 h. The solvent was removed under reduced pressure. The residue was dissolved in EtOAc (30 mL) and washed with water (3×15 mL). The organic layer was dried over Na₂SO₄, filtered and concentrated. The residue was purified by preparative HPLC, PrepMethod D (gradient: 2-20%), to give the title compound (50 mg, 33%). HRMS (ESI) m/z [M+H]⁺ calcd for C₂₄H₂₄N₅O₂S: 446.1646, found: 446.1632; ¹H NMR (400 MHz, CD₃OD) δ 8.89 (d, 1H), 8.52-8.35 (m, 2H), 8.02 (d, 1H), 7.83-7.68 (m, 2H), 7.63 (dd, 1H), 7.26 (t, 1H), 5.44-5.33 (m, 1H), 4.84-4.75 (m, overlapping with solvent peak), 4.54-4.35 (m, 3H), 3.53-3.34 (m, 2H), 2.49 (s, 3H), 1.80 (d, 3H).

### Example 52: N-(2-((R)-4-Cyanothiazolidin-3-yl)-2-oxoethyl)-6-((RS)-1-(2-(trifluoromethyl)pyridin-4-yl)ethyl)quinoline-4-carboxamide

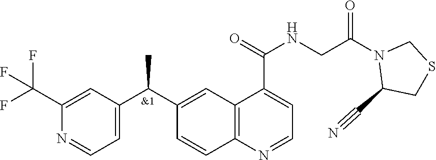 [SMILES: C[C@H](c1ccnc(C(F)(F)F)c1)c1ccc2nccc(C(=O)NCC(=O)N3CSC[C@H]3C#N)c2c1]

A solution of rac-(R)-6-(1-(2-(trifluoromethyl)pyridin-4-yl)ethyl)quinoline-4-carboxylic acid Intermediate 109 (100 mg, 0.29 mmol), (R)-3-glycylthiazolidine-4-carbonitrile hydrochloride Intermediate 4 (90 mg, 0.43 mmol), EDC (111 mg, 0.58 mmol), HOBt (88 mg, 0.58 mmol) and DIPEA (151 μL, 0.87 mmol) in EtOAc (5 mL) and MeCN (5 mL) was stirred at 50° C. for 2 h. The solvent was removed under reduced pressure. The reaction mixture was diluted with EtOAc, and washed with water. The organic layer was dried over Na₂SO₄, filtered and concentrated. The residue was purified by preparative HPLC PrepMethod T (gradient 32-42%) to give the title compound (80 mg, 56%). HRMS (ESI) m/z [M+H]⁺ calcd for C₂₄H₂₁F₃N₅O₂S: 500.1362, found: 500.1348; ¹H NMR (300 MHz, CD₃OD) δ 8.92 (d, 1H), 8.75 (dd, 1H), 8.59-8.50 (m, 1H), 8.18-8.01 (m, 2H), 7.83-7.71 (m, 2H), 7.64 (d, 1H), 5.47-5.36 (m, 1H), 4.86-4.71 (m, overlapping with solvent peak), 4.62 (q, 1H), 4.50-4.35 (m, 2H), 3.50-3.37 (m, overlapping with solvent peak), 1.87 (d, 3H).

### Example 53: (R)—N-(2-(4-Cyanothiazolidin-3-yl)-2-oxoethyl)-6-(2,2-difluoropropyl)quinoline-4-carboxamide

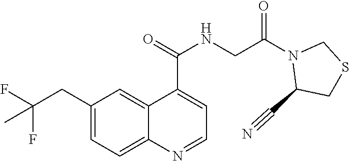 [SMILES: CC(F)(F)Cc1ccc2nccc(C(=O)NCC(=O)N3CSC[C@H]3C#N)c2c1]

A mixture of 6-(2,2-difluoropropyl)quinoline-4-carboxylic acid Intermediate 112 (100 mg, 0.40 mmol), (R)-3-glycylthiazolidine-4-carbonitrile hydrochloride Intermediate 4 (107 mg, 0.52 mmol), EDC (153 mg, 0.80 mmol), HOBt (108 mg, 0.80 mmol) and DIPEA (348 μL, 1.99 mmol) in MeCN (5 mL) and EtOAc (5.00 mL) was stirred at 50° C. for 2 h. The solvent was removed under reduced pressure. The reaction mixture was diluted with EtOAc (25 mL) and washed sequentially with water (2×15 mL), dried over Na₂SO₄, filtered and concentrated. The crude was purified by preparative HPLC, PrepMethod D (gradient 23-34%) to afford the title compound (80 mg, 50%); HRMS (ESI) m/z [M+H]⁺ calcd for C₁₉H₁₉F₂N₄O₂S: 405.1192, found: 405.1198; ¹H NMR (300 MHz, CD₃OD) δ 8.93 (d, 1H), 8.36-8.30 (m, 1H), 8.06 (d, 1H), 7.83-7.76 (m, 1H), 7.67 (d, 1H), 5.37 (dd, 1H), 4.88-4.70 (m, 2H), 4.42 (d, 2H), 3.52-3.35 (m, 4H), 1.60 (t, 3H).

### Example 54: 6-((1r,4R*)-4-Cyanocyclohexyl)-N-(2-((R)-4-cyanothiazolidin-3-yl)-2-oxoethyl)quinoline-4-carboxamide Isomer 1

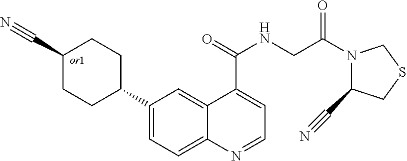 [SMILES: N#C[C@@H]1CSCN1C(=O)CNC(=O)c1ccnc2ccc([C@H]3CC[C@H](C#N)CC3)cc12]

6-((1r,4RS)-4-Cyanocyclohexyl)-N-(2-((R)-4-cyanothiazolidin-3-yl)-2-oxoethyl)quinoline-4-carboxamide Intermediate 116 was purified by preparative chiral HPLC, PrepMethod Q (50% isocratic run over 17 min and a flow rate of 15 mL/min) to afford the title compound as the first eluted compound (45 mg, 45%); HRMS (ESI) m/z [M+H]⁺ calcd for C₂₃H₂₄N₅O₂S: 434.1646, found: 434.1654; ¹H NMR (400 MHz, CD₃OD) δ 8.90 (d, 1H), 8.40-8.25 (m, 1H), 8.07 (d, 1H), 7.85-7.75 (m, 1H), 7.64 (d, 1H), 5.53-5.45 (m, 1H), 4.90-4.78 (m, overlapping with solvent peak), 4.52-4.36 (m, 2H), 3.55-3.37 (m, 2H), 3.25-3.11 (m, 1H), 2.94-2.78 (m, 1H), 2.25-1.77 (m, 8H).

### Example 55: 6-((1r,4R*)-4-Cyanocyclohexyl)-N-(2-((R)-4-cyanothiazolidin-3-yl)-2-oxoethyl)quinoline-4-carboxamide Isomer 2

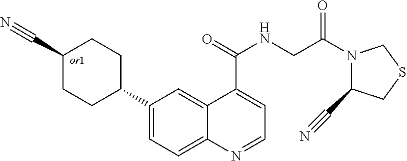 [SMILES: N#C[C@@H]1CSCN1C(=O)CNC(=O)c1ccnc2ccc([C@H]3CC[C@H](C#N)CC3)cc12]

6-((1r,4RS)-4-Cyanocyclohexyl)-N-(2-((R)-4-cyanothiazolidin-3-yl)-2-oxoethyl)quinoline-4-carboxamide Intermediate 116 was purified by preparative chiral HPLC, PrepMethod Q (50% isocratic run over 17 min and a flow rate of 15 mL/min) to afford the title compound as the second eluted compound (18 mg, 18%); HRMS (ESI) m/z [M+H]⁺ calcd for C₂₃H₂₄N₅O₂S: 434.1646, found: 434.1620; ¹H NMR (400 MHz, CD₃OD) δ 8.89 (d, 1H), 8.41-8.35 (m, 1H), 8.04 (d, 1H), 7.77 (dd, 1H), 7.62 (d, 1H), 5.38 (dd, 1H), 4.90-4.71 (m, overlapping with solvent peak), 4.43 (s, 2H), 3.55-3.37 (m, 2H), 2.94-2.66 (m, 2H), 2.35-2.20 (m, 2H), 2.12-1.97 (m, 2H), 1.88-1.68 (m, 4H).

### Example 56: (R)—N-(2-(4-Cyanothiazolidin-3-yl)-2-oxoethyl)-6-((tetrahydro-2H-pyran-4-yl)methyl)quinoline-4-carboxamide

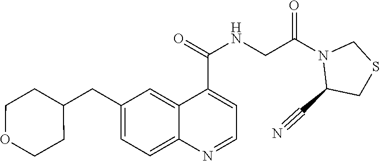 [SMILES: N#C[C@@H]1CSCN1C(=O)CNC(=O)c1ccnc2ccc(CC3CCOCC3)cc12]

A mixture of 6-((tetrahydro-2H-pyran-4-yl)methyl)quinoline-4-carboxylic acid Intermediate 118 (30 mg, 0.11 mmol), (R)-3-glycylthiazolidine-4-carbonitrile hydrochloride Intermediate 4 (30 mg, 0.14 mmol), EDC (42 mg, 0.22 mmol), HOBt (30 mg, 0.22 mmol) and DIPEA (97 μL, 0.55 mmol) in MeCN (2 mL) and EtOAc (2 mL) was stirred at 50° C. for 2 h. The solvent was removed under reduced pressure. The residue was taken up in EtOAc (25 mL) and washed with water (3×10 mL). The organic layer was dried over Na₂SO₄, filtered and concentrated. The crude product was purified by preparative HPLC, PrepMethod F (gradient 24-34%) to afford the title compound (20 mg, 43%); HRMS (ESI) m/z [M+H]⁺ calcd for C₂₂H₂₅N₄O₃S: 425.1642, found: 425.1624; ¹H NMR (400 MHz, DMSO-d₆) δ 9.08 (t, 1H), 8.92 (d, 1H), 8.12 (d, 1H), 8.00 (d, 1H), 7.67 (dd, 1H), 7.53 (d, 1H), 5.35 (dd, 1H), 4.90 (d, 1H), 4.72 (d, 1H), 4.33 (d, 2H), 3.81 (dd, 2H), 3.45-3.34 (m, overlapping with solvent peak), 3.31-3.19 (m, 2H), 2.72 (d, 2H), 1.98-1.82 (m, 1H), 1.59-1.46 (m, 2H), 1.34-1.15 (m, 2H).

### Example 57: (R)—N-(2-(4-Cyanothiazolidin-3-yl)-2-oxoethyl)-6-(4-ethynyl-tetrahydro-2H-pyran-4-yl)quinoline-4-carboxamide

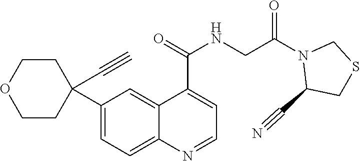 [SMILES: C#CC1(c2ccc3nccc(C(=O)NCC(=O)N4CSC[C@H]4C#N)c3c2)CCOCC1]

DIPEA (1.93 mL, 11.0 mmol) was added to a mixture of 6-(4-ethynyltetrahydro-2H-pyran-4-yl)quinoline-4-carboxylic acid Intermediate 120 (155 mg, 0.55 mmol), (R)-3-glycylthiazolidine-4-carbonitrile hydrochloride Intermediate 4 (114 mg, 0.55 mmol), HOBt (745 mg, 5.51 mmol) and EDC (1.06 g, 5.51 mmol) in MeCN (6 mL) and EtOAc (6 mL) at rt. The reaction mixture was stirred at rt for 16 h under N₂ (g). The solvent was removed under reduced pressure. The residue was diluted with sat NaHCO₃ (aq, 250 mL) and extracted with EtOAc (3×250 mL). The combined organic layers were washed sequentially with water (3×50 mL). The organic layer was dried over Na₂SO₄, filtered and concentrated under reduced pressure. The crude was purified by preparative HPLC, PrepMethod D (gradient 21-35%) to afford the title compound (0.116 g, 49%); HRMS (ESI) m/z [M+H]⁺ calcd for C₂₃H₂₃N₄O₃S: 435.1486, found: 435.1494; ¹H NMR (400 MHz, DMSO-d₆) δ 9.15 (t, 1H), 8.98 (d, 1H), 8.48 (d, 1H), 8.11 (d, 1H), 8.04 (dd, 1H), 7.58 (d, 1H), 5.34 (dd, 1H), 4.90 (d, 1H), 4.73 (d, 1H), 4.47-4.19 (m, 2H), 3.98-3.90 (m, 2H), 3.82 (td, 2H), 3.51-3.38 (m, overlapping with solvent peak), 2.15-1.90 (m, 5H).

### Example 58: (R)—N-(2-(4-Cyanothiazolidin-3-yl)-2-oxoethyl)-6-(3,3,3-trifluoropropyl)quinoline-4-carboxamide

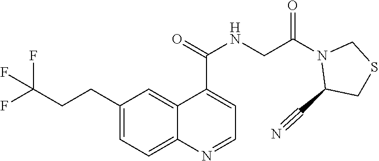 [SMILES: N#C[C@@H]1CSCN1C(=O)CNC(=O)c1ccnc2ccc(CCC(F)(F)F)cc12]

A mixture of 6-(3,3,3-trifluoropropyl)quinoline-4-carboxylic acid Intermediate 122 (130 mg, 0.48 mmol), (R)-3-glycylthiazolidine-4-carbonitrile hydrochloride Intermediate 4 (130 mg, 0.63 mmol), EDC (185 mg, 0.97 mmol), HOBt (130 mg, 0.97 mmol) and DIPEA (422 μL, 2.41 mmol) in MeCN (6 mL) and EtOAc (6 mL) was stirred at 50° C. for 2 h. The solvent was removed under reduced pressure. The residue was taken up in EtOAc (25 mL) and washed sequentially with water (3×10 mL). The organic layer was dried over Na₂SO₄, filtered and concentrated. The crude was purified by preparative HPLC, PrepMethod D (gradient 25-36%) to afford the title compound (0.12 g, 59%); HRMS (ESI) m/z [M+H]⁺ calcd for C₁₉H₁₈F₃N₄O₂S: 423.1096, found: 423.1106; ¹H NMR (400 MHz, CD₃OD) δ 8.92 (d, 1H), 8.37-8.30 (d, 1H), 8.07 (d, 1H), 7.79 (dd, 1H), 7.67 (d, 1H), 5.39 (dd, 1H), 4.89-4.78 (m, overlapping with solvent peak), 4.51-4.37 (m, 2H), 3.49-3.35 (m, 2H), 3.18-3.10 (m, 2H), 2.76-2.59 (m, 2H).

### Example 59: (R)-6-(4-Cyanotetrahydro-2H-pyran-4-yl)-N-(2-(4-cyanothiazolidin-3-yl)-2-oxoethyl)quinoline-4-carboxamide

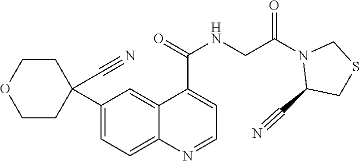 [SMILES: N#C[C@@H]1CSCN1C(=O)CNC(=O)c1ccnc2ccc(C3(C#N)CCOCC3)cc12]

DIPEA (254 μL, 1.45 mmol) was added to 6-(4-cyanotetrahydro-2H-pyran-4-yl)quinoline-4-carboxylic acid Intermediate 123 (21 mg, 0.07 mmol), (R)-3-glycylthiazolidine-4-carbonitrile hydrochloride Intermediate 4 (23 mg, 0.11 mmol), HOBt (98 mg, 0.73 mmol) and EDC (139 mg, 0.73 mmol) in MeCN (2 mL) and EtOAc (2 mL) at 30° C. The resulting solution was stirred at 30° C. for 16 h under N₂ (g). The solvent was removed under reduced pressure. The residue was diluted with sat NaHCO₃ (aq, 200 mL) and extracted with EtOAc (3×200 mL). The organic layers were combined and washed sequentially with water (3×50 mL). The organic layer was dried over Na₂SO₄, filtered and concentrated under reduced pressure. The crude was purified by preparative HPLC, PrepMethod F (gradient 25-35%) to afford the title compound (17 mg, 53%); HRMS (ESI) m/z [M+H]⁺ calcd for C₂₂H₂₂N₅O₃S: 436.1438, found: 436.1420; ¹H NMR (300 MHz, DMSO-d₆) δ 9.19 (t, 1H), 9.04 (d, 1H), 8.58 (d, 1H), 8.19 (d, 1H), 8.06 (dd, 1H), 7.64 (d, 1H), 5.34 (dd, 1H), 4.92 (d, 1H), 4.73 (d, 1H), 4.36 (dd, 2H), 4.16-4.00 (m, 2H), 3.72 (td, 2H), 3.48-3.35 (m, overlapping with solvent peak), 2.41-2.06 (m, 4H).

### Example 60: (R)-6-(1-Cyanocyclohexyl)-N-(2-(4-cyanothiazolidin-3-yl)-2-oxoethyl)quinoline-4-carboxamide

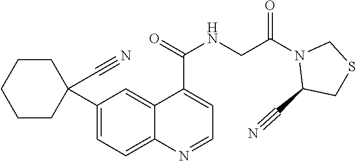 [SMILES: N#C[C@@H]1CSCN1C(=O)CNC(=O)c1ccnc2ccc(C3(C#N)CCCCC3)cc12]

DIPEA (262 μL, 1.50 mmol) was added to 6-(1-cyanocyclohexyl)quinoline-4-carboxylic acid Intermediate 124 (21 mg, 0.07 mmol), (R)-3-glycylthiazolidine-4-carbonitrile hydrochloride Intermediate 4 (23 mg, 0.11 mmol), HOBt (101 mg, 0.75 mmol) and EDC (144 mg, 0.75 mmol) in MeCN (2 mL) and EtOAc (2 mL). The reaction mixture was stirred at 30° C. for 16 h under N₂ (g). The solvent was removed under reduced pressure. The residue was diluted with sat NaHCO₃ (aq, 100 mL) and extracted with EtOAc (3×100 mL). The combined organic layers were washed sequentially with water (3×50 mL), dried over Na₂SO₄, filtered and concentrated. The crude was purified by preparative HPLC, PrepMethod D (gradient 31-45%) to afford the title compound (13 mg, 39%); HRMS (ESI) m/z [M+H]⁺ calcd for C₂₃H₂₄N₅O₂S: 434.1646, found: 434.1656; ¹H NMR (300 MHz, DMSO-d₆) δ 9.18 (t, 1H), 9.02 (d, 1H), 8.60 (d, 1H), 8.17 (d, 1H), 8.03 (dd, 1H), 7.62 (d, 1H), 5.34 (dd, 1H), 4.91 (d, 1H), 4.73 (d, 1H), 4.49-4.20 (m, 2H), 3.44-3.35 (m, overlapping with solvent peak), 2.33-2.15 (m, 2H), 2.09-1.56 (m, 7H), 1.49-1.28 (m, 1H).

### Example 61: N-(2-((R)-4-Cyanothiazolidin-3-yl)-2-oxoethyl)-6-(5,5-dimethyltetrahydrofuran-3-yl)quinoline-4-carboxamide

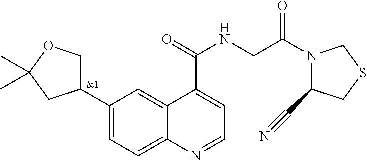 [SMILES: CC1(C)CC(c2ccc3nccc(C(=O)NCC(=O)N4CSC[C@H]4C#N)c3c2)CO1]

DIPEA (523 μL, 2.99 mmol) was added to 6-(5,5-dimethyltetrahydrofuran-3-yl)quinoline-4-carboxylic acid Intermediate 129 (41 mg, 0.15 mmol), (R)-3-glycylthiazolidine-4-carbonitrile hydrochloride Intermediate 4 (31 mg, 0.15 mmol), HOBt (202 mg, 1.50 mmol) and EDC (287 mg, 1.50 mmol) in MeCN (3 mL) and EtOAc (3 mL). The reaction mixture was stirred at 30° C. for 16 h under N₂ (g). The solvent was removed under reduced pressure. The residue was diluted with sat NaHCO₃ (aq, 150 mL) and extracted with EtOAc (3×150 mL). The combined organic layers were washed sequentially with water (3×50 mL), dried over Na₂SO₄, filtered and concentrated under reduced pressure. The crude was purified by preparative HPLC, PrepMethod D (gradient 24-48%) to afford the title compound (12 mg, 19%); HRMS (ESI) m/z [M+H]⁺ calcd for C₂₂H₂₅N₄O₃S: 425.1642, found: 425.1658; ¹H NMR (300 MHz, DMSO-d₆) δ 9.10 (t, 1H), 8.96 (d, 1H), 8.33-8.16 (m, 1H), 8.07 (d, 1H), 7.80 (dd, 1H), 7.57 (d, 1H), 5.41-5.29 (m, 1H), 5.10 (t, 1H), 4.90 (d, 1H), 4.72 (d, 1H), 4.45-4.19 (m, 2H), 3.40-3.36 (m, overlapping with solvent peak), 2.47-2.34 (m, 1H), 1.94-1.80 (m, 3H), 1.33 (s, 3H), 1.31 (s, 3H).

### Example 62: N-(2-((R)-4-Cyanothiazolidin-3-yl)-2-oxoethyl)-6-((1r,3R*)-1-fluoro-3-methoxycyclobutyl)quinoline-4-carboxamide Isomer 1

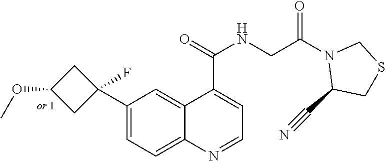 [SMILES: CO[C@H]1C[C@](F)(c2ccc3nccc(C(=O)NCC(=O)N4CSC[C@H]4C#N)c3c2)C1]

A mixture of 6-(1-fluoro-3-methoxycyclobutyl)quinoline-4-carboxylic acid Intermediate 133 (130 mg, 0.47 mmol), (R)-3-glycylthiazolidine-4-carbonitrile hydrochloride Intermediate 4 (127 mg, 0.61 mmol), EDC (181 mg, 0.94 mmol), HOBt (128 mg, 0.94 mmol) and DIPEA (412 μL, 2.36 mmol) in EtOAc (6 mL) and MeCN (6 mL) was stirred at 50° C. for 2 h. The solvent was removed under reduced pressure. The crude was taken up in EtOAc (30 mL) and washed sequentially with water (3×15 mL). The organic layer was dried over Na₂SO₄, filtered and concentrated under reduced pressure. The crude was purified by preparative HPLC, PrepMethod D (gradient 22-32%). The first eluted compound was collected and purified by preparative chiral HPLC, PrepMethod U (20% isocratic run and a flow rate of 1 mL/min) to afford the title compound (20 mg, 10%); HRMS (ESI) m/z [M+H]⁺ calcd for C₂₁H₂₂FN₄O₃S: 429.1390, found: 429.1384; ¹H NMR (300 MHz, CD₃OD) δ 9.27 (d, 1H), 8.85-8.75 (m, 1H), 8.32 (d, 1H), 8.27 (dd, 1H), 8.10 (d, 1H), 5.36 (dd, 1H), 4.88-4.84 (m, overlapping with solvent peak), 4.79 (d, 1H), 4.49 (s, 2H), 4.10-3.95 (m, 1H), 3.54-3.34 (m, 5H), 3.27-3.05 (m, 2H), 2.70 (ddd, 2H).

### Example 63: N-(2-((R)-4-Cyanothiazolidin-3-yl)-2-oxoethyl)-6-((1r,3R*)-1-fluoro-3-methoxycyclobutyl)quinoline-4-carboxamide Isomer 2

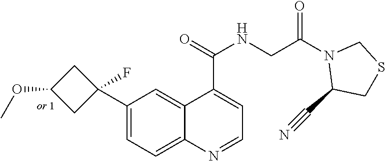 [SMILES: CO[C@H]1C[C@](F)(c2ccc3nccc(C(=O)NCC(=O)N4CSC[C@H]4C#N)c3c2)C1]

For synthesis and work up see description for Example 62. The crude was purified by preparative HPLC, PrepMethod D (gradient 22-32%). The second eluted compound was collected to afford the title compound (60 mg, 30%); HRMS (ESI) m/z [M+H]⁺ calcd for C₂₁H₂₂FN₄O₃S: 429.1390, found: 429.1378; ¹H NMR (300 MHz, CD₃OD) δ 8.95 (d, 1H), 8.51 (dd, 1H), 8.17-8.08 (m, 1H), 7.92 (dd, 1H), 7.68 (d, 1H), 5.39 (dd, 1H), 4.86-4.74 (m, overlapping with solvent peak), 4.52-4.27 (m, 3H), 3.52-3.34 (m, 5H), 3.09-2.89 (m, 2H), 2.77-2.57 (m, 2H).

### Example 64: (R)—N-(2-(4-Cyanothiazolidin-3-yl)-2-oxoethyl)-6-(4-fluorotetrahydro-2H-pyran-4-yl)quinoline-4-carboxamide

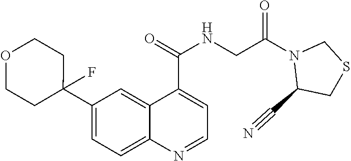 [SMILES: N#C[C@@H]1CSCN1C(=O)CNC(=O)c1ccnc2ccc(C3(F)CCOCC3)cc12]

DIPEA (381 μL, 2.18 mmol) was added to a stirred suspension of 6-(4-fluorotetrahydro-2H-pyran-4-yl)quinoline-4-carboxylic acid Intermediate 135 (150 mg, 0.22 mmol), (R)-3-glycylthiazolidine-4-carbonitrile hydrochloride Intermediate 4 (91 mg, 0.44 mmol), HOBt (147 mg, 1.09 mmol) and EDC (209 mg, 1.09 mmol) in MeCN (3 mL) and EtOAc (3 mL) at 13° C. The reaction mixture was stirred at 50° C. for 2 h. The solvent was removed under reduced pressure and the residue was partitioned between sat NaHCO₃ (aq, 20 mL) and EtOAc (50 mL). The aqueous layer was extracted with EtOAc (5×50 mL). The combined organic layers were washed sequentially with water (3×20 mL). The aqueous layers were combined and extracted with EtOAc (4×15 mL). The organic layers were combined, dried over Na₂SO₄, filtered and concentrated. The crude was purified by preparative HPLC, PrepMethod D (gradient 22-38%) to afford the title compound (70 mg, 75%); HRMS (ESI) m/z [M+H]⁺ calcd for C₂₁H₂₂FN₄O₃S: 429.1390, found: 429.1412; ¹H NMR (300 MHz, DMSO-d₆) δ 9.16 (t, 1H), 9.02 (d, 1H), 8.52-8.40 (m, 1H), 8.14 (d, 1H), 7.94 (dd, 1H), 7.61 (d, 1H), 5.35 (dd, 1H), 4.91 (d, 1H), 4.73 (d, 1H), 4.35 (d, 2H), 3.91 (dd, 2H), 3.75 (td, 2H), 3.45-3.34 (m, overlapping with solvent peak), 2.45-2.15 (m, 2H), 2.05-1.90 (m, 2H).

### Example 65: N-(2-((R)-4-Cyanothiazolidin-3-yl)-2-oxoethyl)-6-(((RS)-tetrahydrofuran-3-yl)methyl)quinoline-4-carboxamide

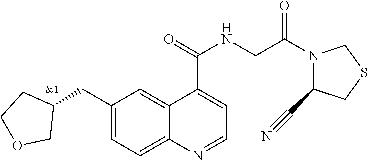 [SMILES: N#C[C@@H]1CSCN1C(=O)CNC(=O)c1ccnc2ccc(C[C@H]3CCOC3)cc12]

EDC (179 mg, 0.93 mmol) was added portion wise to rac-(R)-6-((tetrahydrofuran-3-yl)methyl)quinoline-4-carboxylic acid Intermediate 139 (80 mg, 0.31 mmol), (R)-3-glycylthiazolidine-4-carbonitrile hydrochloride Intermediate 4 (97 mg, 0.47 mmol), HOBt (126 mg, 0.93 mmol) and TEA (433 μL, 3.11 mmol) in DCM (5 mL) at rt. The reaction mixture was stirred at rt for 15 h. The solvent was removed under reduced pressure. The residue was diluted with sat NaHCO₃ (aq, 100 mL) and extracted with EtOAc (3×100 mL). The combined organic layers were washed sequentially with water (3×50 mL). The organic layer was dried over Na₂SO₄, filtered and concentrated. The crude was purified by preparative HPLC, PrepMethod D (gradient 15-30%) to afford the title compound (24 mg, 18%); HRMS (ESI) m/z [M+H]⁺ calcd for C₂₁H₂₃N₄O₃S: 411.1486, found: 411.1504; ¹H NMR (400 MHz, DMSO-d₆) δ 9.10 (t, 1H), 8.93 (d, 1H), 8.20-8.14 (m, 1H), 8.02 (d, 1H), 7.72 (dd, 1H), 7.54 (d, 1H), 5.41-5.32 (m, 1H), 4.90 (d, 1H), 4.73 (d, 1H), 4.33 (d, 2H), 3.84-3.71 (m, 2H), 3.70-3.60 (m, 1H), 3.44-3.35 (m, 3H), 2.92-2.78 (m, 2H), 2.66-2.55 (m, 1H), 2.03-1.87 (m, 1H), 1.68-1.53 (m, 1H).

### Example 66: (R)—N-(2-(4-Cyanothiazolidin-3-yl)-2-oxoethyl)-6-(4-methoxy-tetrahydro-2H-pyran-4-yl)quinoline-4-carboxamide

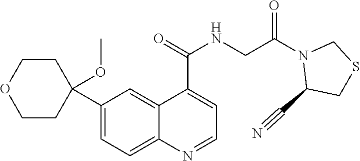 [SMILES: COC1(c2ccc3nccc(C(=O)NCC(=O)N4CSC[C@H]4C#N)c3c2)CCOCC1]

A mixture of 6-(4-methoxytetrahydro-2H-pyran-4-yl)quinoline-4-carboxylic acid Intermediate 140 (180 mg, 0.63 mmol), (R)-3-glycylthiazolidine-4-carbonitrile hydrochloride Intermediate 4 (195 mg, 0.94 mmol), EDC (240 mg, 1.25 mmol), HOBt (169 mg, 1.25 mmol) and DIPEA (547 μL, 3.13 mmol) in EtOAc (10 mL) and MeCN (10 mL) was stirred at 50° C. for 3 h. The solvent was removed under reduced pressure. The residue was taken up in EtOAc (25 mL) and washed sequentially with water (3×10 mL). The organic layer was dried over Na₂SO₄, filtered and concentrated. The crude was purified by preparative HPLC, PrepMethod D (gradient 30-35%) to afford the title compound (90 mg, 33%); HRMS (ESI) m/z [M+H]⁺ calcd for C₂₂H₂₅N₄O₄S: 441.1590, found: 441.1576; ¹H NMR (300 MHz, CD₃OD) δ 8.93 (d, 1H), 8.46 (d, 1H), 8.12 (d, 1H), 7.97 (dd, 1H), 7.65 (d, 1H), 5.35 (dd, 1H), 4.83-4.60 (m, overlapping with solvent peak), 4.42 (s, 2H), 3.98-3.79 (m, 4H), 3.52-3.36 (m, 2H), 3.00 (s, 3H), 2.31-2.10 (m, 4H).

### Example 67: N-(2-((R)-4-Cyanothiazolidin-3-yl)-2-oxoethyl)-6-((RS)-tetrahydrofuran-3-yl)quinoline-4-carboxamide

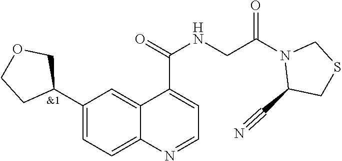 [SMILES: N#C[C@@H]1CSCN1C(=O)CNC(=O)c1ccnc2ccc([C@H]3CCOC3)cc12]

rac-(R)-6-(Tetrahydrofuran-3-yl)quinoline-4-carboxylic acid Intermediate 142 (32 mg, 0.13 mmol), HATU (75 mg, 0.20 mmol) and DIPEA (92 μL, 0.53 mmol) were mixed in MeCN (1 mL) and EtOAc (1 mL). (R)-3-Glycylthiazolidine-4-carbonitrile hydrochloride Intermediate 4 (33 mg, 0.16 mmol) was added and the reaction mixture was stirred at rt for 1 h. DCM (8 mL) and sat NaHCO₃ (aq, 4 mL) were added, the mixture was stirred, filtered through a phase separator and concentrated under reduced pressure. The crude was purified by preparative HPLC, PrepMethod C (gradient 5-50%) to afford the title compound (28 mg, 54%); HRMS (ESI) m/z [M+H]⁺ calcd for C₂₀H₂₁N₄O₃S: 397.1328, found: 397.1332; ¹H NMR (500 MHz, DMSO-d₆) δ 9.18-9.10 (m, 1H), 9.01-8.95 (m, 1H), 8.29-8.25 (m, 1H), 8.10-8.04 (m, 1H), 7.83-7.78 (m, 1H), 7.63-7.57 (m, 1H), 5.39-5.34 (m, 1H), 4.90 (d, 1H), 4.72 (d, 1H), 4.39-4.28 (m, 2H), 4.13 (t, 1H), 4.07-3.97 (m, 1H), 3.85 (q, 1H), 3.70-3.55 (m, 2H), 3.37-3.32 (m, overlapping with solvent peak), 2.45-2.35 (m, 1H), 2.13-2.03 (m, 1H).

### Example 68: N-(2-((R)-4-Cyanothiazolidin-3-yl)-2-oxoethyl)-6-((RS)-2,2-dimethyltetrahydro-2H-pyran-4-yl)quinoline-4-carboxamide

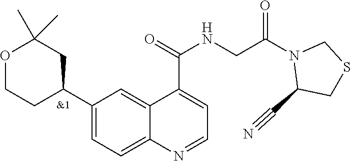 [SMILES: CC1(C)C[C@@H](c2ccc3nccc(C(=O)NCC(=O)N4CSC[C@H]4C#N)c3c2)CCO1]

rac-(R)-6-(2,2-Dimethyltetrahydro-2H-pyran-4-yl)quinoline-4-carboxylic acid Intermediate 144 (40 mg, 0.14 mmol), HATU (80 mg, 0.21 mmol) and DIPEA (98 μL, 0.56 mmol) were dissolved in MeCN (1 mL) and EtOAc (1 mL). (R)-3-glycylthiazolidine-4-carbonitrile hydrochloride Intermediate 4 (35 mg, 0.17 mmol) was added and the reaction mixture was stirred at rt for 1 h. DCM (8 mL) and sat NaHCO₃ (aq, 4 mL) were added, the mixture was stirred, filtered through a phase separator and concentrated under reduced pressure. The crude was purified by preparative HPLC, PrepMethod C (gradient 10-60%) to afford the title compound (22 mg, 36%); HRMS (ESI) m/z [M+H]⁺ calcd for C₂₃H₂₇N₄O₃S: 439.1798, found: 439.1792; ¹H NMR (500 MHz, CDCl₃) δ 8.94 (d, 1H), 8.15-8.10 (m, 2H), 7.71 (dd, 1H), 7.56 (d, 1H), 7.05-6.98 (m, 1H), 5.34-5.28 (m, 1H), 4.77-4.63 (m, 2H), 4.53 (dd, 1H), 4.40 (dd, 1H), 3.92-3.77 (m, 2H), 3.40-3.32 (m, 2H), 3.23-3.10 (m, 1H), 1.88-1.61 (m, overlapping with solvent peak), 1.35 (s, 3H), 1.30 (s, 3H).

### Example 69: 6-((1RS,6SR)-3-Oxabicyclo[4.1.0]heptan-6-yl)-N-(2-((R)-4-cyanothiazolidin-3-yl)-2-oxoethyl)quinoline-4-carboxamide

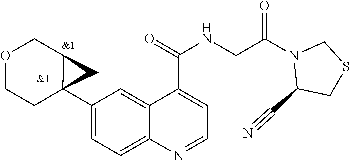 [SMILES: N#C[C@@H]1CSCN1C(=O)CNC(=O)c1ccnc2ccc([C@]34CCOC[C@H]3C4)cc12]

rac-6-((1R,6S)-3-Oxabicyclo[4.1.0]heptan-6-yl)quinoline-4-carboxylic acid Intermediate 147 (24 mg, 0.09 mmol), HATU (51 mg, 0.13 mmol) and DIPEA (62 μL, 0.36 mmol) were mixed in MeCN (0.8 mL) and EtOAc (0.8 mL). (R)-3-glycylthiazolidine-4-carbonitrile hydrochloride Intermediate 4 (22 mg, 0.11 mmol) was added and the reaction mixture was stirred at rt for 1 h. DCM (5 mL) and sat NaHCO₃ (aq, 4 mL) were added, the mixture was stirred, filtered through a phase separator and concentrated under reduced pressure. The crude was purified by preparative SFC, PrepMethod SFC-A (gradient 35-40%) to afford the title compound (15 mg, 39%); HRMS (ESI) m/z [M+H]⁺ calcd for C₂₂H₂₃N₄O₃S: 423.1486, found: 423.1482.

### Example 70: (R)-6-(1-Cyanocyclopropyl)-N-(2-(4-cyanothiazolidin-3-yl)-2-oxoethyl)quinoline-4-carboxamide

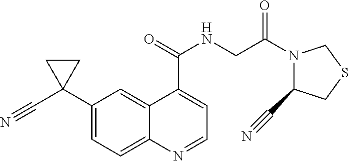 [SMILES: N#C[C@@H]1CSCN1C(=O)CNC(=O)c1ccnc2ccc(C3(C#N)CC3)cc12]

A mixture of 6-(1-cyanocyclopropyl)quinoline-4-carboxylic acid Intermediate 17 (100 mg, 0.42 mmol), (R)-3-glycylthiazolidine-4-carbonitrile hydrochloride Intermediate 4 (174 mg, 0.84 mmol), EDC (161 mg, 0.84 mmol), HOBt (129 mg, 0.84 mmol) and DIPEA (0.367 mL, 2.10 mmol) in DMF (5 mL) was stirred at rt for 15 h. The solvent was removed under reduced pressure. The residue was purified by preparative TLC (DCM:MeOH, 20:1), followed by preparative HPLC, PrepMethod I (gradient 23-33%) to afford the title compound (70 mg, 43%); HRMS (ESI) m/z [M+H]⁺ calcd for C₂₀H₁₈N₅O₂S: 392.1176, found: 392.1176; ¹H NMR (300 MHz, CD₃OD) δ 8.94 (d, 1H), 8.38 (d, 1H), 8.12 (d, 1H), 7.90 (dd, 1H), 7.67 (d, 1H), 5.38 (dd, 1H), 4.85-4.71 (m, overlapping with solvent peak), 4.52-4.32 (m, 2H), 3.55-3.32 (m, overlapping with solvent peak), 1.92-1.65 (m, 4H).

### Example 71: (R)—N-(2-(4-Cyanothiazolidin-3-yl)-2-oxoethyl)-6-(difluoromethyl)-quinoline-4-carboxamide

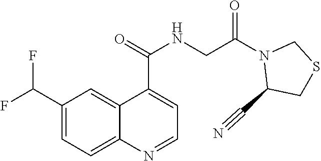 [SMILES: N#C[C@@H]1CSCN1C(=O)CNC(=O)c1ccnc2ccc(C(F)F)cc12]

A mixture of 6-(difluoromethyl)quinoline-4-carboxylic acid Intermediate 149 (60 mg, 0.27 mmol), (R)-3-glycylthiazolidine-4-carbonitrile hydrochloride Intermediate 4 (112 mg, 0.54 mmol), EDC (77 mg, 0.40 mmol), HOBt (62 mg, 0.40 mmol) and DIPEA (0.235 mL, 1.34 mmol) in DMF (5 mL) was stirred at rt for 3 h. The solvent was removed under reduced pressure. The residue was purified by preparative TLC (DCM:MeOH, 10:1), followed by preparative HPLC, PrepMethod I (gradient 25-35%) to afford the title compound (45 mg, 45%); HRMS (ESI) m/z [M+H]⁺ calcd for C₁₇H₁₅F₂N₄O₂S: 377.0878, found: 377.0868; ¹H NMR (400 MHz, CD₃OD) δ 9.04 (d, 1H), 8.66-8.56 (m, 1H), 8.22 (d, 1H), 7.99 (m, 1H), 7.79 (d, 1H), 6.99 (t, 1H), 5.38 (dd, 1H), 4.90-4.69 (m, overlapping with solvent peak), 4.53-4.33 (m, 2H), 3.56-3.35 (m, 2H).

### Example 72: (R)—N-(2-(4-Cyanothiazolidin-3-yl)-2-oxoethyl)-6-(4-(3-methoxy-propoxy)phenyl)quinoline-4-carboxamide

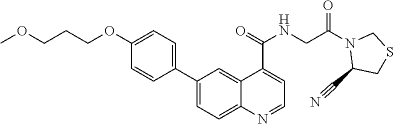 [SMILES: COCCCOc1ccc(-c2ccc3nccc(C(=O)NCC(=O)N4CSC[C@H]4C#N)c3c2)cc1]

6-(4-(3-Methoxypropoxy)phenyl)quinoline-4-carboxylic acid Intermediate 150 (50 mg, 0.15 mmol), HOBt (27 mg, 0.18 mmol) and EDC (43 mg, 0.22 mmol) were mixed in EtOAc (0.7 mL) and MeCN (0.7 mL). (R)-3-glycylthiazolidine-4-carbonitrile hydrochloride Intermediate 4 (62 mg, 0.30 mmol) and DIPEA (0.078 mL, 0.44 mmol) were added. The reaction mixture was stirred at rt overnight. The mixture was diluted with EtOAc, washed with sat NaHCO₃ (aq) and brine, dried over Na₂SO₄, filtered and concentrated under reduced pressure. The residue was purified by preparative HPLC, PrepMethod G (gradient 5-95%) to afford the title compound (13 mg, 18%); HRMS (ESI) m/z [M+H]⁺ calcd for C₂₆H₂₇N₄O₄S: 491.1748, found: 491.1730; ¹H NMR (600 MHz, DMSO-d₆) δ 9.16 (t, 1H), 8.95 (d, 1H), 8.72 (d, 1H), 8.17 (dd, 1H), 8.13 (d, 1H), 7.89-7.84 (m, 2H), 7.57 (d, 1H), 7.11-7.07 (m, 2H), 5.38 (dd, 1H), 4.91 (d, 1H), 4.74 (d, 1H), 4.41-4.30 (m, 2H), 4.09 (t, 2H), 3.50 (t, 2H), 3.45-3.35 (m, overlapping with solvent peak), 3.27 (s, 3H), 1.99 (p, 2H).

### Example 73: (R)—N-(2-(4-Cyanothiazolidin-3-yl)-2-oxoethyl)-6-propoxyquinoline-4-carboxamide

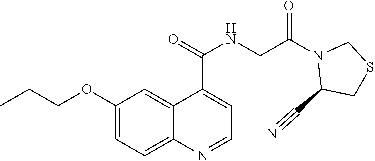 [SMILES: CCCOc1ccc2nccc(C(=O)NCC(=O)N3CSC[C@H]3C#N)c2c1]

DIPEA (2.1 mL, 12 mmol) was added to a solution of 6-propoxyquinoline-4-carboxylic acid Intermediate 156 (138 mg, 0.60 mmol), (R)-3-glycylthiazolidine-4-carbonitrile hydrochloride Intermediate 4 (186 mg, 0.90 mmol), HOBt (806 mg, 5.97 mmol) and EDC (1.14 g, 5.97 mmol) in MeCN (6 mL) and EtOAc (6 mL) at 25° C. and the reaction mixture was stirred at 25° C. for 16 h. The solvent was removed under reduced pressure and the reaction mixture was diluted with sat NaHCO₃ (aq, 250 mL) and extracted with EtOAc (3×250 mL). The combined organic layer was dried over Na₂SO₄, filtered and evaporated. The crude product was purified by preparative HPLC, PrepMethod D (gradient: 25-35%) to give the title compound (0.11 g, 48%); HRMS (ESI) m/z [M+H]⁺ calcd for C₁₉H₂₁N₄O₃S: 385.1328, found: 385.1320; ¹H NMR (400 MHz, DMSO-d₆) δ 9.09 (t, 1H), 8.80 (d, 1H), 7.98 (d, 1H), 7.89 (d, 1H), 7.50 (d, 1H), 7.46 (dd, 1H), 5.33 (dd, 1H), 4.90 (d, 1H), 4.72 (d, 1H), 4.46-4.20 (m, 2H), 4.11 (t, 2H), 3.47-3.33 (m, overlapping with solvent peak), 1.87-1.76 (m, 2H), 1.03 (t, 3H).

### Example 74: (R)—N-(2-(4-Cyanothiazolidin-3-yl)-2-oxoethyl)-6-(2-fluoroethoxy)-quinoline-4-carboxamide

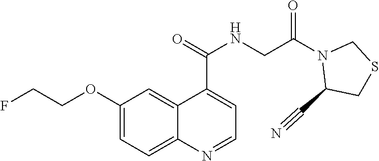 [SMILES: N#C[C@@H]1CSCN1C(=O)CNC(=O)c1ccnc2ccc(OCCF)cc12]

DIPEA (1.83 mL, 10.5 mmol) was added to 6-(2-fluoroethoxy)quinoline-4-carboxylic acid Intermediate 158 (123 mg, 0.52 mmol), (R)-3-glycylthiazolidine-4-carbonitrile hydrochloride Intermediate 4 (163 mg, 0.78 mmol), HOBt (707 mg, 5.23 mmol) and EDC (1.0 g, 5.2 mmol) in MeCN (6 mL) and EtOAc (6 mL) at 25° C., and the reaction mixture was stirred at 50° C. for 2 h. The solvent was removed under reduced pressure and the reaction mixture was diluted with sat NaHCO₃ (aq, 250 mL). The reaction mixture was extracted with EtOAc (3×250 mL) and the combined organic layer was dried over Na₂SO₄, filtered and evaporated. The crude product was purified by preparative HPLC, PrepMethod D (gradient: 15-32%) to give the title compound (0.141 g, 69%); HRMS (ESI) m/z [M+H]⁺ calcd for C₁₈H₁₈FN₄O₃S: 389.1078, found: 389.1070; ¹H NMR (400 MHz, DMSO-d₆) δ 9.10 (t, 1H), 8.83 (d, 1H), 8.01 (d, 1H), 7.96 (d, 1H), 7.56-7.47 (m, 2H), 5.35 (dd, 1H), 4.95-4.85 (m, 2H), 4.77 (t, 1H), 4.72 (d, 1H), 4.51-4.29 (m, 4H), 3.47-3.33 (m, overlapping solvent peak).

### Example 75: (R)—N-(2-(4-Cyanothiazolidin-3-yl)-2-oxoethyl)-6-(1-fluoro-cyclobutyl)quinoline-4-carboxamide

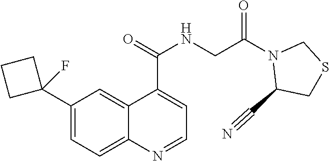 [SMILES: N#C[C@@H]1CSCN1C(=O)CNC(=O)c1ccnc2ccc(C3(F)CCC3)cc12]

A solution of 6-(1-fluorocyclobutyl)quinoline-4-carboxylic acid Intermediate 162 (100 mg, 0.41 mmol), (R)-3-glycylthiazolidine-4-carbonitrile hydrochloride Intermediate 4 (110 mg, 0.53 mmol), EDC (117 mg, 0.61 mmol), HOBt (94 mg, 0.61 mmol) and DIPEA (214 μL, 1.22 mmol) in EtOAc (5 mL) and MeCN (5 mL) was stirred at 50° C. for 2 h. The solvent was removed under reduced pressure and the crude product was diluted with EtOAc, and washed with water. The organic layer was dried over Na₂SO₄, filtered and evaporated. The crude product was purified by preparative HPLC, PrepMethod D (gradient: 30-40%), and then by preparative HPLC, PrepMethod P (gradient: 45-55%) to give the title compound (90 mg, 55%); HRMS (ESI) m/z [M+H]⁺ calcd for C₂₀H₂₀FN₄O₂S: 399.1286, found: 399.1280; ¹H NMR (300 MHz, CD₃OD) δ 8.95 (d, 1H), 8.60-8.55 (m, 1H), 8.14 (d, 1H), 7.96 (dd, 1H), 7.68 (d, 1H), 5.36 (dd, 1H), 4.87-4.65 (m, overlapping with solvent peak), 4.52-4.32 (m, 2H), 3.56-3.35 (m, 2H), 2.89-2.57 (m, 4H), 2.24-2.02 (m, 1H), 2.00-1.79 (m, 1H).

### Example 76: (R)—N-(2-(4-Cyanothiazolidin-3-yl)-2-oxoethyl)-6-(1-ethoxycyclopropyl)quinoline-4-carboxamide

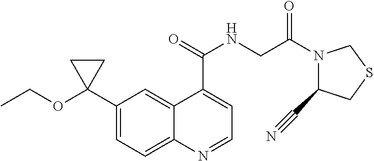 [SMILES: CCOC1(c2ccc3nccc(C(=O)NCC(=O)N4CSC[C@H]4C#N)c3c2)CC1]

A solution of 6-(1-ethoxycyclopropyl)quinoline-4-carboxylic acid Intermediate 165 (50 mg, 0.19 mmol), (R)-3-glycylthiazolidine-4-carbonitrile hydrochloride Intermediate 4 (81 mg, 0.39 mmol), EDC (56 mg, 0.29 mmol), HOBt (45 mg, 0.29 mmol) and DIPEA (0.17 mL, 0.97 mmol) in DMF (4 mL) was stirred at rt for 3 h. The solvent was removed under reduced pressure and the crude product was purified by preparative TLC (DCM:MeOH, 10:1), followed by preparative HPLC, PrepMethod I (gradient: 25-45%) to give the title compound (35 mg, 44%); HRMS (ESI) m/z [M+H]⁺ calcd for C₂₁H₂₃N₄O₃S: 411.1486, found: 411.1468; ¹H NMR (400 MHz, CD₃OD) δ 8.91 (d, 1H), 8.31 (d, 1H), 8.09 (d, 1H), 7.86 (dd, 1H), 7.67 (d, 1H), 5.39 (dd, 1H), 4.89-4.78 (m, overlapping with solvent peak), 4.51-4.36 (m, 2H), 3.54 (q, 2H), 3.48-3.34 (m, overlapping with solvent peak), 1.41-1.14 (m, 7H).

### Example 77: (R)—N-(2-(4-Cyanothiazolidin-3-yl)-2-oxoethyl)-6-cyclopropylquinoline-4-carboxamide

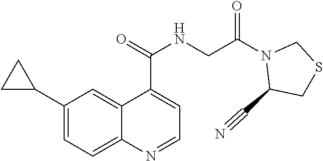 [SMILES: N#C[C@@H]1CSCN1C(=O)CNC(=O)c1ccnc2ccc(C3CC3)cc12]

A solution of 6-cyclopropylquinoline-4-carboxylic acid Intermediate 167 (80 mg, 0.38 mmol), (R)-3-glycylthiazolidine-4-carbonitrile hydrochloride Intermediate 4 (156 mg, 0.75 mmol), EDC (108 mg, 0.56 mmol), HOBt (86 mg, 0.56 mmol) and DIPEA (0.328 mL, 1.88 mmol) in DMF (5 mL) was stirred at rt for 3 h. The solvent was removed under reduced pressure and the crude product was purified by preparative TLC (DCM:MeOH: 10:1), followed by preparative HPLC, PrepMethod I (gradient: 28-40%) to give the title compound (70 mg, 51%); HRMS (ESI) m/z [M+H]⁺ calcd for C₁₉H₁₉N₄O₂S: 367.1224, found: 367.1220; ¹H NMR (300 MHz, CD₃OD) δ 8.82 (d, 1H), 8.09 (d, 1H), 7.97 (d, 1H), 7.63-7.52 (m, 2H), 5.38 (dd, 1H), 4.88-4.68 (m, overlapping with solvent peak), 4.50-4.31 (m, 2H), 3.52-3.33 (m, overlapping with solvent peak), 2.21-2.06 (m, 1H), 1.18-1.02 (m, 2H), 1.00-0.82 (m, 2H).

### Example 78: N-(2-((R)-4-Cyanothiazolidin-3-yl)-2-oxoethyl)-6-((1r,4R*)-4-methoxycyclohexyl)quinoline-4-carboxamide Isomer 1

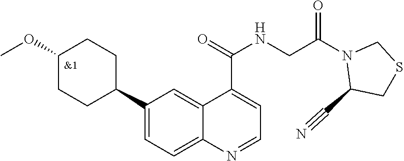 [SMILES: CO[C@H]1CC[C@H](c2ccc3nccc(C(=O)NCC(=O)N4CSC[C@H]4C#N)c3c2)CC1]

N-(2-((R)-4-Cyanothiazolidin-3-yl)-2-oxoethyl)-6-((1r,4RS)-4-methoxycyclohexyl)quinoline-4-carboxamide Intermediate 172 (30 mg) was purified by preparative HPLC, PrepMethod V (gradient: 45-62%) to give the title compound as the first eluted compound (23 mg, 76%); HRMS (ESI) m/z [M+H]⁺ calcd for C₂₃H₂₇N₄O₃S: 439.1798, found: 439.1821; ¹H NMR (300 MHz, DMSO-d₆) δ 9.09 (t, 1H), 8.92 (d, 1H), 8.25-8.17 (m, 1H), 8.01 (d, 1H), 7.74 (dd, 1H), 7.52 (d, 1H), 5.36 (dd, 1H), 4.91 (d, 1H), 4.73 (d, 1H), 4.33 (d, 2H), 3.52-3.33 (m, overlapping with solvent peak), 3.30-3.18 (m, 4H), 2.80-2.63 (m, 1H), 2.21-2.09 (m, 2H), 1.99-1.85 (m, 2H), 1.74-1.52 (m, 2H), 1.39-1.18 (m, 2H).

### Example 79: N-(2-((R)-4-Cyanothiazolidin-3-yl)-2-oxoethyl)-6-((1r,4R*)-4-methoxycyclohexyl)quinoline-4-carboxamide Isomer 2

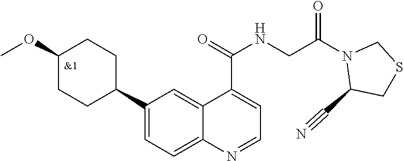 [SMILES: CO[C@H]1CC[C@@H](c2ccc3nccc(C(=O)NCC(=O)N4CSC[C@H]4C#N)c3c2)CC1]

N-(2-((R)-4-Cyanothiazolidin-3-yl)-2-oxoethyl)-6-((1r,4RS)-4-methoxycyclohexyl)quinoline-4-carboxamide Intermediate 172 (30 mg) was purified by preparative HPLC, PrepMethod V (gradient: 45-62%) to give the title compound as the second eluted compound (15 mg, 50%): HRMS (ESI) m/z [M+H]⁺ calcd for C₂₃H₂₇N₄O₃S: 439.1798 found: 439.1806; ¹H NMR (300 MHz, DMSO-d₆) δ 9.09 (t, 1H), 8.93 (d, 1H), 8.12 (d, 1H), 8.02 (d, 1H), 7.72 (dd, 1H), 7.55 (d, 1H), 5.36 (dd, 1H), 4.90 (d, 1H), 4.73 (d, 1H), 4.34 (d, 2H), 3.55-3.36 (m, overlapping with solvent peak), 3.27 (s, 3H), 2.86-2.68 (m, 1H), 2.09-1.93 (m, 2H), 1.91-1.71 (m, 2H), 1.70-1.49 (m, 4H).

### Example 80: (R)-6-((1H-Pyrazol-1-yl)methyl)-N-(2-(4-cyanothiazolidin-3-yl)-2-oxoethyl)quinoline-4-carboxamide

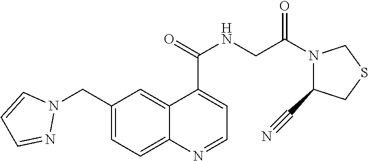 [SMILES: N#C[C@@H]1CSCN1C(=O)CNC(=O)c1ccnc2ccc(Cn3cccn3)cc12]

DIPEA (469 μL, 2.68 mmol) was added to a stirred suspension of Intermediate 10 (34 mg, 0.13 mmol), (R)-3-glycylthiazolidine-4-carbonitrile hydrochloride (56 mg, 0.27 mmol) Intermediate 4, HOBt (206 mg, 1.34 mmol) and EDC (257 mg, 1.34 mmol) in MeCN (3 mL) and EtOAc (3 mL). The reaction mixture was stirred at 20° C. for 16 h and concentrated under reduced pressure. The reaction mixture was diluted with sat NaHCO₃ (aq, 100 mL), and washed with EtOAc (5×100 mL). The organic layer was dried over Na₂SO₄, filtered and evaporated. The residue was purified by preparative HPLC, PrepMethod P (gradient 24-33%) to give the title compound (13 mg, 24%): HRMS (ESI) m/z [M+H]⁺ calcd for C₂₀H₁₉N₆O₂S: 407.1284, found: 407.1272; ¹H NMR (400 MHz, DMSO-d₆) δ 9.12 (t, 1H), 8.98 (d, 1H), 8.31-8.23 (m, 1H), 8.04 (d, 1H), 7.90 (d, 1H), 7.62-7.56 (m, 2H), 7.48 (d, 1H), 6.29 (t, 1H), 5.53 (s, 2H), 5.38 (dd, 1H), 4.91 (d, 1H), 4.74 (d, 1H), 4.33 (d, 2H), 3.46-3.34 (m, overlapping with solvent peak).

### Example 81: (R)—N-(2-(4-cyanothiazolidin-3-yl)-2-oxoethyl)-6-(3-methoxypropyl)quinoline-4-carboxamide

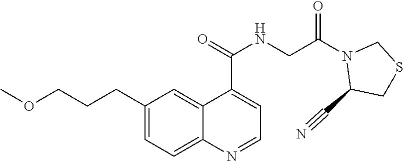 [SMILES: COCCCc1ccc2nccc(C(=O)NCC(=O)N3CSC[C@H]3C#N)c2c1]

DIPEA (278 μL, 1.59 mmol) was added to a solution of Intermediate 12 (130 mg, 0.53 mmol), (R)-3-glycylthiazolidine-4-carbonitrile hydrochloride Intermediate 4 (165 mg, 0.80 mmol), EDC (152 mg, 0.80 mmol) and HOBt (122 mg, 0.80 mmol) in MeCN (5 mL) and EtOAc (5 mL). The reaction mixture was stirred at 50° C. for 2 h and then concentrated under reduced pressure. The reaction mixture was diluted EtOAc and washed with water. The organic layer was dried over Na₂SO₄, filtered and evaporated. The crude product was purified by preparative HPLC, PrepMethod N (gradient: 13-25%) to give the title compound (55 mg, 26%): HRMS (ESI) m/z [M+H]⁺ calcd for C₂₀H₂₃N₄O₃S: 399.1486, found: 399.1492; ¹H NMR (300 MHz, CD₃OD) δ 8.86 (d, 1H), 8.19 (d, 1H), 8.01 (d, 1H), 7.72 (dd, 1H), 7.62 (d, 1H), 5.39 (dd, 1H), 4.88-4.68 (m, overlapping with solvent peak), 4.51-4.34 (m, 2H), 3.54-3.36 (m, 4H), 3.34 (s, 3H), 2.90 (dd, 2H), 2.07-1.91 (m, 2H).

### Example 82: (R)—N-(2-(4-cyanothiazolidin-3-yl)-2-oxoethyl)-6-(2-methoxyethyl)quinoline-4-carboxamide

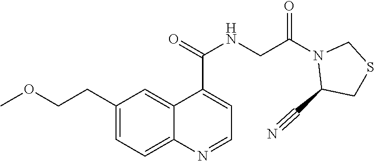 [SMILES: COCCc1ccc2nccc(C(=O)NCC(=O)N3CSC[C@H]3C#N)c2c1]

DIPEA (136 μL, 0.78 mmol) was added to a solution of Intermediate 14 (60 mg, 0.26 mmol), (R)-3-glycylthiazolidine-4-carbonitrile hydrochloride Intermediate 4 (81 mg, 0.39 mmol), EDC (75 mg, 0.39 mmol) and HOBt (60 mg, 0.39 mmol) in MeCN (4 mL) and EtOAc (4 mL). The reaction mixture was stirred at 50° C. for 2 h and then concentrated under reduced pressure. The reaction mixture was diluted EtOAc and washed with water. The organic layer was dried over Na₂SO₄, filtered and evaporated. The crude product was purified by preparative HPLC, PrepMethod R (gradient: 11-21%) to give the title compound (55 mg, 55%): HRMS (ESI) m/z [M+H]⁺ calcd for C₁₉H₂₁N₄O₃S: 385.1328, found: 385.1340; ¹H NMR (400 MHz, CD₃OD) δ 8.88 (d, 1H), 8.28 (d, 1H), 8.03 (d, 1H), 7.77 (dd, 1H), 7.64 (d, 1H), 5.39 (dd, 1H), 4.89-4.76 (m, overlapping with solvent peak), 4.50-4.36 (m, 2H), 3.77 (t, 2H), 3.55-3.36 (m, 5H), 3.11 (t, 2H).

### D. Biological Data

The hFAP protein used in the Examples was either commercially sourced or produced in insect cells as recombinant hFAP (Gp67-6HN-TEV-FAP (M39-A757), MW 89086.7 Da, or cd33-FAP (27-757)-6His, MW85926 Da). Recombinant hFAP protein was secreted from Sf21 cells in media, purified with affinity (batchmode, Ni excel resin, ÄKTA, GE Healthcare) and size exclusion chromatography (Superdex200, ÄKTA, GE Healthcare), concentrated to 19.5 mg/mL, snapfrozen in liquid N2 and stored in −80° C.

### Example 83: FAP Inhibition and Binding Assays

### A. hFAP Inhibition Assay

Compounds were tested in a biochemical inhibition assay using hFAP enzyme at 0.24 nM FAC (Proteros, 38-760 (PR-0071)) and the substrate Ala-Pro-AMC (ARI-3144) at 20 μM FAC. 384 low volume black plates (Greiner #784076) were used. 4 μL, 0.48 nM enzyme solution (100 mM Tris HCl, 100 mM NaCl, 0.05% Chaps, pH 7.4) was added to 40 nL compounds (in DMSO) at 10 CR, 3 fold dilution series from 50 μM FAC. Plates were incubated for 15 min at rt in dark. 4 μL, 40 μM substrate solution (100 mM Tris HCl, 100 mM NaCl, 0.05% Chaps, pH 7.4) was added to each well. Plates were centrifuged at 1000 rpm and incubated for 30 min at rt in dark. The plates were read on a PHERAstar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound A (i.e., (S)—N-(2-(2-cyano-4,4-difluoropyrrolidin-1-yl)-2-oxoethyl)quinoline-4-carboxamide as reported in J. Med. Chem. 2014, 57, 3053) at 50 μM as 100% inhibitor control. Data for the compounds tested are reported in Table 1A.

### B. hFAP Inhibition Assay (Tight Binders)

Compounds were tested in a biochemical inhibition assay using human Fibroblast activation protein alpha (hFAP) enzyme at 2.4 pM FAC (Proteros, 38-760 (PR-0071) and the substrate Ala-Pro-AMC (ARI-3144) at 20 μM FAC. 384 low volume black plates (Greiner #784076) were used. 4 μL, 4.8 pM enzyme solution (100 mM Tris HCl, 100 mM NaCl, 0.05% Chaps, pH 7.4) was added to 40 nL compounds (in DMSO) at 10 CR, 3 fold dilution series from 50 nM FAC. Plates were incubated for 15 min at rt in dark. 4 μL, 40 μM substrate solution (100 mM Tris HCl, 100 mM NaCl, 0.05% Chaps, pH 7.4) was added to each well. Plates were centrifuged at 1000 rpm and incubated for 2.5 h at rt in dark. The plates were read on a PHERAstar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound A (i.e., (S)—N-(2-(2-cyano-4,4-difluoropyrrolidin-1-yl)-2-oxoethyl)quinoline-4-carboxamide as reported in J. Med. Chem. 2014, 57, 3053) at 50 μM as 100% inhibitor control. Data for the compounds tested are reported in Table 1A.

TABLE 1A

| | hFAP inhibition | hFAP inhibition assay |
|---|---|---|
| | assay | (tight binders) |
| Example | IC₅₀ (nM)¹ | IC₅₀ (nM)² |
| 1 | 0.44 | |
| 2 | | 0.096 |
| 3 | | 0.15 |
| 4 | | 0.063 |
| 5 | | 0.042 |
| 6 | | 0.089 |
| 7 | | 0.085 |
| 8 | | 0.12 |
| 9 | | 0.25 |
| 10 | | 0.085 |
| 11 | | 0.070 |
| 12 | | 0.090 |
| 13 | | 0.18 |
| 14 | 0.079 | |
| 15 | 0.45 | 0.12 |
| 16 | 0.60 | 0.26 |
| 17 | 0.79 | 0.43 |
| 18 | 0.81 | 0.28 |
| 19 | 0.22 | 0.11 |
| 20 | 0.23 | 0.095 |
| 21 | | 39 |
| 22 | 0.24 | 0.042 |
| 23 | 0.71 | 0.16 |
| 24 | | 0.19 |
| 25 | 0.66 | 0.25 |
| 26 | 0.84 | 0.45 |
| 27 | | <0.16 |
| 28 | 0.65 | |
| 29 | 0.52 | |
| 30 | 1.7 | 0.40 |
| 31 | | 0.53 |
| 32 | 0.63 | 0.21 |
| 33 | 0.54 | 0.20 |
| 34 | 0.61 | 0.14 |
| 35 | | 0.26 |
| 36 | | 0.24 |
| 37 | | 0.20 |
| 38 | 0.21 | 0.079 |
| 39 | 0.34 | 0.081 |
| 40 | 0.25 | 0.063 |
| 41 | 0.088 | 0.11 |
| 42 | 0.27 | |
| 43 | 0.094 | 0.13 |
| 44 | | 0.12 |
| 45 | | 0.12 |
| 46 | | 0.13 |
| 47 | | 0.15 |
| 48 | | 0.11 |
| 49 | | 0.13 |
| 50 | | 0.064 |
| 51 | | 0.062 |
| 52 | | 0.077 |
| 53 | | 0.085 |
| 54 | | 0.041 |
| 55 | | 0.035 |
| 56 | | 0.079 |
| 57 | | 0.093 |
| 58 | | 0.086 |
| 59 | | 0.039 |
| 60 | | 0.037 |
| 61 | | 0.25 |
| 62 | | 0.094 |
| 63 | | 0.049 |
| 64 | | 0.049 |
| 65 | | 0.084 |
| 66 | 0.15 | 0.14 |
| 67 | 0.30 | |
| 68 | 0.30 | 0.10 |
| 69 | 0.20 | |
| 70 | 0.43 | 0.071 |
| 71 | 0.43 | |
| 72 | 0.20 | 0.026 |
| 73 | | 0.042 |
| 74 | | 0.070 |
| 75 | | 0.061 |
| 76 | 1.3 | 0.67 |
| 77 | 0.59 | |
| 78 | | 0.033 |
| 79 | | 0.067 |
| 80 | | 0.17 |
| 81 | | 0.097 |
| 82 | | 0.12 |

¹IC₅₀ is reported as geometric mean for multiple measurements (n = 2 − 4).
²IC₅₀ is reported after single measurement (n = 1) or as geometric mean for multiple measurements (n = 2 − 5).

### C. hFAP Binding Assay

Compounds were tested in a direct binding assay using 8K surface plasmon resonance biosensor (GE Healthcare) at 20° C. Immobilization of hFAP (M39-A757) on a CMD200M sensor chip (Xantec) was performed using standard amine coupling procedure in immobilization buffer (10 mM HEPES, 150 mM NaCl, 0.05% Tween20, pH 7.4). The surface was washed with 10 mM aq NaOH, 1M NaCl before being activated with EDC/NHS (GE Healthcare), followed by immobilization of hFAP (in 10 mM Acetate pH 5.0). Finally, the surface was deactivated by ethanolamine. Immobilization levels of hFAP were around 4000-6000 RU. The reference spot was treated as described, omitting the injection of hFAP. Compound concentration series were injected over the immobilized protein in increasing concentrations (2-500 nM) using single cycle kinetics in running buffer (20 mM TRIS, 150 mM NaCl, 0.05% Tween20, 1% DMSO, pH 7.4). Interaction models were fitted globally to the experimental traces, enabling determination of kₒₙ, k_{off} and K_{d}. Data for the compounds tested are reported in Table 1B.

TABLE 1B

| | hFAP Binding | hFAP Binding | hFAP Binding |
|---|---|---|---|
| | assay | assay | assay |
| Example | K_{d} (nM)¹ | k₍ₒₙ₎ (M⁻¹s⁻¹)¹ | k_{(off)} (1/s)¹ |
| 1 | 0.33 | 760000 | 0.00025 |
| 2 | 0.052 | 12000000 | 0.00088 |
| 3 | 0.062 | 2900000 | 0.00013 |
| 4 | 0.057 | 1400000 | 0.000082 |
| 12 | 0.19 | 8800000 | 0.0017 |
| 13 | 0.12 | 5100000 | 0.00061 |
| 14 | 0.046 | 4100000 | 0.00039 |
| 15 | 0.068 | 2000000 | 0.00013 |
| 16 | 0.23 | 570000 | 0.00013 |
| 17 | 0.91 | 540000 | 0.00049 |
| 18 | 0.43 | 1200000 | 0.00049 |
| 19 | 0.10 | NV | NV |
| 20 | 0.056 | 1400000 | 0.000073 |
| 21 | 140 | 15000 | 0.0020 |
| 22 | 0.013 | 7600000 | 0.00010 |
| 23 | 0.059 | 340000000 | 0.026 |
| 24 | 0.33 | 850000 | 0.00026 |
| 25 | 0.80 | 510000 | 0.00040 |
| 26 | 4.7 | 140000 | 0.00063 |
| 28 | 1.6 | 580000 | 0.00089 |
| 29 | 1.4 | 440000 | 0.00062 |
| 30 | 0.63 | 550000 | 0.00034 |
| 31 | 0.40 | 880000 | 0.00033 |
| 32 | 0.35 | 220000 | 0.000080 |
| 33 | 0.30 | 460000 | 0.00013 |
| 34 | 0.31 | 340000 | 0.00010 |
| 38 | 0.34 | 1100000 | 0.00027 |
| 39 | 0.18 | 16000000 | 0.0022 |
| 40 | 0.13 | NV | NV |
| 41 | 0.36 | 35000000 | 0.016 |
| 42 | 0.20 | NV | NV |
| 43 | 0.39 | 5200000 | 0.0022 |
| 44 | 0.14 | 6700000 | 0.0014 |
| 45 | 0.13 | 34000000000 | 4.2 |
| 46 | 0.13 | 59000000 | 0.0078 |
| 47 | 0.22 | 3500000 | 0.00078 |
| 48 | 0.12 | 6000000 | 0.00068 |
| 49 | 0.13 | 9400000 | 0.0019 |
| 51 | 0.12 | NV | NV |
| 52 | 0.20 | 460000 | 0.00013 |
| 62 | 0.11 | 2900000 | 0.00029 |
| 63 | 0.097 | 5700000 | 0.00059 |
| 64 | 0.042 | 9200000000 | 0.39 |
| 65 | 0.091 | 1200000 | 0.00011 |
| 66 | 0.15 | NV | NV |
| 67 | 0.11 | 6200000 | 0.00066 |
| 68 | 0.075 | 16000000 | 0.00069 |
| 69 | 0.026 | 3000000 | 0.000077 |
| 70 | 0.62 | 7700000 | 0.0030 |
| 71 | 0.40 | 2100000 | 0.00085 |
| 72 | 0.013 | 5100000 | 0.000065 |
| 76 | 2 | 160000 | 0.00032 |
| 77 | 1.4 | 460000 | 0.00065 |
| 78 | 0.022 | 2300000 | 0.000051 |
| 80 | 0.30 | 1600000 | 0.00062 |

¹K_{d}, k₍ₒₙ₎ and k_{(off)} are reported after single measurement (n = 1) or as geometric mean for multiple measurements (n = 2 − 3).

### D. FAP Plasma Inhibition Assay

This assay was adapted from the method described in Example 89 for detection of FAP target engagement enzyme activity in plasma. Plasma (anticoagulant K2EDTA) was used as the enzyme source: Human plasma (Pooled from AZ Biobank) and Mouse plasma (AZ AST Biobank) and Cynomolgus plasma (BioIVT, #NHP00PLK2FNN, lot CYN222895). 384-Well black fluotrack PS plates (Greiner 781076) were used. 20 μL diluted plasma (Cynomolgus and Human plasma dilution 1:40, Mouse plasma dilution 1:67) in buffer (PBS, 0.1% BSA) was added to 0.6 μL compounds (in DMSO). Compounds were tested using 10 CR, 3 fold dilution series from 500 nM FAC. Two replicates for each assay point were run on the same plate. A fluorescence blank read was taken before substrate addition. Substrate, Ala-Pro-AMC (ARI-3144) stock solution (20 mM in DMSO) was diluted in buffer (PBS, 0.1% BSA) to 150 μM concentration and 20 μL added giving 75 μM FAC. Plates were incubated for 40 min at rt in the dark. The plates were read on a Beckman Paradigm® reader with excitation 360 nm and emission 465 nm. Data were analyzed in Excel (IDBS XLfit Add-In) using a one site dose response model (4-parameter logistic fit). IC₅₀ values were determined by plotting % inhibition versus log compound concentration. Raw data signals were normalized using 1.5% DMSO in diluted plasma as 0% control and 1.5% DMSO in buffer (no plasma) as 100% inhibitor control. Data for the compounds tested are reported in Table 2.

TABLE 2

| | FAP Human | FAP Mouse | FAP Cyno |
|---|---|---|---|
| | Plasma | Plasma | Plasma |
| Example | IC₅₀ (nM)¹ | IC₅₀ (nM)¹ | IC₅₀ (nM)¹ |
| 3 | 0.30 | 0.50 | 0.40 |
| 15 | 0.30 | 0.50 | 0.30 |
| 16 | 0.90 | 0.90 | |
| 18 | 1.5 | 2.1 | |
| 20 | 0.30 | 0.50 | 0.30 |
| 23 | 0.50 | 0.70 | 0.40 |
| 32 | 0.50 | 1 | 0.50 |
| 70 | 0.70 | 0.70 | |
| 78 | 0.10 | 0.20 | 0.10 |

¹IC₅₀ is reported after single measurement (n = 1).

### Example 84: hPrep Inhibition Assay

Compounds were tested in a biochemical inhibition assay using Prolyl endopeptidase, Prolyl Oligopeptidase (hPREP) enzyme at 0.6 nM FAC (R&D Systems, 4308-SE) and the substrate Z-Gly-Pro-amino-methylcoumarin (Bachem, I-1145) at 50 μM FAC. 384 Low volume black plates (Greiner #784076) were used. 4 μL, 1.2 nM enzyme solution (25 nM Tris HCl, 250 mM NaCl, 0.01% Triton X-100, 5 mM Glutathione, pH 7.5) was added to 40 nL compounds (in DMSO) at 10 CR, 3 fold dilution series from 50 μM FAC. Plates were incubated for 15 min at rt in dark. 4 μL, 100 μM substrate solution (25 mM Tris HCl, 250 mM NaCl, 0.01% Triton X-100, 5 mM Glutathione, pH 7.5) was added to each well. Plates were centrifuged at 1000 rpm and incubated for 20 min at rt in dark. The plates were read on a PHERAstar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound B (i.e., (R)—N-(2-(4-cyanothiazolidin-3-yl)-2-oxoethyl)-7-methylquinoline-4-carboxamide) at 50 μM as 100% inhibitor control. Data for the compounds tested are reported in Table 3.

TABLE 3

| | PREP IC₅₀ |
|---|---|
| Example | (nM)¹ |
| 1 | 35 |
| 2 | 190 |
| 3 | 950 |
| 4 | 570 |
| 5 | 400 |
| 6 | 1200 |
| 7 | 150 |
| 8 | 1200 |
| 9 | 870 |
| 10 | 32 |
| 11 | 290 |
| 12 | 110 |
| 13 | 460 |
| 14 | 8.4 |
| 15 | 1100 |
| 16 | 640 |
| 17 | 35 |
| 18 | 860 |
| 19 | 590 |
| 20 | 1000 |
| 21 | 8.3 |
| 22 | 560 |
| 23 | 1100 |
| 24 | 1000 |
| 25 | 440 |
| 26 | 470 |
| 27 | 140 |
| 28 | 34 |
| 29 | 32 |
| 30 | 1700 |
| 31 | 910 |
| 32 | 910 |
| 33 | 680 |
| 34 | 460 |
| 35 | 460 |
| 36 | 170 |
| 37 | 170 |
| 38 | 180 |
| 39 | 530 |
| 40 | 600 |
| 41 | 370 |
| 42 | 180 |
| 43 | 370 |
| 44 | 65 |
| 45 | 150 |
| 46 | 80 |
| 47 | 240 |
| 48 | 240 |
| 49 | 110 |
| 50 | 130 |
| 51 | 170 |
| 52 | 320 |
| 53 | 6.5 |
| 54 | 620 |
| 55 | 330 |
| 56 | 500 |
| 57 | 790 |
| 58 | 64 |
| 59 | 430 |
| 60 | 290 |
| 61 | 640 |
| 62 | 520 |
| 63 | 330 |
| 64 | 300 |
| 65 | 210 |
| 66 | 460 |
| 67 | 87 |
| 68 | 330 |
| 69 | 350 |
| 70 | 120 |
| 71 | 15 |
| 72 | 540 |
| 73 | 160 |
| 74 | 83 |
| 75 | 46 |
| 76 | 580 |
| 77 | 38 |
| 78 | 1000 |
| 79 | 610 |
| 80 | 290 |
| 81 | 62 |
| 82 | 48 |
| B² | 5.2 |
| C³ | 7.1 |

¹IC₅₀ is reported after single measurement (n = 1) or as geometric mean for multiple measurements (n = 2-30).
²Reference Compound B: (R)-N-(2-(4-cyanothiazolidin-3-yl)-2-oxoethyl)-7-methylquinoline-4-carboxamide
³Reference Compound C: (R)-N-(2-(4-cyanothiazolidin-3-yl)-2-oxoethyl)-quinoline-4-carboxamide

### Example 85: hDPP Inhibition Assays

### A. hDPP7 Inhibition Assay

Compounds were tested in a biochemical inhibition assay using human dipeptidylpeptidase 7 (hDPP7) enzyme at 15 nM FAC (BPS Bioscience, #80070) and the substrate Ala-Pro-amino-methylcoumarin (BPS Bioscience, #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3 fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC₅₀ values were determined by plotting % inhibition versus log compound concentration and using a one site dose response model. Raw data signals were normalized using 1% DMSO as 0% control and no enzyme as 100% inhibitor control. Data for the compounds tested are reported in Table 4.

### B. hDPP8 Inhibition Assay

Compounds were tested in a biochemical inhibition assay using human dipeptidylpeptidase 8 (hDPP8) enzyme at 1.5 nM FAC (BPS Bioscience, #80080) and the substrate Ala-Pro-amino-methylcoumarin (BPS Bioscience #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3 fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC50 values were determined by plotting % inhibition versus log compound concentration and using a one site dose response model. Raw data signals were normalized using 1% DMSO as 0% control and no enzyme as 100% inhibitor control. Data for the compounds tested are reported in Table 4.

### C. hDPP9 Inhibition Assay

Compounds were tested in a biochemical inhibition assay using human dipeptidylpeptidase 9 (hDPP9) enzyme at 0.4 nM FAC (BPS Bioscience, #80090) and the substrate Ala-Pro-amino-methylcoumarin (BPS Bioscience #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3 fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC₅₀ values were determined by plotting % inhibition versus log compound concentration and using a one site dose response model. Raw data signals were normalized using 1% DMSO as 0% control and no enzyme as 100% inhibitor control. Data for the compounds tested are reported in Table 4.

TABLE 4

| | hDPP7 | hDPP8 | hDPP9 |
|---|---|---|---|
| Example | IC₅₀ (nM)¹ | IC₅₀ (nM)¹ | IC₅₀ (nM)¹ |
| 3 | >100,000 | 6400 | 1900 |
| 6 | >100,000 | 6400 | 2900 |

¹IC₅₀ is reported after single measurement (n = 1).

### Example 86: Metabolic Stability Assays

### A. Aldehyde Oxidase (AO) Metabolism Assay 1

AO-mediated metabolism was measured essentially as described in Drug Metab Disp 2010, 38, 1322. Human liver cytosol (Corning life sciences, UltraPool Human Cytosol 150, Product 452115) in phosphate buffer, pH 7.4, was pre-incubated for 5 min at 37° C. shaking at 900 rpm. The reactions were initiated by addition of pre-diluted test compounds including positive control, zaleplon, and incubated at 37° C. with final conditions 2.5 mg/mL human liver cytosolic fraction, 1 μM test compound, 0.01% DMSO and 0.09% MeCN. The samples were incubated for 120 min with time points taken at 0, 10, 30, 60, 90 and 120 min. The aliquots (25 μL) were precipitated with 100 μL MeCN containing internal standard (4-((2′-(1H-tetrazol-5-yl)-[1,1′-biphenyl]-4-yl)methoxy)-2-ethylquinoline, (J Med Chem 1992, 35, 4027), centrifuged at 3500 rpm for 10 min and the supernatant diluted 1 in 7 (v/v) with ultra-pure HPLC water before analysis by LC-MS/MS. All incubations were carried out in duplicate. The in vitro elimination rate constant corresponding to parent compound depletion was determined for each reaction using the 1^{st} order decay calculation in Microsoft Excel Sheet. In some cases the experiment was conducted additionally in presence of an aldehyde oxidase inhibitor: The cytosol mix was pre-incubated with 3 μM raloxifene shaking at 900 rpm for 5 min at 37° C. prior to addition of test compound. Data for the compounds tested are reported in Table 5.

### B. Aldehyde Oxidase (AO) Metabolism Assay 2

AO-mediated metabolism was measured essentially as described in Drug Metab Disp. 2010, 38, 1322. Human liver cytosol (BioreclamationIVT, stored at −80° C. prior to use, protein concentration 2.5 mg/mL) and 0.1 M phosphate buffer (with 0.1 mM EDTA) pH 7.4 is preincubated at 37° C. The reaction was initiated by addition of test compound (final substrate concentration 1 μM, final DMSO concentration 0.3% and final incubation volume 500 μL). Phthalazine (known to be metabolized by AO) was used as a control compound. Test compounds were incubated for 0, 5, 15, 30, 60 and 120 min. The reactions were stopped by removing an aliquot of incubate into organic solvent containing internal standard at the appropriate time points. The termination plates were centrifuged at 2500 rpm for 30 min at 4° C. to precipitate the protein. Sample supernatants were combined in cassettes of up to four compounds and analyzed using generic LC MS/MS conditions. From a plot of ln peak area ratio (compound peak area/internal standard peak area) against time, the gradient of the line was determined. Subsequently, half-life and intrinsic clearance were calculated using the equations below:

$$Elimination rate constant ( k ) = ( - gradient )$$

$$Half - life ( {t}_{1 / 2} ) ( min ) = 0.693 / k$$

$$Intrinsic clearance ( {CL}_{int} ) ( μL / min / mg pro tein ) = V \times 0.693 / {t}_{1 / 2}$$

$$where V = Incubation volume ( μL ) / protein ( mg )$$

The percentage of the parent compound remaining at each time point, along with the intrinsic clearance value (CLᵢₙₜ), half-life and standard error of the CLᵢₙₜ were reported. Data for the compounds tested are reported in Table 5.

TABLE 5

| | AO-Assay 1 | AO-Assay 2 |
|---|---|---|
| Example | CLᵢₙₜ (μL/min/mg)¹ | CLᵢₙₜ (μL/min/mg)¹ |
| 1 | 0.26 | |
| 3 | | <0.50 |
| 20 | 0.5 | |
| 30 | 0.67 | |
| 32 | 1.1 | |
| 34 | 0.051 | |
| 70 | | 0.47 |
| 78 | 0.82 | |
| Compound A² | 3.8 | |
| Compound C³ | 5.3 | |

¹CLᵢₙₜ is reported after single measurement (n = 1).
²Compound A ((S)-N-(2-(2-cyano-4,4-difluoropyrrolidin-1-yl)-2-oxoethyl)quinoline-4-carboxamide, J Med Chem 2014, 57, 3053).
³Compound C (R)-N-(2-(4-cyanothiazolidin-3-yl)-2-oxoethyl)quinoline-4-carboxamide.

### C. Human Liver Microsomes (HLM)

Metabolic stability in HLM was measured as described in J Comput Aided Mol Des 2015, 29, 795. Data for the compounds tested are reported in Table 6.

### D. Rat Hepatocytes (rHep)

Metabolic stability in rat hepatocytes was measured as described in J Comput Aided Mol Des 2015, 29, 795. Data for the compounds tested are reported in Table 6.

### E. Human Hepatocytes (hHep)

Metabolic stability in human hepatocytes was measured as described in Xenobiotica 2010, 40, 637. Data for the compounds tested are reported in Table 6.

TABLE 6

| | HLM | rHeps | hHep |
|---|---|---|---|
| | CLᵢₙₜ¹ | CLᵢₙₜ² | CLᵢₙₜ³ |
| Example | (μL/min/mg) | (μL/min/1E6) | (μL/min/1E6) |
| 1 | 98 | 120 | |
| 2 | 36 | 5.9 | |
| 3 | 34 | 6.9 | 2.4 |
| 4 | <3 | <1 | |
| 5 | 90 | 16 | |
| 6 | 12 | <1.6 | 1.1 |
| 7 | 40 | 9.1 | |
| 8 | 45 | 11 | |
| 9 | 28 | 15 | |
| 10 | <3 | 8.4 | |
| 11 | 30 | 11 | |
| 12 | 230 | 110 | |
| 13 | >300 | 140 | |
| 14 | 25 | 11 | |
| 15 | <3 | <1 | <1 |
| 16 | 12 | <1 | <1 |
| 17 | 45 | 14 | |
| 18 | 69 | 26 | |
| 19 | 14 | 5.5 | <1 |
| 20 | 15 | 7.3 | <1 |
| 21 | 100 | 7.9 | |
| 22 | 17 | 7.4 | 1.6 |
| 23 | 72 | 44 | 3.1 |
| 24 | <3 | 1.8 | <1 |
| 25 | 5 | 2.9 | |
| 26 | 41 | 11 | |
| 27 | <3 | 11 | |
| 28 | 7.3 | 7.2 | |
| 29 | 9.8 | 12 | |
| 30 | <3 | 2.9 | |
| 31 | <3 | 4.4 | <1 |
| 32 | 35 | 11 | 3 |
| 33 | <3 | 2.3 | |
| 34 | 16 | 11 | |
| 35 | <3 | 4.3 | |
| 36 | 8.2 | 22 | |
| 37 | 9.1 | 20 | |
| 38 | 43 | 32 | 1.4 |
| 39 | 65 | 35 | |
| 40 | 29 | 21 | |
| 41 | 7.1 | 7.9 | <1 |
| 42 | 12 | 23 | |
| 43 | 100 | 78 | |
| 44 | >300 | 150 | |
| 45 | 70 | 21 | |
| 46 | 53 | 8.3 | |
| 48 | >300 | 27 | |
| 49 | 46 | 24 | |
| 50 | 64 | 18 | |
| 51 | 93 | 44 | |
| 52 | 210 | 49 | |
| 53 | 36 | 45 | |
| 54 | 62 | 32 | |
| 55 | <3 | 11 | |
| 56 | 24 | 6.3 | <1 |
| 57 | 150 | 20 | |
| 58 | 77 | 17 | |
| 59 | 30 | 20 | |
| 60 | >300 | 110 | |
| 61 | 77 | 23 | |
| 62 | 13 | 8.8 | |
| 63 | 19 | 8.8 | |
| 64 | 17 | 7.1 | |
| 65 | 11 | 3.7 | |
| 66 | 31 | 15 | |
| 67 | 16 | 11 | |
| 68 | 46 | 16 | |
| 69 | 36 | 23 | |
| 70 | 18 | 14 | 1.5 |
| 71 | 16 | 8.7 | |
| 72 | 63 | 18 | |
| 73 | 110 | 21 | |
| 74 | 26 | 2.3 | <1 |
| 75 | 240 | 61 | |
| 76 | 140 | 34 | |
| 77 | 58 | 44 | |
| 78 | <15 | 7.7 | 1.9 |
| 79 | 130 | 38 | |
| 80 | <3 | 5.5 | |
| 81 | 22 | 12 | |
| 82 | 29 | 3.5 | |

¹CLᵢₙₜ is reported after single measurement (n = 1) or as an average for multiple measurements (n = 2 − 3).
²CLᵢₙₜ is reported after single measurement (n = 1) or as geometric mean for multiple measurements (n = 2 − 3).
³CLᵢₙₜ is reported after single measurement (n = 1).

### Example 87: Caco-2 Cell Permeability

Caco-2 cell permeability was measured as described in Mol Pharm 2017, 14, 1601. Data for the compounds tested are reported in Table 7.

TABLE 7

| | | Caco2 | Caco2 | |
|---|---|---|---|---|
| | Caco2 | Bidirectional | Bidirectional | |
| | AB | (ABBA) | (ABBA) | Caco2 |
| | Intrinsic | A to B | B to A | Bidirectional |
| | Pₐₚₚ¹ | Pₐₚₚ¹ | Pₐₚₚ¹ | (ABBA) |
| | (1E−6 · | (1E−6 · | (1E−6 · | Efflux |
| Example | cm/s) | cm/s) | cm/s) | Ratio |
| 3 | 35 | 6.3 | 38 | 6 |
| 4 | 10 | | | |
| 6 | 19 | 1.9 | 27 | 14 |
| 9 | | 2.8 | 30 | 11 |
| 10 | | 1.4 | 16 | 12 |
| 11 | | 6.5 | 36 | 5.6 |
| 15 | 1 | 0.21 | 2 | 9.5 |
| 16 | | 0.27 | 10 | 37 |
| 19 | 6 | 0.84 | 23 | 27 |
| 20 | 15 | 1.4 | 29 | 21 |
| 22 | 19 | 1.7 | 25 | 14 |
| 23 | 11 | 0.73 | 22 | 30 |
| 24 | | 0.33 | 9.2 | 28 |
| 25 | | 0.59 | 4.9 | 8.2 |
| 26 | | 2.7 | 19 | 7.1 |
| 27 | | 3 | 20 | 6.6 |
| 30 | 3 | 0.46 | 12 | 28 |
| 31 | 5.7 | 0.79 | 21 | 27 |
| 32 | | 2.8 | 36 | 13 |
| 33 | | 0.56 | 22 | 41 |
| 34 | | 1.6 | 28 | 17 |
| 35 | | 0.64 | 16 | 25 |
| 36 | | 2.3 | 27 | 12 |
| 37 | | 5.3 | 31 | 5.8 |
| 38 | | 0.77 | 25 | 32 |
| 41 | 7.5 | 0.65 | 17 | 27 |
| 56 | 17 | 9.4 | 19 | 2.1 |
| 59 | | 0.24 | 21 | 90 |
| 62 | | 2.8 | 30 | 11 |
| 63 | | 4.3 | 30 | 7.1 |
| 64 | 13 | | | |
| 65 | 12 | | | |
| 66 | | 0.50 | 17 | 34 |
| 70 | | 0.83 | 15 | 18 |
| 74 | | 2.3 | 25 | 11 |
| 78 | 28 | 3.9 | 35 | 8.9 |
| 80 | | 0.59 | 19 | 31 |
| 81 | | 3.9 | 31 | 7.8 |
| 82 | | 4.6 | 20 | 4.3 |

¹Papp is reported after single measurement (n = 1) or as an average for multiple measurements (n = 2).

### Example 88: Kinetic Solubility

Kinetic solubility was measured as described in Comput Aided Mol Des 2015, 29, 795. Data for the compounds tested are reported in Table 8.

TABLE 8

| | Solubility |
|---|---|
| Example | (μM)¹ |
| 1 | 190 |
| 2 | 490 |
| 3 | 38 |
| 4 | >1000 |
| 5 | 230 |
| 6 | >950 |
| 7 | 550 |
| 8 | 8.3 |
| 9 | 820 |
| 10 | 490 |
| 11 | 180 |
| 12 | 25 |
| 13 | 78 |
| 14 | 420 |
| 15 | >1000 |
| 16 | >1000 |
| 17 | >1000 |
| 18 | >1000 |
| 19 | 210 |
| 20 | 580 |
| 21 | 500 |
| 22 | 340 |
| 23 | >1000 |
| 24 | >1000 |
| 25 | >1000 |
| 26 | >1000 |
| 27 | >1000 |
| 28 | >1000 |
| 29 | >1000 |
| 30 | 720 |
| 31 | 730 |
| 32 | 820 |
| 33 | 720 |
| 34 | 690 |
| 35 | >1000 |
| 36 | >1000 |
| 37 | 860 |
| 38 | 740 |
| 39 | 81 |
| 40 | >1000 |
| 41 | 950 |
| 42 | 190 |
| 43 | 60 |
| 44 | 410 |
| 45 | 250 |
| 46 | 410 |
| 48 | 240 |
| 49 | 220 |
| 50 | 180 |
| 51 | 160 |
| 52 | 120 |
| 53 | 190 |
| 54 | 8.5 |
| 55 | 270 |
| 56 | 710 |
| 57 | 430 |
| 58 | 700 |
| 59 | 480 |
| 60 | 180 |
| 61 | >1000 |
| 62 | 300 |
| 63 | 370 |
| 64 | 880 |
| 65 | >1000 |
| 66 | >1000 |
| 67 | 880 |
| 68 | 760 |
| 69 | 680 |
| 70 | 770 |
| 71 | >1000 |
| 72 | 14 |
| 73 | 190 |
| 74 | 190 |
| 75 | 120 |
| 76 | 900 |
| 77 | 470 |
| 78 | 120 |
| 79 | 33 |
| 80 | 340 |
| 81 | >1000 |
| 82 | >1000 |

¹Solubility is reported after single measurement (n = 1) or as an average for multiple measurements (n = 2-4).

### Example 89: FAP Target Engagement Enzyme Activity in Mouse Plasma

The effect of test compound on FAP enzyme activity in mouse plasma was evaluated in an enzymatic assay using the Fibroblast Activation Protein alpha (FAP) specific fluorogenic substrate dipeptide-Coumarin, Ala-Pro-AMC, (ARI-3144). In this assay, FAP cleaves Ala-Pro-AMC to release free AMC which is measured as a fluorescent signal that correlates with enzyme activity.

Male C57Bl/6 mice (Charles River, Germany), 8 weeks of age, were single housed in a temperature-controlled room with a 12-hour light/dark cycle (06:00-18:00 light). The mice had ad libitum access to water and rodent chow diet (R70, Lactamin, Kimstad, Sweden), and were acclimated for 5 days upon arrival. After acclimation, all mice received a single oral dose of test compound (3 or 10 mg/kg). Blood samples for whole blood compound exposure measurements were taken at 0.25, 0.5, 1, 2, 4, 8 and 24 h post oral dosing. Samples were collected in EDTA capillary tubes (20 μL, K2E, REF 19.447) and were transferred to a 96-deep well plate (NUNC, Thermo Discher Scientific) and stored at −20° C. until further analyses were performed. Blood samples for plasma FAP enzyme activity measurements were taken at 0, 0.25, 0.5, 1, 2, 8 and 24 h post dosing. 25 μL of whole blood was collected in EDTA Microvette® CB 300 (K2E, REF 16.444.100) tubes, and were centrifuged at 4,000×g for 5 min. 10 μL of plasma was then transferred to PCR tubes and stored at −20° C. until further analysis was performed. All blood samples were taken by vena saphena puncture.

Recombinant human FAP (PB-17-1837, construct PL-17-0278, cd33-FAP (27-757)-6His, Mw85926 Da) was used as a standard for this assay. Protein was secreted from Sf21 cells (insect cells) in media, purified with affinity (batch mode, Ni excel resin) and size exclusion chromatography (Superdex200), concentrated and aliquoted to be frozen in liquid N₂ for storage at −80° C. Recombinant FAP was diluted in protein buffer (25 mM Tris/HCl, pH 7.6, 150 mM NaCl, 5% glycerol, 1 mM EDTA, 0.25 mM TCEP) and 5 μL aliquots (0.1 mg/mL, 1.15 μM) were stored at −80° C. Standards were prepared using 2-fold dilution steps, 8 concentrations, 4 replicates (FAC: 1.2 nM, 0.6 nM, 0.3 nM). The plates were read on a Beckman Paradigm reader with excitation 360 nm and emission 465 nm. Fluorescence measurements were performed with kinetic read every 5 minutes for 60 min at rt. Data were analyzed in Excel (IDBS XLfit Add-In) using a Linear Regression (y=k*x+m) model to prepare a human recombinant FAP standard curve.

On the day of the assay, plasma was diluted (1:2) to 20 μL volume in buffer (PBS, 0.1% BSA) and 7.5 μL was transferred to the assay plate (384-well black, fluotrack PS, Greiner 781076). Ala-Pro-AMC (stock solution in 10 mM DMSO) was diluted in buffer (PBS, 0.1% BSA) to 150 μM concentration (180 μL stock solution to 12 mL buffer) and 7.5 μL added to the assay plate followed by a pipetting mix. The plates were read on a Beckman Paradigm reader with excitation 360 nm and emission 465 nm. Fluorescence measurements were performed with kinetic read every 5 minutes for 60 minutes at rt. As noted above, FAP cleaves Ala-Pro-AMC to release free AMC which is measured as a fluorescent signal.

The in vivo potency IC50 of each test compound was then estimated by relating the plasma exposure C of the compound to target engagement E in plasma using the following equation:

$$E = {E}_{0} ( 1 - \frac{{I}_{max} C}{{IC}_{50} + C} )$$

where E₀ is the FAP baseline in plasma prior to dosing and Iₘₐₓ is the maximum effect of the compound. Data from each target engagement experiment were considered separately and therefore slightly different estimates of FAP baseline for each compound were obtained. Full inhibition was achieved for all tested compounds at the earlier timepoints and therefore the parameter Iₘₐₓ was fixed to 1 for all compounds. The parameter estimation was done in Phoenix WinNonlin Certara build 8.1.0.3530 with the algorithm ‘Naïve pooled’ as parameter estimation method. In vivo IC₅₀ estimates for the test compounds are reported in Table 9.

TABLE 9

| Example | In Vivo Mouse IC₅₀ (nM) |
|---|---|
| 3 | 7.6 |
| 20 | 8.8 |
| 78 | 2.6 |

Although specific embodiments and examples have been described above, these embodiments and examples are only illustrative and do not limit the scope of the disclosure. Changes and modifications can be made in accordance with ordinary skill in the art without departing from the disclosure in its broader aspects as defined in the following claims. For example, any embodiment described herein can be combined with any other suitable embodiment described herein to provide additional embodiments.

As will be understood by the skilled artisan, all numbers, including those expressing quantities of ingredients, properties such as molecular weight, reaction conditions, and so forth, are approximations and understood as being modified in all instances by the term “about.” These values can vary depending upon the desired properties sought to be obtained by those skilled in the art utilizing the present teachings of the present disclosure. It is also understood that such values inherently contain variability necessarily resulting from the standard deviations found in their respective testing measurements.

One skilled in the art will also readily recognize that where members are grouped together in a common manner, such as in a Markush group, the present disclosure encompasses not only the entire group listed as a whole, but each member of the group individually and all possible subgroups of the main group. Additionally, for all purposes, the present disclosure encompasses not only the main group, but also the main group absent one or more of the group members. The present disclosure also envisages the explicit exclusion or disclaimer of one or more of any of the group members in the claimed disclosure.

As will be understood by one skilled in the art, for any and all purposes, particularly in terms of providing a written description, all ranges disclosed herein also encompass any and all possible subranges and combinations of subranges thereof as well as the individual values making up the range, particularly integer values. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. For example, the range C₍₁₋₆₎, includes the subranges C₍₂₋₆₎, C₍₃₋₆₎, C₍₃₋₅₎, C₍₄₋₆₎, etc., as well as C₁ (methyl), C₂ (ethyl), C₃ (propyl), C₄ (butyl), C₅ (pentyl) and C₆ (hexyl) individually. As will also be understood by one skilled in the art, all language such as “up to,” “at least,” “greater than,” “less than,” “more than,” “or more” and the like include the number recited and refer to ranges which can be subsequently broken down into subranges as discussed above. In the same manner, all ratios disclosed herein also include all subratios falling within the broader ratio.

Reference to a “step” in this disclosure is used for convenience purposes only and does not categorize, define or limit the disclosure as set forth herein.

What is claimed is:

1. A compound having the structure of Formula (I): 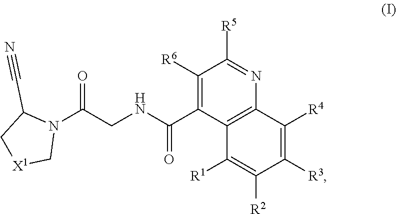
or a pharmaceutically acceptable salt thereof, wherein:
X¹ is selected from the group consisting of —S—, —S(O)—, and —S(O)₂—;
R¹ is selected from the group consisting of hydrogen, halogen, hydroxy, C₁₋₃-alkyl, and C₁₋₆-alkoxy;
R² is selected from the group consisting of:
(a) C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, cyano, C₁₋₆-alkyl, and halo-C₁₋₆-alkyl;
(b) C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(c) phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy; and
(d) a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(e) —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
(ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
(iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy;
R³ is selected from the group consisting of hydrogen, halogen, and C₁₋₃-alkyl;
R⁴ is selected from the group consisting of hydrogen, halogen, and C₁₋₃-alkyl;
R⁵ is selected from the group consisting of hydrogen, halogen, and C₁₋₃-alkyl; and
R⁶ is selected from the group consisting of hydrogen, halogen, and C₁₋₃-alkyl.

2. The compound of claim 1, or a pharmaceutically acceptable salt thereof, wherein the compound has the structure of Formula (II): 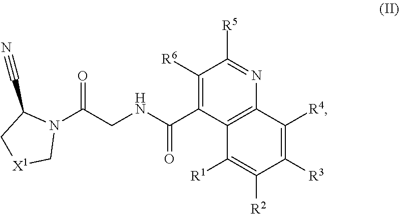
wherein X¹, R¹, R², R³, R⁴, R⁵, and R⁶ are as defined in claim 1.

3. The compound of claim 1 or 2, or a pharmaceutically acceptable salt thereof, wherein X¹ is —S—.

4. The compound of claim 1, or a pharmaceutically acceptable salt thereof, wherein the compound has the structure of Formula (IV-A): 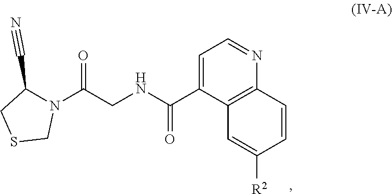
wherein R² is as defined in claim 1.

5. The compound of any of claims 1 to 4, or a pharmaceutically acceptable salt thereof, wherein R² is C₁₋₆-alkyl, wherein the C₁₋₆-alkyl is optionally substituted with one or more substituents independently selected from the group consisting of halogen, hydroxy, cyano, C₃₋₆-cycloalkyl, halo-C₃₋₆-cycloalkyl, C₁₋₆-alkoxy, halo-C₁₋₆-alkoxy, C₃₋₆-cycloalkoxy, halo-C₃₋₆-cycloalkoxy, C₁₋₆-alkoxy-C₁₋₆-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₆-alkyl, and halo-C₁₋₆-alkyl.

6. The compound of claim 1, or a pharmaceutically acceptable salt thereof, wherein the compound is selected from the group consisting of:
N-(2-((R)-4-Cyanothiazolidin-3-yl)-2-oxoethyl)-6-((RS)-1-(3-fluoropyridin-2-yl)-ethyl)quinoline-4-carboxamide;
(R)—N-(2-(4-Cyanothiazolidin-3-yl)-2-oxoethyl)-6-(2-hydroxypropan-2-yl)quinoline-4-carboxamide;
(R)—N-(2-(4-Cyanothiazolidin-3-yl)-2-oxoethyl)-6-(trifluoromethyl)-quinoline-4-carboxamide;
(R)—N-(2-(4-Cyanothiazolidin-3-yl)-2-oxoethyl)-6-(2-methoxypropan-2-yl)quinoline-4-carboxamide;
(R)—N-(2-(4-Cyanothiazolidin-3-yl)-2-oxoethyl)-6-((2,5-dimethyl-2H-1,2,3-triazol-4-yl)-methyl)quinoline-4-carboxamide;
(R)—N-(2-(4-Cyanothiazolidin-3-yl)-2-oxoethyl)-6-(morpholino-methyl)quinoline-4-carboxamide;
N-(2-((R)-4-Cyanothiazolidin-3-yl)-2-oxoethyl)-6-((RS)-1-hydroxyethyl)quinoline-4-carboxamide;
N-(2-((R)-4-Cyanothiazolidin-3-yl)-2-oxoethyl)-6-((RS)-1-methoxyethyl)quinoline-4-carboxamide;
(R)—N-(2-(4-cyanothiazolidin-3-yl)-2-oxoethyl)-6-(ethoxymethyl)-quinoline-4-carboxamide;
(R)—N-(2-(4-cyanothiazolidin-3-yl)-2-oxoethyl)-6-methylquinoline-4-carboxamide;
(R)—N-(2-(4-cyanothiazolidin-3-yl)-2-oxoethyl)-6-(fluoromethyl)-quinoline-4-carboxamide;
N-(2-((R)-4-Cyanothiazolidin-3-yl)-2-oxoethyl)-6-((RS)-1,2-dimethoxyethyl)quinoline-4-carboxamide;
(R)—N-(2-(4-Cyanothiazolidin-3-yl)-2-oxoethyl)-6-((2-methoxyethoxy)-methyl)quinoline-4-carboxamide;
N-(2-((R)-4-Cyanothiazolidin-3-yl)-2-oxoethyl)-6-((((RS)-tetrahydrofuran-3-yl)oxy)-methyl)quinoline-4-carboxamide;
(R)—N-(2-(4-Cyanothiazolidin-3-yl)-2-oxoethyl)-6-((2,2-difluoroethoxy)methyl)quinoline-4-carboxamide;
(R)—N-(2-(4-Cyanothiazolidin-3-yl)-2-oxoethyl)-6-(cyclopropoxy-methyl)quinoline-4-carboxamide;
(R)—N-(2-(4-Cyanothiazolidin-3-yl)-2-oxoethyl)-6-((4-methylisoxazol-3-yl)methyl)-quinoline-4-carboxamide;
(R)—N-(2-(4-Cyanothiazolidin-3-yl)-2-oxoethyl)-6-((4,6-dimethylpyridin-3-yl)-methyl)quinoline-4-carboxamide;
(R)—N-(2-(4-Cyanothiazolidin-3-yl)-2-oxoethyl)-6-((1,3-dimethyl-1H-pyrazol-5-yl)-methyl)quinoline-4-carboxamide;
(R)—N-(2-(4-Cyanothiazolidin-3-yl)-2-oxoethyl)-6-((2-methyl-2H-1,2,3-triazol-4-yl)-methyl)quinoline-4-carboxamide;
(R)—N-(2-(4-Cyanothiazolidin-3-yl)-2-oxoethyl)-6-((5-methylisoxazol-3-yl)-methyl)quinoline-4-carboxamide;
N-(2-((R)-4-Cyanothiazolidin-3-yl)-2-oxoethyl)-6-((RS)-1-(5-methylisoxazol-3-yl)-ethyl)quinoline-4-carboxamide;
6-((RS)-1-(5-Chloropyridin-2-yl)ethyl)-N-(2-((R)-4-cyanothiazolidin-3-yl)-2-oxoethyl)quinoline-4-carboxamide;
N-(2-((R)-4-Cyanothiazolidin-3-yl)-2-oxoethyl)-6-((RS)-1-(3-methylisoxazol-5-yl)-ethyl)quinoline-4-carboxamide;
N-(2-((R)-4-Cyanothiazolidin-3-yl)-2-oxoethyl)-6-((RS)-1-(6-methylpyridin-3-yl)-ethyl)quinoline-4-carboxamide;
N-(2-((R)-4-Cyanothiazolidin-3-yl)-2-oxoethyl)-6-((RS)-1-(2-(trifluoromethyl)pyridin-4-yl)ethyl)quinoline-4-carboxamide;
(R)—N-(2-(4-Cyanothiazolidin-3-yl)-2-oxoethyl)-6-(2,2-difluoropropyl)quinoline-4-carboxamide;
(R)—N-(2-(4-Cyanothiazolidin-3-yl)-2-oxoethyl)-6-((tetrahydro-2H-pyran-4-yl)-methyl)quinoline-4-carboxamide;
(R)—N-(2-(4-Cyanothiazolidin-3-yl)-2-oxoethyl)-6-(3,3,3-trifluoropropyl)quinoline-4-carboxamide;
N-(2-((R)-4-Cyanothiazolidin-3-yl)-2-oxoethyl)-6-(((RS)-tetrahydrofuran-3-yl)-methyl)quinoline-4-carboxamide;
(R)—N-(2-(4-Cyanothiazolidin-3-yl)-2-oxoethyl)-6-(difluoromethyl)-quinoline-4-carboxamide;
(R)-6-((1H-Pyrazol-1-yl)methyl)-N-(2-(4-cyanothiazolidin-3-yl)-2-oxoethyl)quinoline-4-carboxamide;
(R)—N-(2-(4-cyanothiazolidin-3-yl)-2-oxoethyl)-6-(3-methoxypropyl)quinoline-4-carboxamide; and
(R)—N-(2-(4-cyanothiazolidin-3-yl)-2-oxoethyl)-6-(2-methoxyethyl)quinoline-4-carboxamide.

7. The compound of any of claims 1 to 4 or a pharmaceutically acceptable salt thereof, wherein R² is C₃₋₆-cycloalkyl, wherein the C₃₋₆-cycloalkyl is optionally substituted with one or more substituents independently selected from the group consisting of halogen, hydroxy, cyano, C₁₋₆-alkyl, halo-C₁₋₆-alkyl, C₁₋₆-alkoxy, halo-C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl.

8. The compound of claim 1, or a pharmaceutically acceptable salt thereof, wherein the compound is selected from the group consisting of:
(R)—N-(2-(4-Cyanothiazolidin-3-yl)-2-oxoethyl)-6-(1-(2-(trifluoromethyl)pyridin-4-yl)-cyclopropyl)quinoline-4-carboxamide;
(R)—N-(2-(4-Cyanothiazolidin-3-yl)-2-oxoethyl)-6-(1-(6-methylpyridin-3-yl)-cyclopropyl)quinoline-4-carboxamide;
(R)—N-(2-(4-Cyanothiazolidin-3-yl)-2-oxoethyl)-6-(1-(3-fluoropyridin-2-yl)-cyclopropyl)quinoline-4-carboxamide;
(R)—N-(2-(4-Cyanothiazolidin-3-yl)-2-oxoethyl)-6-(1-(5-methylisoxazol-3-yl)-cyclopropyl)quinoline-4-carboxamide;
(R)-6-(1-(5-Chloropyridin-2-yl)cyclopropyl)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(1-(3-methylisoxazol-5-yl)-cyclopropyl)quinoline-4-carboxamide;
6-((1r,4R*)-4-Cyanocyclohexyl)-N-(2-((R)-4-cyanothiazolidin-3-yl)-2-oxoethyl)-quinoline-4-carboxamide;
6-((1r,4R*)-4-Cyanocyclohexyl)-N-(2-((R)-4-cyanothiazolidin-3-yl)-2-oxoethyl)-quinoline-4-carboxamide;
(R)-6-(1-Cyanocyclohexyl)-N-(2-(4-cyanothiazolidin-3-yl)-2-oxoethyl)quinoline-4-carboxamide;
N-(2-((R)-4-Cyanothiazolidin-3-yl)-2-oxoethyl)-6-((1r,3R*)-1-fluoro-3-methoxy-cyclobutyl)quinoline-4-carboxamide Isomer 1;
N-(2-((R)-4-Cyanothiazolidin-3-yl)-2-oxoethyl)-6-((1r,3R*)-1-fluoro-3-methoxy-cyclobutyl)quinoline-4-carboxamide Isomer 2;
(R)-6-(1-Cyanocyclopropyl)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(1-fluoro-cyclobutyl)quinoline-4-carboxamide;
(R)—N-(2-(4-Cyanothiazolidin-3-yl)-2-oxoethyl)-6-(1-ethoxycyclopropyl)quinoline-4-carboxamide;
(R)—N-(2-(4-Cyanothiazolidin-3-yl)-2-oxoethyl)-6-cyclopropylquinoline-4-carboxamide;
N-(2-((R)-4-Cyanothiazolidin-3-yl)-2-oxoethyl)-6-((1r,4R*)-4-methoxycyclohexyl)-quinoline-4-carboxamide Isomer 1; and
N-(2-((R)-4-Cyanothiazolidin-3-yl)-2-oxoethyl)-6-((1r,4R*)-4-methoxycyclohexyl)-quinoline-4-carboxamide Isomer 2.

9. The compound of any of claims 1 to 4, or a pharmaceutically acceptable salt thereof, wherein R² is phenyl, wherein the phenyl is optionally substituted with one or more substituents independently selected from the group consisting of halogen, cyano, C₁₋₆-alkyl, halo-C₁₋₆-alkyl, C₁₋₆-alkoxy, halo-C₁₋₆-alkoxy, and C₁₋₆-alkoxy-C₁₋₆-alkoxy.

10. The compound of any of claims 1 to 4, or a pharmaceutically acceptable salt thereof, wherein R² is a 4-, 5-, 6-, or 7-membered ring heterocyclyl, wherein the heterocyclyl ring: (i) is a saturated, partially saturated, or completely unsaturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy.

11. The compound of any of claims 1 to 4, or a pharmaceutically acceptable salt thereof, wherein R² is selected from the group consisting of tetrahydrofuranyl, furanyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane.

12. The compound of claim 1, or a pharmaceutically acceptable salt thereof, wherein the compound is selected from the group consisting of:
(R)—N-(2-(4-Cyanothiazolidin-3-yl)-2-oxoethyl)-6-(furan-3-yl)quinoline-4-carboxamide;
(R)—N-(2-(4-Cyanothiazolidin-3-yl)-2-oxoethyl)-6-(4-hydroxy-tetrahydro-2H-pyran-4-yl)-quinoline-4-carboxamide;
(R)—N-(2-(4-Cyanothiazolidin-3-yl)-2-oxoethyl)-6-(tetrahydro-2H-pyran-4-yl)quinoline-4-carboxamide;
(R)—N-(2-(4-Cyanothiazolidin-3-yl)-2-oxoethyl)-6-(4-methyl-tetrahydro-2H-pyran-4-yl)-quinoline-4-carboxamide;
(R)—N-(2-(4-Cyanothiazolidin-3-yl)-2-oxoethyl)-6-(3,6-dihydro-2H-pyran-4-yl)quinoline-4-carboxamide;
(i) the C₁₋₆-alkyl is optionally substituted with one or more substituents independently selected from the group consisting of halogen, cyano, C₃₋₆-cycloalkyl, halo-C₃₋₆-cycloalkyl, C₁₋₆-alkoxy, and halo-C₁₋₆-alkoxy;
(ii) the phenyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy; and
(iii) the tetrahydropyranyl is optionally substituted with one or more substituents independently selected from the group consisting of halogen, C₁₋₆-alkyl, halo-C₁₋₆-alkyl, C₁₋₆-alkoxy, and halo-C₁₋₆-alkoxy.

14. The compound of any of claims 1 to 4, or a pharmaceutically acceptable salt thereof, wherein R² is —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

15. The compound of claim 1, or a pharmaceutically acceptable salt thereof, wherein the compound is selected from the group consisting of:
(R)—N-(2-(4-Cyanothiazolidin-3-yl)-2-oxoethyl)-6-phenoxyquinoline-4-carboxamide;
(R)—N-(2-(4-Cyanothiazolidin-3-yl)-2-oxoethyl)-6-(2,2-difluoroethoxy)quinoline-4-carboxamide;
(R)—N-(2-(4-Cyanothiazolidin-3-yl)-2-oxoethyl)-6-(2-fluoro-2-methylpropoxy)quinoline-4-carboxamide;
(R)—N-(2-(4-Cyanothiazolidin-3-yl)-2-oxoethyl)-6-(2-methoxyethoxy)quinoline-4-carboxamide;
(R)—N-(2-(4-Cyanothiazolidin-3-yl)-2-oxoethyl)-6-(cyclopropyl-methoxy)quinoline-4-carboxamide;
(R)—N-(2-(4-Cyanothiazolidin-3-yl)-2-oxoethyl)-6-((tetrahydro-2H-pyran-4-yl)oxy)-quinoline-4-carboxamide;
(R)—N-(2-(4-Cyanothiazolidin-3-yl)-2-oxoethyl)-6-((1,3-difluoropropan-2-yl)oxy)-quinoline-4-carboxamide;
(R)—N-(2-(4-Cyanothiazolidin-3-yl)-2-oxoethyl)-6-((3,3-difluorocyclobutyl)-methoxy)quinoline-4-carboxamide;
N-(2-((R)-4-Cyanothiazolidin-3-yl)-2-oxoethyl)-6-(((RS)-2,2-dimethyltetrahydro-2H-pyran-4-yl)oxy)quinoline-4-carboxamide;
(R)-6-(Cyanomethoxy)-N-(2-(4-cyanothiazolidin-3-yl)-2-oxoethyl)quinoline-4-carboxamide;
(R)—N-(2-(4-Cyanothiazolidin-3-yl)-2-oxoethyl)-6-(2,2-difluoropropoxy)quinoline-4-carboxamide;
(R)—N-(2-(4-Cyanothiazolidin-3-yl)-2-oxoethyl)-6-propoxyquinoline-4-carboxamide; and
(R)—N-(2-(4-Cyanothiazolidin-3-yl)-2-oxoethyl)-6-(2-fluoroethoxy)-quinoline-4-carboxamide.

16. The compound of any of claims 1 to 4, or a pharmaceutically acceptable salt thereof, wherein R² is selected from the group consisting of:
(a) C₁₋₃-alkyl, wherein the C₁₋₃-alkyl is optionally substituted with one or more substituents independently selected from the group consisting of halogen, hydroxy, cyano, C₃₋₆-cycloalkyl, halo-C₃₋₆-cycloalkyl, C₁₋₃-alkoxy, halo-C₁₋₃-alkoxy, C₃₋₆-cycloalkoxy, halo-C₃₋₆-cycloalkoxy, C₁₋₃-alkoxy-C₁₋₃-alkoxy, oxetanyl, tetrahydrofuranyl, tetrahydrofuranyloxy, tetrahydropyranyl, morpholinyl, pyrazolyl, isoxazolyl, triazolyl, and pyridinyl; and wherein the oxetanyl, tetrahydrofuranyl, tetrahydrofuranyloxy, tetrahydropyranyl, morpholinyl, pyrazolyl, isoxazolyl, triazolyl, and pyridinyl are optionally substituted with one or more substituents independently selected from the group consisting of halogen, C₁₋₃-alkyl, and halo-C₁₋₃-alkyl;
(b) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(c) phenyl, wherein the phenyl is optionally substituted with one or more substituents independently selected from the group consisting of halogen, C₁₋₃-alkyl, halo-C₁₋₃-alkyl, C₁₋₃-alkoxy, halo-C₁₋₃-alkoxy, and C₁₋₃-alkoxy-C₁₋₃-alkoxy;
(d) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(e) —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
(ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
(iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

17. The compound of any of claims 1 to 4, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkyl, halo-C₁₋₃-alkyl, C₁₋₃-alkoxy, halo-C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl; and
(b) a 4-, 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, halo-C₁₋₃-alkyl, C₂₋₃-alkynyl, C₁₋₃-alkoxy, and halo-C₁₋₃-alkoxy; and
(c) —OR⁷, wherein R⁷ is selected from the group consisting of C₁₋₄-alkyl, phenyl, and tetrahydropyranyl; wherein:
(R)—N-(2-(4-Cyanothiazolidin-3-yl)-2-oxoethyl)-6-(4-ethoxytetrahydro-2H-pyran-4-yl)-quinoline-4-carboxamide;
N-(2-((R)-4-Cyanothiazolidin-3-yl)-2-oxoethyl)-6-((RS)-tetrahydro-2H-pyran-2-yl)-quinoline-4-carboxamide;
N-(2-((R)-4-Cyanothiazolidin-3-yl)-2-oxoethyl)-6-((RS)-1,4-dioxan-2-yl)quinoline-4-carboxamide;
N-(2-((R)-4-Cyanothiazolidin-3-yl)-2-oxoethyl)-6-((RS)-tetrahydrofuran-2-yl)quinoline-4-carboxamide;
(R)—N-(2-(4-Cyanothiazolidin-3-yl)-2-oxoethyl)-6-(4-ethynyl-tetrahydro-2H-pyran-4-yl)-quinoline-4-carboxamide;
(R)-6-(4-Cyanotetrahydro-2H-pyran-4-yl)-N-(2-(4-cyanothiazolidin-3-yl)-2-oxoethyl)-quinoline-4-carboxamide;
N-(2-((R)-4-Cyanothiazolidin-3-yl)-2-oxoethyl)-6-(5,5-dimethyltetrahydrofuran-3-yl)-quinoline-4-carboxamide;
(R)—N-(2-(4-Cyanothiazolidin-3-yl)-2-oxoethyl)-6-(4-fluorotetrahydro-2H-pyran-4-yl)-quinoline-4-carboxamide;
(R)—N-(2-(4-Cyanothiazolidin-3-yl)-2-oxoethyl)-6-(4-methoxy-tetrahydro-2H-pyran-4-yl)quinoline-4-carboxamide;
N-(2-((R)-4-Cyanothiazolidin-3-yl)-2-oxoethyl)-6-((RS)-tetrahydrofuran-3-yl)quinoline-4-carboxamide;
N-(2-((R)-4-Cyanothiazolidin-3-yl)-2-oxoethyl)-6-((RS)-2,2-dimethyltetrahydro-2H-pyran-4-yl)quinoline-4-carboxamide; and
6-((1RS,6SR)-3-Oxabicyclo[4.1.0]heptan-6-yl)-N-(2-((R)-4-cyanothiazolidin-3-yl)-2-oxoethyl)quinoline-4-carboxamide.

13. The compound of any of claims 1 to 4, or a pharmaceutically acceptable salt thereof, wherein R² is —OR⁷, wherein R⁷ is selected from the group consisting of C₁₋₆-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, C₁₋₃-alkoxy, and halo-C₁₋₃-alkoxy;
(ii) the phenyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy; and
(iii) the tetrahydropyranyl is optionally substituted with one or more substituents independently selected from the group consisting of halogen, C₁₋₃-alkyl, halo-C₁₋₃-alkyl, C₁₋₃-alkoxy, and halo-C₁₋₃-alkoxy.

18. The compound of any of claims 1 to 4, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₆-alkoxy, isoxazolyl, and pyridinyl; wherein the isoxazolyl and pyridinyl are optionally substituted with one or more substituents independently selected from the group consisting of halogen, C₁₋₆-alkyl, and halo-C₁₋₆-alkyl;
(b) a 5-, 6-, or 7-membered ring heterocyclyl, wherein the heterocyclyl ring: (i) is a saturated or partially saturated monocyclic or fused bicyclic ring, (ii) has one or two oxygen ring atoms with the remaining ring atoms being carbon, and (iii) is optionally substituted with one or more substituents independently selected from the group consisting of halogen, hydroxy, cyano, C₁₋₃-alkyl, C₂₋₃-alkynyl, and C₁₋₃-alkoxy; and
(c) —OR⁷, wherein R⁷ is selected from the group consisting of C₁₋₆-alkyl, phenyl, and tetrahydropyranyl; wherein:
(i) the C₁₋₆-alkyl is optionally substituted with one or more substituents independently selected from the group consisting of halogen, cyano, C₃₋₆-cycloalkyl, halo-C₃₋₆-cycloalkyl, and C₁₋₆-alkoxy; and
(ii) the tetrahydropyranyl is optionally substituted with one or C₁₋₆-alkyl.

19. The compound of any of claims 1 to 4, or a pharmaceutically acceptable salt thereof, wherein R² is selected from the group consisting of:
(a) C₃₋₆-cycloalkyl, wherein the C₃₋₆-cycloalkyl is optionally substituted with one or more substituents independently selected from the group consisting of halogen, cyano, C₁₋₃-alkoxy, isoxazolyl, and pyridinyl; wherein the isoxazolyl and pyridinyl are optionally substituted with one or more substituents independently selected from the group consisting of halogen, C₁₋₃-alkyl, and halo-C₁₋₃-alkyl;
(b) a 5-, 6-, or 7-membered ring heterocyclyl selected from the group consisting of tetrahydrofuranyl, dihydropyranyl, tetrahydropyranyl, 1,4-dioxanyl, and 3-oxabicyclo[4.1.0]heptane, wherein the tetrahydrofuranyl, dihydropyranyl, tetrahydropyranyl, 1,4-dioxanyl, and 3-oxabicyclo[4.1.0]heptane are optionally substituted with one or more substituents independently selected from the group consisting of halogen, hydroxy, cyano, C₁₋₆-alkyl, halo-C₁₋₆-alkyl, C₂₋₆-alkynyl, C₁₋₆-alkoxy, and halo-C₁₋₆-alkoxy; and
(c) —OR⁷, wherein R⁷ is selected from the group consisting of C₁₋₄-alkyl, phenyl, and tetrahydropyranyl; wherein:
(i) the C₁₋₄-alkyl is optionally substituted with one or more substituents independently selected from the group consisting of halogen, cyano, C₃₋₆-cycloalkyl, halo-C₃₋₆-cycloalkyl, and C₁₋₃-alkoxy; and
(ii) the tetrahydropyranyl is optionally substituted with one or C₁₋₃-alkyl.

20. The compound of any of claims 1 to 4, or a pharmaceutically acceptable salt thereof, wherein R² is selected from the group consisting of:
(a) optionally substituted C₃₋₆-cycloalkyl selected from the group consisting of cyclopropyl, cyanocyclopropyl, ethoxycyclopropyl, methylisoxazolylcyclopropyl, methylpyridinylcyclopropyl, chloropyridinylcyclopropyl, fluoropyridinylcyclopropyl, trifluoropyridinylcyclopropyl, fluorocyclobutyl, (fluoro)(methoxy)cyclobutyl, methoxycyclohexyl, and cyanocyclohexyl;
(b) optionally substituted 5-, 6-, or 7-membered ring heterocyclyl selected from the group consisting of tetrahydrofuranyl, dimethyltetrahydrofuranyl, dihydropyranyl, tetrahydropyranyl, hydroxytetrahydropyanyl, fluorotetrahydropyanyl, cyanotetrahydropyranyl, methyltetrahydropyanyl, dimethyltetrahydropyanyl, methoxytetrahydropyanyl, ethoxytetrahydropyanyl, 1,4-dioxanyl, and 3-oxabicyclo[4.1.0]heptane; and
(c) —OR⁷, wherein R⁷ is selected from the group consisting of cyanomethyl, fluoroethyl, difluoroethyl, propyl, difluoropropyl, fluorobutyl, methoxyethyl, cyclopropylmethyl, difluorocyclobutylmethyl, phenyl, tetrahydropyranyl, and dimethyltetrahydropyranyl.

21. A pharmaceutical composition comprising a compound of any of claims 1 to 20, or a pharmaceutically acceptable salt thereof, and one or more pharmaceutically acceptable excipients.

22. The use of a compound of any of claims 1 to 21, or a pharmaceutically acceptable salt thereof, for the manufacture of a medicament for treating or preventing an FAP-mediated condition.
